(19) United States
(12) Patent Application Publication
(10) Pub. No.: US 2026/0317781 A1
(43) Pub. Date: Oct. 15, 2026

(54) RIBOSWITCHES FOR REGULATING GENE EXPRESSION AND THERAPEUTIC METHODS OF USING THE SAME

(71) Applicant: KRIYA THERAPEUTICS, INC., Morrisville, NC (US)

(72) Inventors: Nicholas DIETRICH, Morrisville, NC (US); Joseph RABINOWITZ, Morrisville, NC (US); Jennifer ISRAEL, Morrisville, NC (US)

(21) Appl. No.: 19/479,115

(22) PCT Filed: Apr. 26, 2024
(86) PCT No.: PCT/US2024/026643

§ 371 Date: Oct. 27, 2025

Related U.S. Application Data
(60) Provisional application No. 63/499,181, filed on Apr. 28, 2023.

(51) Int. Cl. C12N 15/86; A61K 48/00

(52) U.S. Cl. CPC C12N 15/86; A61K 48/0066; C12N 2750/14143; C12N 2830/42; C12N 2830/50

(57) ABSTRACT

The present disclosure provides an expression cassette comprising from 5′ to 3′: (i) promoter, (ii) an upstream exon, (iii) an upstream intron, (iv) an alternatively spliced exon comprising a translation initiation sequence, (v) a downstream intron, (vi) a downstream exon, and (vii) a heterologous nucleic acid sequence. In some aspects, the upstream exon, the upstream intron, the alternatively spliced exon, the downstream intron, and the downstream exon, each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

### CROSS REFERENCE TO RELATED APPLICATIONS

The present application claims the priority benefit of U.S. Provisional Application No. 63/499,181, filed Apr. 28, 2023, the contents of which are hereby incorporated by reference in their entirety.

### REFERENCE TO SEQUENCE LISTING SUBMITTED ELECTRONICALLY

The content of the electronically submitted sequence listing (Name: 4525_105PC01_Seqlisting_ST26.xml; Size: 178,656 bytes; and Date of Creation: Apr. 25, 2024), filed with the application, is incorporated herein by reference in its entirety.

### FIELD OF DISCLOSURE

The present disclosure pertains generally to the field of molecular biology and medicine (e.g., gene therapy). More particularly it relates to compositions and methods for regulating gene expression.

### BACKGROUND

Gene therapy represents a robust tool for treating human disease. While gene therapy technologies have developed in recent decades, there is a need to further control gene expression, for example, to regulate symptoms and reduce protein toxicity.

A riboswitch is a regulatory segment of a messenger RNA (mRNA) molecule that binds a small molecule, which can result in a change in production of the proteins encoded by the mRNA. Most known riboswitches occur in bacteria, but functional riboswitches (e.g., the TPP riboswitch) have been discovered in archaea, plants and certain fungi. Riboswitches can operate using different mechanisms. For example, certain riboswitch structures can affect the splicing of pre-mRNA. Bacteria make extensive use of riboswitches to sense metabolites and control gene expression, and typically do so by modulating premature transcription termination or translation initiation. The most widespread riboswitch class known in bacteria responds to the coenzyme thiamine pyrophosphate (TPP), which is a derivative of vitamin B1. See Cheah et al., Nature volume 447, 497-500 (2007). Methods for controlling gene expression using drug inducible alternative splicing switches have been investigated (see, e.g., Int. Publ. No. WO2021163556A1). To use a riboswitch in a therapeutic setting, the small molecule must be both effective at regulating the gene of interest and non-toxic to the patient.

### BRIEF SUMMARY

Certain aspects of the disclosure are directed to an expression cassette comprising from 5′ to 3′: (i) promoter, (ii) an upstream exon, (iii) an upstream intron, (iv) an alternatively spliced exon comprising a translation initiation sequence, (v) a downstream intron, (vi) a downstream exon, and (vii) a heterologous nucleic acid sequence.

In some aspects, the upstream intron, the alternatively spliced exon, and the downstream intron each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the upstream exon and the downstream exon each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the upstream exon, the upstream intron, the alternatively spliced exon comprising a translation initiation sequence, the downstream intron, the a downstream exon each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the upstream exon and the downstream exon each comprise a portion of a gene, which is different from the gene for the portion of the upstream intron, the alternatively spliced exon, and the downstream intron.

In some aspects, the upstream exon and the downstream exon comprise synthetic exons.

In some aspects, the inclusion of the alternatively spliced exon is regulatable (e.g., regulated) by a splicing modulator.

In some aspects, the splicing modulator is kinetin or homocarbonyltopsentin.

In some aspects, the gene is TRPV3, STK31, or USP25; and the splicing modulator is Kinetin.

In some aspects, the gene is GTF2B, LYPD3, IK, or TEX14; and the splicing modulator is Homocarbonyltopsentin.

In some aspects, the splicing modulator promotes inclusion of the alternatively spliced exon thereby driving expression of the heterologous nucleic acid sequence.

In some aspects, the promoter is selected from the group consisting of a CBA promoter, a smCBA promoter, a CMV promoter, an EF-1a (Elongation Factor 1a) promoter, a RSV (Rous Sarcoma Virus) promoter, an Ubiquitin (UbC) promoter, a CAG promoter, a SV40 (simian vacuolating virus 40) promoter, a PGK (phosphoglycerate kinase) promoter, a human beta actin promoter, a H1 (human polymerase III RNA) promoter, a human U6 small nuclear promoter, tetracycline responsive element, and any combination thereof.

In some aspects, the expression cassette further comprises an enhancer.

In some aspects, the enhancer is selected from the group consisting of a CMV enhancer, a SV40 enhancer, and any combination thereof.

In some aspects, the expression cassette further comprises a cleavage site between the downstream exon and the heterologous nucleic acid sequence.

In some aspects, the cleavage site is selected from the group consisting of a furin cleavage site, a foot-and-mouth disease virus 18 2A peptide (F2A), an equine rhinitis A virus 2A peptide (E2A), a porcine teschovirus-1 2A peptide (P2A), a Thosea asigna virus 2A peptide (T2A), and any combination thereof.

In some aspects, the expression cassette further comprises a leader sequence between the downstream exon and the heterologous nucleic acid sequence.

In some aspects, the leader sequence is selected from the group consisting of an opticin (e.g., a human opticin (hOPT)) leader sequence, an interleukin-2 (e.g., human interleukin-2 (hIL-2)) leader sequence, an interleukin-12 (e.g., human interleukin-12 (hIL-12)) leader sequence, an interleukin-6 (e.g., a human or mouse interleukin-6 (hIL-6 or mIL-6)) leader sequence, an insulin (e.g., human insulin) leader sequence, a serum albumin (e.g., human serum albumin (HSA)) leader sequence, and any combination thereof.

In some aspects, the combined length of the upstream exon, the upstream intron, the alternatively spliced exon, the downstream intron, and the downstream exon is between about 300 basepairs to about 2100 basepairs.

In some aspects, the length of the heterologous nucleic acid sequence is less than about 4100 basepairs, less than about 4000 basepairs, or less than about 3900 basepairs.

In some aspects, the heterologous nucleic acid sequence encodes a protein. In some aspects, the protein is a therapeutic protein.

In some aspects, the upstream exon comprises a sequence selected from the group consisting of SEQ ID NO: 8, 13, 18, 23, 28, 33, and 38.

In some aspects, the alternatively spliced exon comprises a sequence selected from the group consisting of SEQ ID NO: 10, 15, 20, 25, 30, 35, and 40.

In some aspects, the downstream exon comprises a sequence selected from the group consisting of SEQ ID NO: 12, 17, 22, 27, 32, 37, and 42.

In some aspects, the expression cassette comprises a sequence selected from the group consisting of SEQ ID NO: 8-42.

In some aspects, the expression cassette comprises a sequence selected from the group consisting of one or more of SEQ ID NO: 1-7 and 43-66.

In some aspects, the expression cassette further comprises an untranslated region (UTR), a microRNA binding site, a polyA (pA) sequence, an intron sequence, or any combination thereof.

In some aspects, the expression cassette comprises a poly (A) (pA) sequence.

In some aspects, the pA sequence is a synthetic pA sequence, a bovine growth hormone (bGH) pA sequence, or a human growth hormone (hGH) pA sequence.

In some aspects, provided herein is a delivery vector comprising any of the expression cassettes disclosed herein.

In some aspects, the expression cassette is flanked by inverted terminal repeat (ITR) sequences.

In some aspects, the delivery vector is selected from the group consisting of a viral vector, a plasmid, a lipid, a protein particle, a bacterial vector, a lysosome, a virus-like particle, a polymeric particle, an exosome, and a vault particle.

In some aspects, the delivery vector is a viral vector.

In some aspects, the viral vector is a lentiviral vector, an adenoviral vector, an adeno-associated viral (AAV) vector, or a retroviral vector.

In some aspects, the viral vector is an adeno-associated viral (AAV) vector.

In some aspects, the delivery vector is a recombinant AAV (rAAV) vector comprising an AAV serotype selected from the group consisting of AAV1, AAV2, AAV3, AAV4, AAV5, AAV6, AAV7, AAV8, AAVrh8, AAV9, AAV10, AAVrh10, AAV11, and AAV12.

In some aspects, provided herein is a cell comprising any of the expression cassettes disclosed herein or any of the delivery vectors disclosed herein.

In some aspects, provided herein is a pharmaceutical composition comprising any of the expression cassettes disclosed herein, any of the delivery vectors disclosed herein, or any of the cells disclosed herein.

In some aspects, the pharmaceutical composition further comprises a pharmaceutically acceptable excipient.

Certain aspects of the disclosure are directed to a kit comprising i) any of the expression cassettes disclosed herein, any of the delivery vectors disclosed herein, any of the cells disclosed herein, or any of the pharmaceutical compositions disclosed herein; and (ii) optionally instructions for use.

In some aspects, the kit further comprises (iii) a splicing modulator molecule that promotes the inclusion of the alternatively spliced exon.

In some aspects, the splicing modulator molecule is kinetin.

In some aspects, the splicing modulator molecule is homocarbonyltopsentin.

Certain aspects of the disclosure are directed to a method of regulating expression of a protein in a mammalian cell comprising administering to the cell any of the expression cassettes disclosed herein, any of the delivery vectors disclosed herein, or any of the pharmaceutical compositions disclosed herein, followed by administering the splicing modulator molecule that promotes the inclusion of the alternatively spliced exon.

Certain aspects of the disclosure are directed to a method of expressing a protein in a cell or subject comprising administering to the cell or subject any of the expression cassettes disclosed herein, any of the delivery vectors disclosed herein, any of the cells disclosed herein, or any of the pharmaceutical compositions disclosed herein, followed by administering the splicing modulator molecule that promotes the inclusion of the alternatively spliced exon.

Certain aspects of the disclosure are directed to a method of treating a disease in a mammal comprising administering to a subject any of the expression cassettes disclosed herein, any of the delivery vectors disclosed herein, any of the cells disclosed herein, or any of the pharmaceutical compositions disclosed herein, followed by administering the splicing modulator molecule that promotes the inclusion of the alternatively spliced exon.

In some aspects, the administration of the expression cassette, delivery vector, or cell is systemic or tissue specific. In some aspects, the administration is intravenous, intramuscular, intrahepatic, intraventricular, intraparenchymal, intrathecal, subcutaneous, oral, intraocular, periocular, or intratumoral.

In some aspects, the administration of the molecule that promotes the inclusion of the alternatively spliced exon is systemic.

In some aspects, the molecule that promotes the inclusion of the alternatively spliced exon is administered at least 1 day after administration of the expression cassette.

In some aspects, the method further comprises administering a second dose of the molecule that promotes the inclusion of the alternatively spliced exon.

Certain aspects of the disclosure are directed to a method of regulating transgene expression post-transcriptionally, comprising administering to a subject (a) a nucleic acid construct comprising (i) an upstream exon, (ii) an upstream intron, (iii) an alternatively spliced exon comprising a translation initiation sequence, (iv) a downstream intron, (v) a downstream exon, and (vi) a heterologous nucleic acid sequence; and (b) a splicing modulator.

In some aspects, the upstream intron, the alternatively spliced exon, and the downstream intron each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the upstream exon and the downstream exon each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the upstream exon, the upstream intron, the alternatively spliced exon comprising a translation initiation sequence, the downstream intron, the a downstream exon each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the upstream exon and the downstream exon each comprise a portion of a gene, which is different from the gene for the portion of the upstream intron, the alternatively spliced exon, and the downstream intron. In some aspects, the upstream exon and the downstream exon comprise synthetic exons.

In some aspects, the splicing modulator is Kinetin or Homocarbonyltopsentin.

Certain aspects of the disclosure are directed to a method of producing an adeno-associated viral (AAV) vector comprising: (a) introducing a first plasmid comprising an adenovirus helper gene and a second plasmid comprising a heterologous nucleic acid sequence into a host cell; and
(b) culturing the host cell in the presence of a splicing modulator to produce AAV particles.

In some aspects, the host cell comprises an AAV capsid (Cap) gene and a nucleic acid construct comprising (i) an upstream exon, (ii) an upstream intron, (iii) an alternatively spliced exon comprising a translation initiation sequence, (iv) a downstream intron, (v) a downstream exon, and (vi) an AAV replication (Rep) gene.

In some aspects, the host cell comprises an AAV replication (Rep) gene and a nucleic acid construct comprising (i) an upstream exon, (ii) an upstream intron, (iii) an alternatively spliced exon comprising a translation initiation sequence, (iv) a downstream intron, (v) a downstream exon, and (vi) an AAV capsid (Cap) gene.

In some aspects, the upstream intron, the alternatively spliced exon, and the downstream intron each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the upstream exon and the downstream exon each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the upstream exon, the upstream intron, the alternatively spliced exon comprising a translation initiation sequence, the downstream intron, the a downstream exon each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the upstream exon and the downstream exon each comprise a portion of a gene, which is different from the gene for the portion of the upstream intron, the alternatively spliced exon, and the downstream intron. In some aspects, the upstream exon and the downstream exon comprise synthetic exons.

In some aspects, the method further comprises (c) harvesting the AAV particles from the cultured host cell.

In some aspects, the method further comprises (d) purifying the harvested AAV particles.

In some aspects, the host cell is a HEK293 cell.

In some aspects, the splicing modulator is Kinetin or Homocarbonyltopsentin.

In some aspects, the adenovirus helper gene is selected from the group consisting of an Early 4 (E4) gene, an Early 2A (E2A) gene, and a viral associated (VA) gene.

In some aspects, the nucleic acid construct is stably integrated into the host cell genome.

In some aspects, the nucleic acid construct comprises a sequence selected from the group consisting of SEQ ID NO: 59-62.

Certain aspects of the disclosure are directed to a method of producing an AAV particle comprising contacting a host cell comprising rep genes, cap genes, and adenoviral helper genes with a topsentin alkaloid or derivative thereof.

In some aspects, the method: i) reduces viral response genes, ii) increases immediate early genes, iii) increases a NF-κB signaling pathway, iv) increases a TNF signaling pathway, v) increases a IL-17 signaling pathway, vi) inhibits apoptosis, vii) inhibits viral defense and/or vii) increases histone levels.

In some aspects, the apoptosis is inhibited by altering a signaling pathway.

In some aspects, the signaling pathway comprises a NF-κB signaling pathway, a TNF signaling pathway, IL-17 signaling pathway, a JAK/STAT signaling pathway, a P38 MAPK signaling pathway, PI3K-Akt signaling pathway, or any combination thereof.

In some aspects, the increased histone levels result in increased metabolic activity and/or increased host cell replication.

In some aspects, the host cell comprises an AAV capsid (Cap) gene, an AAV replication (Rep) gene, an AAV helper gene, or any combination thereof.

In some aspects, the host cell is a HEK293 cell.

In some aspects, the topsentin alkaloid or derivative thereof is selected from the group consisting of Homocarbonyltopsentin, Topsentin, Bromotopsentin, Bromodeoxytopsentin, Deoxytopsentin, and any combination thereof. In some aspects, the topsentin alkaloid or derivative thereof is Homocarbonyltopsentin.

In some aspects, the host cell is contacted with about 5 μM to about 50 μM (e.g., about 5 μM, about 10 μM, about 20 μM, or about 40 μM) of the topsentin alkaloid or derivative thereof (e.g., Homocarbonyltopsentin).

In some aspects, the host cell is contacted with the topsentin alkaloid or derivative thereof (e.g., Homocarbonyltopsentin) for at least 24 hours, at least 48 hours, or at least 72 hours (e.g., 24 to 90 hours).

In some aspects, the method further comprises harvesting the AAV particle.

In some aspects, the method further comprises purifying the harvested AAV particle.

### BRIEF DESCRIPTION OF FIGURES

FIGS. 1A-1B show an exemplary schematic of a riboswitch, both with (FIG. 1B) and without (FIG. 1A) a cleavage site between the downstream exon and the heterologous nucleic acid sequence (GFP).

FIG. 2 shows the effect of homocarbonyltopsentin (Drug H) and kinetin (Drug K) on alternatively spliced exons in HEK293T cells as measured by mRNA-seq.

FIGS. 3A-3C show the effect of kinetin as measured by RNA-seq on alternatively spliced exons in three genes: TRPV3 (FIG. 3A), STK31 (FIG. 3B), and USP25 (FIG. 3C).

FIGS. 4A-4D show the effect of homocarbonyltopsentin as measured by RNA-seq on alternatively spliced exons in four genes: IK (FIG. 4A), TEX14 (FIG. 4B), GTF2B (FIG. 4C), and LYPD3 (FIG. 4D).

FIGS. 5A-5C show immunofluorescent staining in cells transfected with USP25 Full (FIG. 5A), STK31 Full (FIG. 5B) or TRPV3 mini (FIG. 5C) after treatment with DMSO, Low Drug K, or High Drug K.

FIGS. 6A-6D show immunofluorescent staining in cells transfected with LYPD3 Full (FIG. 6A), TEX14 Full (FIG. 6B), GTF2B Mini (FIG. 6C), or IK Mini (FIG. 6D) after treatment with DMSO, Low Drug H, or High Drug H.

FIGS. 7A-7E show immunofluorescent staining (FIGS. 7A-7D) or qPCR measurements (FIG. 7E) of Drug K or Drug H driven splicing. GTF2B Minimal Reporter (FIG. 7A-Drug H), IK Minimal Reporter (FIG. 7B-Drug H), TRPV3 Minimal Reporter (FIG. 7C), and USP25 Minimal Reporter (FIG. 7D) were analyzed. Fold change comparing Drug H or Drug K treated samples with vehicle was analyzed by qPCR in FIG. 7E for cells transfected with Gene G (GTF2B Minimal Reporter), Gene I (IK Minimal Reporter), Gene T (TRPV3 Minimal Reporter), and Gene U (USP25 Minimal Reporter).

FIGS. 8A-8H show immunofluorescent staining in triplicate for DMSO, 10 μM Drug H, or 40 μM Drug H for cells transfected with GTF2B Full Reporter (FIGS. 8A-8B), GTF2B Mini Reporter (FIGS. 8C-8D), IK Mini Reporter (FIGS. 8E-8F), or LYPD3 Mini Reporter (FIGS. 8G-8H).

FIG. 9 shows an RNA PCR splicing assay in cells transfected with LYPD3 Mini Reporter, IK Mini Reporter, and GTF2B Full Reporter after increasing Drug H treatment.

FIGS. 10A-10C show immunofluorescent staining 24 hours after treatment with DMSO, Low Drug H (10 μM), or High Drug H (40 μM) for cells transfected with Full Gene L (LYPD3) (FIG. 10A), Minimum Gene L (version 1-779 bp) (FIG. 10B), or Minimum Exon/Intron Gene L (version 2-414 bp) (FIG. 10C).

FIGS. 11A-11G show immunofluorescent staining 48 hours after treatment with DMSO, Low Drug K (50 μM), High Drug K (200 μM), Low Drug H (10 μM), or High Drug H (40 μM) for cells transfected with constitutively expressed GFP (FIG. 11A), Full Gene L (LYPD3) (FIG. 11B), Minimum Gene L (version 1-779 BP) (FIG. 11C), Minimum Exon/Intron Gene L (version 2-414 bp) (FIG. 11D), Full Gene T (TRPV3) (FIG. 11E), Minimum Gene T (version 1 906 bp) (FIG. 11F), or Minimum Exon/Intron Gene T (version 2-410 bp) (FIG. 11G).

FIGS. 12A-12C show immunofluorescent staining and mRNA fold change after treatment with vehicle, Low Drug H (10 μM), or High Drug H (40 μM) for cells transfected with constitutively expressed GFP (FIG. 12A) or a KT Splice-Drug H-GFP construct (FIG. 12B).

FIG. 12C shows mRNA fold change of GFP expression relative to Actin and GAPDH.

FIGS. 13A-13D show immunofluorescent staining and mRNA fold change after treatment with vehicle, Low Drug H (10 μM), or High Drug H (40 μM) for cells transfected with constitutively expressed iRFP (FIG. 13A) a KT Splice-Drug H-iRFP construct (FIG. 13B), or a KT Splice-Constitutively Expressed Exon (FIG. 13C), which does not have a splicing requirement (a control for similar expression to constitutively expressed iRFP). FIG. 13D shows mRNA fold change of iRFP expression relative to Actin and GAPDH.

FIG. 14 shows immunofluorescent staining of cells after transfection with inducible green fluorescent protein (GFP) construct “VIA” or “V2A” and treatment with vehicle or with small molecule “Drug 1” (Drug H).

FIG. 15 shows ITR titer after triple transfection of “WT Rep” or inducible Rep78/52 constructs (“V2A” or “V2B”) after treatment with Drug K (“Drug 2”) or Drug H (“Drug 1”).

FIG. 16 shows fold change of induction of capsid titer after triple transfection of inducible Rep78/52 after treatment with Drug K (“Drug 2”) or after treatment with Drug H (“Drug 1”).

FIGS. 17A-17B show a western blot of AAV Rep78 levels (FIG. 17A) or VP1, VP2, and VP3 levels (FIG. 17B) after transfection with Drug K (“Drug 2”) or Drug H (“Drug 1”).

FIG. 18 shows an exemplary scheme for the inducible Rep system in comparison to the Triple Transfection system.

FIGS. 19A-19B show ITR titer after triple transfection of “WT Rep” after treatment with Drug K (FIG. 19A) or Drug H (FIG. 19B).

FIGS. 20A-20B show fold change of induction of capsid titer after triple transfection of WT Rep. FIG. 20A shows capsid titer after treatment with Drug K. FIG. 20B shows capsid titer after treatment with Drug H.

FIGS. 21A-21B show a western blot of AAV VP1, VP2, and VP3 levels after treatment with Drug K (FIG. 21A) or Drug H (FIG. 21B).

FIG. 22A shows the expression of Cap mRNA expression after treatment with Drug H (5 μM, 10 μM, 20 μM, and 40 μM) after 0 hours, 24 hours, and 48 hours.

FIG. 22B shows the expression of Rep mRNA expression after treatment with Drug H (5 μM, 10 μM, 20 μM, and 40 μM) at 0 hour, 24 hours, and 48 hours.

FIGS. 23A-23H show changes in the transcriptome after treatment with Drug H at 24 hours (FIGS. 23A-23D) and 48 hours (FIGS. 23E-23H). Concentrations of Drug H analyzed include 5 μM (FIG. 23A and FIG. 23E), 10 μM (FIG. 23B and FIG. 23F), 20 μM (FIG. 23C and FIG. 23G), and 40 μM (FIG. 23D and FIG. 23H).

FIG. 24 shows a pathway analysis of differentially expressed genes driven by Drug H treatment, comparing triple transfection with Drug H relative to triple transfection without Drug H.

FIG. 25A shows a heat map of a gene set involved in the inflammatory response after treatment with Drug H for 24 hours or 48 hours.

FIG. 25B shows a heat map of a gene set involved in the immediate early response after treatment with Drug H for 24 hours or 48 hours.

FIG. 25C shows a heat map of a gene set involved in the Defense to Virus pathway after treatment with Drug H for 24 hours or 48 hours.

FIGS. 25D-25G show fold change of genes involved in the Defense to Virus pathway after treatment with Drug H for 24 hours or 48 hours. Genes analyzed include BST2 (FIG. 25D), EXOSC5 (FIG. 25E), CGAS (FIG. 25F), and GBP1 (FIG. 25G).

FIG. 26A shows a heat map of a gene set involved in nucleosome assembly genes after treatment with Drug H for 24 hours or 48 hours.

FIGS. 26B-26E show fold change of genes involved in nucleosome assembly after treatment with Drug H for 24 hours or 48 hours. Genes analyzed include H1-3 (FIG. 26B), H2BC8 (FIG. 26C), H3C1 (FIG. 26D), and H4C3 (FIG. 26E).

FIG. 27A shows a heat map of a gene set involved in the TNF signaling pathway after treatment with Drug H for 24 hours or 48 hours.

FIGS. 27B-27C show normalized gene counts of TNFAIP3 (FIG. 27B) and FOS (FIG. 27C) after treatment with Drug H at 0 hours, 24 hours, and 48 hours.

FIG. 28A shows expression of genes within the JAK/STAT signaling pathway 48 hours after treatment with Drug H.

FIG. 28B shows expression of genes within the IL-17 and NF-κB signaling pathways 48 hours after treatment with Drug H.

FIG. 28C shows expression of genes in parallel and downstream pathways to IL-17 signaling 48 hours after treatment with Drug H (e.g. P38 MAPK signaling and PI3K-Akt signaling pathways).

FIG. 28D shows expression of genes in pathways that converge on apoptosis 48 hours after treatment with Drug H.

FIG. 28E shows expression of genes within the TNF signaling pathway after treatment with Drug H.

FIG. 29 shows an exemplary scheme for the Mode of Action for Drug H on host cells during AAV production.

FIGS. 30A-30C shows the epifluorescence of non-transduced cells (FIG. 30A) and cells transduced with AAV2 packaged Positive Control Construct carrying an expression construct including enhanced GFP (eGFP) operably linked aRous Sarcoma Virus (RSV) promoter (FIGS. 30B and 30C) in HEK293 cells 24 hours after treatment with the amount indicated of DMSO, Drug H (Homocarb), or Drug K (Kinetin). Multiplicity of infection of each AAV construct was either 1e5 or 5e5 vector genomes per cell as indicated in figure title.

FIGS. 31A-31D shows the epifluorescence of cells transduced with AAV2 packaged TRPV3 construct #3 (FIGS. 31A and 31B), or cells transduced with AAV2 packaged LYPD3 construct #3 (FIGS. 31C and 31D), each carrying an expression construct including enhanced GFP (eGFP) operably linked aRous Sarcoma Virus (RSV) promoter, in HEK293 cells 24 hours after treatment with the amount indicated of DMSO, Drug H (Homocarb), or Drug K (Kinetin). Multiplicity of infection of each AAV construct was either 1e5 or 5e5 vector genomes per cell as indicated in figure title.

FIGS. 32A-32C shows the epifluorescence of non-transduced cells (FIG. 32A), cells transduced with AAV2 packaged Positive Control Construct (FIGS. 32B and 32C) carrying an expression construct including enhanced GFP (eGFP) operably linked aRous Sarcoma Virus (RSV) promoter in Neuro2A cells 24 hours after treatment with the amount indicated of DMSO, Drug H (Homocarb), or Drug K (Kinetin). Multiplicity of infection of each AAV construct was either 1e5 or 5e5 vector genomes per cell as indicated in figure title.

FIGS. 33A-33D shows the epifluorescence of cells transduced with AAV2 packaged TRPV3 construct #3 (FIGS. 33A and 33B), or cells transduced with AAV2 packaged LYPD3 construct #3 (FIGS. 33C and 33D), each carrying an expression construct including enhanced GFP (eGFP) operably linked aRous Sarcoma Virus (RSV) promoter, in Neuro2A cells 24 hours after treatment with the amount indicated of DMSO, Drug H (Homocarb), or Drug K (Kinetin). Multiplicity of infection of each AAV construct was either 1e5 or 5e5 vector genomes per cell as indicated in figure title.

FIGS. 34A-34C shows the epifluorescence of non-transduced cells, cell transduced with AAV2 packaged Positive Control Construct (FIG. 34A), cells transduced with AAV2 packaged TRPV3 construct #3 (FIGS. 34B and 34C), each carrying an expression construct including enhanced GFP (eGFP) operably linked aRous Sarcoma Virus (RSV) promoter, in Neuro2A cells 24 hours after treatment with the amount indicated of DMSO or Drug K (Kinetin). Multiplicity of infection of each AAV construct was either 1e5 or 5e5 vector genomes per cell as indicated in figure title.

FIGS. 35A-35B shows the epifluorescence of cells transduced with AAV2 packaged LYPD3 construct #3 (FIGS. 35A and 35B) carrying an expression construct including enhanced GFP (eGFP) operably linked aRous Sarcoma Virus (RSV) promoter in Neuro2A cells 24 hours after treatment with the amount indicated of DMSO or Drug H (Homocarb). Multiplicity of infection of each AAV construct was either 1e5 or 5e5 vector genomes per cell as indicated in figure title.

FIGS. 36A-36B shows the epifluorescence of non-transduced cells (FIG. 36A), cell transduced with AAV2 packaged Positive Control Construct (FIG. 36B) carrying an expression construct including enhanced GFP (eGFP) operably linked aRous Sarcoma Virus (RSV) promoter in Neuro2A cells 24 hours after treatment with the amount indicated of DMSO, Drug H (Homocarb), or Drug K (Kinetin). Multiplicity of infection of each AAV construct was either 1e5 or 5e5 vector genomes per cell as indicated in figure title.

FIGS. 37A-37D show the epifluorescence cells transduced with AAV2 packaged TRPV3 construct #3 (FIGS. 37A and 37B), or cells transduced with AAV2 packaged LYPD3 construct #3 (FIGS. 37C and 37D) carrying an expression construct including enhanced GFP (eGFP) operably linked aRous Sarcoma Virus (RSV) promoter in Neuro2A cells 24 hours after treatment with the amount indicated of DMSO, Drug H (Homocarb), or Drug K (Kinetin). Multiplicity of infection of each AAV construct was either 1e5 or 5e5 vector genomes per cell as indicated in figure title.

FIGS. 38A-38B shows the quantification of the DNA of each vector in mouse livers. Each dot represents the vector genomes per nanogram of total DNA quantified by a standard curve of input DNA. Positive control represents AAV2 packaged Positive Control Construct, TRPV3 represents AAV2 packaged TRPV3 Construct #3, and LYPD3 represents AAV2 packaged LYPD3 Construct #3, each carrying an expression construct including enhanced GFP (eGFP) operably linked aRous Sarcoma Virus (RSV) promoter, at 1e11 vector genomes per animal (FIG. 38A) and 1e12 vector genomes per animal (FIG. 38B).

FIGS. 39A-39F show the effect of homocarbonyltopsentin (Drug H) and kinetin (Drug K) measured by qPCR on the alternatively spliced exon of either AAV2 packaged TRPV3 Construct #3 or AAV2 packaged LYPD3 Construct #3, carrying an expression construct including enhanced GFP (eGFP) operably linked aRous Sarcoma Virus (RSV) promoter, in mouse tissues. The relative expression of the alternatively spliced exon was compared to the expression of AAV2 packaged Positive Control Construct for TRPV3 Construct #3 of RNA from mouse liver (FIG. 39A-39B) or for LYPD3 Construct #3 of RNA from mouse liver (FIG. 39C-39D) or mouse heart (FIG. 39E-39F). Stars indicate p-value of less than 0.05 as determined by one-way ANOVA.

### DETAILED DESCRIPTION OF THE DISCLOSURE

In some aspects, provided herein is an expression cassette comprising from 5′ to 3′: (i) promoter, (ii) an upstream exon, (iii) an upstream intron, (iv) an alternatively spliced exon comprising a translation initiation sequence, (v) a downstream intron, (vi) a downstream exon, and (vii) a heterologous nucleic acid sequence. In some aspects, the upstream intron, the alternatively spliced exon, and the downstream intron each comprise a portion of a gene (reference or endogenous gene) selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the upstream exon and the downstream exon, each comprise a portion of a gene (reference or endogenous gene) selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the upstream exon, the upstream intron, the alternatively spliced exon comprising a translation initiation sequence, the downstream intron, the a downstream exon each comprise a portion of a gene (reference or endogenous gene) selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the upstream exon and the downstream exon each comprise a portion of a gene (reference or endogenous gene), which is different from the gene for the portion of the upstream intron, the alternatively spliced exon, and the downstream intron.

In some aspects, the upstream exon and the downstream exon comprise synthetic exons.

In some aspects, the portion of the gene comprises fragments or shortened regions of a reference or endogenous gene (e.g., upstream exon of the gene, upstream intron of the gene, an alternatively spliced exon comprising a translation initiation sequence of the gene, downstream intron of the gene, and/or downstream exon of the gene) or regions missing sections of the endogenous gene. In some aspects, the reference or endogenous gene is selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the portion of the gene comprises a truncated region of the gene (e.g., upstream exon of the gene, upstream intron of the gene, an alternatively spliced exon comprising a translation initiation sequence of the gene, downstream intron of the gene, and/or downstream exon of the gene).

In some aspects, the portion of the gene (e.g., a truncated portion) comprises two or more portions of a region of the gene (e.g., upstream intron but missing an intervening sequence contained in the endogenous upstream intron or downstream intron but missing an intervening sequence contained in the endogenous downstream intron).

In some aspects, the portion of the gene comprises 15 to 400 basepairs. In some aspects, the portion of the gene comprises less than 400 basepairs, less than 350 basepairs, or less than 300 basepairs. In some aspects, the portion of the gene comprises at least 15 basepairs, at least 18 basepairs, at least 21 basepairs, at least 24 basepairs, at least 27 basepairs, at least 30 basepairs, at least 45 basepairs, at least 60 basepairs, at least 75 basepairs, or at least 90 basepairs.

In some aspects, the portion of the gene is 15 to 400 basepairs. In some aspects, the portion of the gene is less than 400 basepairs, less than 350 basepairs, or less than 300 basepairs. In some aspects, the portion of the gene is at least 15 basepairs, at least 18 basepairs, at least 21 basepairs, at least 24 basepairs, at least 27 basepairs, at least 30 basepairs, at least 45 basepairs, at least 60 basepairs, at least 75 basepairs, or at least 90 basepairs.

In some aspects, the expression cassette comprises (i) a promoter; (ii) a truncated upstream exon comprising the 3′ end of the endogenous upstream exon; (iii) a truncated upstream intron comprising the 5′ and 3′ end of the endogenous upstream intron but missing an intervening sequence contained in the endogenous upstream intron; (iv) an alternatively spliced exon comprising a translation initiation sequence, (v) a truncated downstream intron comprising the 5′ and 3′ end of the endogenous downstream intron but missing an intervening sequence contained in the endogenous downstream intron, (vi) a truncated downstream exon comprising the 5′ end of the endogenous downstream exon, and (vii) a heterologous nucleic acid sequence.

In some aspects, provided herein is a delivery vector comprising any of the expression cassettes disclosed herein.

In some aspects, provided herein is a cell comprising any of the expression cassettes disclosed herein or any of the delivery vectors disclosed herein.

In some aspects, provided herein is a pharmaceutical composition comprising any of the expression cassettes disclosed herein, any of the delivery vectors disclosed herein, or any of the cells disclosed herein.

In some aspects, provided herein is a kit comprising i) any of the expression cassettes disclosed herein, any of the delivery vectors disclosed herein, any of the cells disclosed herein, or any of the pharmaceutical compositions disclosed herein; and (ii) optionally instructions for use.

In some aspects, provided herein is a method of regulating expression of a protein in a mammalian cell comprising administering to the cell any of the expression cassettes disclosed herein, any of the delivery vectors disclosed herein, or any of the pharmaceutical compositions disclosed herein, followed by administering the splicing modulator molecule that promotes the inclusion of the alternatively spliced exon.

In some aspects, provided herein is a method of expressing a protein in a subject comprising administering to the subject any of the expression cassettes disclosed herein, any of the delivery vectors disclosed herein, any of the cells disclosed herein, or any of the pharmaceutical compositions disclosed herein, followed by administering the splicing modulator molecule that promotes the inclusion of the alternatively spliced exon.

In some aspects, provided herein is a method of treating a disease in a mammal comprising administering to a subject any of the expression cassettes disclosed herein, any of the delivery vectors disclosed herein, any of the cells disclosed herein, or any of the pharmaceutical compositions disclosed herein, followed by administering the splicing modulator molecule that promotes the inclusion of the alternatively spliced exon.

In some aspects, provided herein is a method of regulating transgene expression post-transcriptionally, comprising administering to a subject (a) a nucleic acid construct comprising (i) an upstream exon, (ii) an upstream intron, (iii) an alternatively spliced exon comprising a translation initiation sequence, (iv) a downstream intron, (v) a downstream exon, and (vi) a heterologous nucleic acid sequence; and (b) a splicing modulator. In some aspect, the upstream intron, the alternatively spliced exon, and the downstream intron each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene. In some aspects, the splicing modulator is Kinetin or Homocarbonyltopsentin.

In some aspects, provided herein is a method of producing an adeno-associated viral (AAV) vector comprising: (a) introducing a first plasmid comprising an adenovirus helper gene and a second plasmid comprising a heterologous nucleic acid sequence into a host cell; and (b) culturing the host cell in the presence of a splicing modulator to produce AAV particles. In some aspects, the host cell comprises an AAV capsid (Cap) gene and a nucleic acid construct comprising (i) an upstream exon, (ii) an upstream intron, (iii) an alternatively spliced exon comprising a translation initiation sequence, (iv) a downstream intron, (v) a downstream exon, and (vi) an AAV replication (Rep) gene. In some aspects, the host cell comprises an AAV replication (Rep) gene and a nucleic acid construct comprising (i) an upstream exon, (ii) an upstream intron, (iii) an alternatively spliced exon comprising a translation initiation sequence, (iv) a downstream intron, (v) a downstream exon, and (vi) an AAV capsid (Cap) gene. In some aspects, the upstream intron, the alternatively spliced exon, and the downstream intron each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, provided herein is a method of producing an AAV particle comprising contacting a host cell comprising rep genes, cap genes, and adenoviral helper genes with a topsentin alkaloid or derivative thereof (e.g., Homocarbonyltopsentin).

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order that the present disclosure can be more readily understood, certain terms are first defined. Additional definitions are set forth throughout the detailed disclosure.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “pharmaceutical composition,” as used herein, represents a composition comprising a compound or molecule described herein, e.g., a vector disclosed herein, formulated with a pharmaceutically acceptable excipient, and can be manufactured or sold with the approval of a governmental regulatory agency as part of a therapeutic regimen for the treatment of disease in a mammal.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient.

The terms “operatively linked,” “operatively inserted,” “operatively positioned,” “under control” or “under transcriptional control” means that the promoter is in the correct location and orientation in relation to the nucleic acid to control RNA polymerase initiation and expression of the gene. The term “operably linked” means that a DNA sequence and a regulatory sequence(s) are connected in such a way as to permit gene expression when the appropriate molecules (e.g., transcriptional activator proteins) are bound to the regulatory sequence(s).

“Nucleic acid,” “polynucleotide,” and “oligonucleotide,” are used interchangeably in the present application. These terms refer only to the primary structure of the molecule. Thus, these terms include double- and single-stranded DNA, as well as double- and single-stranded RNA. The terms “nucleic acid,” “polynucleotide,” and “oligonucleotide,” as used herein, are defined as it is generally understood by the skilled person as a molecule comprising two or more covalently linked nucleosides. Such covalently bound nucleosides can also be referred to as nucleic acid molecules or oligomers. Polynucleotides can be made recombinantly, enzymatically, or synthetically, e.g., by solid-phase chemical synthesis followed by purification. When referring to a sequence of the polynucleotide or nucleic acid, reference is made to the sequence or order of nucleobase moieties, or modifications thereof, of the covalently linked nucleotides or nucleosides.

The term “mRNA” as used herein, refers to a single stranded RNA that encodes the amino acid sequence of one or more polypeptide chains.

A “coding sequence” or a sequence “encoding” a particular molecule (e.g., a therapeutic protein or peptide) is a nucleic acid that is transcribed (in the case of DNA) or translated (in the case of mRNA) into polypeptide, in vitro or in vivo, when operably linked to an appropriate regulatory sequence, such as a promoter. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the coding sequence.

As used herein, the term “polypeptide” is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with any of these terms. The term further includes polypeptides which have undergone post-translational or post-synthesis modifications, for example, conjugation of a palmitoyl group, glycosylation, acetylation, phosphorylation, amidation, derivatization by known protecting/blocking groups, proteolytic cleavage, or modification by non-naturally occurring amino acids. The term “peptide,” as used herein encompasses full length peptides and fragments, variants or derivatives thereof. A “peptide” as disclosed herein, can be part of a fusion polypeptide comprising additional components such as, e.g., an Fc domain or an albumin domain, to increase half-life. A peptide as described herein can also be derivatized in a number of different ways. A peptide described herein can comprise modifications including e.g., conjugation of a palmitoyl group.

“Percent (%) sequence identity” or “Percent (%) identity” or “sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values can be generated using the sequence comparison computer program BLAST.

As used herein, the term “promoter” refers to a DNA sequence recognized by the machinery of the cell, or introduced synthetic machinery, that can initiate the transcription of a nucleic acid (e.g., a gene of interest). The term “promoter” can also encompass those nucleic acid elements sufficient for promoter-dependent gene expression controllable for cell-type specific, tissue-specific or inducible by external signals or agents; such elements can be located in the 5′ or 3′ regions of the native gene. In some aspects, the promoter is a constitutive promoter, a cell-type specific promoter, or an inducible promoter.

As used herein, the term “enhancer” is a cis-acting element that stimulates or inhibits transcription of adjacent genes. An enhancer that inhibits transcription is also referred to as a “silencer.” Enhancers can function (e.g., can be associated with a coding sequence) in either orientation, over distances of up to several kilobase pairs (kb) from the coding sequence and from a position downstream of a transcribed region.

The term “expression vector”, “expression construct”, or “expression cassette” means any type of genetic construct containing a nucleic acid in which part or all of the nucleic acid encoding sequence is capable of being transcribed.

As used herein, the term “delivery vector” or “vector” refers to any vehicle for the cloning of and/or transfer of a nucleic acid into a host cell, such as a plasmid, phage, transposon, cosmid, chromosome, artificial chromosome, virus, virion, etc. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “delivery vector” or “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. In some aspects, insertion of a polynucleotide into a suitable vector can be accomplished by ligating the appropriate polynucleotide fragments into a chosen vector that has complementary cohesive termini. Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentenyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters. In some aspects, the delivery vector is selected from the group consisting of a viral vector (e.g., an AAV vector), a plasmid, a lipid, a protein particle, a bacterial vector, and a lysosome.

Some aspects of the disclosure are directed to biological vectors, which can include viruses, particularly attenuated and/or replication-deficient viruses.

A “viral vector” refers to a sequence that comprises one or more polynucleotide regions encoding or comprising a molecule of interest, e.g., a protein, a peptide, and an oligonucleotide or a plurality thereof. Viral vectors can be used to deliver genetic materials into cells. Viral vectors can be modified for specific applications. In some aspects, the delivery vector of the disclosure is a viral vector selected from the group consisting of an adeno-associated viral (AAV) vector, an adenoviral vector, a lentiviral vector, or a retroviral vector.

The term “adeno-associated virus vector” or “AAV vector” or “adeno-associated viral vector” as used herein refers to any vector that comprises or derives from components of an adeno-associated vector and is suitable to infect mammalian cells, preferably human cells. The term AAV vector typically designates an AAV-type viral particle or virion comprising a payload. The AAV vector can be derived from various serotypes, including combinations of serotypes (i.e., “pseudotyped” AAV) or from various genomes (e.g., single stranded or self-complementary). In addition, the AAV vector can be replication defective and/or targeted. As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh8, AAVrh10, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an “AAV vector” includes a derivative of a known AAV vector. In some aspects, an “AAV vector” includes a modified or an artificial AAV vector. In some aspects, an AAV vector includes a hybrid vector (e.g., AAV-DJ, AAV-PHP.B, AAV2-ESGHGYF, AAVM41, AAV-LK03, AAV2-BR1, AAV587MTP, AAV-Anc80L65, AAV2-7m8, AAV2HBKO, AAV2YF, AAV6-RGD or AAV6.2). In some aspects, the terms “AAV genome” and “AAV vector” can be used interchangeably. In some aspects, the AAV vector is modified relative to the wild-type AAV serotype sequence. In some aspects, the modified AAV vector is a modified AAV6, e.g., an AAV6 vector comprising the RGD peptide (an AAV6-RGD vector) or an AAV6 vector comprising mutations of surface exposed tyrosine residues as described, for example, in Sayroo et al. Gene Ther. 2016 January; 23 (1): 18-25. In some aspects, the AAV6-RGD vector further comprises modified amino acids corresponding to Y705, Y731, T492, and K531, (e.g., Y705, Y731F, T492V, and K531E; also referred to as AAV-RGD-Y705-731F+T492V+K531E).

As used herein, a “recombinant AAV particle”, “recombinant AAV vector”, “rAAV particle”, or “rAAV vector” refers to an AAV virus that comprises a capsid protein and a vector genome (or an AAV genome) comprising at least one heterologous polynucleotide encoding a protein of interest and at least one inverted terminal repeat (ITR) region.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, “transcription” refers to the process of copying a segment of DNA into RNA.

As used herein, “messenger RNA” or “mRNA” refer to a form of mRNA molecule that only includes the exons that code for the amino acids that make up a protein. Thus, any introns and/or any exons that are not required for the particular protein have been removed.

As used herein, “mature messenger RNA” or “mature mRNA” refer to a final form of RNA molecule that is transcribed from a single strand of DNA and carries the genetic information needed to make a protein.

As used herein the term “intron” refers to a non-coding nucleotide portion of a gene sequence that is transcribed into a precursor mRNA (e.g., pre-mRNA) and subsequently removed by splicing during formation of the mature messenger RNA. Thus, introns are not present post-splicing in the final mature mRNA.

As used herein the term “exon” refers to a nucleotide portion of a gene or DNA sequence that is transcribed and can form part of a mature messenger RNA derived from the gene or DNA sequence, e.g., after the introns have been removed by RNA splicing.

As used herein, the terms “splicing” or “splice” refer to the process of removing introns and joining exons from pre-mRNA to generate mRNA. The terms “splice site” and “splice junction” are used interchangeably to refer to a region where a splicing event takes place, for example, at an exon-intron junction in a pre-mRNA molecule. For example, a slice donor site may be present at the 5′ end of an intron and a splice acceptor site may be present at the 3′ end of an intron.

As used herein, the term “alternative splicing” refers to a process where exons from the same gene are joined in different combinations leading to different but related mRNA. In some aspects, mRNAs can be translated to produce different proteins, e.g., with distinct structures and/or functions from a single gene.

As used herein, the terms “regulating” or “regulate” refer to controlling the activity of a molecule or biological function, such as enhancing or diminishing the activity or function (e.g., transgene or protein expression).

As used herein, the term “splicing modulator” refers to a molecule that is capable of enhancing or diminishing splicing. In some aspects, splicing can be regulated by the presence or absence of the splicing modulator, resulting in the presence or absence of particular mRNA variants (e.g., those that contain alternatively spliced exons).

As used herein, the term “stably integrated” refers to a process wherein a heterologous or foreign gene (e.g., an AAV Rep gene) becomes part of a host cell genome and is copied during replication such that descendants of the host cell also express the heterologous or foreign gene.

As used herein, the term “a portion of a gene” or “gene portion” refers to a nucleic acid sequence derived from a reference gene (e.g., an endogenous gene), wherein the portion includes a region or fragment of the reference gene. In some aspects, the gene portion can comprise a 5′ splice site, a 3′ splice site, an exon or fragment thereof, an intron or fragment thereof, or any combination thereof. In some aspects, the portion of the gene can comprise two or more fragments from a region of the gene (e.g., upstream exon of the gene, upstream intron of the gene, an alternatively spliced exon comprising a translation initiation sequence of the gene, downstream intron of the gene, and/or downstream exon of the gene).

As used herein, the term “gene” refers to a nucleic acid region, also referred to as a transcribed region, which expresses a polynucleotide, such as an RNA. The transcribed polynucleotide can have a sequence encoding a polypeptide, such as a functional protein, which can be translated into the encoded polypeptide when placed under the control of an appropriate regulatory region. In some aspects, the protein coding sequences a genes can be broken into smaller portions or pieces of coding sequences (exons) separated by non-coding sequences (introns). When such genes are transcribed, those exons and introns can be included in the initial messenger RNA products.

As used herein, the term “derived from” refers to a component that is isolated from or made using a specified molecule or organism, or information (e.g., amino acid or nucleic acid sequence) from the specified molecule or organism. In some aspects, the derived species can be obtained by, for example, naturally occurring mutagenesis, artificial directed mutagenesis or artificial random mutagenesis.

As used herein, the term “Drug H” refers to homocarbonyltopsentin.

As used herein, the term “Drug K” refers to kinetin.

As used herein, the term “Gene G” refers to the General Transcription Factor IIB gene (i.e., GTF2B).

As used herein, the term “Gene L” refers to the LY6/PLAUR Domain Containing 3 gene (i.e., LYPD3).

As used herein, the term “Gene I” refers to the IK cytokine gene (i.e., IK).

As used herein, the term “Gene T” refers to the Transient Receptor Potential Cation Channel Subfamily V Member 3 gene (i.e., TRPV3).

As used herein, the term “Gene U” refers to the Ubiquitin Specific Peptidase 25 gene (i.e., USP25).

As used herein, the term “Gene S” refers to the Serine/Threonine Kinase 31 gene (i.e., STK31).

### II. Alternatively Spliced Exons

Certain aspects of the disclosure are directed to an expression cassette comprising from 5′ to 3′: (i) promoter, (ii) an upstream exon, (iii) an upstream intron, (iv) an alternatively spliced exon comprising a translation initiation sequence, (v) a downstream intron, (vi) a downstream exon, and (vii) a heterologous nucleic acid sequence. In some aspects, the upstream intron, the alternatively spliced exon, and the downstream intron each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the upstream intron and the downstream intron can each comprise a portion of a gene, which is different from the gene for the portion of the alternatively spliced exon. In some aspects, the upstream intron and the downstream intron can comprise synthetic introns.

In some aspects, the upstream exon, the upstream intron, the alternatively spliced exon comprising a translation initiation sequence, the downstream intron, and the downstream exon are not in the protein coding sequence of the target gene.

In some aspects, the expression cassette comprises from 5′ to 3′: (i) promoter, (ii) an upstream exon, (iii) an upstream intron, (iv) an alternatively spliced exon comprising an expression modulator, (v) a downstream intron, (vi) a downstream exon, and (vii) a heterologous nucleic acid sequence. In some aspects, the upstream intron, the alternatively spliced exon, and the downstream intron each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene. In some aspects, the expression modulator is a translation initiation sequence.

In some aspects, the upstream exon and the downstream exon, each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the upstream exon, the upstream intron, the alternatively spliced exon comprising a translation initiation sequence, the downstream intron, the a downstream exon each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the upstream exon and the downstream exon each comprise a portion of a gene, which is different from the gene for the portion of the upstream intron, the alternatively spliced exon, and the downstream intron.

In some aspects, the upstream exon and the downstream exon comprise synthetic exons.

In some aspects, the portion of the gene comprises fragments or shortened regions of a reference or endogenous gene (e.g., upstream exon of the gene, upstream intron of the gene, an alternatively spliced exon comprising a translation initiation sequence of the gene, downstream intron of the gene, and/or downstream exon of the gene) or regions missing sections of the endogenous gene. In some aspects, the reference or endogenous gene is selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the portion of the gene comprises a truncated region of the gene (e.g., upstream exon of the gene, upstream intron of the gene, an alternatively spliced exon comprising a translation initiation sequence of the gene, downstream intron of the gene, and/or downstream exon of the gene).

In some aspects, the portion of the gene (e.g., a truncated portion) comprises two or more portions of a region of the gene (e.g., upstream intron but missing an intervening sequence contained in the endogenous upstream intron or downstream intron but missing an intervening sequence contained in the endogenous downstream intron).

In some aspects, the portion of the gene comprises 15 to 400 basepairs. In some aspects, the portion of the gene comprises less than 400 basepairs, less than 350 basepairs, or less than 300 basepairs. In some aspects, the portion of the gene comprises at least 15 basepairs, at least 18 basepairs, at least 21 basepairs, at least 24 basepairs, at least 27 basepairs, at least 30 basepairs, at least 45 basepairs, at least 60 basepairs, at least 75 basepairs, or at least 90 basepairs.

In some aspects, the portion of the gene is 15 to 400 basepairs. In some aspects, the portion of the gene is less than 400 basepairs, less than 350 basepairs, or less than 300 basepairs. In some aspects, the portion of the gene is at least 15 basepairs, at least 18 basepairs, at least 21 basepairs, at least 24 basepairs, at least 27 basepairs, at least 30 basepairs, at least 45 basepairs, at least 60 basepairs, at least 75 basepairs, or at least 90 basepairs.

In some aspects, the expression cassette comprises
(i) a promoter;
(ii) a truncated upstream exon comprising the 3′ end of the endogenous upstream exon;
(iii) a truncated upstream intron comprising the 5′ and 3′ end of the endogenous upstream intron but missing an intervening sequence contained in the endogenous upstream intron;
(iv) an alternatively spliced exon comprising a translation initiation sequence,
(v) a truncated downstream intron comprising the 5′ and 3′ end of the endogenous downstream intron but missing an intervening sequence contained in the endogenous downstream intron,
(vi) a truncated downstream exon comprising the 5′ end of the endogenous downstream exon, and
(vii) a heterologous nucleic acid sequence.

In some aspects, the alternatively spliced exon is regulatable (e.g., regulated) by a splicing modulator.

In some aspects, the splicing modulator is Kinetin or Homocarbonyltopsentin.

In some aspects, the gene is TRPV3, STK31, or USP25; and the splicing modulator is Kinetin.

In some aspects, the gene is GTF2B, LYPD3, IK, or TEX14; and the splicing modulator is Homocarbonyltopsentin.

In some aspects, the splicing modulator promotes inclusion of the alternatively spliced exon thereby driving expression of the heterologous nucleic acid sequence.

In some aspects, the promoter is selected from the group consisting of a CBA promoter, a smCBA promoter, a CMV promoter, an EF-1a (Elongation Factor 1a) promoter, a RSV (Rous Sarcoma Virus) promoter, an Ubiquitin (UbC) promoter, a CAG promoter, a SV40 (simian vacuolating virus 40) promoter, a PGK (phosphoglycerate kinase) promoter, a human beta actin promoter, a H1 (human polymerase III RNA) promoter, a human U6 small nuclear promoter, tetracycline responsive element, and any combination thereof.

In some aspects, the expression cassette further comprises an enhancer.

In some aspects, the enhancer is selected from the group consisting of a CMV enhancer, a SV40 enhancer, and any combination thereof.

In some aspects, the expression cassette further comprises a cleavage site between the downstream exon and the heterologous nucleic acid sequence.

In some aspects, the cleavage site is selected from the group consisting of a furin cleavage site, a foot-and-mouth disease virus 18 2A peptide (F2A), an equine rhinitis A virus 2A peptide (E2A), a porcine teschovirus-1 2A peptide (P2A), a Thosea asigna virus 2A peptide (T2A), and any combination thereof.

In some aspects, the expression cassette further comprises a leader sequence between the downstream exon and the heterologous nucleic acid sequence. In some aspects, the leader sequence is selected from the group consisting of a human opticin (hOPT) leader sequence, an interleukin-2 (IL-2) leader sequence, an interleukin-12 (IL-12) leader sequence, a human or mouse interleukin-6 (hIL-6 or mIL-6) leader sequence, a human insulin leader sequence, a human serum albumin (HSA) leader sequence, and any combination thereof.

In some aspects, the combined length of the upstream exon, the upstream intron, the alternatively spliced exon, the downstream intron, and the downstream exon is between about 300 basepairs to about 2100 basepairs.

In some aspects, the combined length of the upstream exon, the upstream intron, the alternatively spliced exon, the downstream intron, and the downstream exon is between about 300 basepairs to about 2300 basepairs, about 400 basepairs to about 2300 basepairs, about 500 basepairs to about 2300 basepairs, about 600 basepairs to about 2300 basepairs, about 700 basepairs to about 2300 basepairs, about 800 basepairs to about 2300 basepairs, about 900 basepairs to about 2300 basepairs, about 1000 basepairs to about 2300 basepairs, about 1100 basepairs to about 2300 basepairs, about 1200 basepairs to about 2300 basepairs, about 1300 basepairs to about 2300 basepairs, about 1400 basepairs to about 2300 basepairs, about 1500 basepairs to about 2300 basepairs, about 1600 basepairs to about 2300 basepairs, about 1700 basepairs to about 2300 basepairs, about 1800 basepairs to about 2300 basepairs, about 1900 basepairs to about 2300 basepairs, about 2000 basepairs to about 2300 basepairs, about 2100 basepairs to about 2300 basepairs, about 2200 basepairs to about 2300 basepairs, about 1500 basepairs to about 2200 basepairs, about 1500 basepairs to about 2100 basepairs, about 1500 basepairs to about 2000 basepairs, about 1500 basepairs to about 1900 basepairs, about 1500 basepairs to about 1800 basepairs, about 1500 basepairs to about 1700 basepairs, about 1500 basepairs to about 1600 basepairs, about 1800 basepairs to about 2200 basepairs, about 1700 basepairs to about 2100 basepairs, about 300 basepairs to about 2200 basepairs, about 300 basepairs to about 2100 basepairs, about 300 basepairs to about 2000 basepairs, about 300 basepairs to about 1900 basepairs, about 300 basepairs to about 1800 basepairs, about 300 basepairs to about 1700 basepairs, about 300 basepairs to about 1600 basepairs, or about 1700 basepairs to about 2200 basepairs.

In some aspects, the combined length of the upstream exon, the upstream intron, the alternatively spliced exon, the downstream intron, and the downstream exon is about 300 basepairs, about 400 basepairs, about 500 basepairs, about 600 basepairs, about 700 basepairs, about 800 basepairs, about 900 basepairs, about 1000 basepairs, about 1100 basepairs, about 1200 basepairs, about 1300 basepairs, about 1400 basepairs, about 1500 basepairs, about 1600 basepairs, about 1700 basepairs, about 1800 basepairs, about 1900 basepairs, about 2000 basepairs, about 2100 basepairs, about 2200 basepairs, or about 2300 basepairs.

In some aspects, the combined length of the upstream exon, the upstream intron, the alternatively spliced exon, the downstream intron, and the downstream exon is between 300 basepairs to 2300 basepairs, 400 basepairs to 2300 basepairs, 500 basepairs to 2300 basepairs, 600 basepairs to 2300 basepairs, 700 basepairs to 2300 basepairs, 800 basepairs to 2300 basepairs, 900 basepairs to 2300 basepairs, 1000 basepairs to 2300 basepairs, 1100 basepairs to 2300 basepairs, 1200 basepairs to 2300 basepairs, 1300 basepairs to 2300 basepairs, 1400 basepairs to 2300 basepairs, 1500 basepairs to 2300 basepairs, 1600 basepairs to 2300 basepairs, 1700 basepairs to 2300 basepairs, 1800 basepairs to 2300 basepairs, 1900 basepairs to 2300 basepairs, 2000 basepairs to 2300 basepairs, 2100 basepairs to 2300 basepairs, 2200 basepairs to 2300 basepairs, 1500 basepairs to 2200 basepairs, 1500 basepairs to 2100 basepairs, 1500 basepairs to 2000 basepairs, 1500 basepairs to 1900 basepairs, 1500 basepairs to 1800 basepairs, 1500 basepairs to 1700 basepairs, 1500 basepairs to 1600 basepairs, 1800 basepairs to 2200 basepairs, 1700 basepairs to 2100 basepairs, 300 basepairs to 2200 basepairs, 300 basepairs to 2100 basepairs, 300 basepairs to 2000 basepairs, 300 basepairs to 1900 basepairs, 300 basepairs to 1800 basepairs, 300 basepairs to 1700 basepairs, 300 basepairs to 1600 basepairs, or 1700 basepairs to 2200 basepairs.

In some aspects, the combined length of the upstream exon, the upstream intron, the alternatively spliced exon, the downstream intron, and the downstream exon is 300 basepairs, 400 basepairs, 500 basepairs, 600 basepairs, 700 basepairs, 800 basepairs, 900 basepairs, 1000 basepairs, 1100 basepairs, 1200 basepairs, 1300 basepairs, 1400 basepairs, 1500 basepairs, 1600 basepairs, 1700 basepairs, 1800 basepairs, 1900 basepairs, 2000 basepairs, 2100 basepairs, 2200 basepairs, or 2300 basepairs.

In some aspects, the length of the heterologous nucleic acid sequence is less than about 4100 basepairs, less than about 4000 basepairs, less than about 3900 basepairs, less than about 3800 basepairs, less than about 3600 basepairs, less than about 3400 basepairs, less than about 3200 basepairs, less than about 3000 basepairs, less than about 2800 basepairs, less than about 2600 basepairs, less than about 2400 basepairs, or less than about 2200 basepairs.

In some aspects, the length of the heterologous nucleic acid sequence is less than about 2600 basepairs, less than about 2400 basepairs, less than about 2200 basepairs, less than about 2000 basepairs, less than about 1800 basepairs, less than about 1600 basepairs, less than about 1400 basepairs, less than about 1200 basepairs, less than about 1000 basepairs, less than about 800 basepairs, less than about 600 basepairs, less than about 400 basepairs, or less than about 200 basepairs.

In some aspects, the length of the heterologous nucleic acid sequence is between about 200 basepairs to about 4100 basepairs, between about 400 basepairs to about 4100 basepairs, between about 600 basepairs to about 4100 basepairs, between about 800 basepairs to about 4100 basepairs, between about 1000 basepairs to about 4100 basepairs, between about 1200 basepairs to about 4100 basepairs, between about 1400 basepairs to about 4100 basepairs, between about 1600 basepairs to about 4100 basepairs, between about 1800 basepairs to about 4100 basepairs, between about 2000 basepairs to about 4100 basepairs, between about 2200 basepairs to about 4100 basepairs, between about 2400 basepairs to about 4100 basepairs, between about 2600 basepairs to about 4100 basepairs, between about 2800 basepairs to about 4100 basepairs, between about 3000 basepairs to about 4100 basepairs, between about 3200 basepairs to about 4100 basepairs, between about 3400 basepairs to about 4100 basepairs, between about 3600 basepairs to about 4100 basepairs, between about 3800 basepairs to about 4100 basepairs, between about 4000 basepairs to about 4100 basepairs, about 200 basepairs to about 2400 basepairs, between about 400 basepairs to about 2400 basepairs, between about 600 basepairs to about 2400 basepairs, between about 800 basepairs to about 2400 basepairs, between about 1000 basepairs to about 2400 basepairs, between about 1200 basepairs to about 2400 basepairs, between about 1400 basepairs to about 2400 basepairs, between about 1600 basepairs to about 2400 basepairs, between about 1800 basepairs to about 2400 basepairs, between about 2000 basepairs to about 2400 basepairs, between about 2200 basepairs to about 2400 basepairs, between about 200 basepairs to about 2200 basepairs, between about 200 basepairs to about 2000 basepairs, between about 200 basepairs to about 1800 basepairs, between about 200 basepairs to about 1600 basepairs, between about 200 basepairs to about 1400 basepairs, between about 200 basepairs to about 1200 basepairs, between about 200 basepairs to about 1000 basepairs, between about 200 basepairs to about 800 basepairs, between about 200 basepairs to about 600 basepairs, between about 200 basepairs to about 400 basepairs, between about 1000 basepairs to about 2000 basepairs, between about 1200 basepairs to about 2000 basepairs, between about 1400 basepairs to about 2000 basepairs, or between about 1600 basepairs to about 2000 basepairs.

In some aspects, the length of the heterologous nucleic acid sequence is less than 4100 basepairs, less than 4000 basepairs, less about 3900 basepairs, less than 3800 basepairs, less than 3600 basepairs, less than 3400 basepairs, less than 3200 basepairs, less than 3000 basepairs, less than 2800 basepairs, less than 2600 basepairs, less than 2400 basepairs, or less than 2200 basepairs. In some aspects, the length of the payload sequence is less than 2600 basepairs, less than 2400 basepairs, less than 2200 basepairs, less than 2000 basepairs, less than 1800 basepairs, less than 1600 basepairs, less than 1400 basepairs, less than 1200 basepairs, less than 1000 basepairs, less than 800 basepairs, less than 600 basepairs, less than 400 basepairs, or less than 200 basepairs.

In some aspects, the length of the heterologous nucleic acid sequence is between 200 basepairs to 4100 basepairs, between 400 basepairs to 4100 basepairs, between 600 basepairs to 4100 basepairs, between 800 basepairs to 4100 basepairs, between 1000 basepairs to 4100 basepairs, between 1200 basepairs to 4100 basepairs, between 1400 basepairs to 4100 basepairs, between 1600 basepairs to 4100 basepairs, between 1800 basepairs to 4100 basepairs, between 2000 basepairs to 4100 basepairs, between 2200 basepairs to 4100 basepairs, between 2400 basepairs to 4100 basepairs, between 2600 basepairs to 4100 basepairs, between 2800 basepairs to 4100 basepairs, between 3000 basepairs to 4100 basepairs, between 3200 basepairs to 4100 basepairs, between 3400 basepairs to 4100 basepairs, between 3600 basepairs to 4100 basepairs, between 3800 basepairs to 4100 basepairs, between 4000 basepairs to 4100 basepairs, 200 basepairs to 2400 basepairs, between 400 basepairs to 2400 basepairs, between 600 basepairs to 2400 basepairs, between 800 basepairs to 2400 basepairs, between 1000 basepairs to 2400 basepairs, between 1200 basepairs to 2400 basepairs, between 1400 basepairs to 2400 basepairs, between 1600 basepairs to 2400 basepairs, between 1800 basepairs to 2400 basepairs, between 2000 basepairs to 2400 basepairs, between 2200 basepairs to 2400 basepairs, between 200 basepairs to 2200 basepairs, between 200 basepairs to 2000 basepairs, between 200 basepairs to 1800 basepairs, between 200 basepairs to 1600 basepairs, between 200 basepairs to 1400 basepairs, between 200 basepairs to 1200 basepairs, between 200 basepairs to 1000 basepairs, between 200 basepairs to 800 basepairs, between 200 basepairs to 600 basepairs, between 200 basepairs to 400 basepairs, between 1000 basepairs to 2000 basepairs, between 1200 basepairs to 2000 basepairs, between 1400 basepairs to 2000 basepairs, or between 1600 basepairs to 2000 basepairs.

In some aspects, the heterologous nucleic acid sequence encodes a protein. In some aspects, the protein is a therapeutic protein.

In some aspects, the upstream exon comprises a sequence selected from the group consisting of SEQ ID NOs: 8, 13, 18, 23, 28, 33, and 38.

In some aspects, the upstream exon comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 8, 13, 18, 23, 28, 33, and 38.

In some aspects, the upstream exon comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 8, 13, 18, 23, 28, 33, and 38.

In some aspects, the alternatively spliced exon comprises a sequence selected from the group consisting of SEQ ID NO: 10, 15, 20, 25, 30, 35, and 40.

In some aspects, the alternatively spliced exon comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NO: 10, 15, 20, 25, 30, 35, and 40.

In some aspects, the alternatively spliced exon comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 10, 15, 20, 25, 30, 35, and 40.

In some aspects, the downstream exon comprises a sequence selected from the group consisting of SEQ ID NO: 12, 17, 22, 27, 32, 37, and 42.

In some aspects, the downstream exon comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NO: 12, 17, 22, 27, 32, 37, and 42.

In some aspects, the downstream exon comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 12, 17, 22, 27, 32, 37, and 42.

In some aspects, the upstream intron comprises a sequence selected from the group consisting of SEQ ID NOs: 9, 14, 19, 24, 29, 34, and 39.

In some aspects, the upstream intron comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 9, 14, 19, 24, 29, 34, and 39.

In some aspects, the upstream intron comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 9, 14, 19, 24, 29, 34, and 39.

In some aspects, the downstream intron comprises a sequence selected from the group consisting of SEQ ID NO: 11, 16, 21, 26, 31, 36, and 41.

In some aspects, the downstream intron comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NO: 11, 16, 21, 26, 31, 36, and 41.

In some aspects, the downstream intron comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 11, 16, 21, 26, 31, 36, and 41.

In some aspects, the expression cassette comprises a sequence selected from the group consisting of SEQ ID NOs: 1-7 or 43-66, or fragments thereof.

In some aspects, the expression cassette comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NO: 1-7 or 43-66, or fragments thereof.

In some aspects, the expression cassette comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 1-7 or 43-66, or fragments thereof.

In some aspects, the expression cassette comprises a sequence selected from the group consisting of SEQ ID NOs: 8-42.

In some aspects, the expression cassette comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NO: 8-42.

In some aspects, the expression cassette comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 8-42.

In some aspects, the expression cassette comprises the upstream exon, the upstream intron, the alternatively spliced exon comprising a translation initiation sequence, the downstream intron, the downstream exon, wherein each comprises a portion of a gene, wherein the gene is a Transient Receptor Potential Cation Channel Subfamily V Member 3 (TRPV3) gene.

In some aspects, the expression cassette comprises a sequence selected from the group consisting of SEQ ID NOs: 1, 8-12, and 43-45.

In some aspects, the expression cassette comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NO: 1, 8-12, and 43-45.

In some aspects, the expression cassette comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 1, 8-12, and 43-45.

In some aspects, the expression cassette comprises a sequence corresponding to SEQ ID NO: 45.

In some aspects, the expression cassette comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 45.

In some aspects, the expression cassette comprises a sequence having at least 85%, at least 90%, at least 91%, at least 92%, at least 93%, at least 94%, at least 95%, at least 96%, at least 97%, at least 98%, at least 99%, or 100% sequence identity to SEQ ID NO: 45.

In some aspects, the expression cassette comprises the upstream exon, the upstream intron, the alternatively spliced exon comprising a translation initiation sequence, the downstream intron, the downstream exon, wherein each comprises a portion of a gene, wherein the gene is a Serine/Threonine Kinase 31 (STK31) gene.

In some aspects, the expression cassette comprises a sequence selected from the group consisting of SEQ ID NOs: 2, 13-17, 46 and 47.

In some aspects, the expression cassette comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NO: 2, 13-17, 46 and 47.

In some aspects, the expression cassette comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 2, 13-17, 46 and 47.

In some aspects, the expression cassette comprises the upstream exon, the upstream intron, the alternatively spliced exon comprising a translation initiation sequence, the downstream intron, the downstream exon, wherein each comprises a portion of a gene, wherein the gene is an Ubiquitin Specific Peptidase 25 (USP25) gene.

In some aspects, the expression cassette comprises a sequence selected from the group consisting of SEQ ID NOs: 3, 18-22, 48 and 49.

In some aspects, the expression cassette comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NO: 3, 18-22, 48 and 49.

In some aspects, the expression cassette comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 3, 18-22, 48 and 49.

In some aspects, the expression cassette comprises the upstream exon, the upstream intron, the alternatively spliced exon comprising a translation initiation sequence, the downstream intron, the downstream exon, wherein each comprises a portion of a gene, wherein the gene is a General Transcription Factor IIB (GTF2B) gene.

In some aspects, the expression cassette comprises a sequence selected from the group consisting of SEQ ID NOs: 4, 23-27, 50 and 51.

In some aspects, the expression cassette comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NO: 4, 23-27, 50 and 51.

In some aspects, the expression cassette comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 4, 23-27, 50 and 51.

In some aspects, the expression cassette comprises the upstream exon, the upstream intron, the alternatively spliced exon comprising a translation initiation sequence, the downstream intron, the downstream exon, wherein each comprises a portion of a gene, wherein the gene is a LY6/PLAUR Domain Containing 3 (LYPD3) gene.

In some aspects, the expression cassette comprises a sequence selected from the group consisting of SEQ ID NOs: 5, 28-32, and 52-54.

In some aspects, the expression cassette comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NO: 5, 28-32, and 52-54.

In some aspects, the expression cassette comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 5, 28-32, and 52-54.

In some aspects, the expression cassette comprises a sequence corresponding to SEQ ID NO: 54.

In some aspects, the expression cassette comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 54.

In some aspects, the expression cassette comprises a sequence having at least 85%, at least 90%, at least 91%, at least 92%, at least 93%, at least 94%, at least 95%, at least 96%, at least 97%, at least 98%, at least 99%, or 100% sequence identity to SEQ ID NO: 54.

In some aspects, the expression cassette comprises the upstream exon, the upstream intron, the alternatively spliced exon comprising a translation initiation sequence, the downstream intron, the downstream exon, wherein each comprises a portion of a gene, wherein the gene is a IK gene.

In some aspects, the expression cassette comprises a sequence selected from the group consisting of SEQ ID NOs: 6, 33-37, 55 and 56.

In some aspects, the expression cassette comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NO: 6, 33-37, 55 and 56.

In some aspects, the expression cassette comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 6, 33-37, 55 and 56.

In some aspects, the expression cassette comprises the upstream exon, the upstream intron, the alternatively spliced exon comprising a translation initiation sequence, the downstream intron, the downstream exon, wherein each comprises a portion of a gene, wherein the gene is a Testis Expressed 14, Intercellular Bridge Forming Factor (TEX14) gene.

In some aspects, the expression cassette comprises a sequence selected from the group consisting of SEQ ID NOs: 7, 38-42, 57 and 58.

In some aspects, the expression cassette comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NO: 7, 38-42, 57 and 58.

In some aspects, the expression cassette comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 7, 38-42, 57 and 58.

In some aspects, the expression cassette comprises a sequence selected from the group consisting of SEQ ID NO: 43 to 66.

In some aspects, the expression cassette comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NO: 43 to 66.

In some aspects, the expression cassette comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 43 to 66.

In some aspects, the expression cassette comprises a sequence selected from the group consisting of SEQ ID NO: 59-62.

In some aspects, the expression cassette comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NO: 59-62.

In some aspects, the expression cassette comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 59-62.

In some aspects, the expression cassette comprises a sequence selected from the group consisting of SEQ ID NO: 63-66.

In some aspects, the expression cassette comprises a sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NO: 63 to 66.

In some aspects, the expression cassette comprises a sequence having at least 85%, at least 90%, at least 91%, at least 92%, at least 93%, at least 94%, at least 95%, at least 96%, at least 97%, at least 98%, at least 99%, or 100% sequence identity to any one of SEQ ID NO: 63 to 66.

In some aspects, the expression cassette comprises one or more of any of the sequences in Table 1.

TABLE 1
Genomic Sequences

| | Upstream | | Alternatively | | |
|---|---|---|---|---|---|
| Gene | Exon | Upstream Intron | Spliced Exon | Downstream Intron | Downstream Exon |
| TRPV3 | ttttatccaagc | gtgcgttgagagctgcgccggcgg | gtcattttgcatga | gtaattgatttataaataatagta | Ccttggcctcgctgatcg |
| (SEQ ID | tgtgcttgtgat | gagggccctcggaggatggccatt | tgttctgaagttctt | actcccacatttataaacacttt | agaagtgtcccaaagaca |
| NO: 1) | actgtctgtctt | tttcagatggggaaactgagatctg | gtttgtatatatcgt | atacagatgtcaaaatggcaa | acaaggactgcagctcct |
| | cttgtacttgttt | gagaggaagaggggtttaccaag | gtttttgcttggatt | agtgcatggcggccttggcaa | acggcagcttcagcgac |
| | gcctacaaag | gggcaggtcactgctctttctcccaa | tggagtag | caattagaccaactaccagtg | gcagtgctggaactcttca |
| | agtacctcgcc | atgccatgcactccctttgctttcgg | (SEQ ID NO: | gggcacttgccatgcttggcat | agctcaccataggcctgg |
| | tgcctcgtgct | ccaggctttaagtgtcaagaccttaa | 10) | tttccaagcattacctttatatat | gtgacctgaacatccagc |
| | ggccatggcc | atcccccaggcttgtggggtgctgg | | atacatatatattttattatacttt | agaactccaagtatcccat |
| | ctgggctggg | ggaggggtacagtttgtggaggaa | | aagttctagggtacatgtgcac | tctctttctgttcctgctcat |
| | cgaacatgctc | tgttctcacccctgggcagtggcgt | | aacatgcaggtttgttacatatg | cacctatgtcatcctcacct |
| | tactatacgcg | gctggtaaacatttaacgactgttct | | tatacatgtgccatgttggtatg | ttgttctcctcctcaacatg |
| | gggtttccagt | gtgtgtgtgtgtgtgtatgtgtgtgtg | | ctgcacccattaactcgtcattt | ctcattgctctgatgggcg |
| | ccatgggcat | tgtaggtggaatatgtatattccatat | | acattaggtatatctcctaatca | agactgtggagaacgtct |
| | gtacagcgtc | atatacacaaaagacttatcatacatt | | agcattacctttttacttctctca | ccaaggagagcgaacgc |
| | atgatccagaa | ttactatataaacgatgtgtatcacac | | acaacttgatcaagtgggaat | atctggcgcctgc |
| | g | aatttgcaaataatgaaaatatacca | | catcaccccgatcataaggta | (SEQ ID NO: 12) |
| | (SEQ ID | tgcagtttattgtaaattccacatagc | | actgaggcttagagactccag | |
| | NO: 8) | cagttgagtctcagaataatttgttgc | | gattcaaaccaggccagcca | |
| | | tttttttttttttttttggagaccaagtctt | | ctcataatccaacttcaccatgt | |
| | | gctctgctgcccaggccagagtaa | | ggtatatacctctggctttccca | |
| | | agtggtgcgatctcggctccctgca | | ttcattgcttacgtggccctcac | |
| | | acctctgcctcctgggttcaagcagt | | gacctaagataggttctgcaa | |
| | | tctcctgcctcagcctccagagtag | | gtgttttcagccccattttaaca | |
| | | ctgggatcacaggcacgcaccacc | | gatgcggaaactaaagtccac | |
| | | atgttggctaatttttgtatttttagtag | | agaaactcaactgatggtcttt | |
| | | agacagggtttcagcatgttggcca | | gcaaagtaggagcaaactaat | |
| | | ggattgtcttgaactcctgacctcag | | accctgaggtgatttcctttgg | |
| | | gtgatccacctgcctcggcctccca | | atttcacatcacgttactctcac | |
| | | aagtgctgggattacaggcatgaac | | agtttgctttagatacaactgtg | |
| | | caccgcgccaggcctatttgttgctt | | agagcctctctggtctgctgg | |
| | | tttattgaattctagtttttgttgtcaact | | gaccaggaaggagccccctc | |
| | | tttggttgcaattgacaaaagagtgc | | ttaaaatctctccatttacaaca | |
| | | tatcagacatgaatgttggttgagag | | gagggaacagcacgatctgg | |
| | | ttttctttataagcagtgattgaagtg | | ttgtatttctaacctctgcataga | |
| | | aaacaattaagatttatgttgaacttt | | gagatatcattttcccccacgt | |
| | | acttgtttatcaatgatgtgagcaact | | accagttgcttttttttcttttcttc | |
| | | tctttgctgaattggatactagtttgc | | agtgaccattgacatttgaaatt | |
| | | aaataccagaataatattctgttgata | | aagtagctgtttccatctgtctg | |
| | | tttttgtgctatacataatgtaaaggct | | gtaccttttctgggtcacaattt | |
| | | gcagacagaacacattttaaaattta | | gcttatttatttgctccttcacct | |
| | | aactggattaatagtttctccatcact | | gcttgtttattcatccatctattta | |
| | | gtcttaaatcatttagatctagatatc | | ctctctcatttaatttgctcatcc | |
| | | aacaaaacaatgagtcaatcaatct | | acctgtctatccgtccatccatc | |
| | | ctgatttgtggcatttaccaattttcat | | catccatccatccatccatcca | |
| | | gatgtaaatacagtagtcccccctta | | tccatccaatcattcatccatcg | |
| | | ttcttgggggagatgttctaagaccc | | atccatccattgatctatccatc | |
| | | cccatgaatgcctgaaactgcagat | | tatccatccatccatccatccat | |
| | | agtaccaaacattatataatatgctat | | ccatccatctaatcatccatcc | |
| | | gttttttcctatacatatacatctatgc | | aatcatccatccatccatccat | |
| | | aaataatgaaaatatactatacaata | | ccatccatccatccatccagtc | |
| | | cagtttattgtaaattccacttagcca | | atccatccattcatccatttaatc | |
| | | gttgagtcttagaataatttttgcttttt | | atccatccatcgatcaatcggt | |
| | | ttttttttttttgtttgtgactgagtctcg | | ccatccatccagctatccatcc | |
| | | ctctgttgcccaggctggagtgcaa | | atccatccatccatctaatcatt | |
| | | tagtgcaatctcggctcactgcaac | | catccatcaatccatccatcca | |
| | | ctctgcctcccgggctcaagcaatt | | tccatccatccatccatccatc | |
| | | ctcccgccccagcctcctgagtagc | | catccatctatccatccatcca | |
| | | tgggactacaggcatgcaccatgc | | atcatccatccatccatcgatc | |
| | | ccagctaatttttgtatttttagtagag | | aatcagtccatccatccatcta | |
| | | atggggtttcgccatgttggccagg | | atcatccatctaatcatccatcc | |
| | | ctggtctccaactcttgacctcaggt | | atccatccatccatccatccag | |
| | | gatccgtccgcctcggcctcccaaa | | tcatccatccattcatccatttaa | |
| | | gtgctggggggtgggggggggaa | | tcatccatccatccatcgatca | |
| | | tcaacaaattattctaagactcaact | | attggtccatccatccatctatc | |
| | | ggctatgtggaatttataaaatttattt | | catccatccatccatccatcta | |
| | | tatcaattaggcatagtgagattaat | | atcacccatccatccatccatc | |
| | | ggcaataactaacagaacaatgata | | catccaatcatctatccatccat | |
| | | acaatatactgtaataaaagtatgtg | | ccatctaatcatctatccatcca | |
| | | aatgttgtctctctcagaatattgtatt | | tccatccatccatccatccaat | |
| | | gtgtgtaatatttctgggtcatagttg | | catccatccatccatccatcca | |
| | | accacaggcaaccaaaactgcaga | | tccctccatctaatcatccatcc | |
| | | aagtgaaatcgaagagaagggcg | | ctccatccatccctccatatcc | |
| | | gactactgttctcctctcagggccaa | | atccatccatccatccatccat | |
| | | tttccagctaccaacattatgttactg | | ccatccaaccattcagcagtg | |
| | | gacatagagttagggagaaacatg | | aacctctactgtggtgtgttgg | |
| | | cagatccacatcgctctagaattcct | | aaagaagggaagtgtgagcc | |
| | | gccatatggctatgatagagacaag | | tggtcttgctcacagggaactc | |
| | | taacctcaaaagcacacacaattgt | | aaaacagagcattaggagga | |
| | | aaatatagtgaaataactggacggg | | gactcaggatcttacagccttg | |
| | | gatgagttttgaacatttattgtctttgt | | agggaggtgacccaggctag | |
| | | tttatttaattaaattaattaattaattaa | | aggaagtcaggagctcaaag | |
| | | ttttttgagacagagtctggctctgcc | | actagctggggagccaggga | |
| | | ccccaggctggagtgcagtggtgc | | cacacacatggattataattag | |
| | | gatctaggctcactgcaaccccttc | | tggctctgcaaggctgcaagt | |
| | | ctcctgggttcaagcaattctcccgc | | gatcaagggcagactgcggg | |
| | | ctcagcctctggagtagctgggatc | | gtaaaggaaggagagaagac | |
| | | acaggtgtgtgccaccacacccag | | tctgggctttgttaggtggaag | |
| | | ctaatttttgtatttttagtagagacgg | | ggctttctgcaggaggccagg | |
| | | ggtttcgccatgttggccaagctgg | | ccagagaagccagcctgatg | |
| | | cctccaactcctgacctcaggcgat | | aggctttgagaggacctgaaa | |
| | | ccacccacctcagcctctcaaagtg | | ctggagatgggatttaatggc | |
| | | ttgggattacaggcgtgagtcactg | | cagagagaaggaggcagca | |
| | | caccctggcctgtctttgttttaaatat | | ggcttgtcatgtaggcaaatac | |
| | | aacttattttatttaattgtaagtttatat | | acgcaagcttgtagagtaatta | |
| | | ggtttaattttttggcaatacaaataat | | ccaggcagagagctgagga | |
| | | aatttctttacccccagctttattgag | | gagcaccctgttatgttccctta | |
| | | gtacagctgacatatataattgtataa | | g | |
| | | atttaagatgtacaatgtgaggatttg | | (SEQ ID NO: 11) | |
| | | atataggtatatattgtgaaatgatttc | | | |
| | | caatttccaattaacacacccatcac | | | |
| | | ctcacatagttgccatttctttgtgtgt | | | |
| | | ggtgagaacatttatgatctattctctt | | | |
| | | agcaaatttctagtacacaacacgg | | | |
| | | tattgttcactatagtcatcatgctgtc | | | |
| | | cattagatccccagaacttattcatct | | | |
| | | tattcattgatttagttctgaataatgg | | | |
| | | ctgtgtttaacaaccagctcacaag | | | |
| | | cttcatgagaatttaacgatcagctct | | | |
| | | tgcgagctcttctgagctggctcca | | | |
| | | gcatgccactgcccccagcaagtg | | | |
| | | tgaggattccagtgtgagaccctat | | | |
| | | ggtgaaaggtacagggtgaatcac | | | |
| | | agggagaagagaacctggggctg | | | |
| | | gagcagaaacagaaggcttcctgg | | | |
| | | aggaggcaggatccagtgttgagc | | | |
| | | cttggagcatcaggaggatttggct | | | |
| | | gggggaagggagagtggcccaa | | | |
| | | gtgaagagacagttagggtggggg | | | |
| | | gtgggagagggtgggaagcagga | | | |
| | | agatcagggtgccgtgaatatgtgc | | | |
| | | ttgcaccttacacttgaattatgtttatt | | | |
| | | tccttatag | | | |
| | | (SEQ ID NO: 9) | | | |
| STK31 | aactctggtgg | gtaagtctaacttcttcttacttgtgg | ggctattctgtgg | gtaaagttcttatgctaatctctt | tctgatcctatggcttatct |
| (SEQ ID | tctccttacaat | gagattcagcagcataaaggatagt | atgttgacacaga | gcagaaaccattttatcttgca | gatggtcccatactaccct |
| NO: 2) | gagcttggaa | ggtgctgttacatttcaagaaagaaa | agccaaggtgatt | gaaactattttatttaaaataaat | agggcaaacctgaatgct |
| | cgagatcttctt | aaacctcactgttagttttctaaagtg | gagagagcagc | tttagctccaggttttagagca | gttcaagccaacatgcctt |
| | gatgctgagc | tataagttgactggcatgataattgtg | cacctaccataga | gcagttcattccccttaacataa | taaattcagaa |
| | ccatgaagga | cttgtagtttggttgccagaactaatt | gcttggagagaa | aagaaaaattatcccatagaat | (SEQ ID NO: 17) |
| | acttagcagca | ttcacttttaaagtttccctcaattgaa | gctgaaggagac | caatgtatgctaaaaatatgttg | |
| | agcgtcctttg | caatgagaacacttggacacaggg | tcagggttactgc | aagaagtttttacttggagtcac | |
| | gtacgttctga | cggggaacatcacccgtgggggc | cattgatattcctg | attctctgttttgcttcttag | |
| | ggttaatgggc | ctgtcatggggtggggggcaggg | tttttatgtaag | (SEQ ID NO: 16) | |
| | agataattctgt | ggagggatagcattaggagaaata | (SEQ ID NO: | | |
| | taaag | cctaatgtaaatgatgagttaatggg | 15) | | |
| | (SEQ ID | tgcagcaaaccaacatggcacatgt | | | |
| | NO: 13) | atacatatgtaacaaacctgcacgtt | | | |
| | | gtgcacatgtaccctagaacttaaa | | | |
| | | gtataataataaaaataagtatataa | | | |
| | | aagtcattaaataaagctttgtctcac | | | |
| | | aagaaaaaaaaataaagtttccctc | | | |
| | | atttccctaaatagaatatgtccttaat | | | |
| | | aggatatactttcctttttttttgggtga | | | |
| | | acttattatagtcatccaatatcttttta | | | |
| | | aaattcttgattttcttacatttctcctt | | | |
| | | gaaatctcagctgtgttatcagtaca | | | |
| | | atcttctactctttaatttcttcttctcat | | | |
| | | tgatctgggaaactgttaatcatgat | | | |
| | | aataagataattaatttggccttagtt | | | |
| | | gctacacattctagaatgtgtagcag | | | |
| | | gctacaggtagaattgcactttccttt | | | |
| | | tccaaattatagcattcaaagaagat | | | |
| | | agaaaagtacttataaaattaaaaaa | | | |
| | | taaaattagatttgatttaacttaaaat | | | |
| | | aatgaattatgtttaaattggaatgat | | | |
| | | gtataattataatgagattttcatcttg | | | |
| | | gaattacgagtgcctctttctttggta | | | |
| | | caag | | | |
| | | (SEQ ID NO: 14) | | | |
| USP25 | gtgtcacaaca | gtaatgtgctttcttagaggttaaact | tgcaggctgtatg | gttagtgaacagtataaagact | gagtggcatcaggattata |
| (SEQ ID | taatgaaagtt | acagatttagggattctgtaattcact | aaaattccagcaa | ttttaataaatggttctgggaaa | ggaaattcagggaaacaa |
| NO: 3) | gctcaagcca | gatattcctttcgtttcagcccaactg | gatgttttatggag | taaataaaatttgatttcttcttta | ctatgtatctcataattggg |
| | aactggaaat | aagttgaatttgttatgaatgatgagt | cttgataagccaa | ttccatatgcaaaaattcatttc | ctagaaaattttcaaagag |
| | gataaaacctg | aaaaaaattaagtttccaagtcaatg | ctgaaaaatgtta | aattgtgaaagactcctgtgta | aaa |
| | aagaagtaaa | ctcaagtaattaataattttatatgtga | cggaaatgcaaa | aagctgtttagaagtcaacata | (SEQ ID NO: 22) |
| | cttggaggaat | ttttaggattaacatgatatgatagca | gatcccagtccac | ggtagaagcccaaagacattc | |
| | atgag | tgtagaccaaaaagtttaaaatcaaa | ctcccatctctga | atttatccataagaggcactca | |
| | (SEQ ID | aaaggaagatttttggttttttatttca | caagcaa | ctgaccagatatttttgaccag | |
| | NO: 18) | atactaattctcaagtactttttaaatg | SEQ ID NO: | gatatgtaatctagagccaga | |
| | | aacttttctagcttaaaaacagaata | 20) | gaaattcaaaatttgttttctctt | |
| | | acaaatgcattgttaattttgaaatca | | gaccctgaggagcaagtcaa | |
| | | acttagaaattgctgttacataatacc | | gtcatcttttagctaataatgact | |
| | | aacaagtaatatttatcaagacccta | | ccaaatctgtgccctccacatt | |
| | | ctatatgccaggcatttgtctaagcc | | gattgagagctcttcccttgtcc | |
| | | ctttacatatattaactgatttaattca | | ccaagattcgtgttcatggaac | |
| | | gacagtaacactgtaagataggtag | | ttgatttttaattgtgacttgattc | |
| | | tgttcttattcccatttgacacacgag | | tgcaatgcttccttcagtttgctt | |
| | | aaaataaggcatagagcagtgaag | | caacagattggactcattatgtt | |
| | | taacctgcctaaggttttcacagcca | | ttttttcataaaatcttctcatatt | |
| | | caagtttacagcttggctaatagaac | | ctaactgcagggctgttaaatc | |
| | | ctacattcttaactgttgcactacact | | atacaagtatttaaatgacttgtt | |
| | | gtgcaactgtaccctgtgggctgtat | | tttagattctgcatattcctccta | |
| | | tttttccttcactttgattcatcagatat | | tctttgtcttgctctaagttcagg | |
| | | attttggggaagttaattcattttagtc | | gttcagtaaaccatagcctgca | |
| | | cctatatgccatatatgtttgtcatga | | gaccagcttaccacctgtttttg | |
| | | gacagtgttgtgtgtgagagagcac | | taaataaacttttattggaacat | |
| | | ttacataggtacttgtacatgtgcttg | | ggccatttgtatacatattgtct | |
| | | tgggatttttttgtgcacaaaattatttt | | gtggatactttcacactgcagtt | |
| | | ttgtcaactttggctttaaaatattcttt | | acagagttgagtaactgaaga | |
| | | attttaattgatgaattcatacaccta | | agatgaggcttgcaaagtcta | |
| | | gtatttcttcacatttttaaatttaatctt | | aaatattatctggccttttacag | |
| | | atgcattgtcttttttttaatttataagtc | | aaaaagtttgcctactcacaac | |
| | | tagtaaattttaagttgttttcttgggt | | ttctaaggtgattctaatgacaa | |
| | | gcctttgcttaatatttagttccatttac | | caaggaatgacagtttgaata | |
| | | aaacttaattatggtactttagattcc | | ccttccccagaaaagctaactt | |
| | | ctaaataattagtgtaattcactacac | | catacttgcaggacaaatttta | |
| | | aaacagggaagaggaaagagaga | | cctttatttgttatgaagatctac | |
| | | gaaaacactcattattgatgaaaacc | | taatagcatgaaaattatgtctt | |
| | | ttagctagtttggcgtataaggtaact | | gaaaattaaaattaagactgga | |
| | | accttaattcactaaagagtgtctcta | | tgtggtggctcatgcttataatc | |
| | | aaaacaaaaataggaaataataaaa | | ccagcacttttggaaatcaag | |
| | | ctgcagcaaatatatagaattgtgag | | gcacagggagttgtttgaggc | |
| | | cactgaaagcttaaaatttttgttgtc | | caggaatttgagaccggcctg | |
| | | aaaagcaaggcaatgaaaataagc | | gccaacatagcaagaccctgt | |
| | | gaaaagtaaaatactggaaaagaa | | ctatacaaaacagattaagtaa | |
| | | gaaacaaagacatcattatttgtagtt | | ttaactggaactgggagcatg | |
| | | ttttttaccttgaaattcaaataaaatc | | cacctgtaattcaagctacttg | |
| | | tacaactgatgtattagaactagtaa | | ggagacagaggtaggaggat | |
| | | gacagctttgcagcatgcaaacttgt | | tgctggagcccagaaggtca | |
| | | gtcagacatcaattttattcccttttgc | | agtctgcagttagccgtgatcg | |
| | | cagtaaaaaaagtatgagaaagtat | | tgccactgcactgcagactgg | |
| | | aaaagtgagaccatttacaatagtat | | gcaactgagtgagaccctgtc | |
| | | ctgaaactatggagtatatagaaata | | tccaaaaaaaaaaaaaaaagt | |
| | | aatttaacaaaaggtgcgtgtgactt | | ttatattcaaggagaatatcgtt | |
| | | ctgtggggaaaattataaaacttatt | | ggctaataactatgtttcttatttt | |
| | | gggagacattaacaaagacctaaat | | tgtttataatgtttatataaaagc | |
| | | aaatgggcatacatatggtatttgga | | cttaacacttttgttatcagtatt | |
| | | aaacttgatattattaatatatagatat | | cactttccaatttatcctacattg | |
| | | cacttttcccccaaatgctttataaatt | | aatttcagttatttttcttgagtca | |
| | | cag | | attattataaagtaagctaaga | |
| | | (SEQ ID NO: 19) | | aaatcttgactttggtatcacctt | |
| | | | | agaattatttagtcctattacatg | |
| | | | | ccctatagttgttttgggggtg | |
| | | | | gggaggggcattgaaatgtg | |
| | | | | aactgcagttattcttttaccata | |
| | | | | tctcatgtaaataaactattcgc | |
| | | | | ctacacacttaaagtcaaattta | |
| | | | | ccacatatttgcgaagttaaaa | |
| | | | | ggttaaataaatctaacttttttt | |
| | | | | agtttcttaaaattatgtattaag | |
| | | | | tatgtgtccaagctttgattcat | |
| | | | | actcagcgtaatatttcattttct | |
| | | | | tccatcagttgatgtagatataa | |
| | | | | taatagttcaataatatgatttta | |
| | | | | tactgatgaatttttgagatgac | |
| | | | | ctgattgattatcctgcctttata | |
| | | | | tttctgttatctgctagtaaaatc | |
| | | | | tttattgtttaaaacatttcatatc | |
| | | | | catgcctaataatttttagattta | |
| | | | | atgttgtattaataaatagtcttg | |
| | | | | tgtgccagaaagtaaagtcttg | |
| | | | | ttttagttctgatgtatcttcatttt | |
| | | | | gaactctttatttctattaattca | |
| | | | | gatagtagcgaaacagtgcat | |
| | | | | tactttttgtacagtttcatagta | |
| | | | | ctttttaaatcttactctgaacatt | |
| | | | | tttaatattttctttcacctacag | |
| | | | | (SEQ ID NO: 21) | |
| GTF2B | gatcgaacaa | gtaagtttttagctttgtttttttgtgttt | tttgcagttacatg | gtgagtatttattttttatgttttct | aaatatgtgccgtatcacg |
| (SEQ ID | ataatttattca | gtttgtttgtttgtttgagatagggtct | tgaatatgtgataa | tacgtaaaagtgaagaggtga | aatttctaagaaagaaatt |
| NO: 4) | agcaagtatat | cactttgtcgcccaggccagagtgc | tttgttcagtgttgc | tggcctttatcatcatcttttttgt | ggtcggtgttttaaacttatt |
| | gaacagaaga | agtggcgcaatcttgactcactgca | tcactgacttcca | tgtttttataactaaaacaactttt | ttgaaagcgctagaaacc |
| | gcctgaaggg | acctctgcctcccaggttcaagctat | ctaccaaagctca | gttaaatttcttctgaaagtgtgt | agtgtggatttgattacaa |
| | aagagctaat | actcctgcctcagcctcctgagtag | gtttagctgaaga | ggtggttgcatctgggaatag | ctggggacttcatgtccag |
| | gatgctatagc | ctgggattacaggcatgtgccacca | ctatacttaaccct | cagggtcagatattttatctcat | gttctgttccaacctttgtct |
| | ttctgcttgtct | cacctggttaacttttgtatttttagta | ttattcatcca | tgagctacagcccataatcgc | tcctaaacaagtacagatg |
| | ctatattgcctg | gagacagggtttcgccatgttggcc | (SEQ ID NO: | agggatttcatgcacacatga | gcagctacacatatagcc |
| | tagacaagaa | aggctggtctcaaacttctgacctca | 25) | cattgtgttgcctaagaggcat | cgtaaagctgtggaactg |
| | ggggttcctag | ggtgatctgcccactttggcctccca | | cctttagcttgagtattttctttca | gacttggttcctgggagg |
| | gacatttaaag | aagtgctagaattaacaggcgtgag | | attccttggtaacactgcttaca | agccccatctctgtggca |
| | (SEQ ID | gcactgtgcccatcctaaaataacat | | acagattaatacataaacatca | gcggcagctatttacatgg |
| | NO: 23) | tttctacatcatattaaagaaacaga | | aagaatttttcatttaaagaaatt | cctcacaggcatcagctg |
| | | ccaatgtcaggcagctttgacactta | | tagttgtatgaattaatccttag | aaaagaggacccaaaaa |
| | | gaagctaagtcatctttattgtgacta | | agcagttaatattttgaaagatt | g |
| | | cagtaatccacttgagcatcatttgc | | aagacctaaatttagccaggc | (SEQ ID NO: 27) |
| | | caatgcctacttatttcattccttgaca | | acgatggctcacaccggtaat | |
| | | gcaaattcatagcatgttcttttaggc | | ctcagcactttgtgaggccga | |
| | | tgttttcccatgtagactcattttagtc | | ggcaggtagatcacgaggtc | |
| | | cacgtaagcccactatgttcttttcta | | aggagttcaagaccagcctg | |
| | | gtgagatcttttgatggactgttgcttt | | gccaagatggtgaaaccccat | |
| | | gtgggctttcagaaggcctataatc | | ctctactaaaaatacaaaaaat | |
| | | gtaaaccaaagtaggccgtttatac | | tagccgggtgtggtggcagg | |
| | | atttggtttacatggaaacagtttcca | | cgcctgtaatcccaactactcg | |
| | | tttctagcactggtctctgtagatgag | | ggtggcaggagaatcgcttga | |
| | | ggagtcaagctttagaaagtatcag | | acccagggggcagaggttgc | |
| | | tgaaggccaggtatggtgtctcaca | | agtgagccaagatggtgtcac | |
| | | cctgtaatcccagaactttgggagg | | tgcactccagtctggacgaca | |
| | | ccgaggtggttgggtcatgaggcc | | gagtgaaatttcatctcaaaaa | |
| | | aggagttcaagaccagcctggcca | | acaaaaaaactctaaattgaaa | |
| | | atatggtgaaaccccgtctctactaa | | gagtgaagaacattaggcca | |
| | | aaatacaaaaattagccaggcgtg | | ggcgcagtggctcatgcctgt | |
| | | gtggtgcgcgcctgtagtcccagct | | aatctcagcactttgggaggc | |
| | | actcaggaggctgaggcagaaga | | cgaggtaggaggatcacctg | |
| | | atcgcttgaaaccgggaggcagag | | aggtcaggagttctagacagg | |
| | | gttgcagtgagccgagatcacgcc | | cctggccaacatggtgaaacc | |
| | | actacactccagcctgggcgacag | | ccatctctactaaaaatacaaa | |
| | | agtgagacttcatctcaaaaaaaaa | | agttagccaggtgtggtggtg | |
| | | aaaaaaaaagttatcagttaagttttt | | cacgcctgtagtcccagctact | |
| | | tctcttgaggtgtattgtttgcactttta | | cgggaggctgaggcaggag | |
| | | cacttcagttaagaactggtatttttc | | aatcacttgagcctaggaggt | |
| | | atttag | | ggaggttgcagtgagctgag | |
| | | (SEQ ID NO: 24) | | atcatgccactacccttagtct | |
| | | | | gggcgacagagtgagacttc | |
| | | | | atctcaaaaaaaaaggtgtgat | |
| | | | | gaacattaaagttaactgagaa | |
| | | | | atttgcagttaactctgtggga | |
| | | | | agtatcactgagatgattaggg | |
| | | | | tttcatctttaaggtatgattgaa | |
| | | | | tgttttacaattagaatgtatatt | |
| | | | | atgtttataattagaaatggtag | |
| | | | | attttaagatgaactatatggct | |
| | | | | taagtgaattgatttatttaatttt | |
| | | | | ttttcttttag | |
| | | | | (SEQ ID NO: 26) | |
| LYPD3 | atggaccccg | gtgggtctgctgcccggagaggtg | gagcgcaggcc | gtgagtggggacccccgggc | tccacggacaattctcgct |
| (SEQ ID | ccaggaaagc | gagggggtagagtgggggctctta | ctggagtgctaca | cacgcacaccactgcaggct | ggcagtgcggggttgcg |
| NO: 5) | aggtgcccag | gggaggaggctcctctggccccgt | gctgcgtgcaga | cctccggcccagccccgcct | gttcgggactccccggca |
| | gccatgatctg | acagagaaggctgtgggaaggatc | aagcagatgacg | ccttatagtcctttctggatctg | agaatgaccgcggcctg |
| | gactgcaggc | cccacataagcagagggcactgtg | gatgctccccga | gccacgcccctttaacgcggc | gatcttcacgggcttctgg |
| | tggctgctgct | ggcaaagttcccccatcctaagcaa | acaagatgaaga | cccacccctccctttcagcctg | cgttcatccagctgcagc |
| | gctgctgcttc | gggaggcagagggaagggtcctc | cagtgaagtgcg | gccgggaccctacgacacag | aatgcgctcaggatcgct |
| | gcggag | caccatgggccgtaggaagggttc | cgccgggcgtgg | ccttgcaggccaggccttctct | gcaacgccaagctcaac |
| | (SEQ ID | cccaccacggacaaggggagctg | acgtctgcaccg | attaacaattacaagcgcgag | ctcacctcgcgggcgctc |
| | NO: 28) | tgggcaaaattttcctatcctgaagc | aggccgtgggg | gagcagagagttgagaactg | gacccggcag |
| | | tgcagaaagaagtcccctgttccta | gcggtggagacc | ggtggggcggacggggaga | (SEQ ID NO: 32) |
| | | ggccgggcgcggtggttcatgcct | a | gctcgggtttggaacgaggtc | |
| | | gtaatctcagcactttgggaggccg | (SEQ ID NO: | acgtcctatgcactttttctgctc | |
| | | aggcaggcggatcatgaggtcaaa | 30) | caccaatgagacctccccatc | |
| | | cgatcgagaccagcctggccaaca | | cctgcctgtcaaccaggctgt | |
| | | tggtgaaaccctgtctctactaaaaa | | ctcctccgactcagctctttga | |
| | | tacaaaaattagctgggcgcggtag | | ggttccgcccccgttccgtga | |
| | | cgcgagcctgtagtcccagctactc | | ccccgctcctccctgcttcggt | |
| | | gggaggctgaggcaggagaatcg | | cgttctttccgctctgagcggc | |
| | | tttgaatccaggaggggaggttgca | | tctttgggggcgtccatccgcc | |
| | | gtgagccgagattgcgccactgca | | aattcctgcctgtcggtcctgt | |
| | | ctccagcctggcgacagagcgag | | ggtgttggcctatttgaggccc | |
| | | actccgtctcaaaaaaaaaaaaaag | | cgcccctcccagaccctaccc | |
| | | aaaaaaagaaaaaaagaaaaaagt | | aactctgggccacgccccatc | |
| | | gttccctgtcctgggcagagaagct | | tgagaccacgcccacagcta | |
| | | gtgggtagtgttctctcatttgagca | | atcccgcccttgtccctctccct | |
| | | gaagggggctgtgggaagggatc | | ag | |
| | | cctcactgagcacagtctactgtgg | | (SEQ ID NO: 31) | |
| | | gaaaagttctcccacacactgtggg | | | |
| | | aaaagttctctcattctgagcagaaa | | | |
| | | accgtgaggaggtatccttcctacc | | | |
| | | ccgggatgaggggcttcatggcgg | | | |
| | | ggatcgcctacagtgggcatacag | | | |
| | | ggctgtcggaaggatgtgcccaca | | | |
| | | gttggcatagtgggtgtgaaggaaa | | | |
| | | agggtccagtgattcccgtgcttcc | | | |
| | | agatttgtggaaagcttttccctactc | | | |
| | | ctgccctggaaaagtgtgtgcaata | | | |
| | | agactgtctaatcccgggacagag | | | |
| | | ggcatgggaaggactatttctccttg | | | |
| | | ggggtggacgctttagcaaggagg | | | |
| | | gctcaggtctattcggctccgctccc | | | |
| | | ccag | | | |
| | | (SEQ ID NO: 29) | | | |
| IK | gtgagccgttc | gtgagtattttgtgctaggaccatgg | gctggaatgcagt | gttccactacgcccggctaatt | atcaaaactcaccaatga |
| (SEQ ID | tccaacccttt | aaatgagttgggcaatgaatagaaa | ggtgcgatctcg | ttgtatttttagtagagatgggg | agacttcaggaaacttctc |
| NO: 6) | ggcccccgat | agtggtggtgatggtgaaagaattc | gctcaccacaac | tttctccatgttgatcaggctgg | atgacccccagggctgca |
| | ggccacgatg | tgaatgtcttgtcatcaaggatggta | ctccgcctcccg | tcttgaaatcccgacttcaggt | cctacctctgcaccacctt |
| | tggatgatcct | aaaaattatacccgctggccctttat | ggttcaagcaatt | gatccacccgcctctgcctcc | ctaagtcacgtcaccatga |
| | cactccttcca | ctctgtgagccttaactggatcagg | ctcctgcctcagc | caaagtggtgggagtacagg | (SEQ ID NO: 37) |
| | cca | ccaagtgcataagggtattagaagg | cttccgagtagct | cgtgagccaccgtgcctggc | |
| | (SEQ ID | gaaattcctgcaggaaaggtgttaa | gggattacaggc | ccccgataaaatcatctttgata | |
| | NO: 33) | gtttttttttttttttatacgtgttgtgtaa | at | acatccccaattccagtccattt | |
| | | ttacacattatataggaattacttcttc | (SEQ ID NO: | ctttttttttttcttttcttttcttttctt | |
| | | caatagcaaattaaaacataggaaa | 35) | tctttttttttttttttgagacggag | |
| | | taagggtaaaatcatctttttatttttttt | | tctcactgttacccaggctgga | |
| | | gagactgagtttcgctcttgttgctca | | gtgcagtgatgcaatcttggct | |
| | | g | | cactgcaacctctgcctcctga | |
| | | (SEQ ID NO: 34) | | gttcaagcggttctcctgcctc | |
| | | | | agcctcccaagtagctgggac | |
| | | | | tacaggcatgtgccaccatgc | |
| | | | | acgactgatttttttttttgtattttt | |
| | | | | agtagagacagggtttcactgt | |
| | | | | gttcgtcaggctggtcttgaac | |
| | | | | tcctggcctgaagtgatccac | |
| | | | | ccgcctcagcctcctaaagtg | |
| | | | | ctggcattacaggcgtgagcc | |
| | | | | accgtgcccagcctcagtcctt | |
| | | | | ttcttagaagtaaccacagtta | |
| | | | | ccttttttggcatatcttttcctcc | |
| | | | | cgttgcccaggctggagttca | |
| | | | | atggcattatttctgctcacagc | |
| | | | | aacctttgcctcctgggctcaa | |
| | | | | gcaattcttctgcctcagtttcc | |
| | | | | ccagtagctgggattacaggc | |
| | | | | atgccccaccatgcttggctaa | |
| | | | | tttttgtattagtagagacaggg | |
| | | | | tttcgctatgttggccaggctg | |
| | | | | gtcttgaactcctgacctcaaa | |
| | | | | tgatctgcccgtctcggcggc | |
| | | | | ccaaagtgctgggattacagg | |
| | | | | cgtgagccactgcgcctggc | |
| | | | | cctcctactcttttttaaatgcatt | |
| | | | | tatacacatacatgcatatctga | |
| | | | | ggaaaacacaacattgctttgt | |
| | | | | gatttaaaatatttgagttaaat | |
| | | | | gatattatgctgtatatatcatttt | |
| | | | | tgcaacttgcttctttttaaaccc | |
| | | | | tgctcagtgtttctgagattatct | |
| | | | | ttcagagcagtgacaagcatt | |
| | | | | ccctccttggctaaactttagtc | |
| | | | | aggttccccaaagagtcccatt | |
| | | | | tttcagcaggactcatccgtgg | |
| | | | | cctgctgagcacagttttaaca | |
| | | | | aagaaatcaaccaccgctcca | |
| | | | | cccccatacctaaccaagttg | |
| | | | | cttttagtaattttctataaattct | |
| | | | | cacttgtccctgttgtattcgga | |
| | | | | attaagttcagtctctctactcta | |
| | | | | ttacaatggtcttgactcttgtta | |
| | | | | cagtagtcttcaataaagtcttc | |
| | | | | cttggggagaagaggaacttg | |
| | | | | attagatatcaagttggattgg | |
| | | | | atatcaagagtgggaaaacttt | |
| | | | | ctgtgaactgacttaggattcct | |
| | | | | ttttaaaactgaggtcagcagg | |
| | | | | ccaaggaagagccctagtca | |
| | | | | gaagagagctttaaaggagtc | |
| | | | | tgactttaatttgatcaagagag | |
| | | | | gagtctttgttagtgtaccaatt | |
| | | | | aaaataatctcttgtaccattag | |
| | | | | tgctgtctttccctctttgggaa | |
| | | | | acaccatcatagtttagtcagt | |
| | | | | ctccagctaataaacgcttagt | |
| | | | | ctgtttcagactgattcttttcttt | |
| | | | | tttttatttttttgagacagagtct | |
| | | | | tgctctttttgcccaggctgga | |
| | | | | gtgtaatacctagatctcggct | |
| | | | | cactgcaaactccgcctccca | |
| | | | | ggttcaagcgattttcctgcctc | |
| | | | | agcctcctgagtagctaggatt | |
| | | | | acaggcgcccaccacaacgc | |
| | | | | ccagctaatttttttgtatttttag | |
| | | | | tagagatgggatatcaccatgt | |
| | | | | tggccatgctggtcttgaactc | |
| | | | | ctgacctcgggtgatccgcct | |
| | | | | gcgtcagcctcccaaagtgcg | |
| | | | | gcgattataggtgtgagccact | |
| | | | | gcacccggccagactgattat | |
| | | | | tttcaaggctataactgaattaa | |
| | | | | atttacaggccttcttatatacat | |
| | | | | atgtcagtaatggaccaggct | |
| | | | | aaatataaataaatggagttgc | |
| | | | | tggctcataggatatatatactt | |
| | | | | aaattttaacaaatactgcaaa | |
| | | | | attgcccttcaaattggttatact | |
| | | | | ggtttacattctcatcagcattat | |
| | | | | atgagagagtccccttcccaa | |
| | | | | attctcactcacacttgatttatc | |
| | | | | aaactttctggtagttaacactc | |
| | | | | tgatgcattaaaagtggcatgg | |
| | | | | catatctggtcgggcgtggtg | |
| | | | | gcttacacctataatcccagca | |
| | | | | ctttgggaggctgaggtgggc | |
| | | | | agatcacctgaggtcagaagt | |
| | | | | ttgggaccagcctggccaaca | |
| | | | | cctctaataaaaatacaaaaat | |
| | | | | tagctgggtgtgttggcgggc | |
| | | | | acctgtattcccagctacttgtg | |
| | | | | aggctgaggcaggagaatta | |
| | | | | cttgaacccaggaggcagag | |
| | | | | gttgcagtgggctgagatcat | |
| | | | | gccactgcactccagccggg | |
| | | | | gcaacagagggagactctgt | |
| | | | | cttaaaaaaaaaaaaaaaaag | |
| | | | | ctgggtgcggtggctcatccc | |
| | | | | tataatcccagcactttgggag | |
| | | | | gccgaggtgggtggatcaca | |
| | | | | aggtcaggagttcaagacca | |
| | | | | gcctggccaatatgggtgaca | |
| | | | | gtgagactccgtctcaaaaaa | |
| | | | | aaaaaaacagtgtcatatcttt | |
| | | | | gttttaatttgtatttccctgattt | |
| | | | | ctgttaatttaatcatgatttttac | |
| | | | | ttgttcctttttcatttcttattgag | |
| | | | | tcctaatatttaaaatctttgcctt | |
| | | | | ttcttctag | |
| | | | | (SEQ ID NO: 36) | |
| TEX14 | tggaggtgaa | gtaagttcacttgcgggaatcagga | gtccctttgtccaa | gtaggccatcactgcctactg | gtatcaggcacccatctcc |
| (SEQ ID | agaaatggca | taggtgaactcaatcaagtatcagtc | aggaagtaaaat | cccatgtctctgcattgtattcta | cagacaaaaggagcaac |
| NO: 7) | aagaaagcag | gggcgaggtggctcagcctgtaatt | aatcaaatactga | agctgaaattaaattctcccag | cagagcatagtgaagcct |
| | ctactggaca | ccagcactttgggaggtgaggcga | ccctggttttggc | aatttatttagaatgtattctaag | tccaagcaagttctgacac |
| | gctcacagtac | gcggatcacctgagcccaggaattt | gaggaatcgatc | ctggcattaaatttttccagaga | attggtggctgtagagaa |
| | ctccttggcat | gggaccagcctggtcaacatgatg | cttctttacctggg | gtctatgctggaggaacctag | atcttacagt |
| | cctcagagta | aaacctggtctctacaaaaaaaaaa | gatagag | agacacatgacatttattgtcat | (SEQ ID NO: 42) |
| | gtctgactttag | aaaaaaaaaaaaaaaacattagctg | (SEQ ID NO: | cggtttatagatttacaggttaa | |
| | agagcgagg | ggcgtggtggcacacgcctctagtt | 40) | aaaaaatgaaggctgtatatttt | |
| | ctgaaaatga | ccaggtacccaggaggctgaggtg | | cagttgataatttgcttataatca | |
| | gcccgacgcc | ggaggatcacttggacctgagagg | | gggcatggatattgtttcacag | |
| | ctgctgcagc | tggaggttgcagtgagctgtgatca | | ttgtttataagttaaagaaagat | |
| | cccccattagg | tgaaactgcactccagcttgggtga | | acatctaaacttgtcatttgtctt | |
| | agcccagaaa | cagtgagaccctgtctcaaaataaa | | aggcagaaaatctattggcttg | |
| | acacggattg | aataaaagtaaaaaaaagttatcag | | ggtattatttgtatttagaatctg | |
| | gcagcgagtt | ctctcaaaaaagtaaactttccagca | | tttcttgtcaagcatcactctcg | |
| | attgagtatcat | tattttttttggtttggtcctctctccttt | | aatggggggcagatgatcca | |
| | agggaaaatg | cctacttttctggaaaccttgaattcc | | gggctgcattttggggtcagg | |
| | atgagcccag | aggggaagacagtatagccctatat | | ctaaactaacagtctctactctc | |
| | aggaaatggc | ctcattgcatttcccacaggcaaaca | | tggactgaacctaacgtatgg | |
| | aagtttgacaa | gatccagacactgatcagtttttatat | | gacatggccactcttggcaga | |
| | gacgggcaac | tactgaaacag | | atatgtatacatctcttctttcat | |
| | aatgactgtga | (SEQ ID NO: 39) | | gccacctttatcctcttccatgtt | |
| | cagtgaccag | | | gtctgaatatcactgaatgactt | |
| | catggcagac | | | tgaccctgactgtgttacaatc | |
| | agcccaggct | | | attag | |
| | tggaagcttca | | | (SEQ ID NO: 41) | |
| | cca | | | | |
| | (SEQ ID | | | | |
| | NO: 38) | | | | |

In some aspects, the expression cassette further comprises an untranslated region (UTR), a microRNA binding site, a polyA (pA) sequence, an intron sequence, or any combination thereof.

In some aspects, the expression cassette comprises a poly (A) (pA) sequence.

In some aspects, the pA sequence is a synthetic pA sequence, a bovine growth hormone (bGH) pA sequence, or a human growth hormone (hGH) pA sequence.

In some aspects, the upstream exon, the upstream intron, the alternatively spliced exon, the downstream intron, and the downstream exon, are derived from TRPV3. TRPV3 is also known as Transient Receptor Potential Cation Channel Subfamily V Member 3 (see ncbi.nlm.nih.gov/gene/162514). In some aspects, the alternatively spliced exon is derived from a sequence between exon 13 and exon 15 of TRPV3. In some aspects, the upstream exon, the upstream intron, the alternatively spliced exon, the downstream intron, and/or the downstream exon has been modified relative to WT TRPV3. In some aspects, the upstream exon, the upstream intron, the alternatively spliced exon, the downstream intron, and/or the downstream exon have been shortened relative to WT TRPV3.

In some aspects, the upstream exon, the upstream intron, the alternatively spliced exon, the downstream intron, and the downstream exon, are derived from STK31. STK31 is also known as Serine/Threonine Kinase 31 (see ncbi.nlm.nih.gov/gene/56164). In some aspects, the alternatively spliced exon is derived from a sequence between exon 18 and exon 20 of STK31. In some aspects, the upstream exon, the upstream intron, the alternatively spliced exon, the downstream intron, and/or the downstream exon has been modified relative to WT STK31. In some aspects, the upstream exon, the upstream intron, the alternatively spliced exon, the downstream intron, and/or the downstream exon have been shortened relative to WT STK31.

In some aspects, the upstream exon, the upstream intron, the alternatively spliced exon, the downstream intron, and the downstream exon, are derived from USP25. USP25 is also known as Ubiquitin Specific Peptidase 25 (see ncbi.nlm.nih.gov/gene/29761). In some aspects, the alternatively spliced exon is derived from a sequence between exon 22 and exon 23 of USP25. In some aspects, the upstream exon, the upstream intron, the alternatively spliced exon, the downstream intron, and/or the downstream exon has been modified relative to WT USP25. In some aspects, the upstream exon, the upstream intron, the alternatively spliced exon, the downstream intron, and/or the downstream exon have been shortened relative to WT USP25.

In some aspects, the upstream exon, the upstream intron, the alternatively spliced exon, the downstream intron, and the downstream exon, are derived from IK. IK is also known as IK cytokine (see ncbi.nlm.nih.gov/gene/3550). In some aspects, the alternatively spliced exon is derived from a sequence between exon 2 and exon 3 of IK. In some aspects, the upstream exon, the upstream intron, the alternatively spliced exon, the downstream intron, and/or the downstream exon has been modified relative to WT IK. In some aspects, the upstream exon, the upstream intron, the alternatively spliced exon, the downstream intron, and/or the downstream exon have been shortened relative to WT IK.

In some aspects, the upstream exon, the upstream intron, the alternatively spliced exon, the downstream intron, and the downstream exon, are derived from TEX14. TEX14 is also known as Testis Expressed 14, Intracellular Bridge Forming Factor (see ncbi.nlm.nih.gov/gene/56155). In some aspects, the alternatively spliced exon is derived from a sequence between exon 18 and exon 19 of TEX14. In some aspects, the upstream exon, the upstream intron, the alternatively spliced exon, the downstream intron, and/or the downstream exon has been modified relative to WT TEX14. In some aspects, the upstream exon, the upstream intron, the alternatively spliced exon, the downstream intron, and/or the downstream exon have been shortened relative to WT TEX14.

In some aspects, the upstream exon, the upstream intron, the alternatively spliced exon, the downstream intron, and the downstream exon, are derived from GTF2B. GTF2B is also known as General Transcription Factor IIB (see ncbi.nlm.nih.gov/gene/2959). In some aspects, the alternatively spliced exon is derived from a sequence between exon 5 and exon 6 of GTF2B. In some aspects, the upstream exon, the upstream intron, the alternatively spliced exon, the downstream intron, and/or the downstream exon has been modified relative to WT GTF2B. In some aspects, the upstream exon, the upstream intron, the alternatively spliced exon, the downstream intron, and/or the downstream exon have been shortened relative to WT GTF2B.

In some aspects, the upstream exon, the upstream intron, the alternatively spliced exon, the downstream intron, and the downstream exon, are derived from LYPD3. LYPD3 is also known as LY6/PLAUR Domain Containing 3 (see ncbi.nlm.nih.gov/gene/27076). In some aspects, the alternatively spliced exon is derived from a sequence between exon 1 and exon 3 of LYPD3. In some aspects, the upstream exon, the upstream intron, the alternatively spliced exon, the downstream intron, and/or the downstream exon has been modified relative to WT LYPD3. In some aspects, the upstream exon, the upstream intron, the alternatively spliced exon, the downstream intron, and/or the downstream exon have been shortened relative to WT LYPD3.

### III. Splicing Modifiers or Modulators

Certain aspects of the disclosure are directed to expression constructs comprising a gene of interest which is regulatable (e.g., regulated) by a molecule (e.g., a splicing modifier or modulator). In some aspects, the molecule promotes the inclusion of an alternatively spliced exon. In some aspects, the molecule is kinetin. In some aspects, the molecule is homocarbonyltopsentin.

Certain aspect of the disclosure are directed to increasing AAV production by contacting or culturing a host cell with the molecule (e.g., an indole alkaloid). In some aspects, the molecule is a topsentin alkaloid or derivative thereof (e.g., Homocarbonyltopsentin, Topsentin, Bromotopsentin, Bromodeoxytopsentin, Deoxytopsentin, and any combination thereof). In some aspects, the topsentin alkaloid or derivative thereof is homocarbonyltopsentin. In some aspects, the molecule is homocarbonyltopsentin.

### Kinetin

Kinetin or Drug K is a member of the class of 6-aminopurines that is adenine carrying a (furan-2-ylmethyl) substituent at the exocyclic amino group (PubChem CID 3830). Kinetin is a member of furans and a member of 6-aminopurines. In some aspects, kinetin has a role as a geroprotector and a cytokinin.

In some aspects, kinetin has been shown to increase inclusion of exon 20 in IKBKAP (see Hims M M, et al., J Mol Med (Berl). 85 (2): 149-61 (February 2007)).

In some aspects, kinetin is represented by the structure:

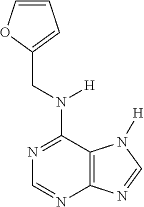 [SMILES: c1coc(CNc2ncnc3nc[nH]c23)c1]

### Homocarbonyltopsentin

In some aspects, homocarbonyltopsentin, or Drug H (see PubChem CID 11326167), binds to TSL2 and converts from it from a pentaloop to triloop conformation. TSL2 is a RNA structure that regulates exon skipping, promoting skipping in the pentaloop formation (See Garcia-Lopez, A., et al., Nat Commun 9, 2032 (2018)), thus homocarbonyltopsentin leads to exon inclusion.

In some aspects, homocarbonyltopsentin is represented by the structure:

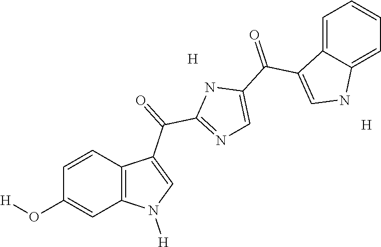

Indole alkaloids are a class of alkaloids containing a structural moiety of indole. In some aspects, the idole alkaloid is a topsentin alkaloid or derivative thereof (e.g., Homocarbonyltopsentin, Topsentin, Bromotopsentin, Bromodeoxytopsentin, Deoxytopsentin, and any combination thereof). In some aspects, the topsentin alkaloid or derivative thereof is homocarbonyltopsentin.

### IV. Delivery Vectors

In some aspects, provided herein is a delivery vector comprising any of the expression cassettes disclosed herein. In some aspects, the expression cassette is flanked by inverted terminal repeat (ITR) sequences.

In some aspects, the delivery vector is selected from the group consisting of a viral vector, a plasmid, a lipid, a protein particle, a bacterial vector, a lysosome, a virus-like particle, a polymeric particle, an exosome, and a vault particle.

In some aspects, the vector is a viral vector.

In some aspects, the vector is a lentiviral vector, an adenoviral vector, an adeno-associated viral (AAV) vector, or a retroviral vector.

In some aspects, the vector is an adeno-associated viral (AAV) vector.

In some aspects, the vector is a lentiviral vector.

In some aspects, provided herein is a cell comprising any of the expression cassettes disclosed herein or any of the delivery vectors disclosed herein.

In some aspects, provided herein is a recombinant viral vector (e.g., rAAV) comprising any of the expression cassettes disclosed herein or any of the delivery vectors disclosed herein.

### IV.A. Non-Viral Vectors

In some aspects, the expression cassettes can be administered using a non-viral vector. “Non-viral vector,” as used herein is meant to include naked DNA, chemical formulations containing naked DNA (e.g., a formulation of DNA and cationic compounds (e.g., dextran sulfate)), and naked DNA mixed with an adjuvant such as a viral particle (i.e., the DNA of interest is not contained within the viral particle, but the transforming formulation is composed of both naked DNA and viral particles (e.g., AAV particles) (see e.g., Curiel et al., Am. J. Respir. Cell Mol. Biol. 6:247-52 (1992)). Thus the “non-viral vector” can include vectors composed of DNA plus viral particles where the viral particles do not contain the DNA of interest within the viral genome.

In some aspects, the non-viral vector is a bacterial vector. See e.g., Baban et al., Bioeng Bugs., 1 (6): 385-394 (2010).

In some aspects, the expression cassettes can be complexed with polycationic substances such as poly-L-lysine or DEAC-dextran, targeting ligands, and/or DNA binding proteins (e.g., histones). DNA- or RNA-liposome complex formulations comprise a mixture of lipids which bind to genetic material (DNA or RNA) and facilitate delivery of the nucleic acid into the cell. Liposomes which can be used in accordance with the disclosure include DOPE (dioleyl phosphatidyl ethanol amine), CUDMEDA (N-(5-cholestrum-3-β-ol 3-urethanyl)-N′,N′-dimethylethylene diamine).

Lipids which can be used in accordance with the disclosure include, but are not limited to, DOPE (Dioleoyl phosphatidylethanolamine), cholesterol, and CUDMEDA (N-(5-cholestrum-3-ol 3 urethanyl)-N′,N′-dimethylethylenediamine). As an example, DNA can be administered in a solution containing one of the following cationic liposome formulations: Lipofectin™ (LTI/BRL), Transfast™ (Promega Corp), Tfx50™ (Promega Corp), Tfx10™ (Promega Corp), or Tfx20™ (Promega Corp). The concentration of the liposome solutions range from about 2.5% to 15% volume:volume, preferably about 6% to 12% volume:volume. Further exemplary methods and compositions for formulation of nucleic acids (e.g., DNA, including DNA or RNA not contained within a viral particle) for delivery according to the method of the disclosure are described in U.S. Pat. Nos. 5,892,071; 5,744,625; 5,925,623; 5,527,928; 5,824,812; 5,869,715.

In some aspects, protein particles can be used in accordance with the disclosure for polymer-based gene delivery. See e.g., Putnam et al., PNAS 98 (3): 1200-1205 (2001).

In some aspects, the expression cassettes can be administered as a chemical formulation of DNA or RNA coupled to a carrier molecule (e.g., an antibody or a receptor ligand) which facilitates delivery to host cells for the purpose of altering the biological properties of the host cells. The term “chemical formulations” refers to modifications of nucleic acids to allow coupling of the nucleic acid compounds to a carrier molecule such as a protein or lipid, or derivative thereof.

### IV.B Viral Vectors

In some aspects, the expression cassettes can be administered using a viral vector (e.g., a recombinant viral vector). In general, recombinant viral vectors used in accordance with the disclosure can comprise a viral particle derived from a naturally-occurring virus which has been genetically altered to render the virus replication-defective and to express a recombinant gene of interest in accordance with the disclosure. Once the virus delivers its genetic material to a cell, it does not generate additional infectious virus but does introduce exogenous recombinant genes into the cell, preferably into the genome of the cell.

In some aspects, the viral vector is a retrovirus, an adenovirus, an adeno-associated virus (AAV), a herpes simplex virus (HSV), a cytomegalovirus (CMV), a vaccinia or a poliovirus vectors. In some aspects, retroviral vectors are less preferred since retroviruses require replicating cells and secretory glands are composed of mostly slowly replicating and/or terminally differentiated cells. In some aspects, adenovirus and AAV are preferred viral vectors since this virus efficiently infects slowly replicating and/or terminally differentiated cells. In some aspects, the delivery vector (e.g., viral vector) is selected from the group consisting of an adeno-associated viral (AAV) vector, an adenoviral vector, a lentiviral vector, or a retroviral vector.

Where a replication-deficient virus is used as the viral vector, the production of infective virus particles containing either DNA or RNA corresponding to the DNA of interest can be produced by introducing the viral construct into a recombinant cell line which provides the missing components essential for viral replication. In some aspects, transformation of the recombinant cell line with the recombinant viral vector will not result in production of replication-competent viruses, e.g., by homologous recombination of the viral sequences of the recombinant cell line into the introduced viral vector. Methods for production of replication-deficient viral particles containing a nucleic acid of interest are well known in the art and are described in, e.g., Rosenfeld et al., Science 252:431-434 (1991) and Rosenfeld et al., Cell 68:143-155 (1992) (adenovirus); U.S. Pat. No. 5,139,941 (adeno-associated virus); U.S. Pat. No. 4,861,719 (retrovirus); and U.S. Pat. No. 5,356,806 (vaccinia virus).

### IV.C Adeno-Associated Virus

AAV, a parvovirus belonging to the genus Dependovirus, has several attractive features not found in other viruses. For example, AAV can infect a wide range of host cells, including non-dividing cells. Furthermore, AAV can infect cells from different species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

The AAV genome, a linear, single-stranded DNA molecule containing approximately 4700 nucleotides (the AAV-2 genome consists of 4681 nucleotides), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, respectively: replication and capsid gene products (i.e., proteins) allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, requiring co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV will replicate in canine cells that have been co-infected with a canine adenovirus.

In some aspects, to produce recombinant AAV (rAAV) virions containing the HNA, a suitable host cell line is transfected with an AAV vector containing the HNA, but lacking rep and cap. The host cell is then infected with wild-type (wt) AAV and a suitable helper virus to form rAAV virions. Alternatively, wt AAV genes (known as helper function genes, comprising rep and cap) and helper virus function genes (known as accessory function genes) can be provided in one or more plasmids, thereby eliminating the need for wt AAV and helper virus in the production of rAAV virions. The helper and accessory function gene products are expressed in the host cell where they act in trans on the rAAV vector containing the heterologous gene. The heterologous gene is then replicated and packaged as though it were a wt AAV genome, forming a recombinant AAV virion. When a patient's cells are transduced with the resulting rAAV virion, the HNA enters and is expressed in the patient's cells. Because the patient's cells lack the rep and cap genes, as well as the accessory function genes, the rAAV virion cannot further replicate and package its genomes. Moreover, without a source of rep and cap genes, wt AAV virions cannot be formed in the patient's cells. See e.g., U.S. Appl. Publ. No. 2003/0147853.

In some aspects, AAV vectors of the present disclosure can comprise or be derived from any natural or recombinant AAV serotype. According to the present disclosure, the AAV serotype can be, but is not limited to, AAV1, AAV2, AAV3, AAV4, AAV5, AAV6, AAV7, AAV8, AAVrh8, AAV9, AAV10, AAVrh10, AAV11, and AAV12. In some aspects, the AAV serotype is AAV2. In some aspects, an AAV vector includes a hybrid vector (e.g., AAV-DJ, AAV-PHP.B, AAV2-ESGHGYF, AAVM41, AAV-LK03, AAV2-BR1, AAV587MTP, AAV-Anc80L65, AAV2-7m8, AAV2HBKO, AAV2YF, AAV6-RGD or AAV6.2). In some aspects, the AAV vector is modified relative to the wild-type AAV serotype sequence. In some aspects, the modified AAV vector is a modified AAV6, e.g., an AAV6 vector comprising the RGD peptide (an AAV6-RGD vector) or an AAV6 vector comprising mutations of surface exposed tyrosine residues as described, for example, in Sayroo et al. Gene Ther. 2016 January; 23 (1): 18-25. In some aspects, the AAV6-RGD vector further comprises modified amino acids corresponding to Y705, Y731, T492, and K531, (e.g., Y705, Y731F, T492V, and K531E; also referred to as AAV-RGD-Y705-731F+T492V+K531E).

The AAV vectors of the present disclosure comprise a viral genome with at least one ITR region and a heterologous payload (e.g., an expression cassette comprising an alternatively spliced exon). In some aspects, the AAV vector comprises two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into AAV vectors of the disclosure can be comprised of naturally occurring polynucleotide sequences or recombinantly derived polynucleotide sequences.

The ITRs can be derived from the same serotype as the capsid, selected from any of the serotypes listed herein, or a derivative thereof. The ITR can be of a different serotype from the capsid. In some aspects, the AAV vector has more than one ITR. In a non-limiting example, the AAV vector has a viral genome comprising two ITRs. In some aspects, the ITRs are of the same serotype as one another. In some aspects, the ITRs are of different serotypes. Non-limiting examples include zero, one or both of the ITRs having the same serotype as the capsid. In some aspects both ITRs of the AAV vector are AAV2 ITRs.

Independently, each ITR can be about 75 to about 175 nucleotides in length. An ITR can be about 100-105 nucleotides in length, about 106-110 nucleotides in length, about 111-115 nucleotides in length, about 116-120 nucleotides in length, about 121-125 nucleotides in length, about 126-130 nucleotides in length, about 131-135 nucleotides in length, about 136-140 nucleotides in length, about 141-145 nucleotides in length or about 146-150 nucleotides in length. In some aspects, the ITRs are about 140-142 nucleotides in length. Non-limiting examples of ITR length are about 102, about 140, about 141, about 142, about 145 nucleotides in length, and those having at least 95% identity thereto.

In some aspects, the AAV vector comprises a nucleic acid sequence disclosed herein which can be located within about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 or more than about 30 nucleotides downstream or upstream from the 5′ or 3′ end of an ITR (e.g., Flip or Flop ITR) in the vector.

In some aspects, the AAV vector comprises one or more filler sequences (also referred to as “stuffer sequences”). In some aspects, the AAV vector comprises one or more filler sequences in order to have the length of the AAV vector be the optimal size for packaging.

In some aspects, the AAV vector is a single stranded (ss) AAV vector.

In some aspects, the AAV vector is a self-complementary (sc) AAV vector.

In some aspects, the AAV vector can comprise one or more filler sequences between one of more regions of the AAV vector. In some aspects, the filler region can be located before a region such as, but not limited to, a payload region, an ITR, a promoter region, an intron region, an enhancer region, and/or a polyadenylation signal sequence region. In some aspects, the filler region can be located after a region such as, but not limited to, a payload region, an ITR, a promoter region, an intron region, an enhancer region, and/or a polyadenylation signal sequence region. In some aspects, the filler region can be located before and after a region such as, but not limited to, a payload region, an ITR, a promoter region, an intron region, an enhancer region, and/or a polyadenylation signal sequence region.

In some aspects, an AAV vector can comprise two filler sequences. The two filler sequences can be located between two regions as described herein.

The present disclosure provides also methods for the generation of AAV particles, by viral genome replication in a viral replication cell comprising contacting the viral replication cell with an AAV polynucleotide or AAV genome (e.g., an AAV vector of the present disclosure).

In some aspects, an AAV particle is produced by a method comprising the steps of:
(1) co-transfecting competent bacterial cells with a bacmid vector and either a viral construct vector and/or AAV payload construct vector,
(2) isolating the resultant viral construct expression vector and AAV payload construct expression vector and separately transfecting viral replication cells, (3) isolating and purifying resultant payload and viral construct particles comprising viral construct expression vector or AAV payload construct expression vector,
(4) co-infecting a viral replication cell with both the AAV payload and viral construct particles comprising viral construct expression vector or AAV payload construct expression vector, and
(5) harvesting and purifying the viral particle comprising a parvoviral genome.

In one aspect, the present disclosure provides a method for producing an AAV particle comprising the steps of (1) simultaneously co-transfecting mammalian cells, such as, but not limited to HEK293 cells, with a synthetic promoter operably linked to a nucleic acid, a construct expressing rep and cap genes and a helper construct, and (2) harvesting and purifying the AAV particle comprising a viral genome.

In one aspect, the present disclosure provides a method for producing an AAV particle comprising the steps of (1) contacting mammalian cells, such as, but not limited to HEK293 cells, comprising i) a synthetic promoter operably linked to a nucleic acid, ii) rep gene(s), iii) cap gene(s) and iv) a helper construct comprising AAV helper genes, with a topsentin alkaloid or derivative thereof, and (2) harvesting and purifying the AAV particle comprising a viral genome.

In some aspects, the cells can be contacted with a topsentin alkaloid or derivative thereof. In some aspects, the cells can be cultured in the presence of a topsentin alkaloid or derivative thereof. In some aspects, the topsentin alkaloid or derivative thereof is homocarbonyltopsentin.

To address limitations of the Triple Transfection system for AAV production (e.g., issues of scale due to the number of cells required, the efficiency of transfection, and the cost of raw materials), efforts have been made to generate producer cell lines that contain one or more of the vector production genes, such as Rep or Cap. A significant barrier to these efforts was the cytotoxic and cytostatic effects of constitutive expression of Rep (See Yang, Q., et al., J Virol. 68 (8): 4847-56 (August 1994); Schmidt, M., et al., J Virol. 74 (20): 9441-50 (October 2000)). To overcome this issue, a system was developed in which Rep RNA was alternatively spliced to generate a message that only contains a start codon upon treatment with a small molecule. The treatment of the small molecule allowed for both the timing and level of expression to result in packaging of viral vectors.

In some aspects, provided herein is a method of producing an adeno-associated viral (AAV) vector comprising: (a) introducing a first plasmid comprising an adenovirus helper gene and a second plasmid comprising a heterologous nucleic acid sequence into a host cell; and (b) culturing the host cell in the presence of a splicing modulator to produce AAV particles.

In some aspects, the host cell comprises an AAV capsid (Cap) gene and a nucleic acid construct comprising (i) an upstream exon, (ii) an upstream intron, (iii) an alternatively spliced exon comprising a translation initiation sequence, (iv) a downstream intron, (v) a downstream exon, and (vi) an AAV replication (Rep) gene.

In some aspects, the host cell comprises an AAV replication (Rep) gene and a nucleic acid construct comprising (i) an upstream exon, (ii) an upstream intron, (iii) an alternatively spliced exon comprising a translation initiation sequence, (iv) a downstream intron, (v) a downstream exon, and (vi) an AAV capsid (Cap) gene.

In some aspects, the upstream exon, the upstream intron, the alternatively spliced exon, the downstream intron, and the downstream exon each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the method further comprises (c) harvesting the AAV particles from the cultured host cell.

In some aspects, the method further comprises (d) purifying the harvested AAV particles.

In some aspects, the host cell is a HEK293 cell.

In some aspects, the splicing modulator is Kinetin or Homocarbonyltopsentin.

In some aspects, the adenovirus helper gene is selected from the group consisting of an Early 4 (E4) gene, an Early 2A (E2A) gene, and a viral associated (VA) gene.

In some aspects, the nucleic acid construct is stably integrated into the host cell genome.

In some aspects, the nucleic acid construct comprises a sequence selected from the group consisting of SEQ ID NO: 59-62.

In some aspects, the AAV particles can be produced in a viral replication cell that comprises an insect cell. Growing conditions for insect cells in culture, and production of heterologous products in insect cells in culture are well-known in the art, see, e.g., U.S. Pat. No. 6,204,059.

The viral replication cell can be selected from any biological organism, including prokaryotic (e.g., bacterial) cells, and eukaryotic cells, including, insect cells, yeast cells and mammalian cells. Viral replication cells can comprise mammalian cells such as A549, WEH1, 3T3, 10T½, BHK, MDCK, COS 1, COS 7, BSC 1, BSC 40, BMT 10, VERO, W138, HeLa, HEK293, Saos, C2C12, L cells, HT1080, HepG2 and primary fibroblast, hepatocyte and myoblast cells derived from mammals. Viral replication cells comprise cells derived from mammalian species including, but not limited to, human, monkey, mouse, rat, rabbit, and hamster or cell type, including but not limited to fibroblast, hepatocyte, tumor cell, cell line transformed cell, etc.

Viral production disclosed herein describes processes and methods for producing AAV particles that contact a target cell to deliver a payload.

In some aspects, the AAV particles can be produced in a viral replication cell that comprises a mammalian cell. Viral replication cells commonly used for production of recombinant AAV particles include, but are not limited to 293 cells, COS cells, HeLa cells, and KB cells.

In some aspects, AAV particles are produced in mammalian cells wherein all three VP proteins are expressed at a stoichiometry approaching 1:1:10 (VP1:VP2:VP3). The regulatory mechanisms that allow this controlled level of expression include the production of two mRNAs, one for VP1, and the other for VP2 and VP3, produced by differential splicing.

In some aspects, AAV particles are produced in mammalian cells using a triple transfection method wherein a payload construct, parvoviral Rep and parvoviral Cap and a helper construct are comprised within three different constructs. The triple transfection method of the three components of AAV particle production can be utilized to produce small lots of virus for assays including transduction efficiency, target tissue (tropism) evaluation, and stability.

In some aspects, the viral construct vector and the AAV payload construct vector can be each incorporated by a transposon donor/acceptor system into a bacmid, also known as a baculovirus plasmid, by standard molecular biology techniques known and performed by a person skilled in the art. Transfection of separate viral replication cell populations produces two baculoviruses, one that comprises the viral construct expression vector, and another that comprises the AAV payload construct expression vector. The two baculoviruses can be used to infect a single viral replication cell population for production of AAV particles.

Baculovirus expression vectors for producing viral particles in insect cells, including but not limited to Spodoptera frugiperda (Sf9) cells, provide high titers of viral particle product. Recombinant baculovirus encoding the viral construct expression vector and AAV payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection (MOI), see, e.g., Urabe, M. et al., J Virol. 2006 February; 80 (4): 1874-85, the contents of which are herein incorporated by reference in their entirety.

Production of AAV particles with baculovirus in an insect cell system can address known baculovirus genetic and physical instability. Baculovirus-infected viral producing cells are harvested into aliquots that can be cryopreserved in liquid nitrogen; the aliquots retain viability and infectivity for infection of large-scale viral producing cell culture (Wasilko D J et al., Protein Expr Purif. 2009 June; 65 (2): 122-32).

In some aspects, stable viral replication cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some aspects, AAV particle production can be modified to increase the scale of production. Transfection of replication cells in large-scale culture formats can be carried out according to any methods known in the art.

In some aspects, cell culture bioreactors can be used for large scale viral production.

In some cases, bioreactors comprise stirred tank reactors.

Cells of the disclosure, including, but not limited to viral production cells, can be subjected to cell lysis according to any methods known in the art. Cell lysis can be carried out to obtain one or more agents (e.g. viral particles) present within any cells of the disclosure.

Cell lysis methods can be chemical or mechanical. Chemical cell lysis typically comprises contacting one or more cells with one or more lysis agent. Mechanical lysis typically comprises subjecting one or more cells to one or more lysis condition and/or one or more lysis force. In some aspects, chemical lysis can be used to lyse cells. As used herein, the term “lysis agent” refers to any agent that can aid in the disruption of a cell. In some cases, lysis agents are introduced in solutions, termed lysis solutions or lysis buffers. As used herein, the term “lysis solution” refers to a solution (typically aqueous) comprising one or more lysis agent. In addition to lysis agents, lysis solutions can include one or more buffering agents, solubilizing agents, surfactants, preservatives, cryoprotectants, enzymes, enzyme inhibitors and/or chelators.

Concentrations of salts can be increased or decreased to obtain an effective concentration for rupture of cell membranes. Lysis agents comprising detergents can include ionic detergents or non-ionic detergents. Detergents can function to break apart or dissolve cell structures including, but not limited to cell membranes, cell walls, lipids, carbohydrates, lipoproteins and glycoproteins.

In some aspects, mechanical cell lysis is carried out. Mechanical cell lysis methods can include the use of one or more lysis condition and/or one or more lysis force. As used herein, the term “lysis condition” refers to a state or circumstance that promotes cellular disruption. Lysis conditions can comprise certain temperatures, pressures, osmotic purity, salinity and the like. In some aspects, lysis conditions comprise increased or decreased temperatures. In some aspects, lysis conditions comprise changes in temperature to promote cellular disruption. Cell lysis carried out according to such aspects can include freeze-thaw lysis.

As used herein, the term “lysis force” refers to a physical activity used to disrupt a cell. Lysis forces can include, but are not limited to mechanical forces, sonic forces, gravitational forces, optical forces, electrical forces and the like. Cell lysis carried out by mechanical force is referred to herein as “mechanical lysis.” Mechanical forces that can be used according to mechanical lysis can include high shear fluid forces.

In some aspects, a method for harvesting AAV particles without lysis can be used for efficient and scalable AAV particle production. In a non-limiting example, AAV particles can be produced by culturing an AAV particle lacking a heparin binding site, thereby allowing the AAV particle to pass into the supernatant, in a cell culture, collecting supernatant from the culture; and isolating the AAV particle from the supernatant, as described in U.S. Patent Application 20090275107.

Cell lysates comprising viral particles can be subjected to clarification. Clarification refers to initial steps taken in purification of viral particles from cell lysates. Clarification serves to prepare lysates for further purification by removing larger, insoluble debris. Clarification steps can include, but are not limited to centrifugation and filtration.

In some aspects, AAV particles can be purified from clarified cell lysates by one or more methods of chromatography. Chromatography refers to any number of methods known in the art for separating out one or more elements from a mixture. Such methods can include, but are not limited to ion exchange chromatography (e.g. cation exchange chromatography and anion exchange chromatography), immunoaffinity chromatography and size-exclusion chromatography.

### V. Host Cells

Certain aspects of the disclosure are directed to a cell comprising any of the expression cassettes disclosed herein or any of the delivery vectors disclosed herein.

In some aspects, the cell is a host cell.

In some aspects, the host cell can be selected from any biological organism, including prokaryotic (e.g., bacterial) cells, and eukaryotic cells, including, insect cells, yeast cells and mammalian cells. Host cells can comprise mammalian cells such as A549, WEH1, 3T3, 10T½, BHK, MDCK, COS 1, COS 7, BSC 1, BSC 40, BMT 10, VERO. W138, HeLa, HEK293, Saos, C2C12, L cells, HT1080, HepG2 and primary fibroblast, hepatocyte and myoblast cells derived from mammals. Host cells comprise cells derived from mammalian species including, but not limited to, human, monkey, mouse, rat, rabbit, and hamster or cell type, including but not limited to fibroblast, hepatocyte, tumor cell, cell line transformed cell, etc.

In some aspects, the host cell is a HEK293 cell. In some aspects, the host cell is derived from a HEK293 cell (e.g., a HEK293T cell).

The expression cassettes of the disclosure can be transfected into the host cells by any methods know in the art. For example, the expression cassettes of the disclosure can be transfected into the viral replication cell by electroporation, cationic lipid transfection, or calcium phosphate transfection.

In some aspects, the host cell comprises a nucleic acid construct comprising (i) an upstream exon, (ii) an upstream intron, (iii) an alternatively spliced exon comprising a translation initiation sequence, (iv) a downstream intron, (v) a downstream exon, and (vi) an AAV replication (Rep) gene. In some aspects, the upstream exon, the upstream intron, the alternatively spliced exon, the downstream intron, and the downstream exon each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

In some aspects, the nucleic acid construct is stably integrated into the host cell genome.

In some aspects, the host cell comprises a nucleic acid construct corresponding to any of SEQ ID NOs: 59-62.

In some aspects, provided herein is a method of increasing production of an adeno-associated virus (AAV) packaging gene in a host cell, the method comprising culturing the host cell in the presence of a small molecule modulator (e.g., an indole alkaloid). In some aspects, the AAV packaging gene is an AAV capsid (Cap) gene. In some aspects, the AAV packaging gene is an AAV replication (Rep) gene.

In some aspects, the host cell is a HEK293 cell.

In some aspects, the small molecule modulator is a topsentin alkaloid or derivative thereof.

In some aspects, the small molecule modulator is Homocarbonyltopsentin.

### VI. Pharmaceutical Constructs

Certain aspects of the disclosure are directed to a pharmaceutical composition comprising any of the expression cassettes disclosed herein, any of the delivery vectors disclosed herein, any of the cells disclosed herein, or any of the viral particles (e.g., rAAV) disclosed herein.

In some aspects, the pharmaceutical further comprises a pharmaceutically acceptable excipient.

Pharmaceutically acceptable excipients or carriers are determined in part by the particular composition being administered, as well as by the particular method used to administer the composition.

Accordingly, there is a wide variety of suitable formulations of pharmaceutical compositions comprising a delivery vector of the present disclosure (e.g., an AAV vector) or a plurality thereof (see, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration. In some aspects, the pharmaceutical composition comprises more than one AAV vector of the present disclosure.

Acceptable carriers, excipients, or stabilizers are nontoxic to recipients (e.g., animals or humans) at the dosages and concentrations employed.

Examples of carriers or diluents include, but are not limited to, water, saline, Ringer's solutions, dextrose solution, and 5% human serum albumin. Except insofar as any conventional media or compound is incompatible with the delivery vectors disclosed herein (e.g., AAV vectors or AAV capsids), use thereof in the compositions is contemplated. In some aspects, a pharmaceutical composition is formulated to be compatible with its intended route of administration. The delivery vectors disclosed herein (e.g., AAV vectors or AAV capsids) can be administered by intravenous, parenteral, topical, intravenous, oral, subcutaneous, intra-arterial, intradermal, transdermal, rectal, intracranial, intraperitoneal, intranasal, intratumoral, intramuscular route or as inhalants.

In some aspects, the pharmaceutical composition is administered intratumorally, e.g. by direct injection. In some aspects, the pharmaceutical composition is administered intravenously.

### VII. Kits and Articles of Manufacture

In some aspects, provided herein is a kit comprising i) any of the expression cassettes disclosed herein, any of the delivery vectors disclosed herein, any of the cells disclosed herein, or any of the pharmaceutical compositions disclosed herein; and (ii) optionally instructions for use.

In some aspects, the kit further comprises (iii) a splicing modulator molecule that promotes the inclusion of the alternatively spliced exon.

In some aspects, the splicing modulator molecule is kinetin.

In some aspects, the splicing modulator molecule is homocarbonyltopsentin.

The disclosure also provides kits and products of manufacture comprising, for example,
(i) an expression cassette of the present disclosure
(ii) a delivery vector of the present disclosure;
(iii) a viral particle (e.g., rAAV) of the present disclosure;
(iv) a cell of the present disclosure;
(v) instructions for use; or
(vi) any combination thereof.

In some aspects, the kit or product of manufacture comprises, e.g., an expression cassette of the present disclosure or delivery vector of the present disclosure, in at least one container, and one or more containers with reagents sufficient for transfection of one or more cells or expression in one or more cells.

In some aspects, the disclosure provides kits and products of manufacture comprising, for example, one or more cells genetically modified to express one or more expression cassettes of the present disclosure, or one or more delivery vectors of the present disclosure, or a pharmaceutical composition comprising the one or more cells, and optionally instructions for use.

One skilled in the art will readily recognize that the expression cassettes of the present disclosure, delivery vector of the present disclosure, cell genetically modified to express an expression cassette of the present disclosure, composition (e.g., a pharmaceutical composition comprising n expression cassette, delivery vector, or cell disclosed herein), or combinations thereof can be readily incorporated into one of the established kit formats which are well known in the art.

Having described the present invention, the same will be explained in greater detail in the following examples, which are included herein for illustration purposes only, and which are not intended to be limiting to the invention.

### VIII. Methods of Use

In some aspects, provided herein is a method of regulating expression of a protein of interest in a mammalian cell comprising administering to the cell any of the expression cassettes disclosed herein, any of the delivery vectors disclosed herein, or any of the pharmaceutical compositions disclosed herein, followed by administering the splicing modulator molecule that promotes the inclusion of the alternatively spliced exon.

In some aspects, provided herein is a method of providing a protein to a subject comprising administering to the subject any of the expression cassettes disclosed herein, any of the delivery vectors disclosed herein, any of the cells disclosed herein, or any of the pharmaceutical compositions disclosed herein, followed by administering the splicing modulator molecule that promotes the inclusion of the alternatively spliced exon.

In some aspects, provided herein is a method of treating a disease in a mammal comprising administering to a subject any of the expression cassettes disclosed herein, any of the delivery vectors disclosed herein, any of the cells disclosed herein, or any of the pharmaceutical compositions disclosed herein, followed by administering the splicing modulator molecule that promotes the inclusion of the alternatively spliced exon.

In some aspects, provided herein is a method of regulating transgene expression post-transcriptionally, comprising (a) a nucleic acid construct comprising (i) an upstream exon, (ii) an upstream intron, (iii) an alternatively spliced exon comprising a translation initiation sequence, (iv) a downstream intron, (v) a downstream exon, and (vi) a heterologous nucleic acid sequence; and (b) a splicing modulator. In some aspect, the upstream exon, the upstream intron, the alternatively spliced exon, the downstream intron, and the downstream exon each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene. In some aspects, the splicing modulator is Kinetin or Homocarbonyltopsentin.

In some aspects, the administration of the expression cassette, delivery vector, or cell is systemic or tissue specific. In some aspects, the administration is intravenous, intramuscular, subcutaneous, oral, intraocular, or intratumoral.

In some aspects, the administration of the splicing modulator molecule that promotes the inclusion of the alternatively spliced exon is systemic.

In some aspects, the splicing modulator molecule that promotes the inclusion of the alternatively spliced exon is kinetin.

In some aspects, the splicing modulator molecule that promotes the inclusion of the alternatively spliced exon is homocarbonyltopsentin.

In some aspects, the splicing modulator molecule that promotes the inclusion of the alternatively spliced exon is administered at least 1 day after administration of the expression cassette.

In some aspects, provided herein is a method of producing an AAV particle comprising contacting a host cell comprising rep genes, cap genes, and adenoviral helper genes with a topsentin alkaloid or derivative thereof (e.g., Homocarbonyltopsentin, Topsentin, Bromotopsentin, Bromodeoxytopsentin, Deoxytopsentin, and any combination thereof).

In some aspects, the topsentin alkaloid or derivative thereof (e.g., Homocarbonyltopsentin): i) reduces one or more viral response genes, ii) increases one or more immediate early genes, iii) increases a NF-κB signaling pathway, iv) increases a TNF signaling pathway, v) increases a IL-17 signaling pathway, vi) inhibits apoptosis, vii) inhibits viral defense and/or vii) increases histone levels.

In some aspects, the apoptosis is inhibited by altering a signaling pathway.

In some aspects, the signaling pathway is the NF-κB signaling pathway.

In some aspects, the signaling pathway is the TNF signaling pathway.

In some aspects, the signaling pathway is the IL-17 signaling pathway.

In some aspects, the signaling pathway is the JAK/STAT signaling pathway.

In some aspects, the signaling pathway is the P38 MAPK signaling pathway.

In some aspects, signaling pathway is the PI3K-Akt signaling pathway.

In some aspects, the increased histone levels result in increased metabolic activity.

In some aspects, the increased histone levels result in increased host cell replication.

In some aspects, the host cell comprises an AAV capsid (Cap) gene.

In some aspects, the host cell comprises an AAV replication (Rep) gene.

In some aspects, the host cell is a HEK293 cell.

In some aspects, the topsentin alkaloid or derivative thereof is Homocarbonyltopsentin.

In some aspects, the host cell is contacted with about 5 μM to about 50 μM (e.g., about 5 μM, about 6 μM, about 7 μM, about 8 μM, about 9 UM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 31 μM, about 32 M, about 33 μM, about 34 μM, about 35 μM, about 36 μM, about 37 μM, about 38 μM, about 39 μM, or about 40 μM) of Homocarbonyltopsentin.

In some aspects, the host cell is contacted with between about 5 μM and about 50 μM of Homocarbonyltopsentin. In some aspects, the host cell is contacted with between about 5 μM and about 40 μM of Homocarbonyltopsentin. In some aspects, the host cell is contacted with between about 10 UM and about 40 μM of Homocarbonyltopsentin. In some aspects, the host cell is contacted with between about 15 μM and about 40 μM of Homocarbonyltopsentin. In some aspects, the host cell is contacted with between about 20 μM and about 40 μM of Homocarbonyltopsentin. In some aspects, the host cell is contacted with between about 25 M and about 40 M of Homocarbonyltopsentin. In some aspects, the host cell is contacted with between about 30 μM and about 40 μM of Homocarbonyltopsentin. In some aspects, the host cell is contacted with between about 35 μM and about 40 μM of Homocarbonyltopsentin. In some aspects, the host cell is contacted with between about 5 μM and about 35 μM of Homocarbonyltopsentin. In some aspects, the host cell is contacted with between about 5 μM and about 30 μM of Homocarbonyltopsentin. In some aspects, the host cell is contacted with between about 5 μM and about 25 μM of Homocarbonyltopsentin. In some aspects, the host cell is contacted with between about 5 μM and about 20 μM of Homocarbonyltopsentin. In some aspects, the host cell is contacted with between about 5 μM and about 15 μM of Homocarbonyltopsentin. In some aspects, the host cell is contacted with between about 5 μM and about 10 μM of Homocarbonyltopsentin. In some aspects, the host cell is contacted with between about 10 μM and about 30 μM of Homocarbonyltopsentin. In some aspects, the host cell is contacted with between about 20 μM and about 30 μM of Homocarbonyltopsentin.

In some aspects, the host cell is contacted with the topsentin alkaloid or derivative (e.g., Homocarbonyltopsentin) for about 24 to 90 hours. In some aspects, the host cell is contacted with the topsentin alkaloid or derivative (e.g., Homocarbonyltopsentin) for at least about 24 hours, at least about 30 hours, at least about 36 hours, at least about 42 hours, at least about 48 hours, at least about 54 hours, at least about 60 hours, at least about 66 hours, or at least about 72 hours.

In some aspects, the host cell is contacted with the topsentin for about 24 hours.

In some aspects, the host cell is contacted with the topsentin for about 48 hours.

In some aspects, the host cell is contacted with the topsentin for about 72 hours.

In some aspects, the host cell is contacted with the topsentin for between about 24 hours and about 72 hours.

In some aspects, the method further comprises harvesting the AAV particle.

In some aspects, the method further comprises purifying the harvested AAV particle.

### EXAMPLES

### Example 1: Identification of Inducible Alternatively Spliced Exons

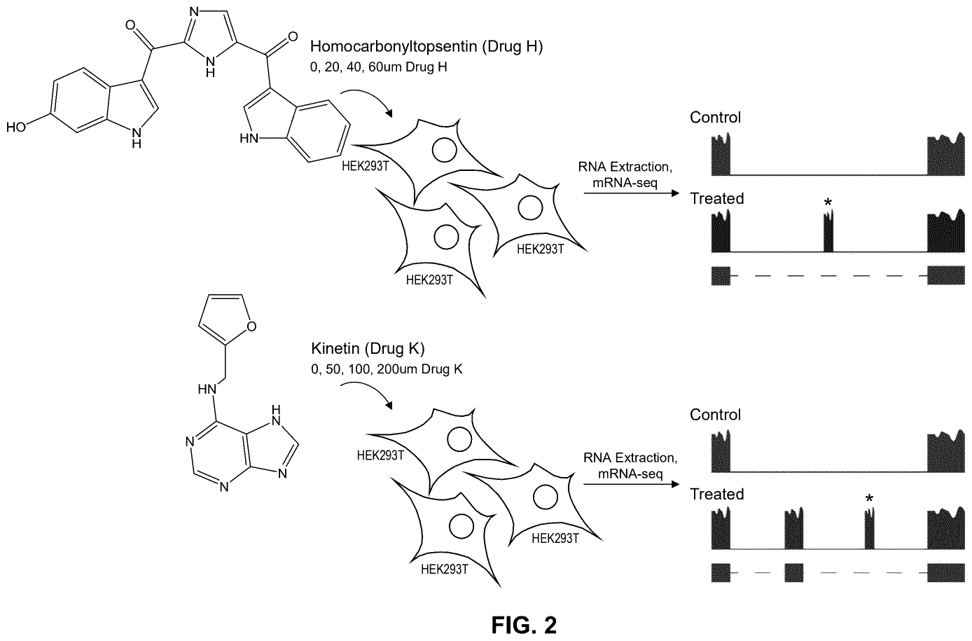

Regions of the genome were identified that undergo alternative splicing events upon small molecule treatment, for the purpose of controlling transgene protein levels. To identify these regions, HEK293T cells were treated with two previously reported splicing modulators, Kinetin (Drug K) and Homocarbonyltopsentin (Drug H) at three different concentrations (low, medium, and high) and isolated total RNA from cells in each of those conditions. Novel splice sites close in proximity were identified in treated compared to control samples. The splice sites were used to identify exons that were alternatively spliced in the presence of homocarbonyltopsentin (Drug H) or kinetin (Drug K) (FIG. 2). Treatment with either of these drugs was assessed for increase the presence of either unused annotated exons or unannotated/novel exons in coding genes. Using RNA sequencing, the presence of alternatively spliced exons was identified in many different coding genes resulting from treatment of either Drug K or Drug H. A list of candidate sequences was generated based on the size of the novel exon as well as the depth of coverage at the novel or unused but annotated exon sequence based on visualization on a genome browser. These results supported that both Drug K and Drug H can drive alternative splicing events across the human genome and that these splicing events could be utilized to control expression of transgenes.

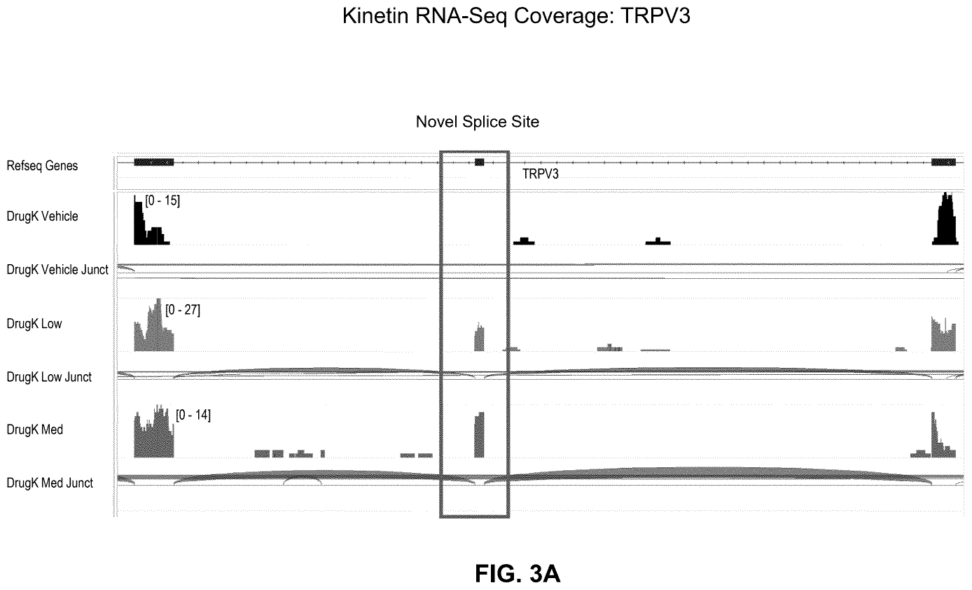

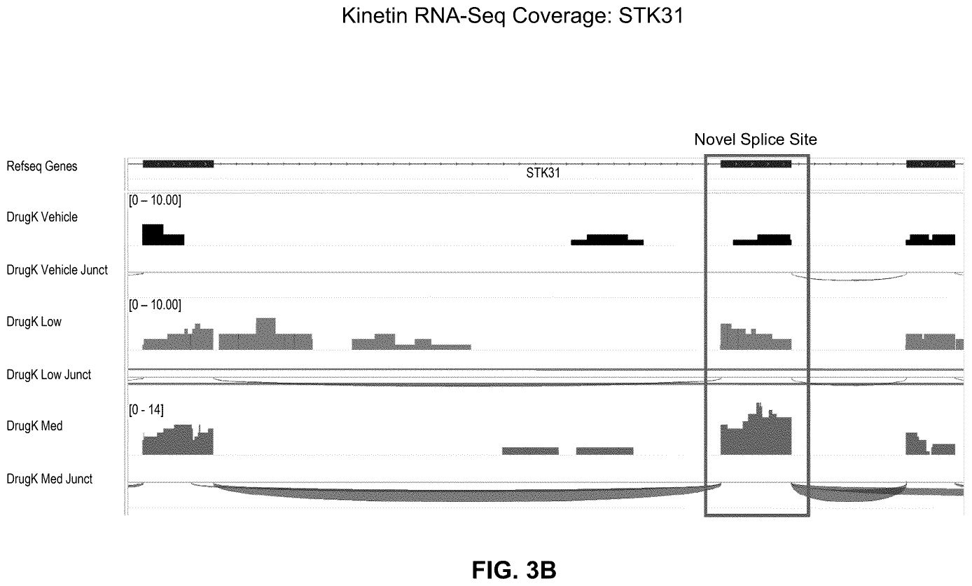

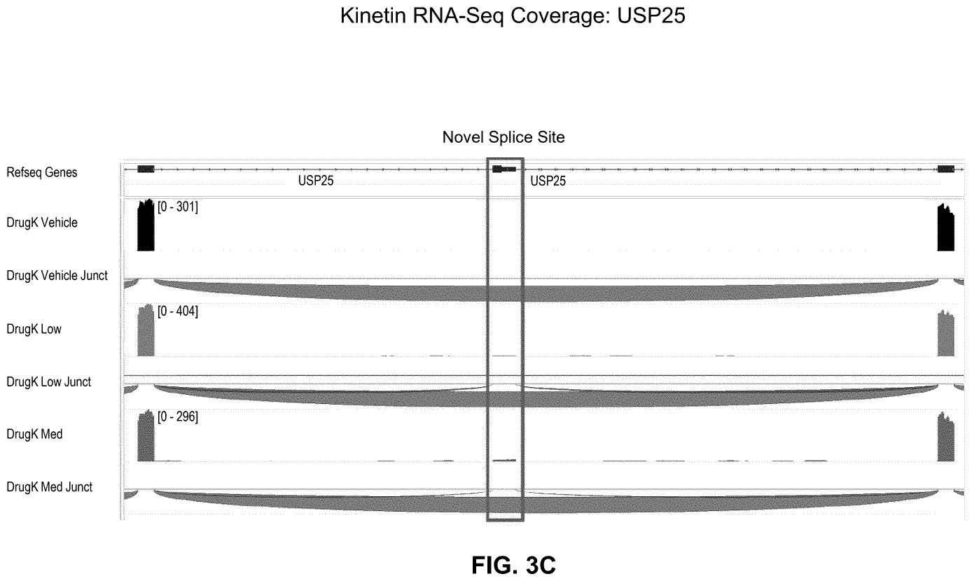

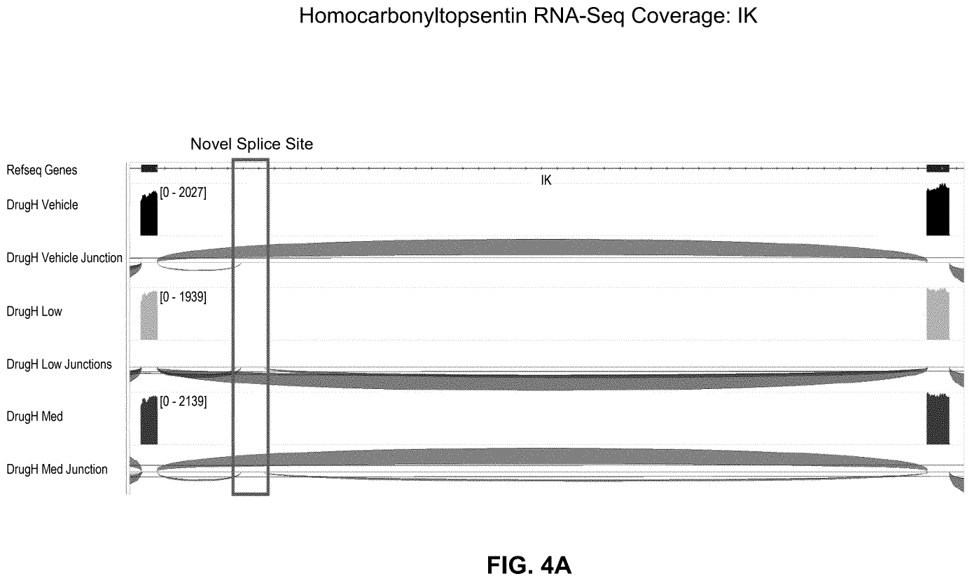

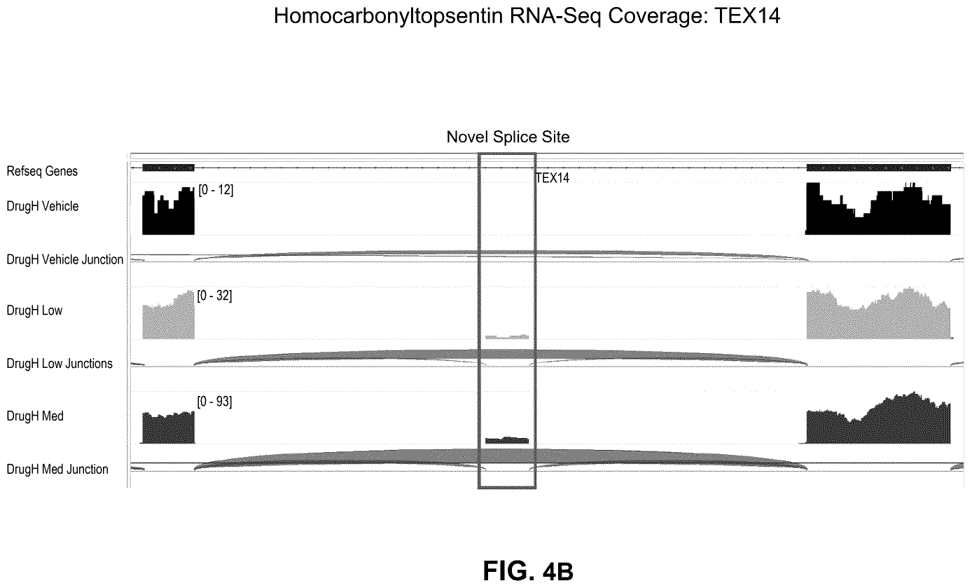

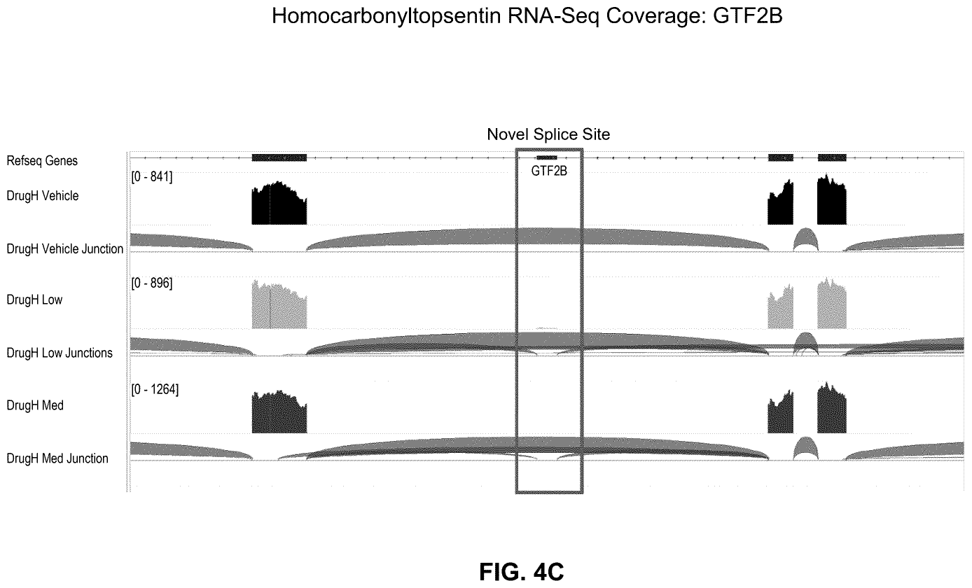

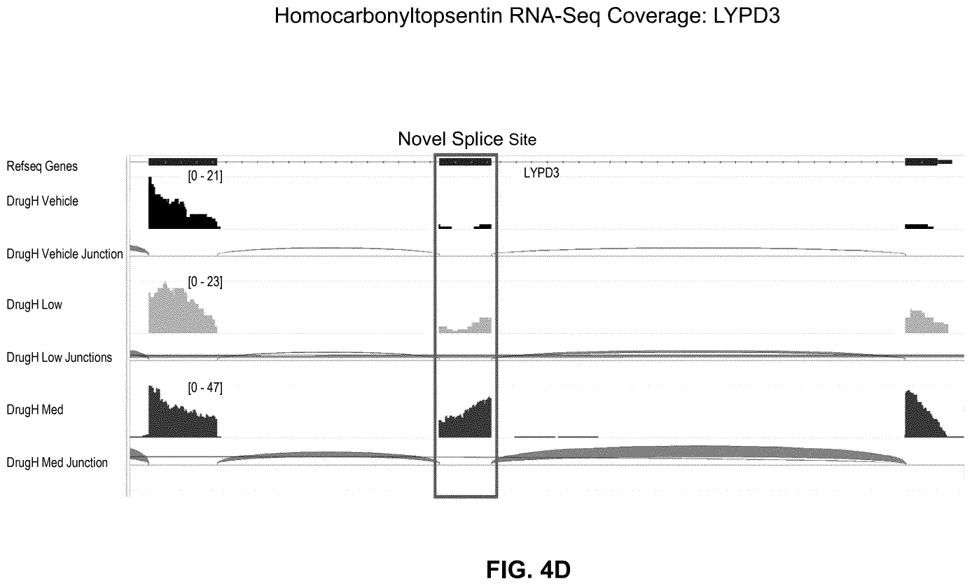

To generate reporter constructs that express a transgenic protein only upon treatment with Drug K or Drug H, 7 total candidate genes were selected. The regions of alternative splicing in genes TRPV3, STK31, and USP25 were selected as Drug-K responsive (FIGS. 3A-3C) and the alternative splicing regions from genes GTF2B, LYPD3, IK, and TEX14 were selected as Drug-H responsive (FIGS. 4A-4D). These genes were selected for several reasons including that they had novel exon sizes of less than 150 base pairs, at least 5 splice junction exon counts in the RNA-sequencing data, as well as low standard of deviation in novel exon splice junction counts, suggesting a conserved mechanism across biological replicates. The overall design of these constructs was to generate transcripts that would be transcribed with any promoter of interest, but only upon treatment with Drug K or Drug H would translation occur, due to the introduction of a start codon in the novel exon inserted by alternative splicing.

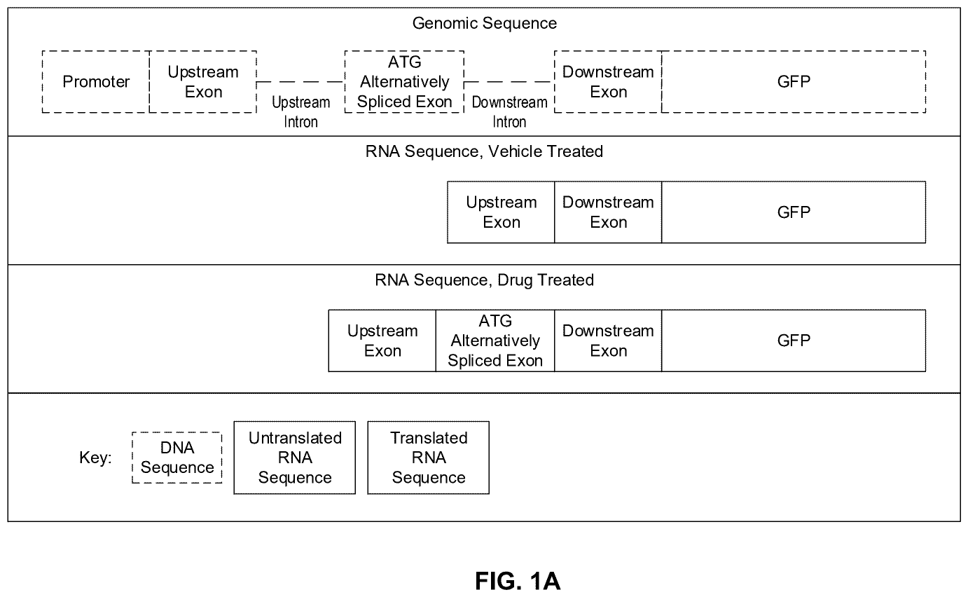

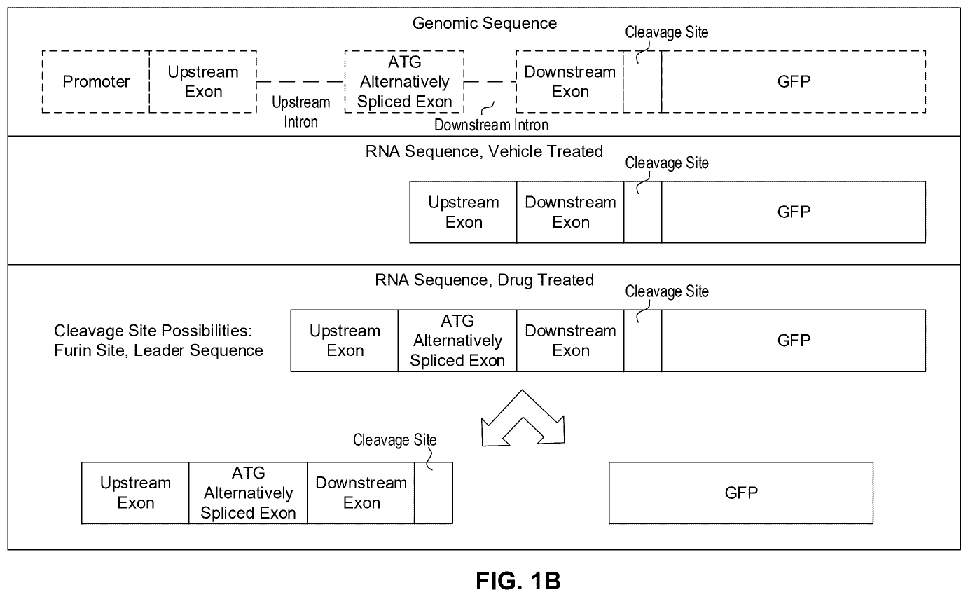

To design the splicing reporters, the entire region of the genome including the upstream exon, the upstream intron, the novel exon, the downstream intron, and the downstream exon were placed upstream of the coding sequence of the green fluorescent protein (GFP) (FIG. 1A-1B). Codons were identified and substituted in the novel exon with a consensus Kozak sequence and start codon so that they would be in frame with the downstream exon and GFP. To minimize background expression, any ATG start codons were removed that could drive GFP expression without drug treatment. To reduce the size of the constructs for eventual packaging into an AAV vector, minimal versions of these constructs were also generated that only contained 200 base pair introns, with only the first 100 bases at the 5′ splice site and the last 100 bases at the 3′ splice site of each intron maintained. Each of these reporters was placed downstream of a Rous sarcoma virus promoter for strong expression and cloned into a mammalian expression vector that included the hygromycin selection marker.

### Example 2: Small Molecule Splicing Modulators can Induce Transgene Expression Via Alternative Splicing

### Materials and Methods

Cell Culture, transfection, and drug treatments: HEK293T Human embryonic kidney cells (ATCC) were cultured in DMEM media containing 10% fetal bovine serum (Gibco) and 1% penicillin/streptomycin at 37 degrees Celsius with 5% carbon dioxide. For plasmid transfections, cells were cultured in 48-well plates and transfected at 70-80% confluence using Lipofectamine LTX reagent according to manufacturer's recommendation. For all experiments, 24 hours after transfection cells were treated with indicated amounts of DMSO, kinetin (MedChemExpress), or homocarbonyltopsentin (MedChemExpress). 24 hours after treatment, cells were washed with Dulbecco's phosphate buffered saline (DPBS) and imaged for GFP expression.

RNA-sequencing: 200,000 HEK293T cells were plated into individual wells of a 24-well plate and cultured for 24 hours. For kinetin treatment, after 24 hours of culture cells were treated with 50 μM, 100 μM, or 200 μM kinetin in DMSO, or DMSO alone as a vehicle control. For homocarbonyltopsentin treatment, after 24 hours of culture, cells were treated with 20 M, 40 μM, or 60 μM homocarbonyltopsentin in DMSO or DMSO alone as a vehicle control. After 24 hours of drug treatment, cells were washed with DPBS, and RNA was isolated using the Qiagen RNeasy kit with on-column DNase digestion according to manufacturer's instructions. RNA-sequencing libraries were constructed using the Illumina Stranded mRNA prep using 1000 nanograms of total RNA as input. 3 replicates of vehicle-treated or each concentration were sequenced on an Illumina NextSeq 2000 using a P3 300-cycle kit. The resulting fastq files were trimmed using Trim Galore! and then aligned to GRCh38 human genome using STAR. The splice junction output files were quantified using an R script (Monteys et al., Nature 596 (7871): 291-295 (2021); see also github.com/DavidsonLabCHOP/Xon) to identify novel splice junctions unique to treatment with Kinetin or Homocarbonyltopsentin. Novel splice isoforms were visualized using IGV. The top candidates from either kinetin or homocarbonyltopsentin treatment were selected by identifying two novel splice events within 500 bases of one another to select and design splicing-reporter constructs, followed by manual inspection of the novel splice site RNA-sequence coverage comparing drug treated samples to control samples.

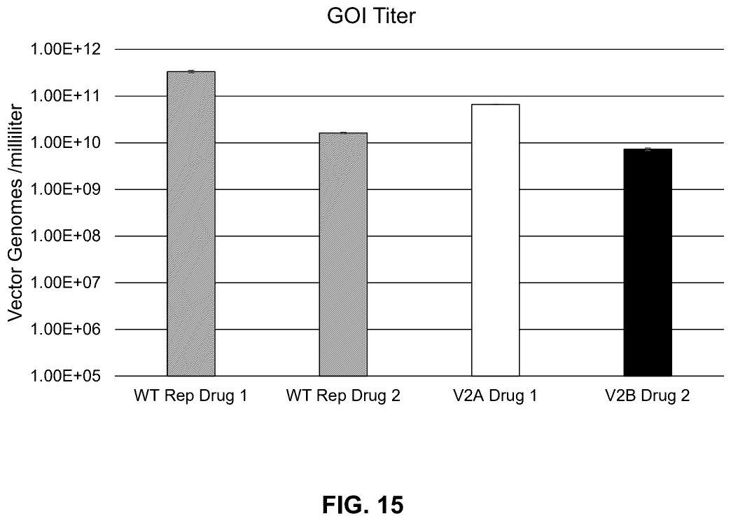

Plasmids: For each gene-splicing reporter, the entire genomic sequence including the Exons upstream and downstream of the novel splice site were obtained from GRCh38. To generate the full gene reporter, a consensus Kozak sequence (ccaccatgg) was inserted into the novel splice sequence so that the coding sequence would be in protein coding frame of an eGFP coding sequence at the 3′ end of the downstream exon sequence. In addition, all in frame ATG codons were mutated to minimize expression in vehicle conditions. Splicing reporter sequences were generated for the following genes: GTF2B, LYPD3, IK, TEX14, TRPV3, USP25, and STK31. To generate minimal versions of each reporter construct, the intron sequences were reduced to only 200 bases each, the 100 bases at the 5′ and 3′ ends of each intron, to include only the essential splicing sequences. The full and minimal sequences were placed downstream of the rous sarcoma virus promoter and then cloned into pcDNA3.1-Hygro(+) between the NruI and ApaI sites. Plasmids were transfected into HEK293T cells according to the method described above. Plasmid constructs were also generated to replace the eGFP coding sequence at the 3′ end of the downstream exon sequence with a Rep78/52 coding sequence (construct V2A and V2B—see FIGS. 15, 16 and 17A-17B).

Splicing Assays: Total RNA was extracted using the RNeasy kit (Qiagen) with on-column DNase digestion according to the manufacturer's protocol. To examine differential splicing, 1 μg of total RNA was converted to cDNA using the High-Capacity cDNA Reverse Transcription Kit (Thermo) according to manufacturer's instructions. For qPCR, splicing determined by either qPCR using a forward primer directed to the novel exon and a reverse primer targeted to eGFP. Real-time PCR was performed using Fast SYBR Green Master Mix (Thermo) according to manufacturer's recommendations on a QuantStudio 5 Real-Time PCR system (Applied Biosystems). The percentage of fold change of novel exon splicing was determined by the delta-delta Ct method, comparing the average Ct values of 3 biological replicates of the novel exon in drug treated conditions compared to vehicle treated conditions relative to the geometric mean of two control genes, ACTB and GAPDH. For gel analysis of splicing, cDNAs from cells treated with vehicle or drug were amplified using KAPA HiFi HotStart ReadyMix and a primer pair targeted towards the upstream constitutive exon of each gene and eGFP. The PCR product was run on a 1% agarose gel and stained with Sybr Gold to examine the size of the bands from the splicing products.

Fluorescent Microscopy: 48 hours after transfection and 24 hours after drug treatment, cells were washed with DPBS and imaged using and EVOS M5000 (Thermo) using identical settings for each well. 3 biological replicates were imaged for each condition.

### Drug H and Drug K Treatment

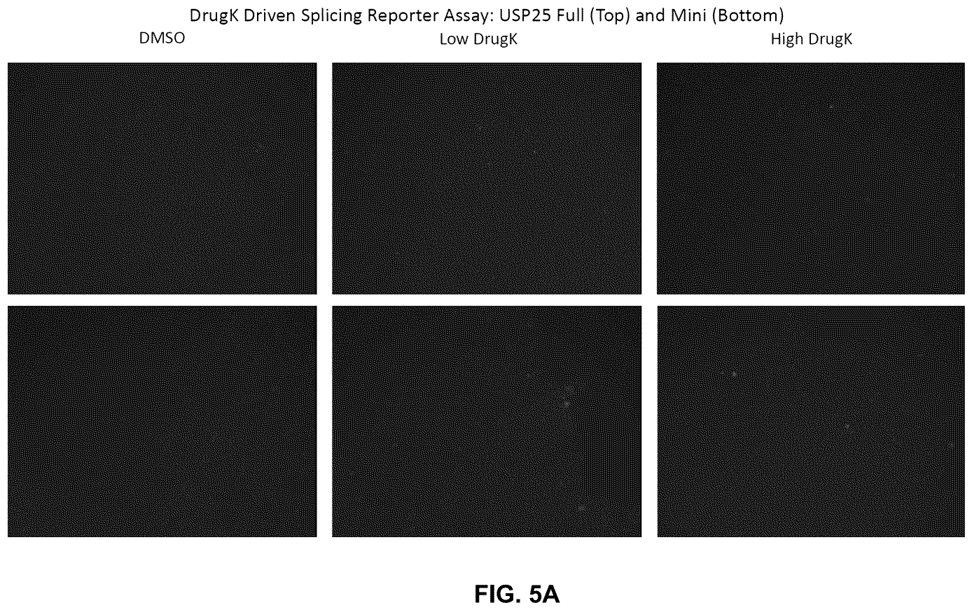

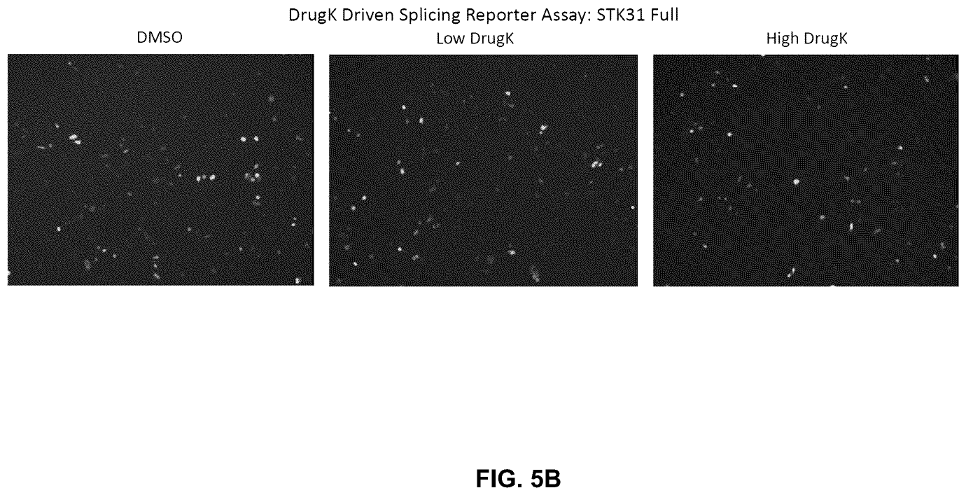

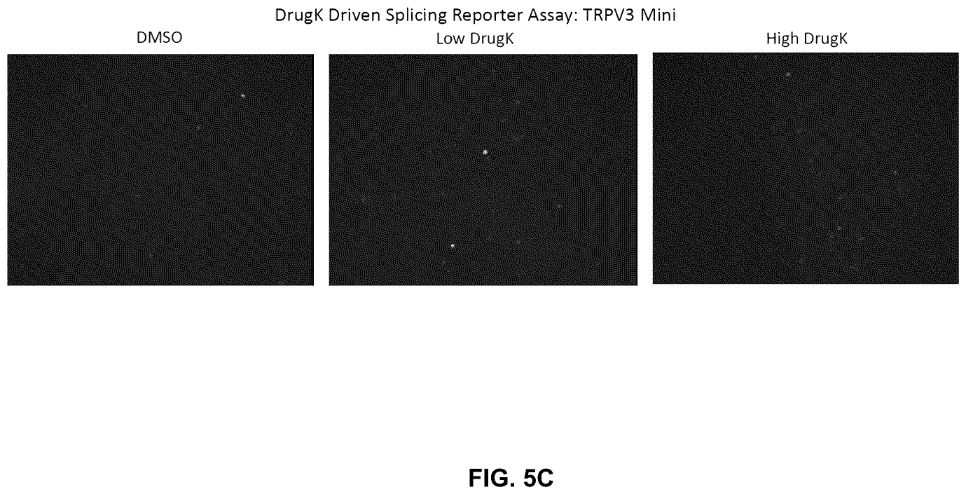

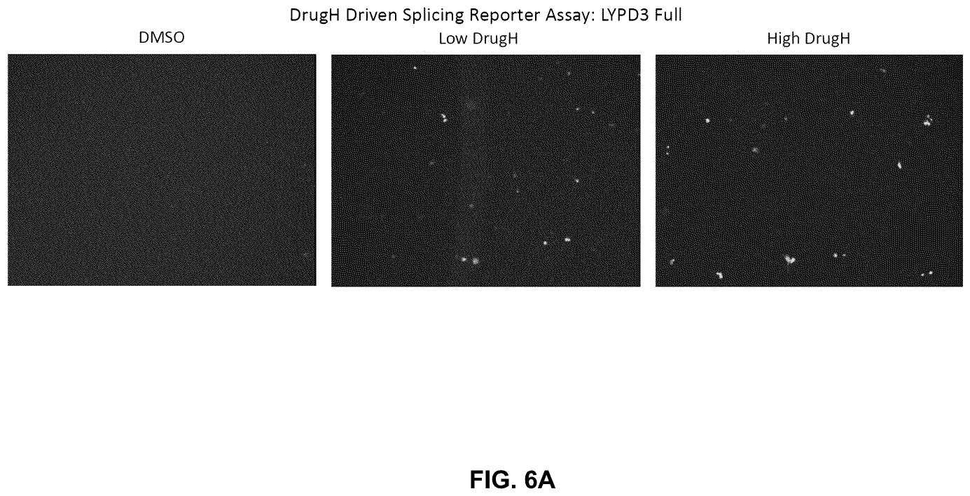

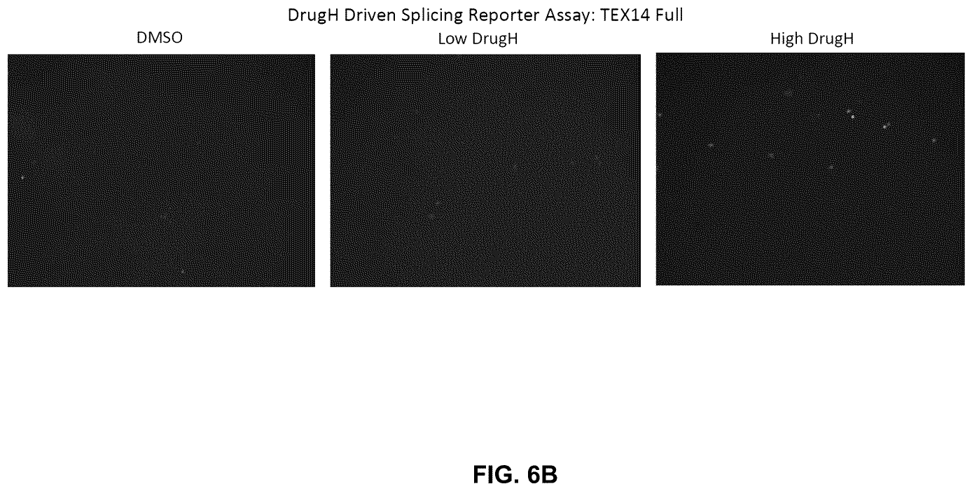

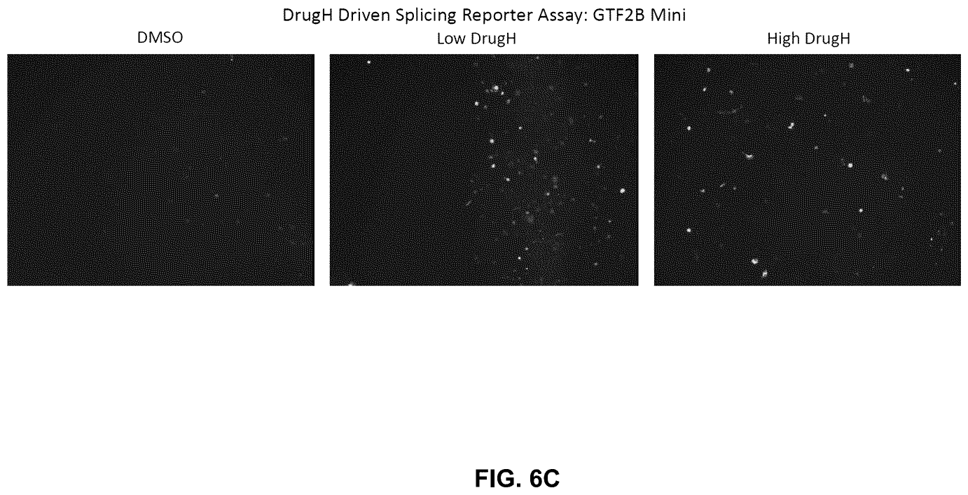

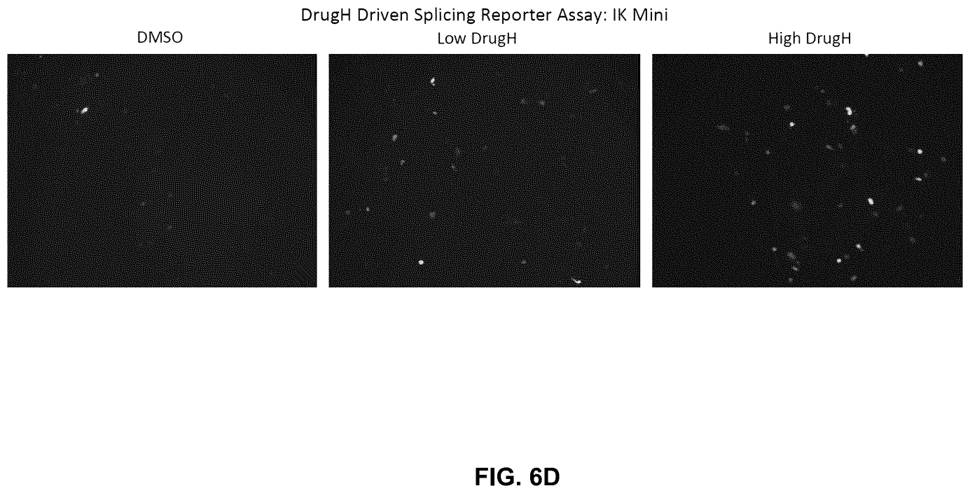

To examine the effects of Drug K or Drug H on the designed splicing reporters, the reporters were transfected into HEK293T cells. The transfected cells were then treated with the corresponding small molecule and monitored for the expression of GFP after treatment. An increase in the expression of GFP was observed by fluorescent microscopy for 6 out of the 7 designed constructs (FIGS. 5A-5C, FIGS. 6A-6D), supporting that Drug K and Drug H can operate as modulators of transgene expression via alternative splicing.

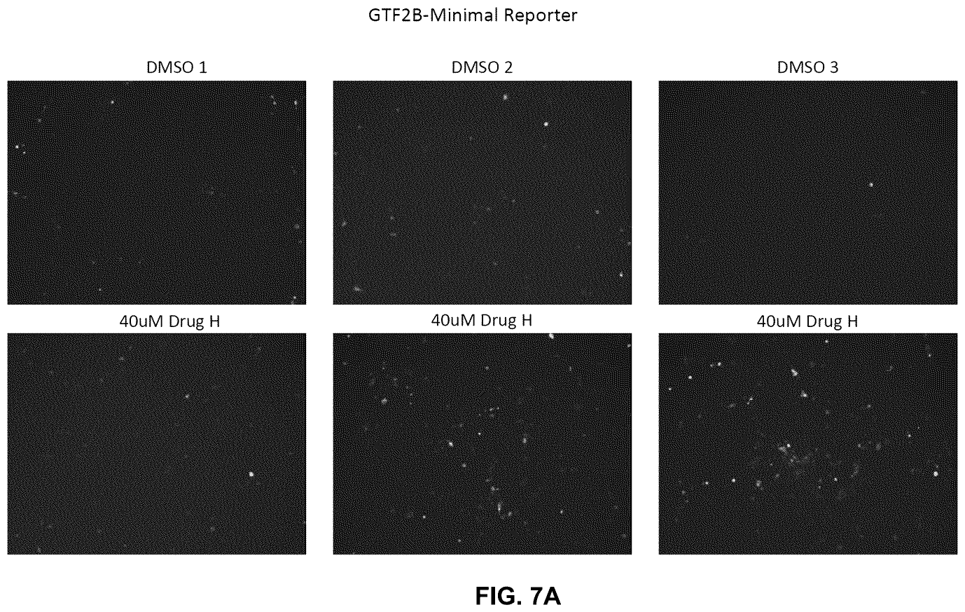

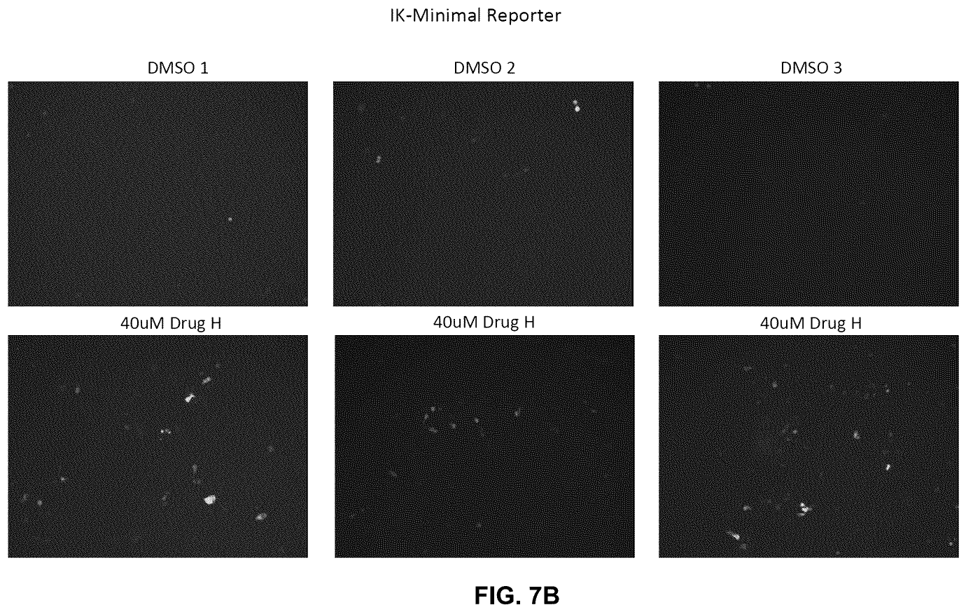

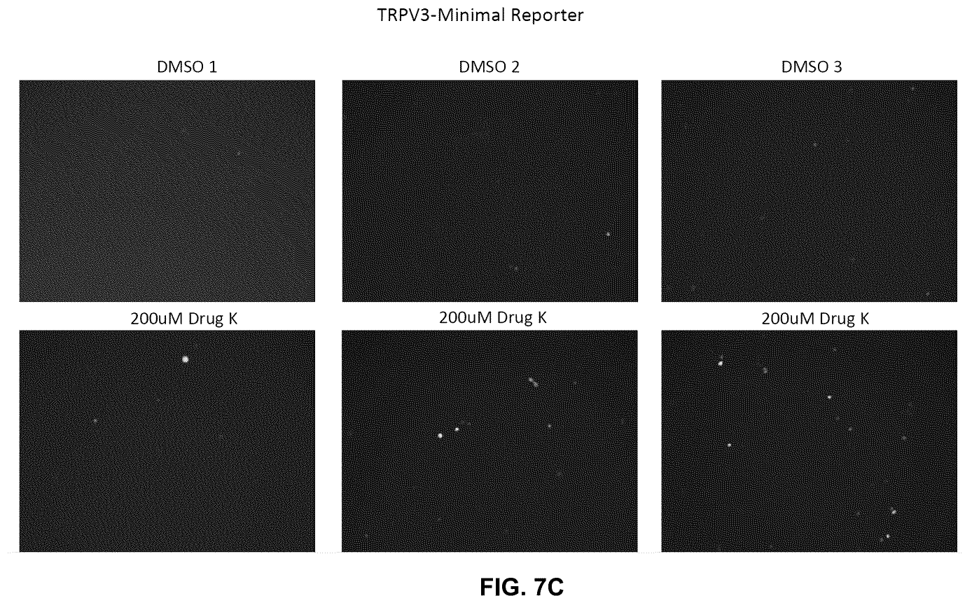

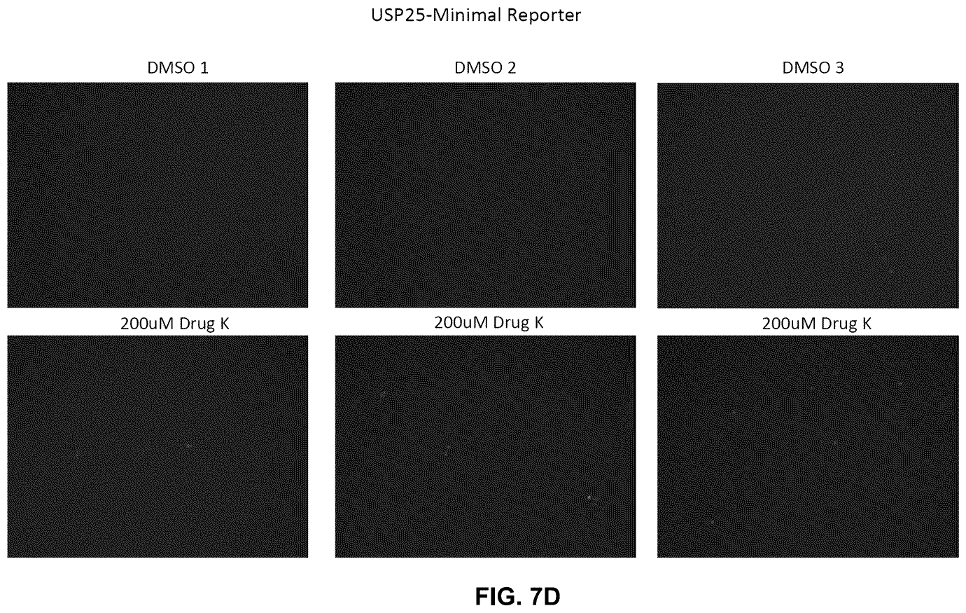

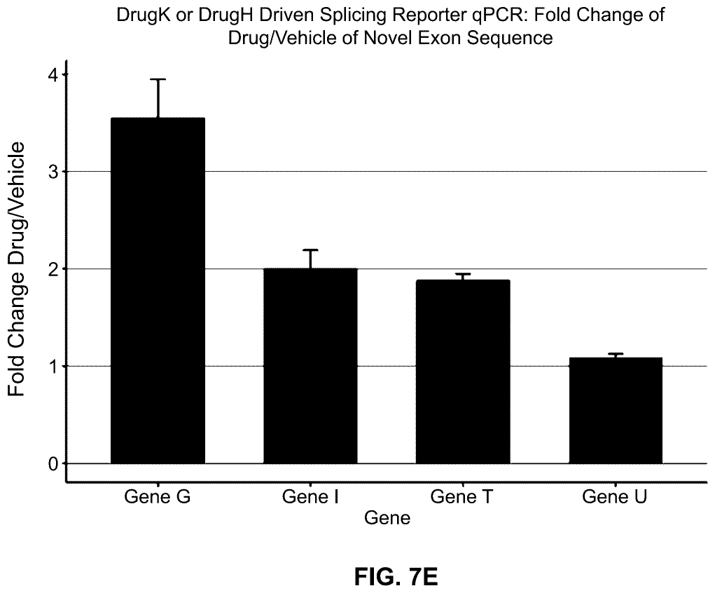

The experiments were repeated with 3 biological replicates of the reporters USP25-Minimal, GTF2B-Minimal, IK-Minimal, and TRPV3-Minimal (FIGS. 7A-7D) and again observed induction for all these constructs, however the GFP expression seen in Drug K treated USP25-Minimal cells was significantly lower than the other reporters.

To quantify the levels of expression, quantitative real-time polymerase chain reactions (q-RT-PCR) was performed on the RNA isolated from these transfected cells, targeted toward the novel exon (FIG. 7E). There was a significant increase in the amount of novel exon RNA detected when comparing either Drug K or Drug H compared to vehicle treated cells for GTF2B-Minimal, IK-Minimal, and TRPV3-Minimal reporters.

### Concentration Dependent

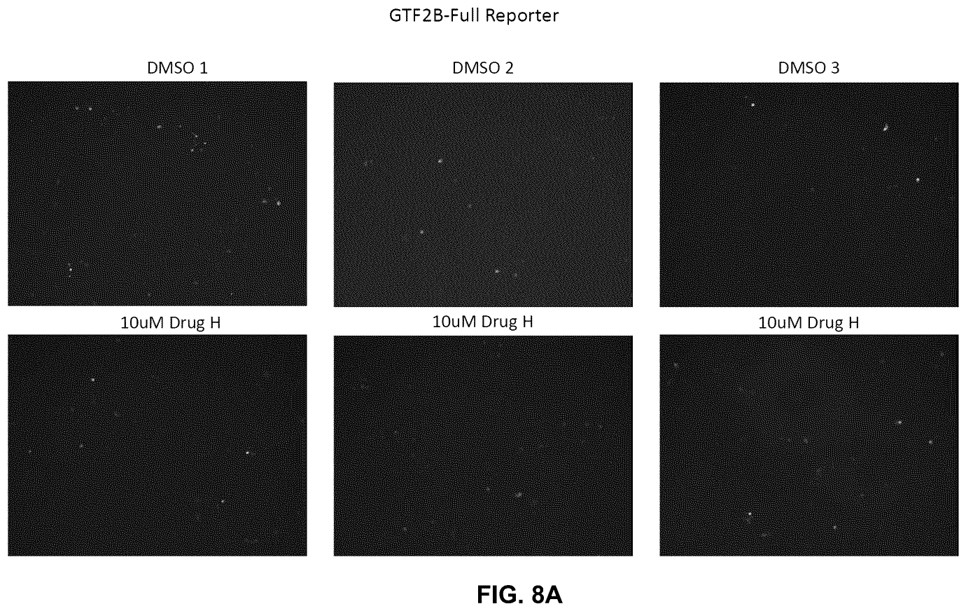

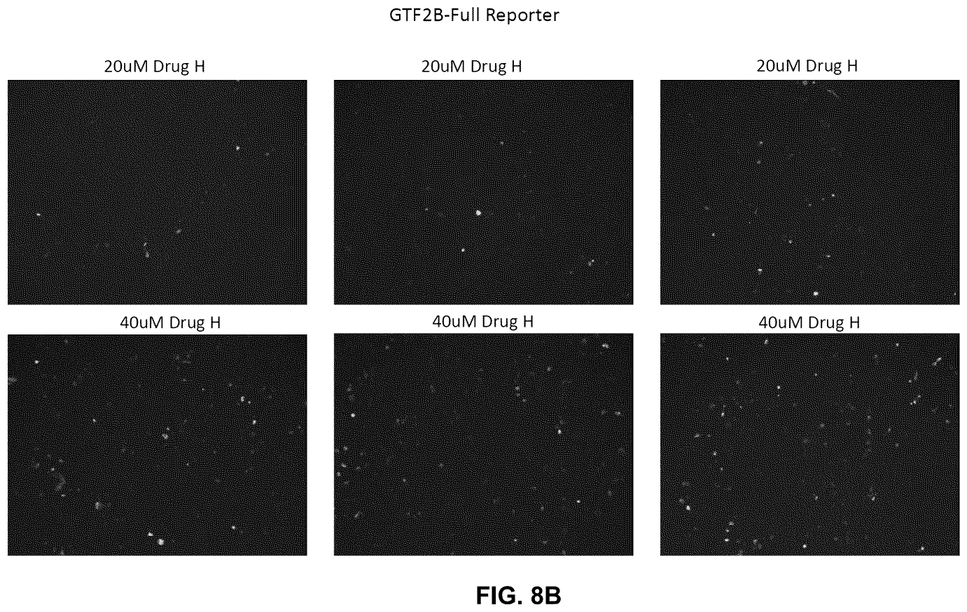

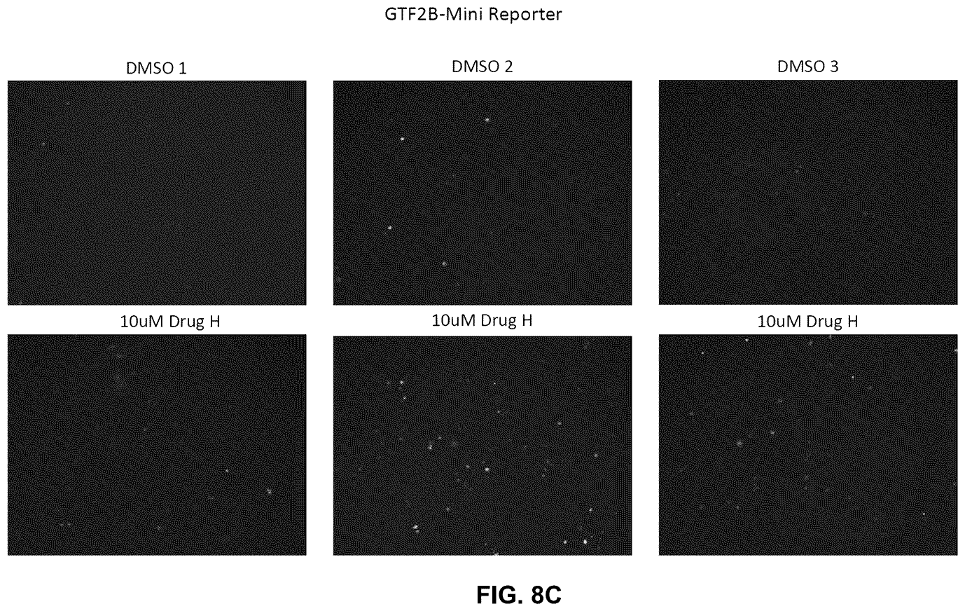

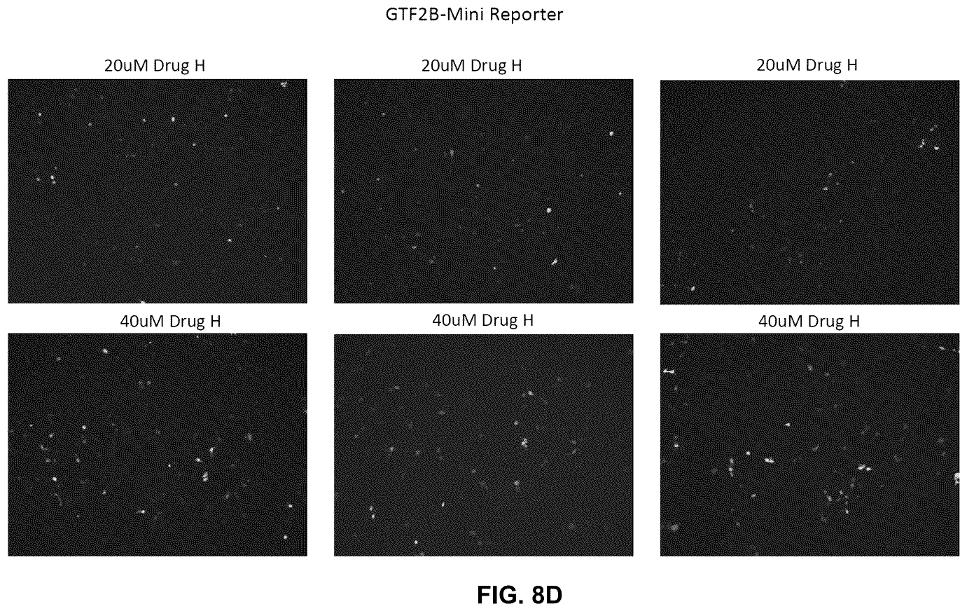

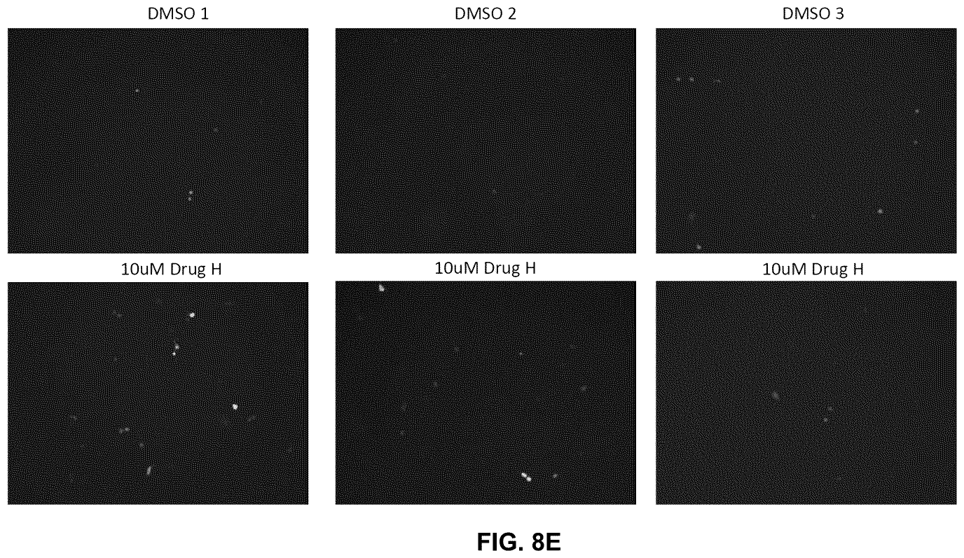

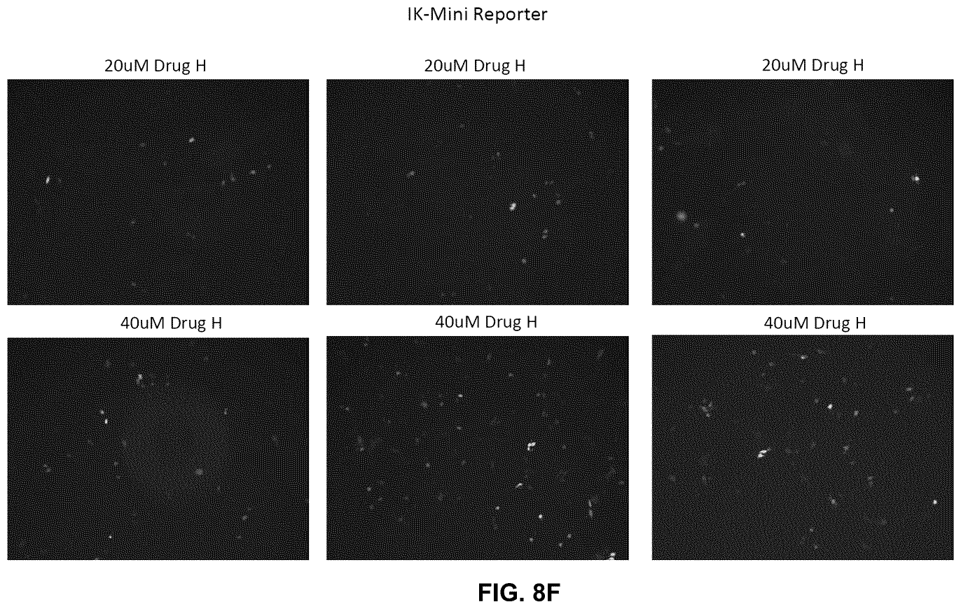

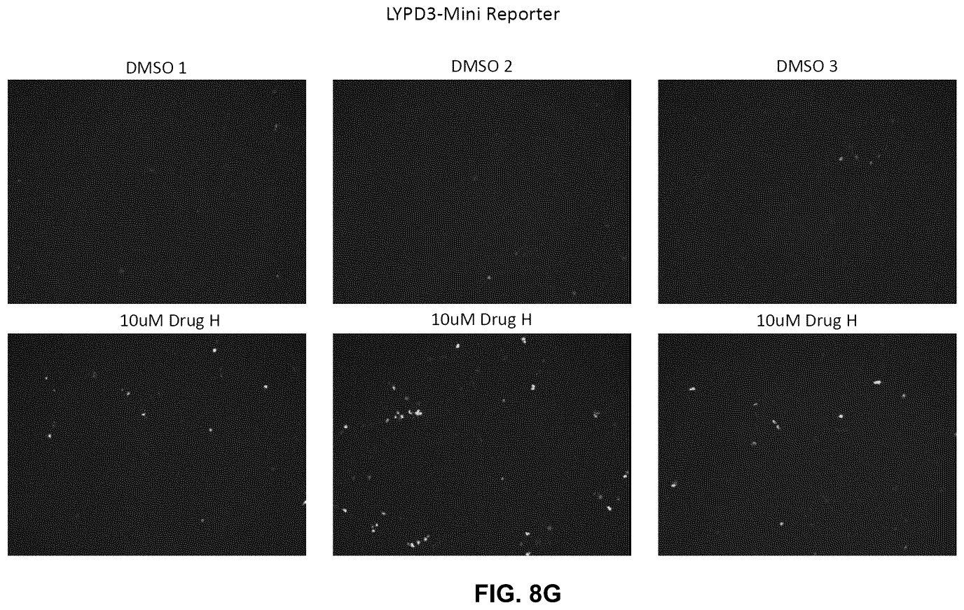

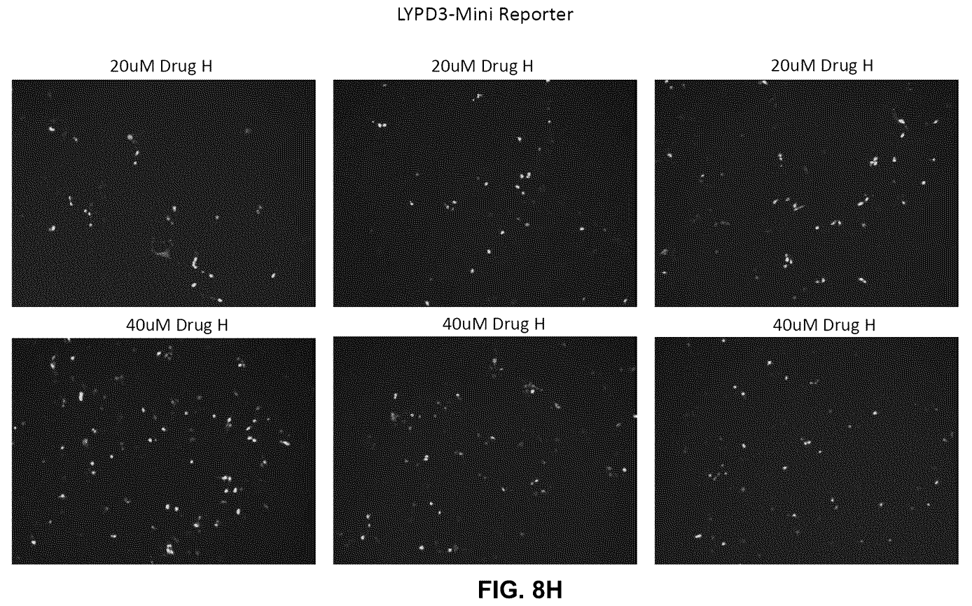

The expression of the GFP transgene was concentration dependent, allowing for control of dosing to modulate the levels of the targeted transgene. Cells were transfected with GTF2B-Full, GTF2B-Minimal, IK-Minimal, or LYPD3-Minimal and treated with increasing amounts of Drug H (FIGS. 8A-8H). Cells transfected with each of these constructs demonstrated a Drug H concentration-dependent increase in GFP expression. These results demonstrated that Drug H can modulate the protein expression level of alternative-splicing driven transgenes in a dose responsive manner.

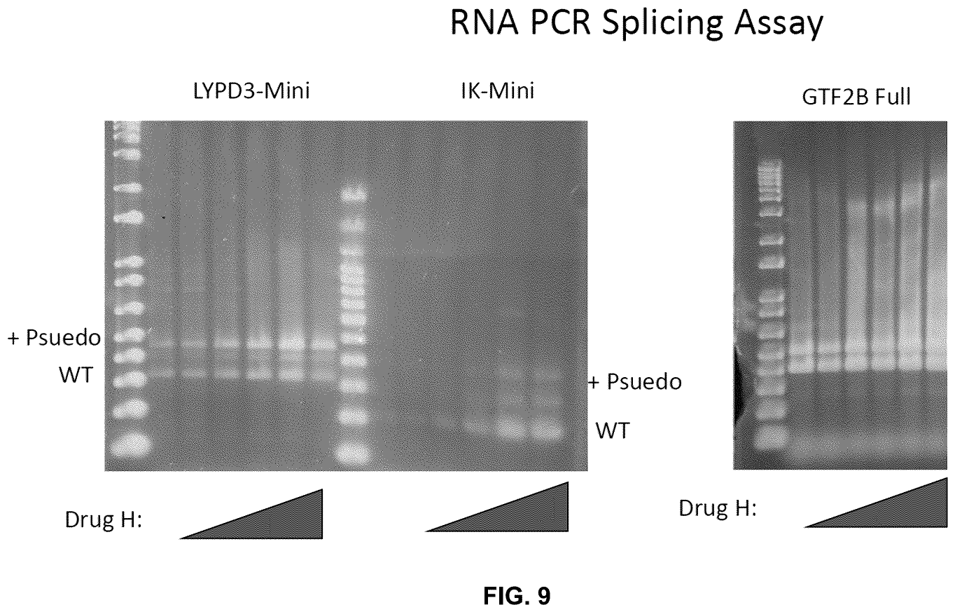

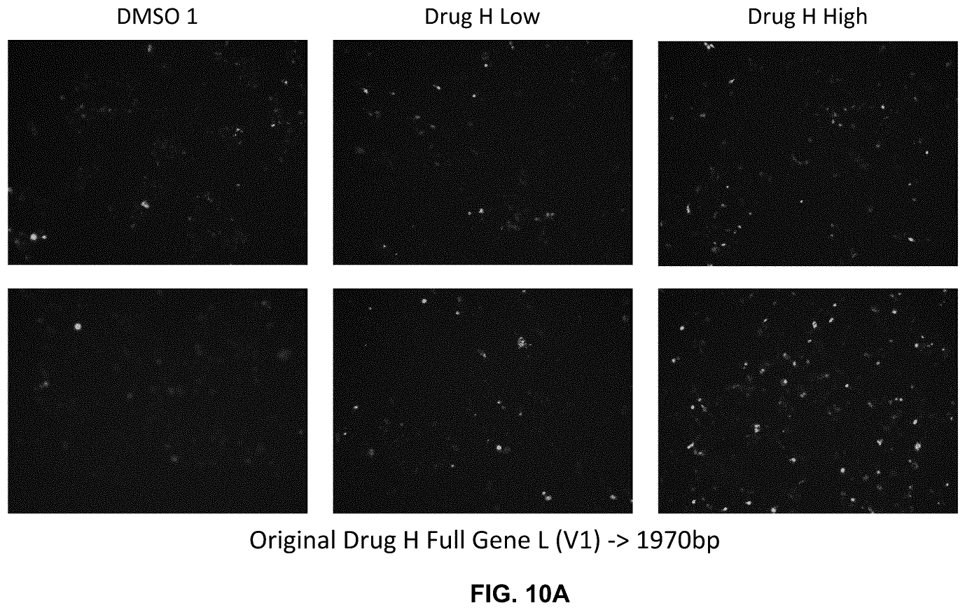

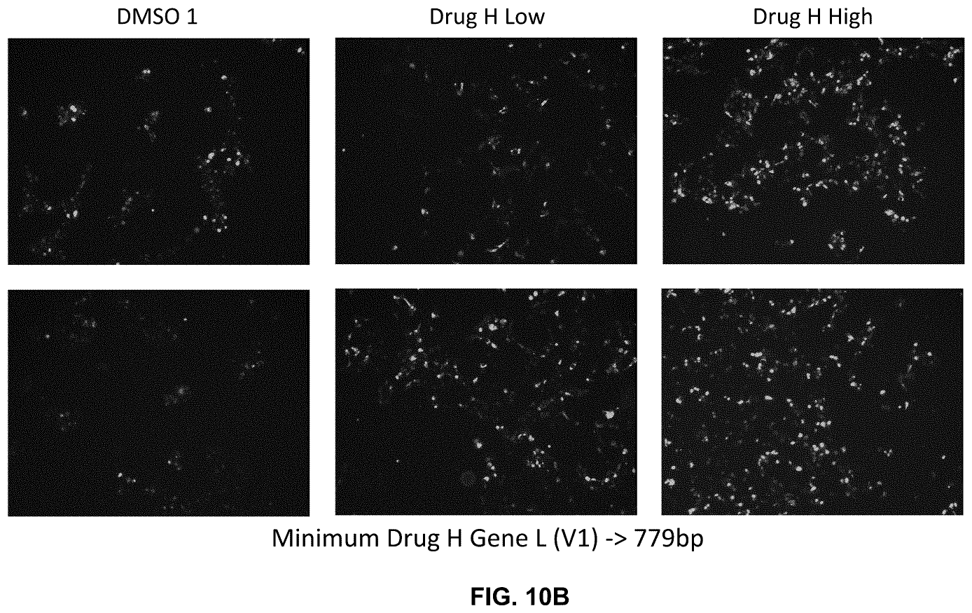

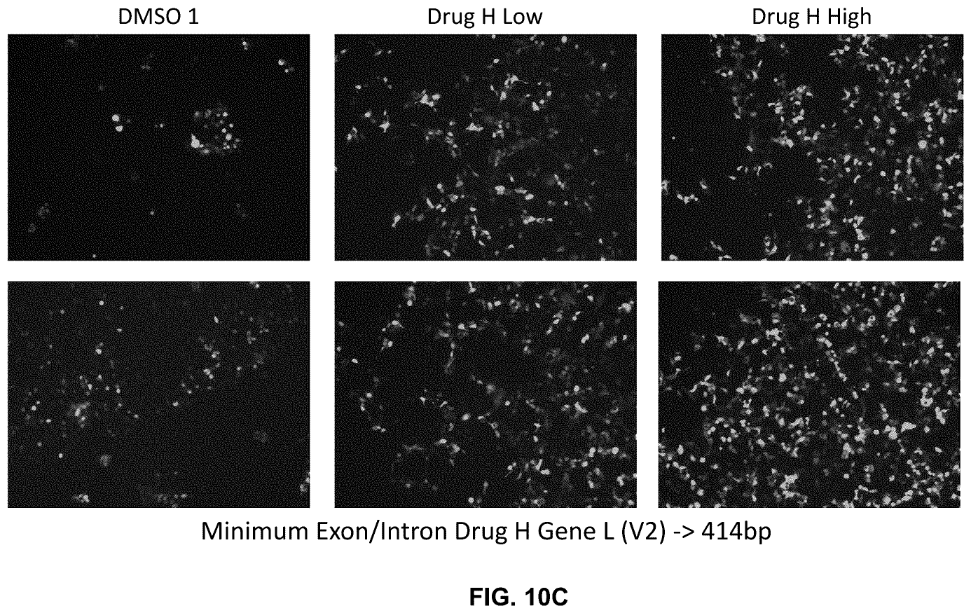

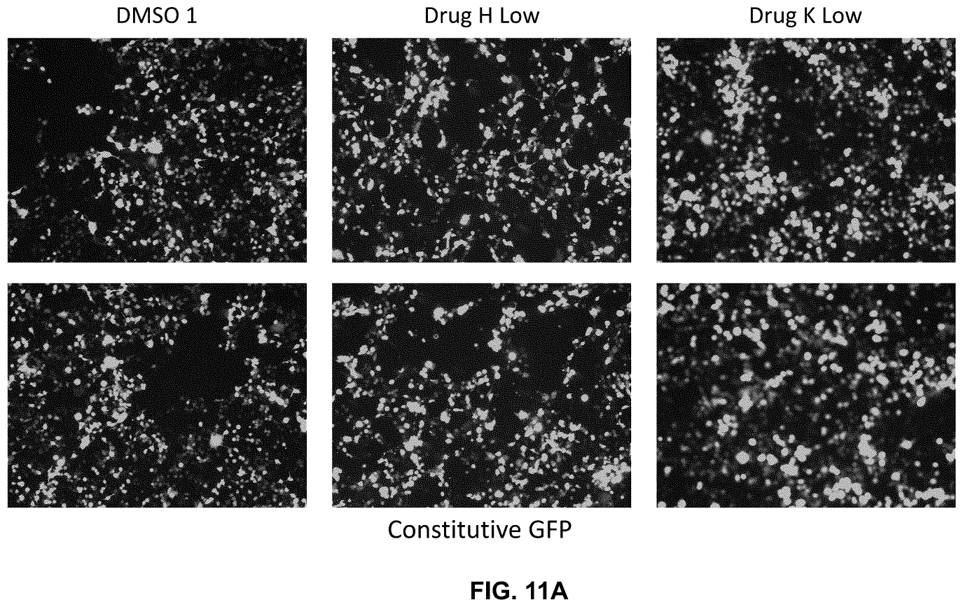

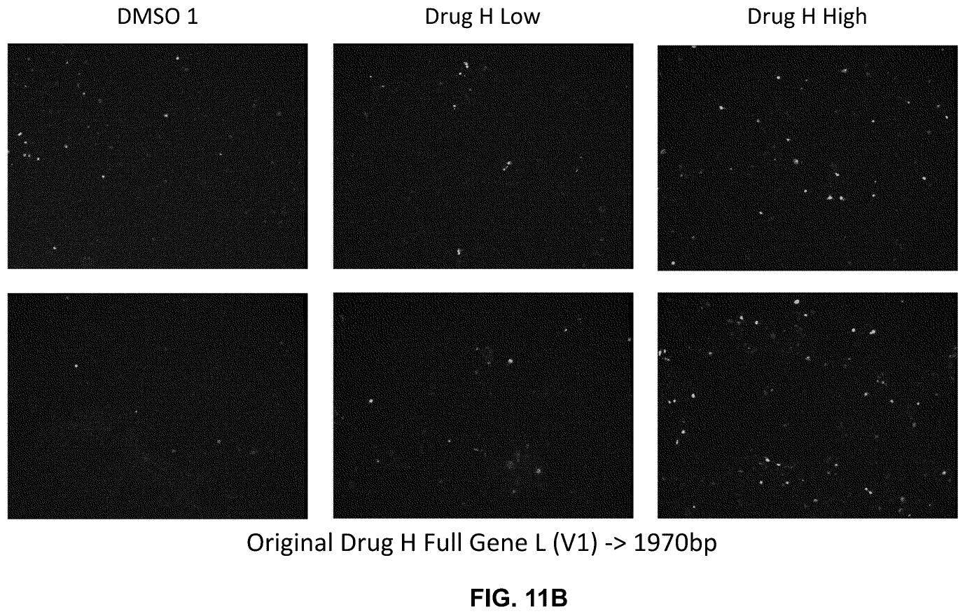

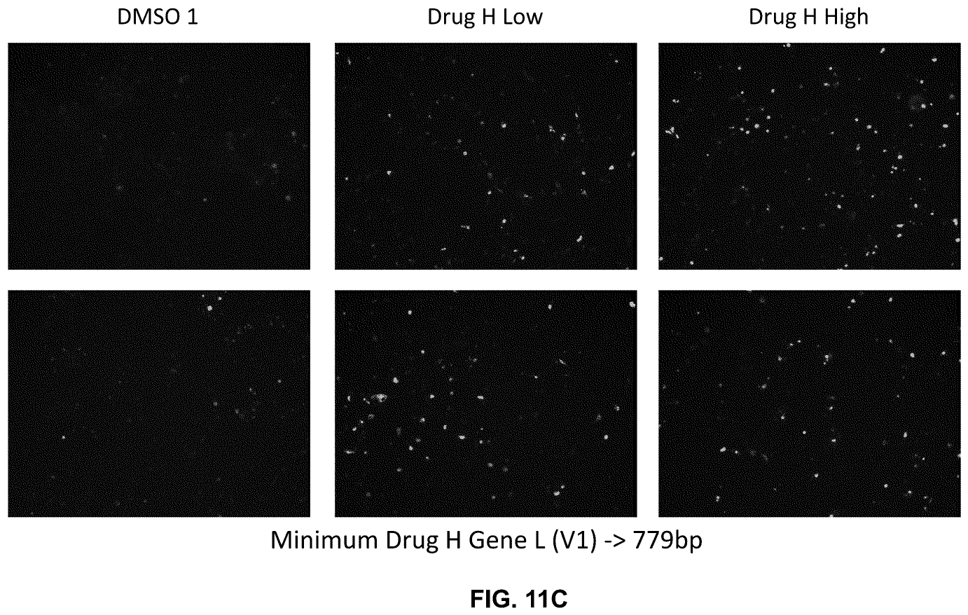

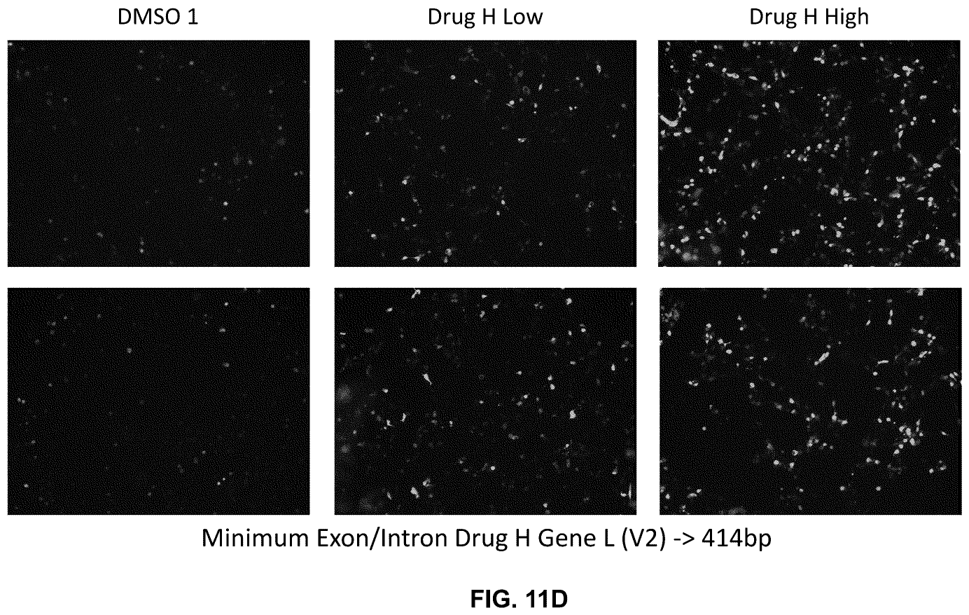

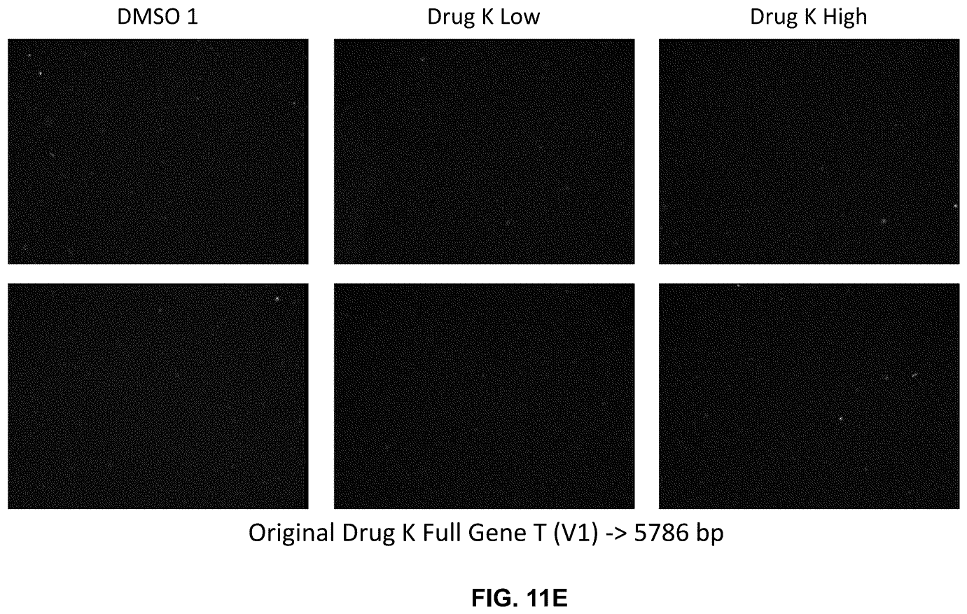

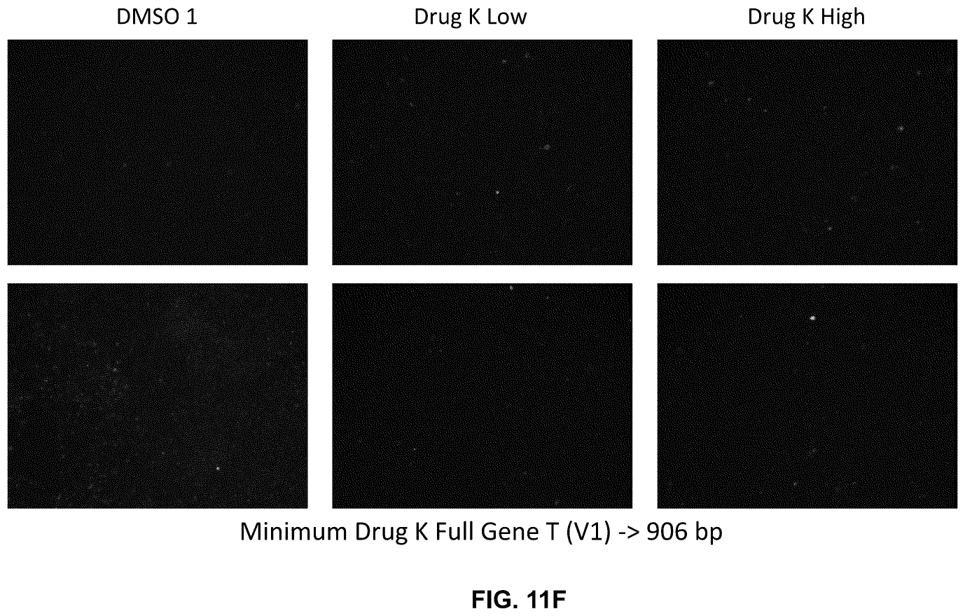

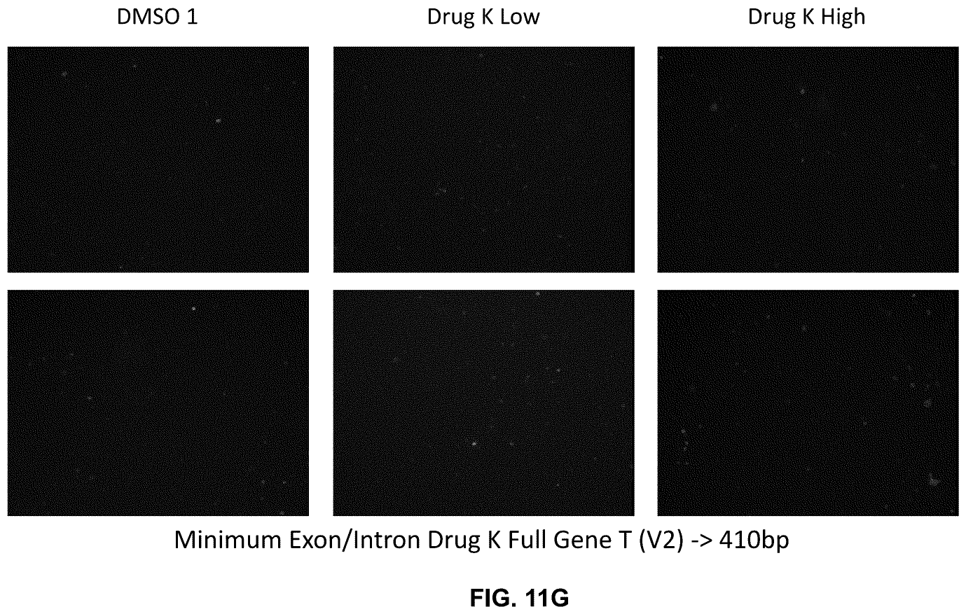

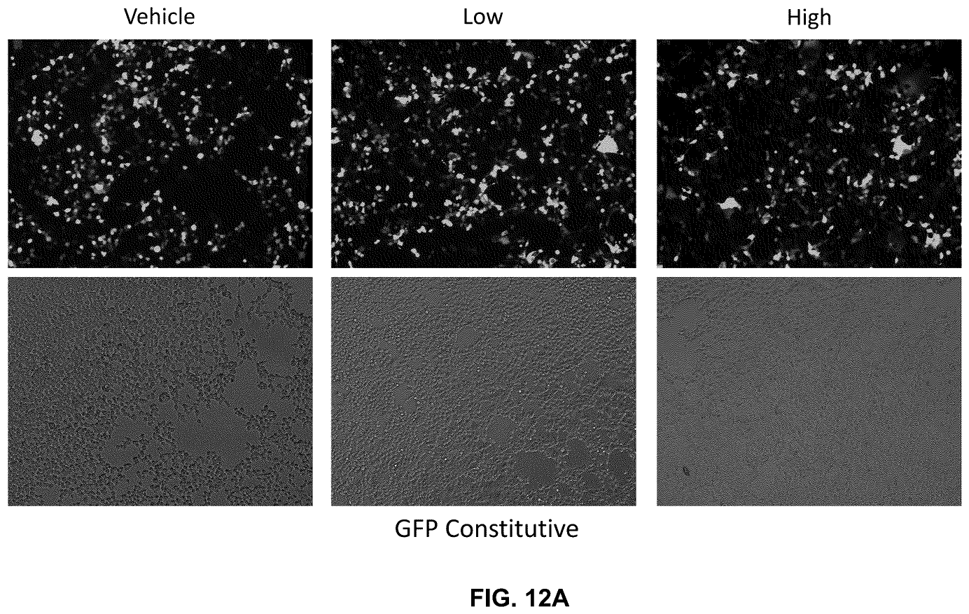

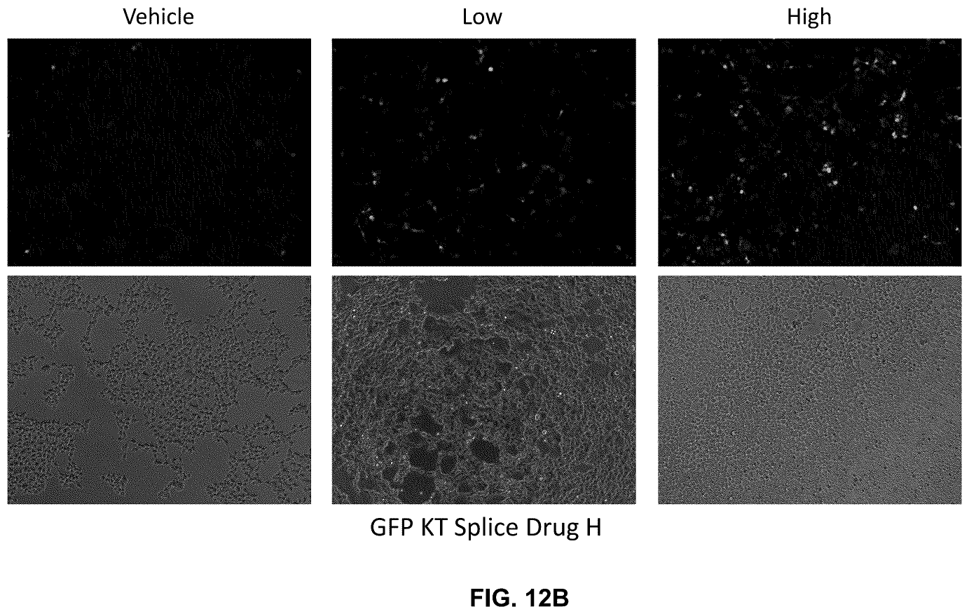

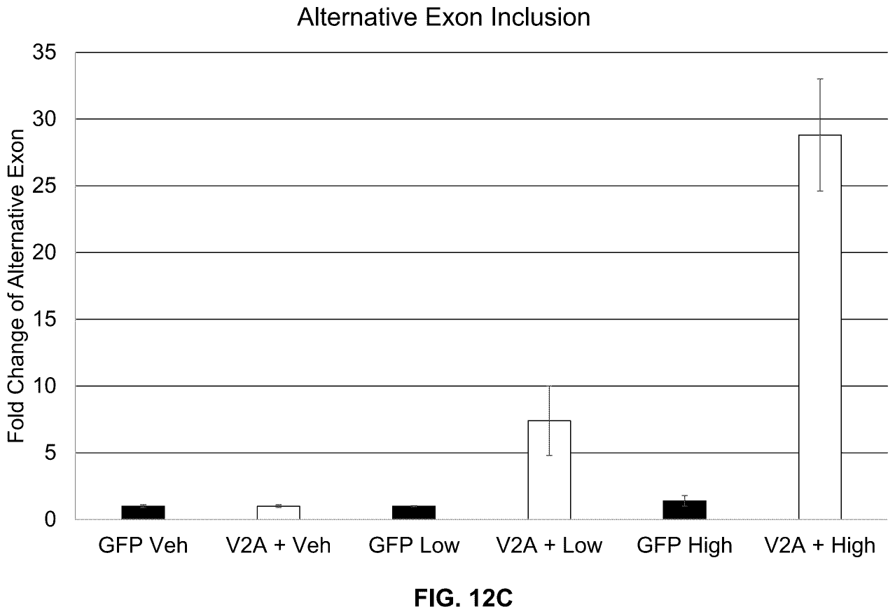

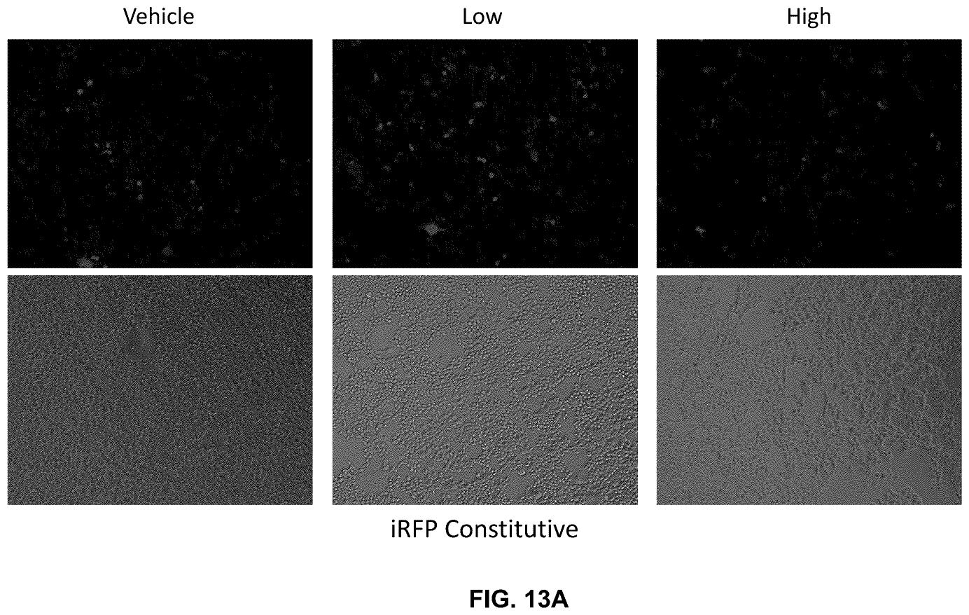

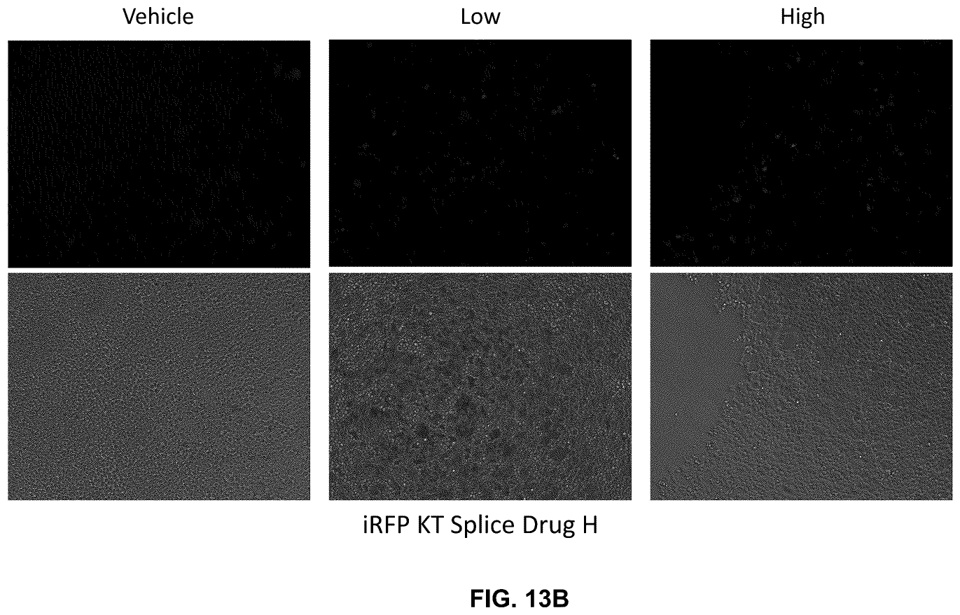

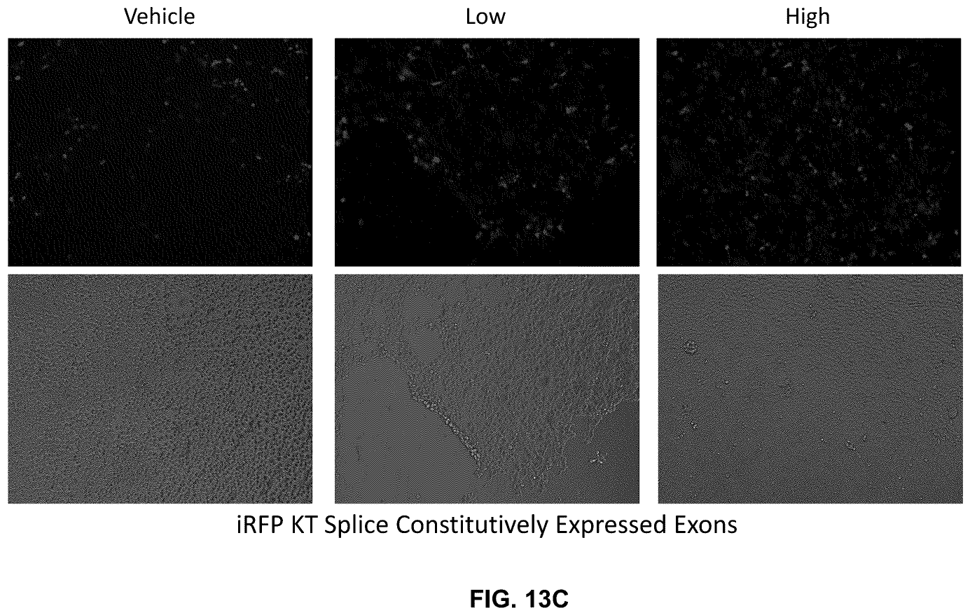

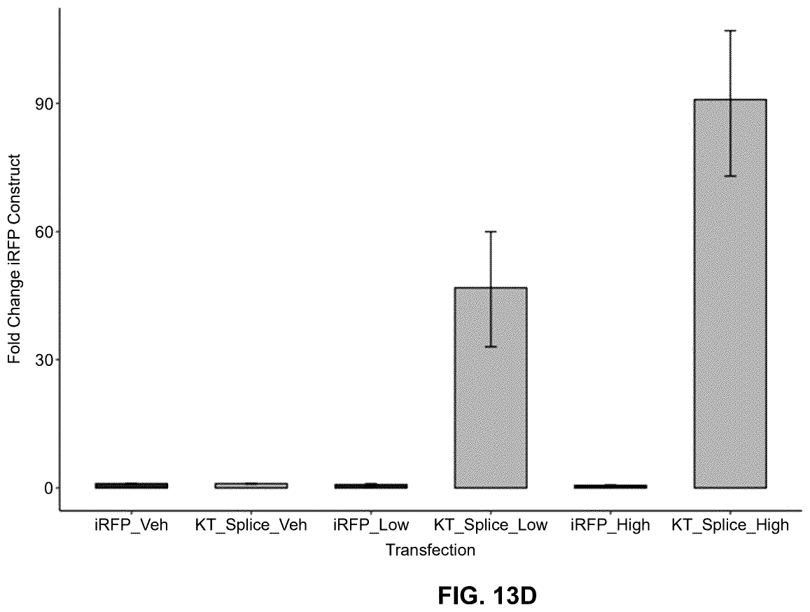

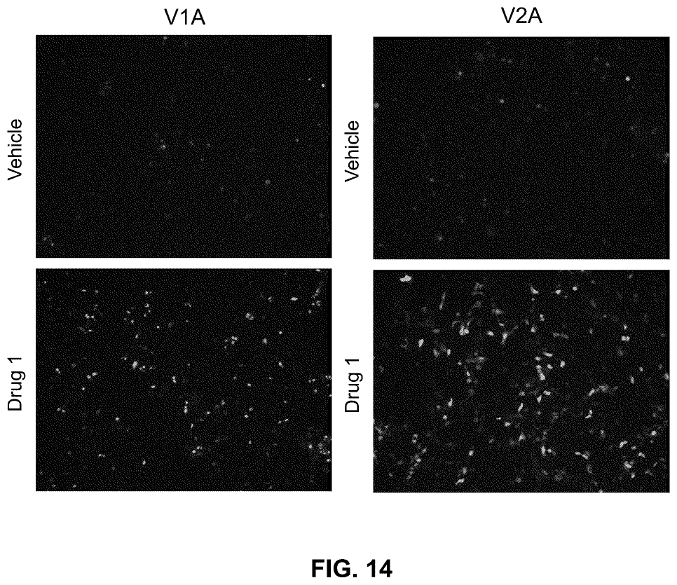

To visualize the effect of these small molecules on the splicing reporter constructs, an additional assay was designed to examine the size of the transgenic RNAs produced in cells transfected with the reporter constructs. PCR was used to amplify the upstream exon region to GFP in treated and untreated cells, expecting that there would be a shift in the size and amount of the amplicon as Drug H or Drug K was added, due to the inclusion of the novel exon. This effect was observed for cells transfected with the LYPD3-Minimal, IK-Minimal, GTF2B-Full or GTF2B-Minimal reporters (FIG. 9, FIGS. 10A-10C, and FIGS. 11A-11F). High Expression was established for Minimum Gene L (LYPD3) (FIG. 10B), Minimum Exon/Intron Gene L (LYPD3) (FIG. 10C) and Minimum Exon/Intron Gene L (LYPD3) (FIG. 11D). Further, increases in GFP and iRFP expression driven by KT Splice Drug H sensitive constructs (e.g., SEQ ID NOs: 63-66) were shown (FIGS. 12A-12C and FIGS. 13A-13D). Drug H was also able to drive high expression in two different minimal LYPD3 constructs driving eGFP expression (VIA and V2A-see FIG. 14).

In summary, the results demonstrate that two small molecule splice modulators were able to drive alternative splicing of specific gene transcripts. These small molecules-transcript pairs were utilized to control expression of transgenes post-transcriptionally in a dose dependent manner. Promoter-independent control of a transgene that could be broadly targeted to various cell types for broad therapeutic usage has been demonstrated.

### Example 3—Treatment of Cells with Small Molecule Modulators Results in Improved Transcriptome for AAV Production

To make sure Drug H and Drug K did not negatively impact packaging, cells were treated in the presence of Drug H or Drug K without the inducible exon constructs. However, it was surprisingly found that they had an impact on the transcriptome of cells during AAV production.

### Cell Culture

VPC2.0 cells (Thermo Fisher) were cultured in Viral Production Medium (Gibco) containing 4 mM GlutaMAX (Gibco) at 37 degrees Celsius with 8% carbon dioxide and shaken at 120 rpm. Triple plasmids were transfected at 0.5 μg/cell in equimolar ratios with PEIpro (Polyplus Transfection) as the DNA transporter. Cells were treated with drug additives or a DMSO vehicle control at 4- and 24-hours post-transfection. Kinetin concentrations were 50 μM, 100 M, 200 μM and 400 μM. Homocarbonyltopsentin concentrations were 5 μM, 10 μM, 20 μM and 40 μM.

### DNA Isolation and Titer

48-hours after transfection cells were lysed using 50 mM Tris HCl, 0.05% Tween-20, pH 8.5 and freeze thawed 3 times. To digest host cell DNA, 10 mM MgCl₂, 500 mM NaCl and Benzonase were added to harvested cell culture and incubated at 37° C. for 1-hour. The crude lysate was then analyzed for capsid titer by GYROS and genome titer by ddPCR.

### RNA Sequencing

RNA was isolated using the Qiagen RNeasy kit with on-column DNase digestion according to manufacturer's instructions. RNA-sequencing libraries generated, and 3 replicates of each concentration were sequenced. The resulting sequencing files were aligned to the human genome and a normalized count table was generated for downstream analysis. The splice junction output files were quantified to identify novel splice junctions.

### Results

Genome and capsid titers and protein levels were determined 48 hours after triple transfection as shown in Table 2 and Table 3. GFPTest is a plasmid containing eGFP driven by a CMV promoter with a SV40 polyA, flanked by AAV2 ITRs. CMVAAV1_Cap is a plasmid containing the AAV1 CAP gene driven by a CMV promoter. AAV2 Rep_Helper is a plasmid containing the AAV2 Rep and adenovirus helper genes.

TABLE 2
Drug K Transfection

| | | | | | | VCD (10\hat{ }6 | ddPCR - | ddPCR - | |
|---|---|---|---|---|---|---|---|---|---|
| | | Sample | | | % Viability | cells/mL) at | ITR - | GFP - | vp/mL |
| Condition | Plasmid | Number | GOI | Capsid | at Harvest | Harvest | Corrected | Corrected | Corrected |
| Negative | CMVAAV1_Cap | 1 | GFP | AAV1 | 87.9% | 4.35 | 3.40E+11 | 2.80E+11 | 2.4E+11 |
| Negative | AAV2 Rep_Helper | 2 | GFP | AAV1 | 87.7% | 4.68 | 4.00E+11 | 3.00E+11 | 2.8E+11 |
| Negative | GFPTest | 3 | GFP | AAV1 | 87.9% | 4.11 | 3.20E+11 | 2.40E+11 | 2.2E+11 |
| Vehicle | CMVAAV1_Cap | 4 | GFP | AAV1 | 90.0% | 4.72 | 2.40E+11 | 1.88E+11 | 5.8E+11 |
| Control | | | | | | | | | |
| Vehicle | AAV2 Rep_Helper | 5 | GFP | AAV1 | 87.7% | 4.73 | 1.98E+11 | 1.40E+11 | 5.4E+11 |
| Control | | | | | | | | | |
| Vehicle | GFPTest | 6 | GFP | AAV1 | 88.3% | 4.68 | 2.40E+11 | 1.76E+11 | 5.2E+11 |
| Control | | | | | | | | | |
| 50 μM | CMVAAV1_Cap | 7 | GFP | AAV1 | 81.6% | 3.30 | 1.08E+10 | 1.20E+10 | 1.3E+11 |
| 50 μM | AAV2 Rep_Helper | 8 | GFP | AAV1 | 82.7% | 4.52 | 2.40E+11 | 2.00E+11 | 3.8E+11 |
| 50 μM | GFPTest | 9 | GFP | AAV1 | 84.0% | 4.38 | 2.40E+11 | 1.74E+11 | 3.4E+11 |
| 100 μM | CMVAAV1_Cap | 10 | GFP | AAV1 | 82.6% | 4.30 | 8.40E+10 | 6.20E+10 | 2.4E+11 |
| 100 μM | AAV2 Rep_Helper | 11 | GFP | AAV1 | 79.6% | 4.32 | 6.00E+10 | 4.00E+10 | 2.2E+11 |
| 100 μM | GFPTest | 12 | GFP | AAV1 | 82.4% | 4.48 | 7.20E+10 | 5.20E+10 | 2.0E+11 |
| 200 μM | CMVAAV1_Cap | 13 | GFP | AAV1 | 75.5% | 3.14 | 6.60E+09 | 4.40E+09 | 5.8E+10 |
| 200 μM | AAV2 Rep_Helper | 14 | GFP | AAV1 | 73.9% | 3.02 | 7.00E+09 | 5.00E+09 | 6.6E+10 |
| 200 μM | GFPTest | 15 | GFP | AAV1 | 70.4% | 2.70 | 6.00E+09 | 4.20E+09 | 3.8E+10 |
| 400 μM | CMVAAV1_Cap | 16 | GFP | AAV1 | 27.0% | 0.89 | 3.20E+09 | 3.40E+09 | — |
| 400 μM | AAV2 Rep_Helper | 17 | GFP | AAV1 | 48.8% | 1.55 | 2.60E+09 | 2.40E+09 | — |
| 400 μM | GFPTest | 18 | GFP | AAV1 | 56.4% | 1.92 | 2.40E+09 | 2.60E+09 | — |

TABLE 3
Drug H Transfection

| | | | | | | VCD | | | |
|---|---|---|---|---|---|---|---|---|---|
| | | | | | % | (10\hat{ }6cells/ | | ddPCR - | |
| | | Sample | | | Viability at | mL) at | ddPCR-ITR - | GFP - | vp/mL |
| Condition | Plasmid | Number | GOI | Capsid | Harvest | Harvest | corrected | Corrected | Corrected |
| Negative | CMVAAV1_Cap | 1 | GFP | AAV1 | 87.0% | 5.20 | 4.20E+11 | 2.80E+11 | 3.0E+11 |
| Negative | AAV2 Rep_Helper | 2 | GFP | AAV1 | 86.9% | 5.29 | 4.20E+11 | 2.80E+11 | 3.2E+11 |
| Negative | GFPTest | 3 | GFP | AAV1 | 87.8% | 5.46 | 4.40E+11 | 3.00E+11 | 3.4E+11 |
| Vehicle | CMVAAV1_Cap | 4 | GFP | AAV1 | 88.4% | 5.51 | 4.40E+11 | 3.00E+11 | 4.6E+11 |
| Control | | | | | | | | | |
| Vehicle | AAV2 Rep_Helper | 5 | GFP | AAV1 | 87.2% | 5.33 | 4.80E+11 | 3.00E+11 | 4.4E+11 |
| Control | | | | | | | | | |
| Vehicle | GFPTest | 6 | GFP | AAV1 | 87.0% | 5.17 | 4.00E+11 | 2.60E+11 | 4.4E+11 |
| Control | | | | | | | | | |
| 5 μM | CMVAAV1_Cap | 7 | GFP | AAV1 | 75.2% | 4.00 | 2.40E+11 | 2.00E+11 | 7.0E+11 |
| 5 μM | AAV2 Rep_Helper | 8 | GFP | AAV1 | 75.2% | 3.84 | 2.60E+11 | 2.20E+11 | 7.2E+11 |
| 5 μM | GFPTest | 9 | GFP | AAV1 | 74.8% | 4.16 | 2.80E+11 | 2.40E+11 | 7.0E+11 |
| 10 μM | CMVAAV1_Cap | 10 | GFP | AAV1 | 68.5% | 3.58 | 1.86E+11 | 1.74E+11 | 1.2E+12 |
| 10 μM | AAV2 Rep_Helper | 11 | GFP | AAV1 | 66.7% | 3.39 | 1.66E+11 | 1.56E+11 | 1.2E+12 |
| 10 μM | GFPTest | 12 | GFP | AAV1 | 66.4% | 3.39 | 1.66E+11 | 1.54E+11 | 1.2E+12 |
| 20 μM | CMVAAV1_Cap | 13 | GFP | AAV1 | 61.6% | 3.10 | 1.20E+11 | 1.14E+11 | 1.6E+12 |
| 20 μM | AAV2 Rep_Helper | 14 | GFP | AAV1 | 60.2% | 3.09 | 1.28E+11 | 1.24E+11 | 1.7E+12 |
| 20 μM | GFPTest | 15 | GFP | AAV1 | 54.3% | 2.70 | 1.08E+11 | 1.06E+11 | 1.8E+12 |
| 40 μM | CMVAAV1_Cap | 16 | GFP | AAV1 | 54.7% | 2.60 | 7.40E+10 | 6.80E+10 | 1.9E+12 |
| 40 μM | AAV2 Rep_Helper | 17 | GFP | AAV1 | 54.5% | 2.54 | 8.00E+10 | 7.00E+10 | 1.9E+12 |
| 40 μM | GFPTest | 18 | GFP | AAV1 | 55.1% | 2.79 | 8.80E+10 | 8.00E+10 | 2.0E+12 |

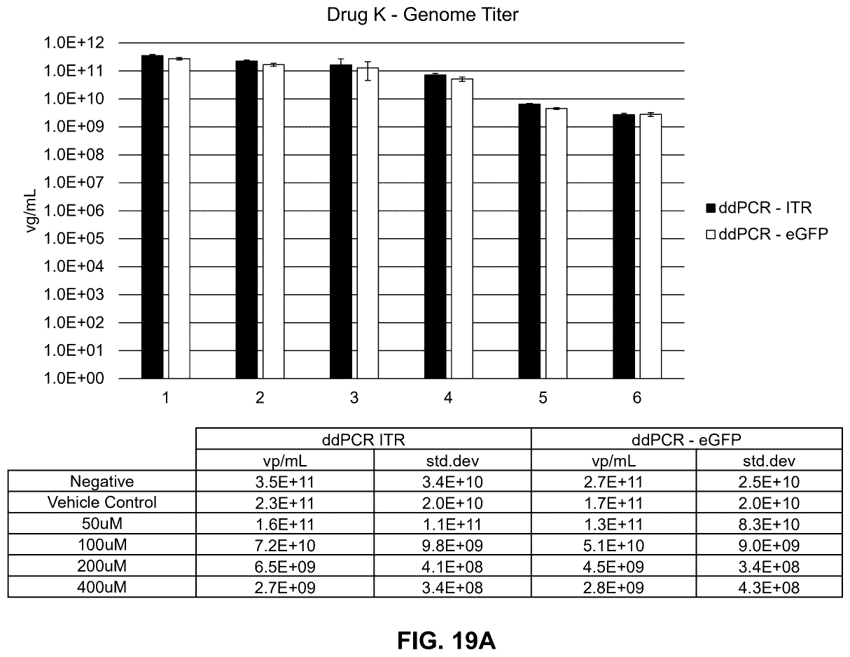

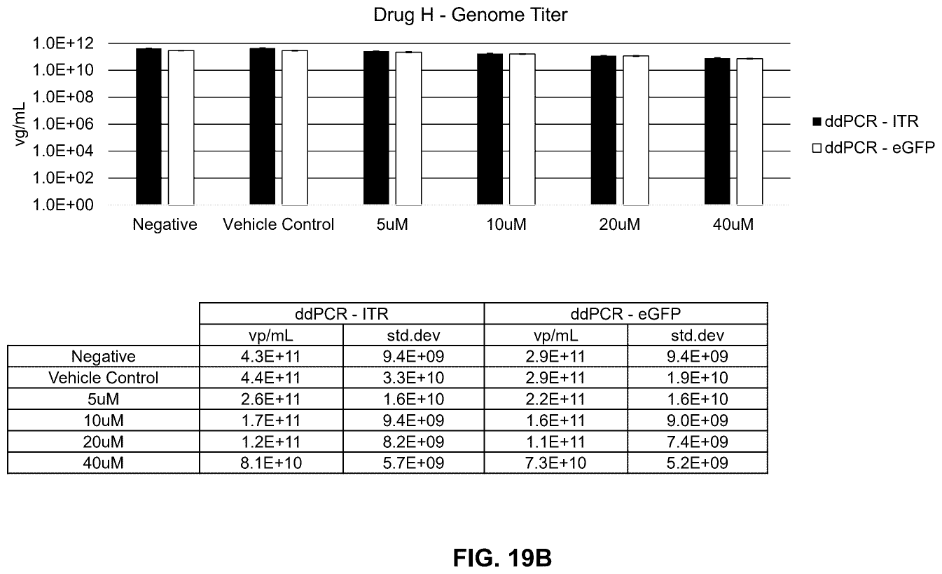

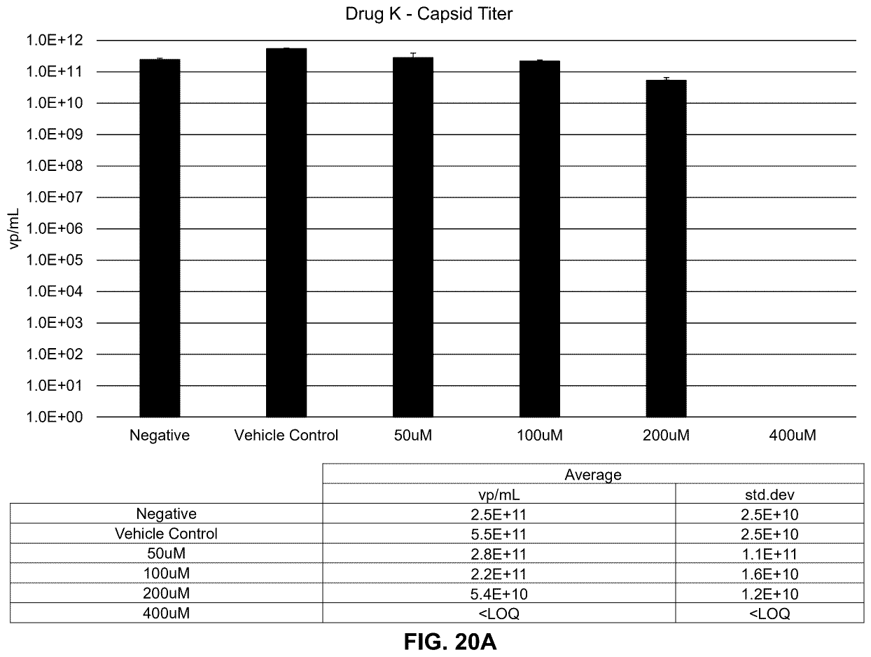

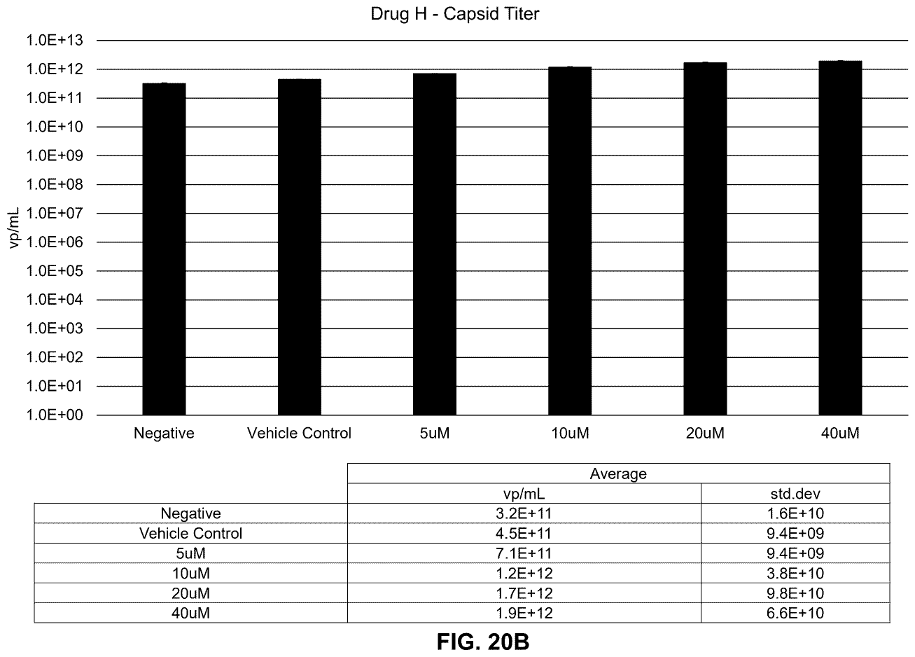

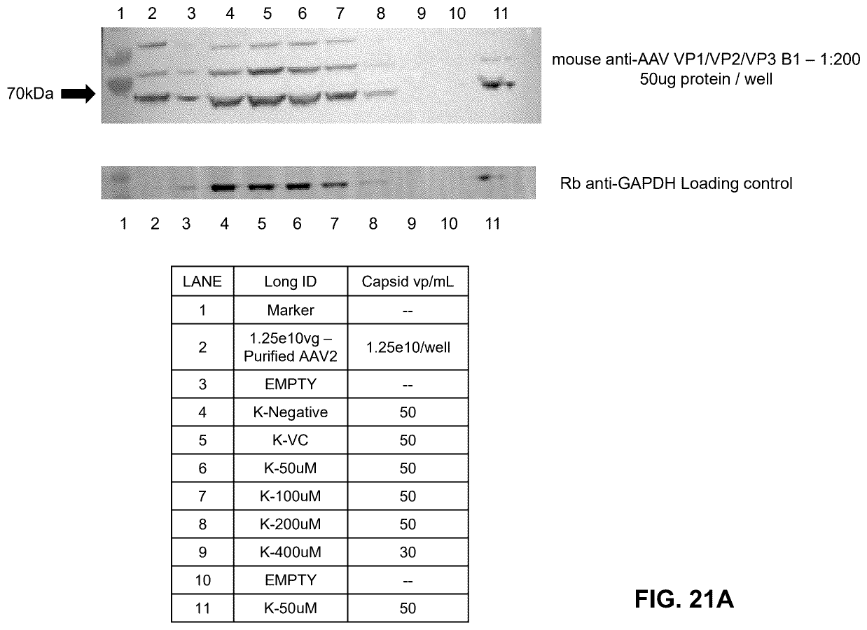

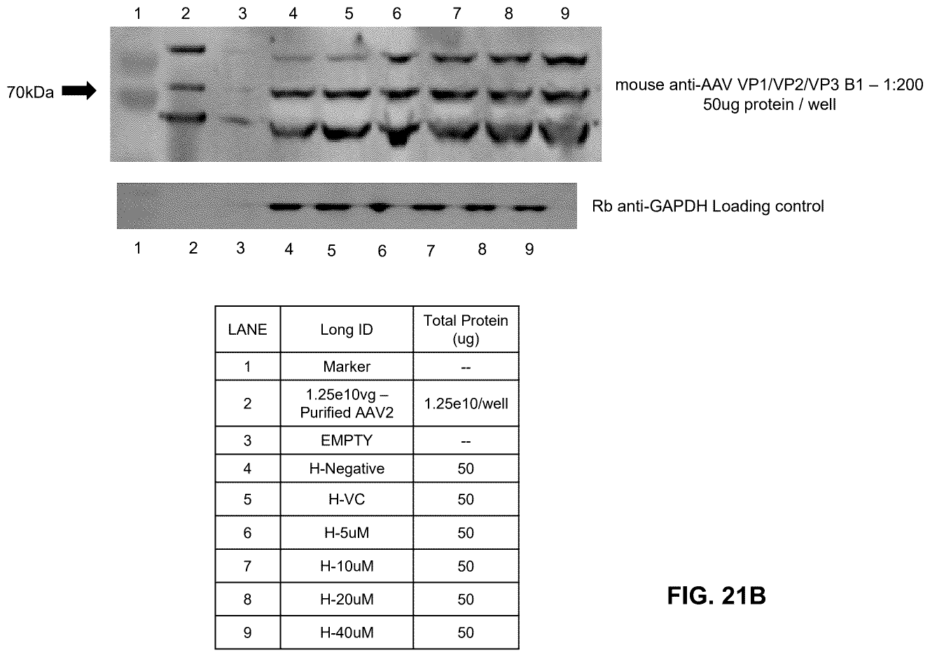

In this system, Drug H and Drug K's effect on AAV production was observed by detecting genome titer (FIGS. 19A-19B) and capsid titer (FIGS. 20A-20B). Effect on genome titer efficiency in normal cells showed that the effect was independent of activating the inducible system. Results (FIG. 20B) showed that Drug H increased capsid titer independent of activating the inducible system as measured by GYROS quantification. Additionally, VP1, VP2, and VP3 levels (FIG. 21A-21B) were observed for both systems. These results showed that Drug H increased capsid proteins (i.e., VP1, VP2, and VP3) independent of activating the inducible system.

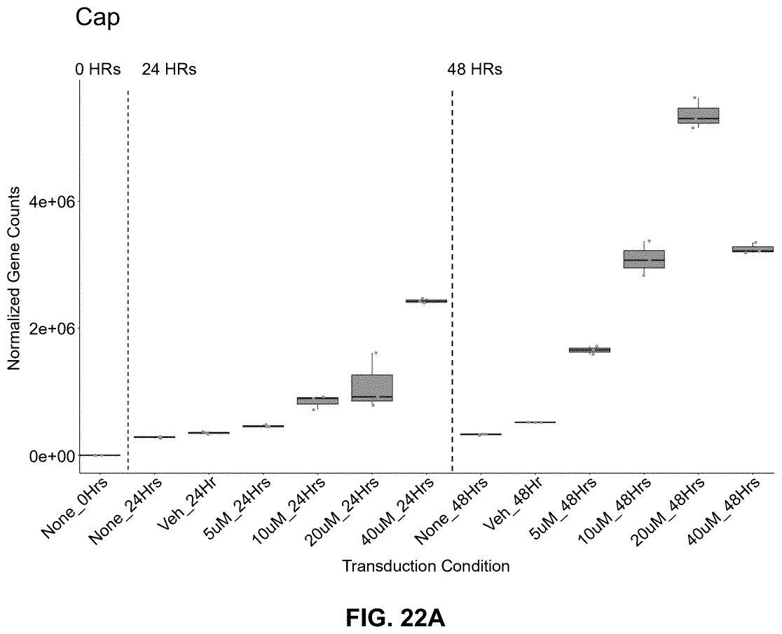

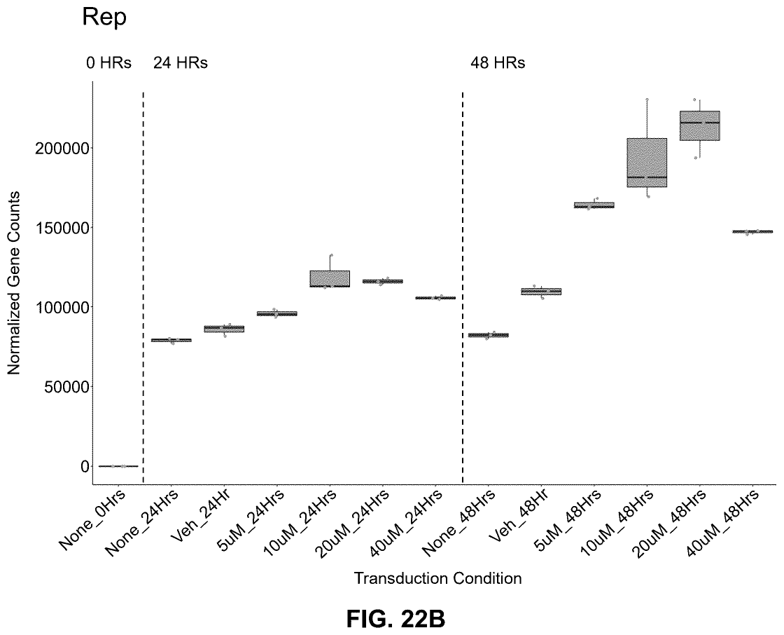

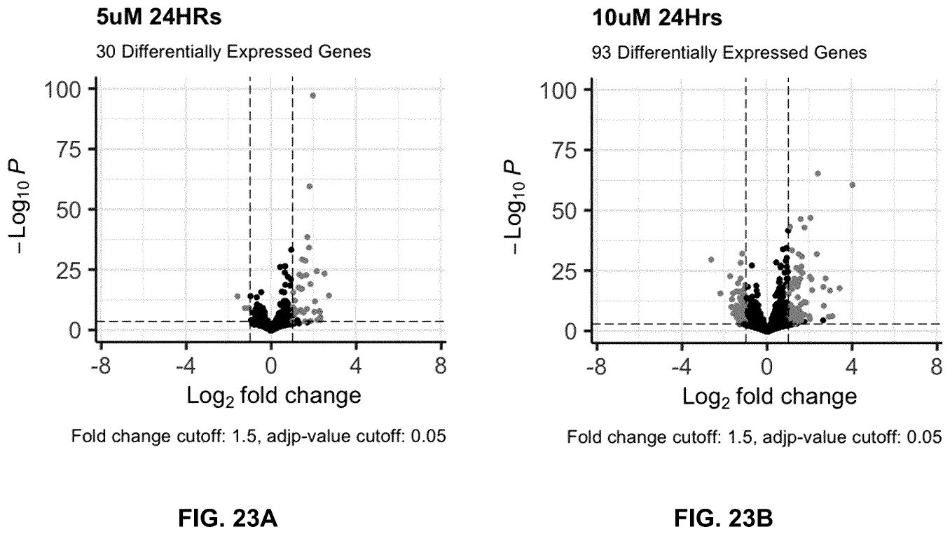

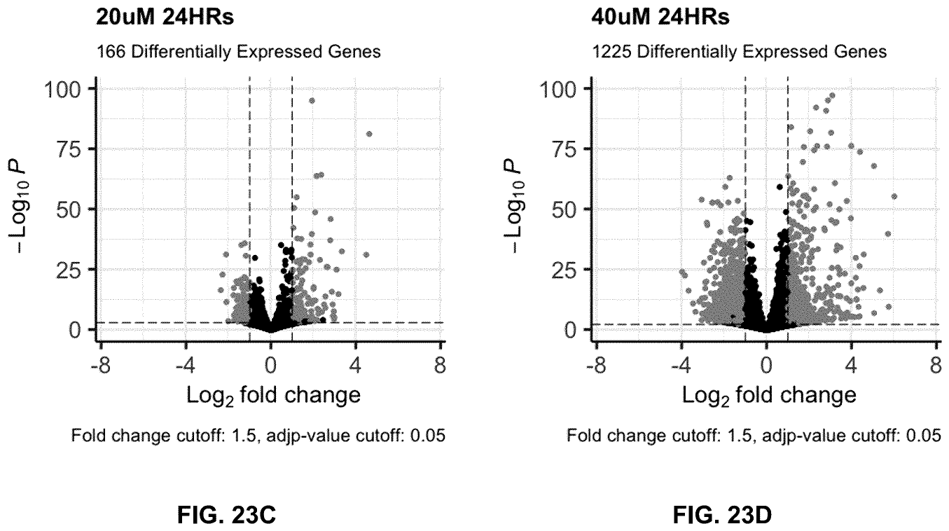

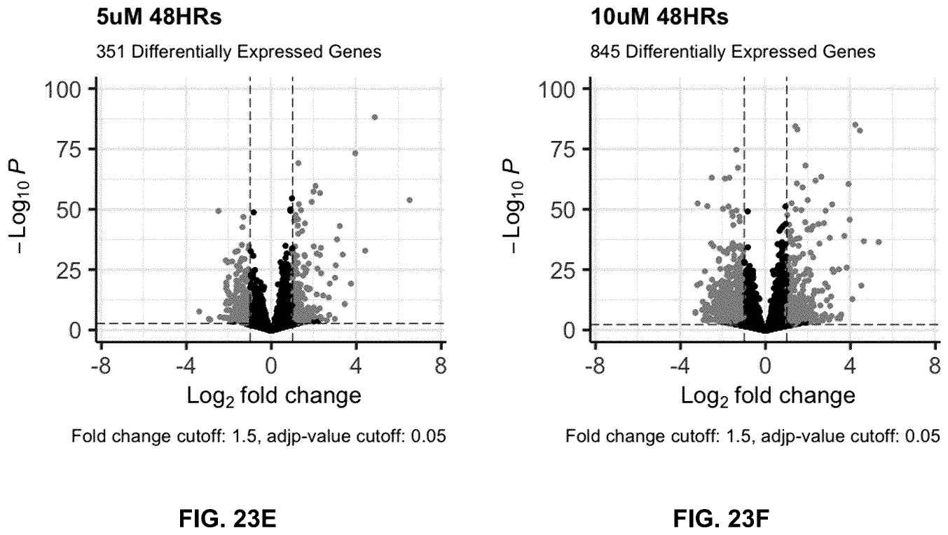

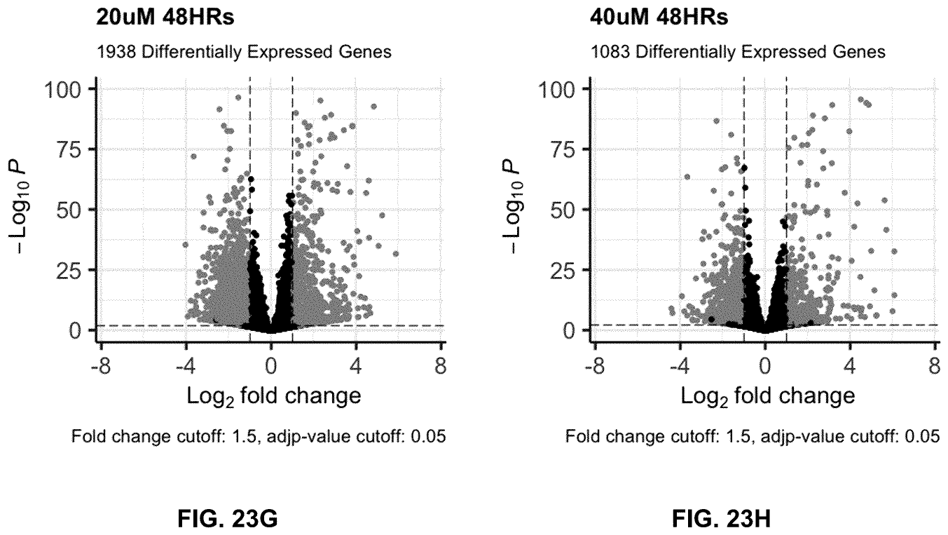

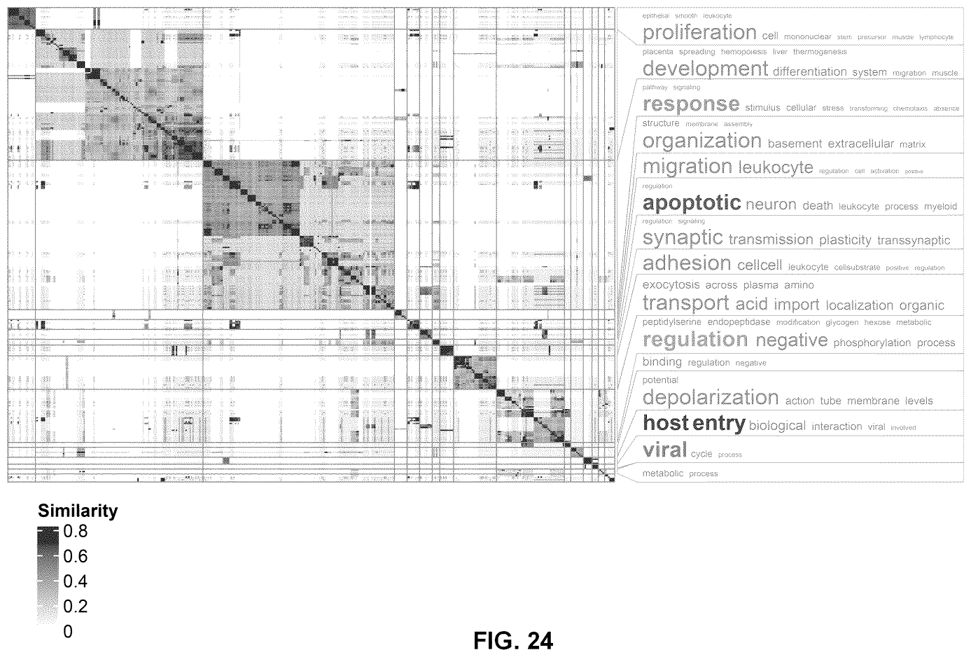

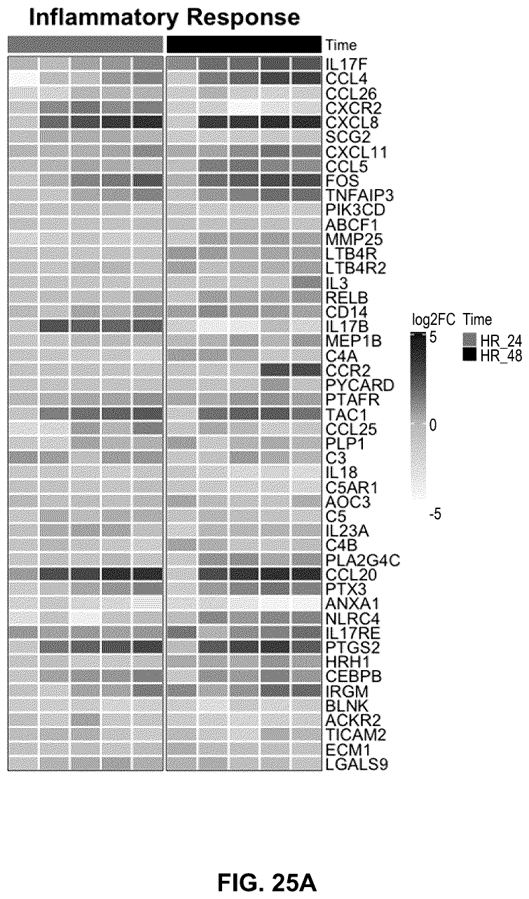

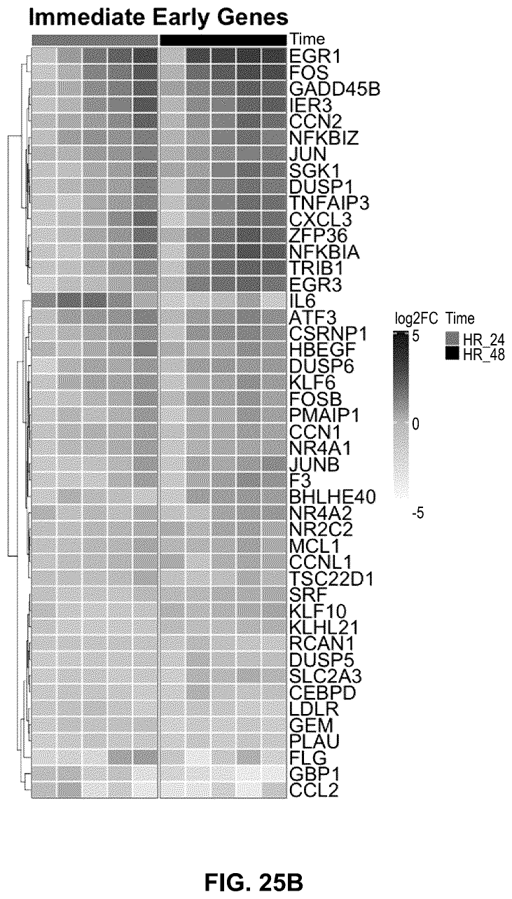

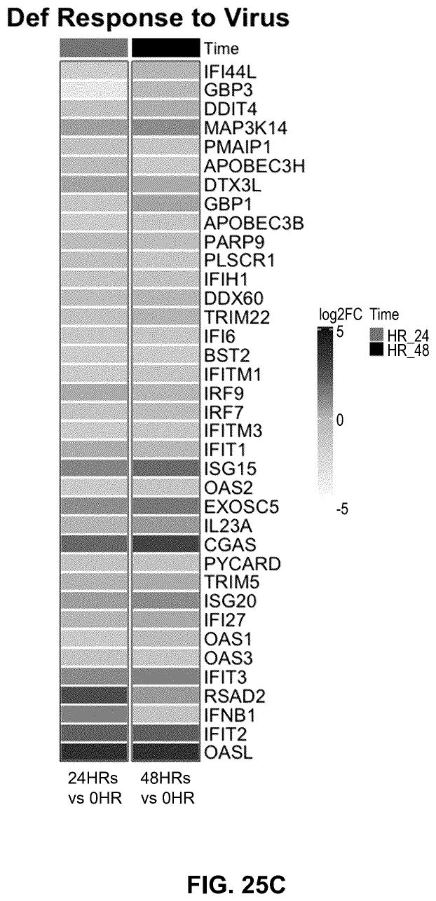

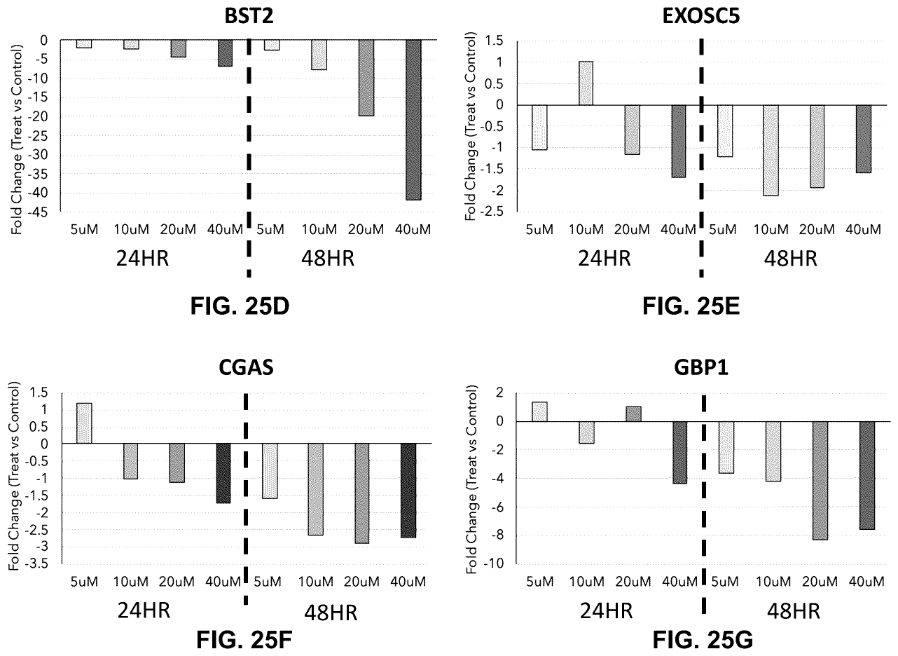

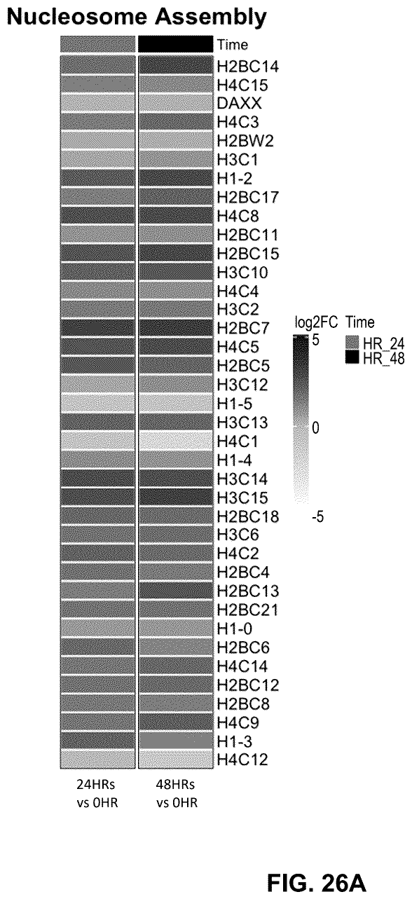

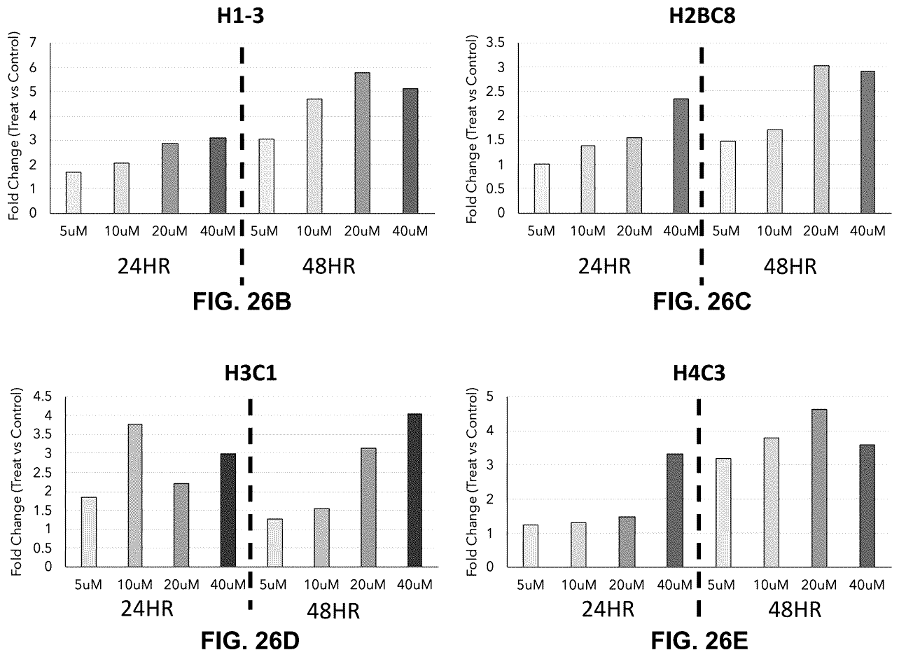

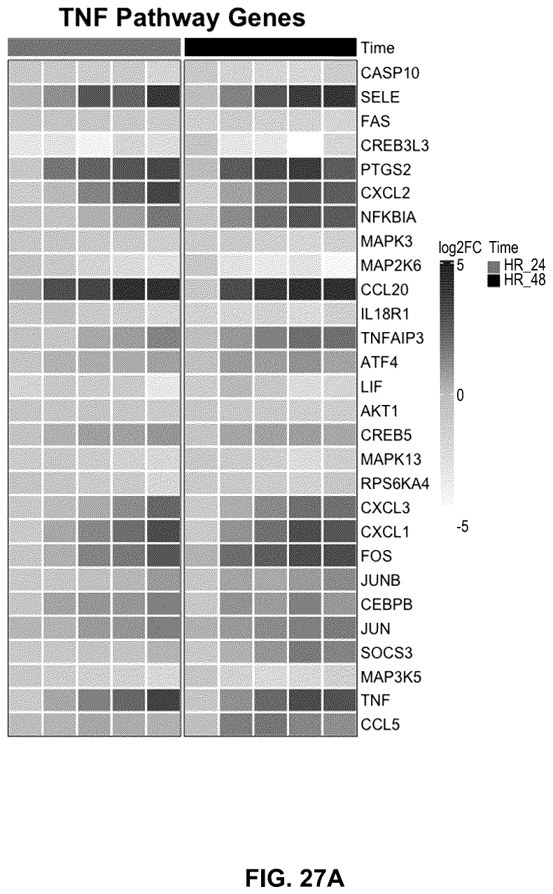

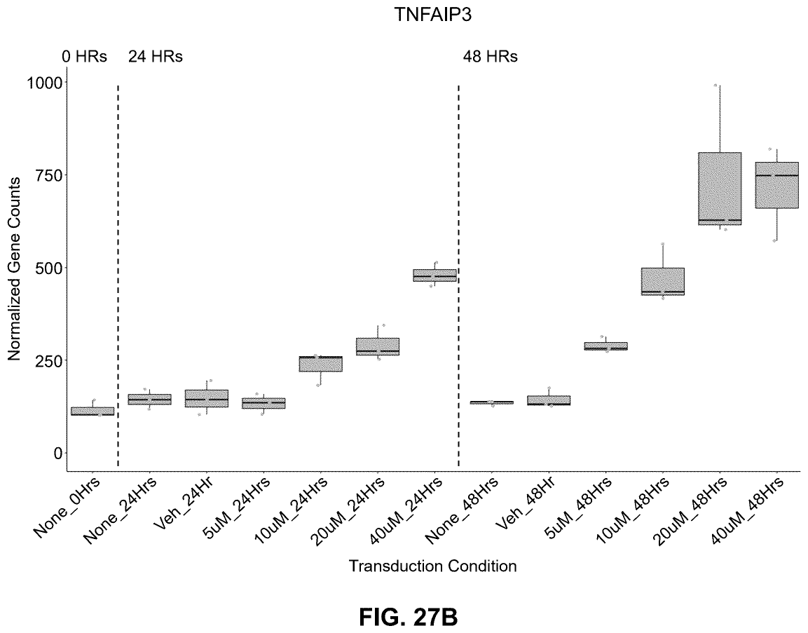

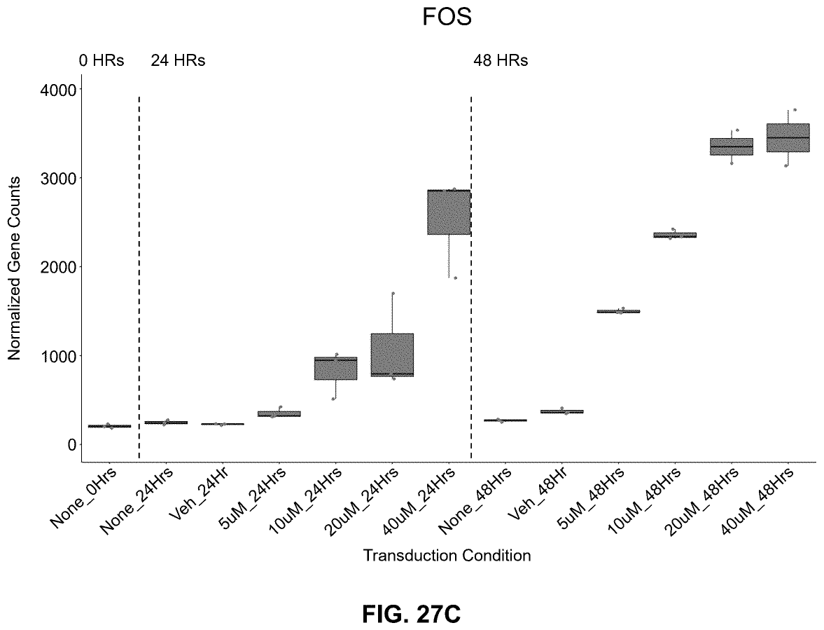

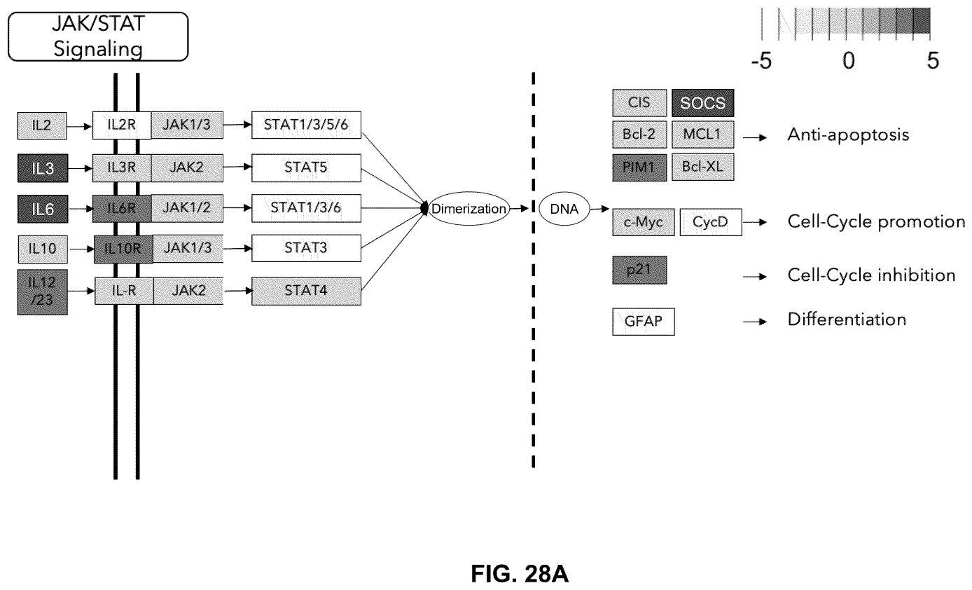

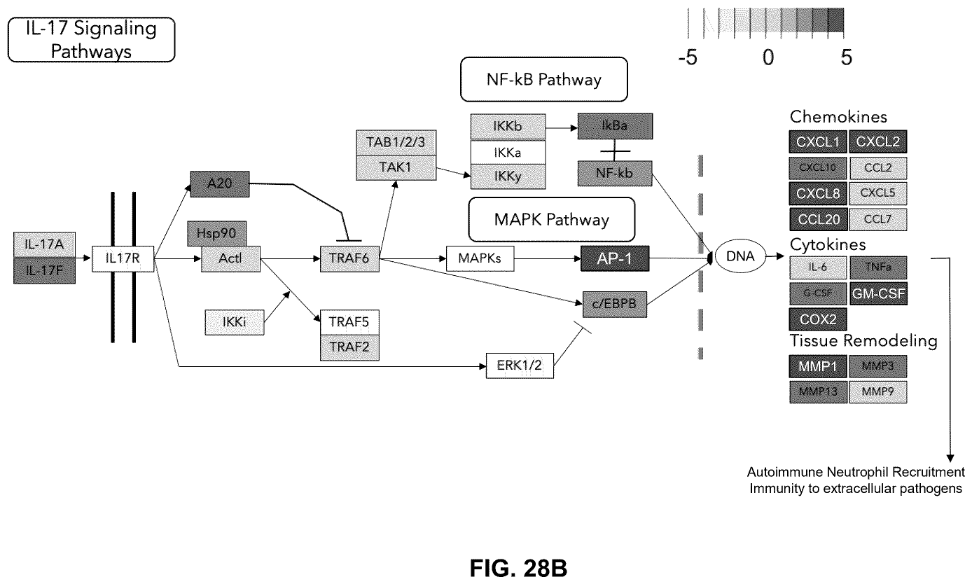

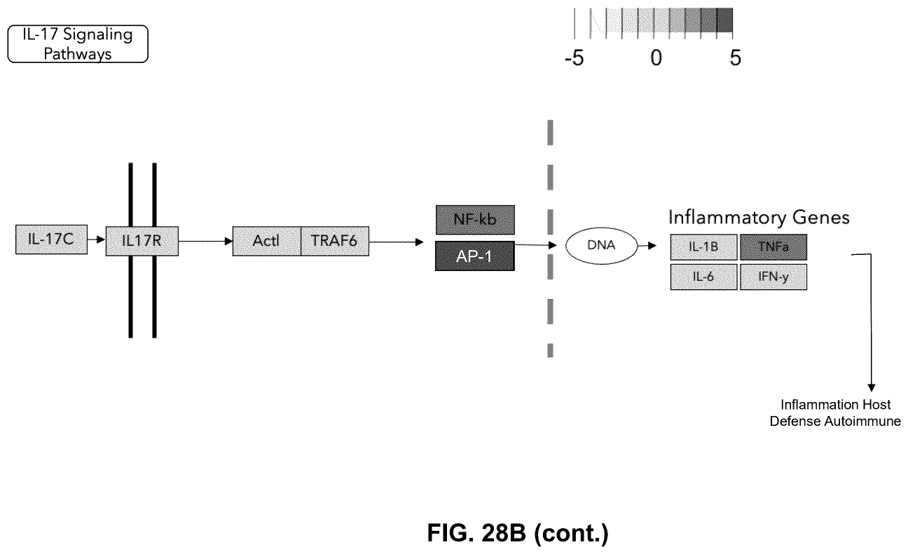

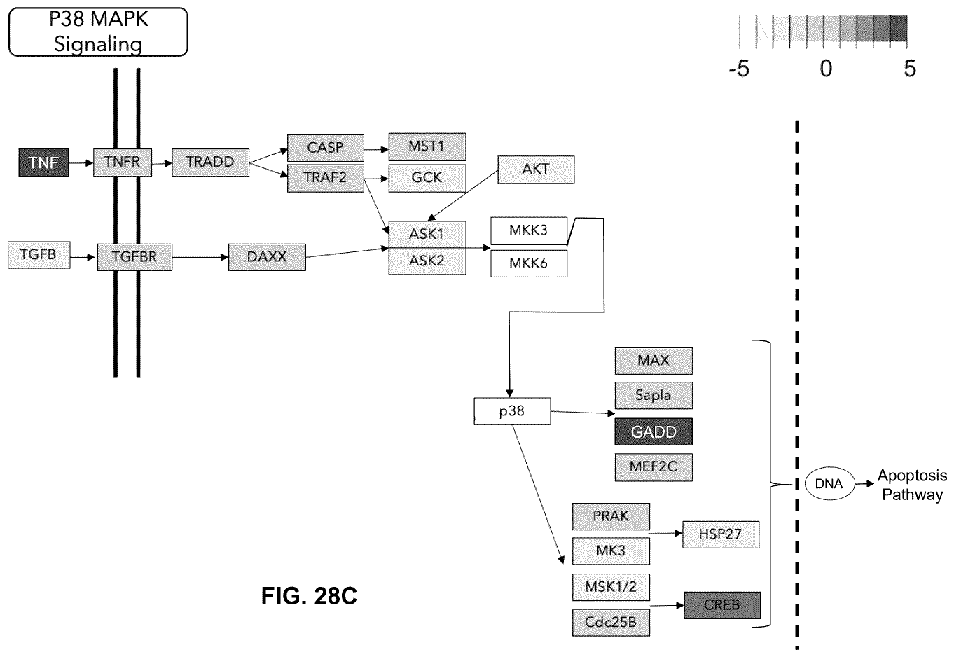

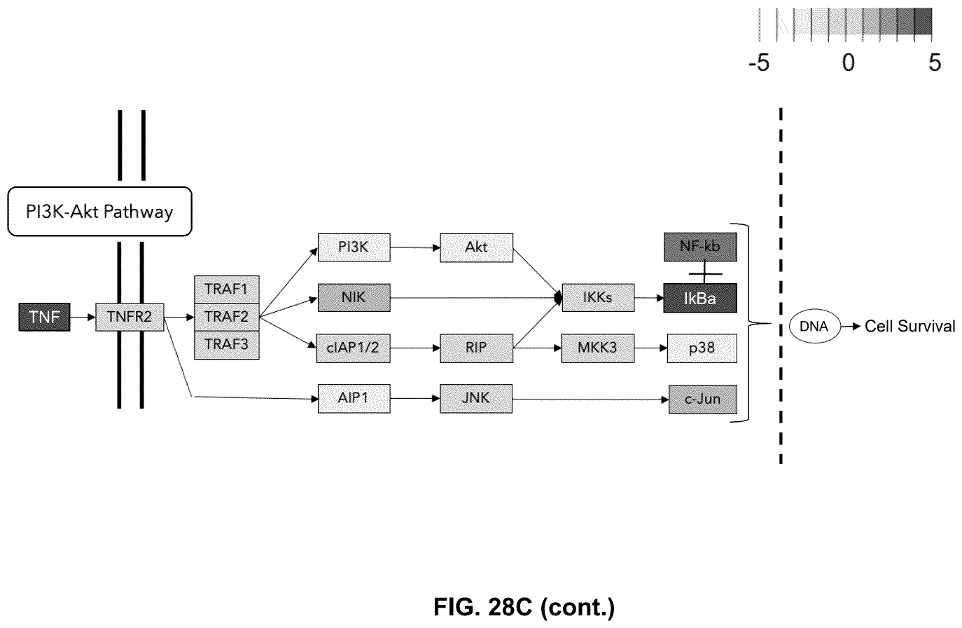

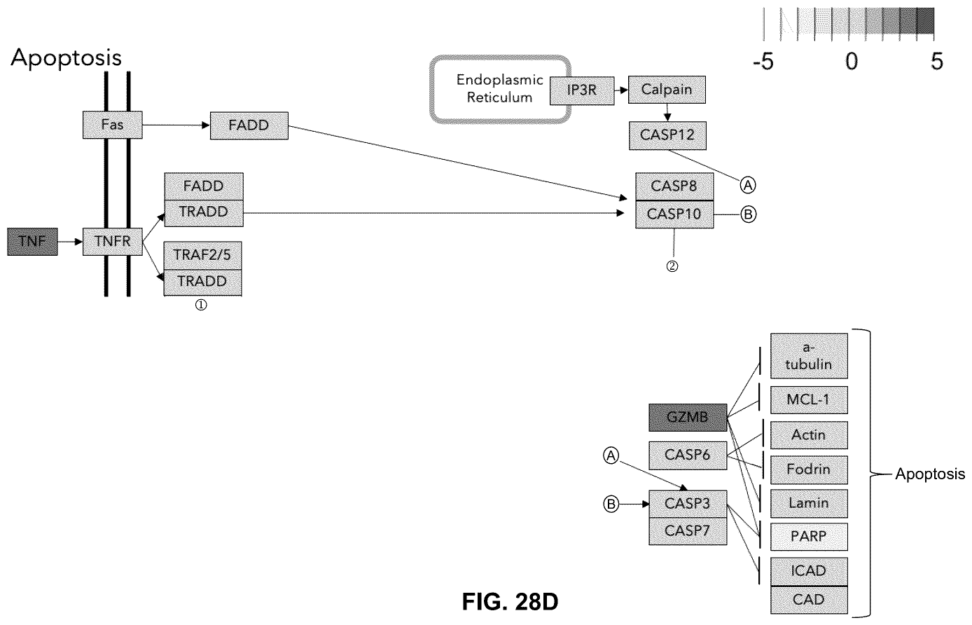

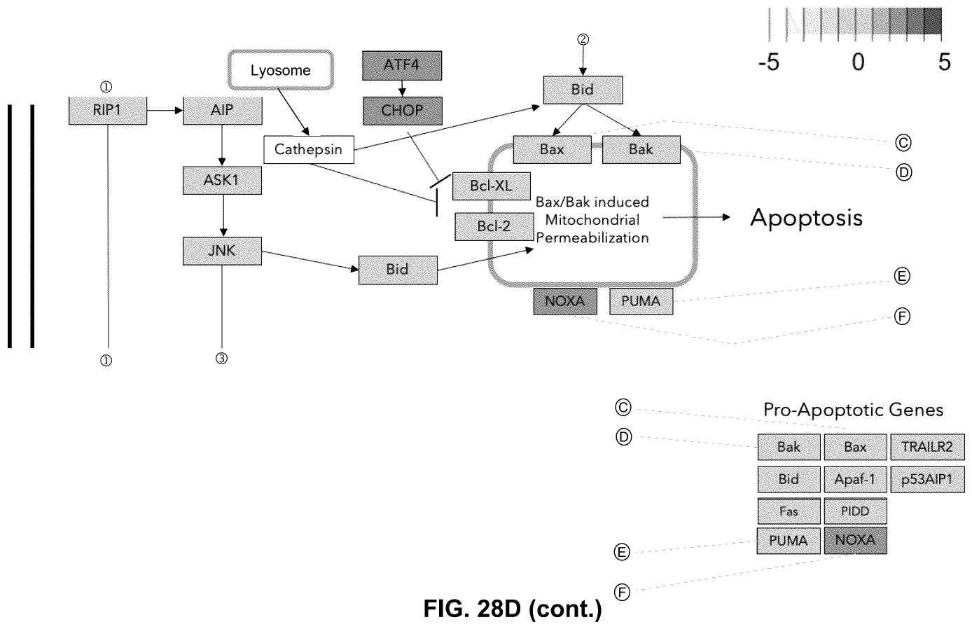

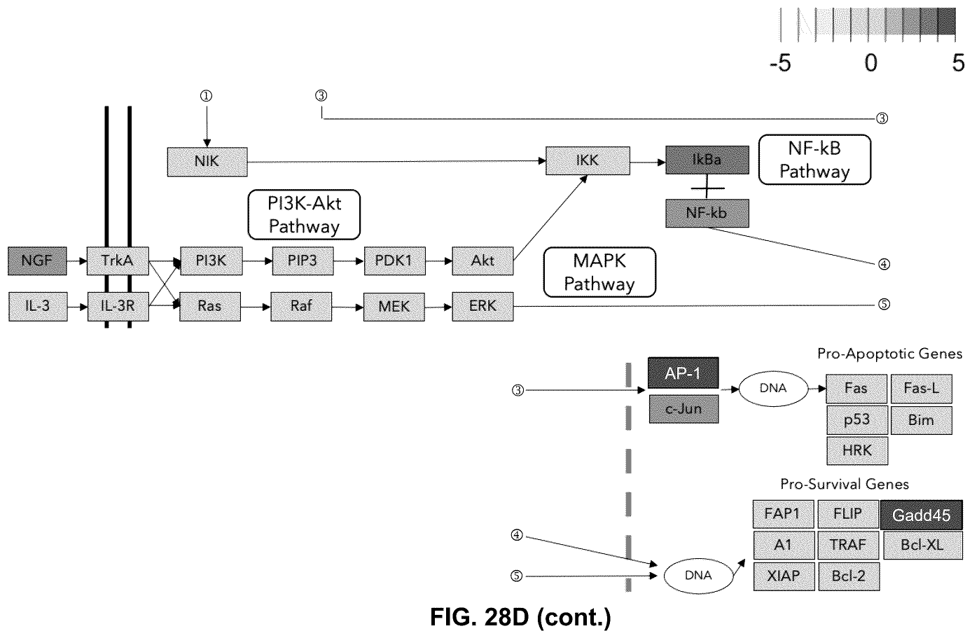

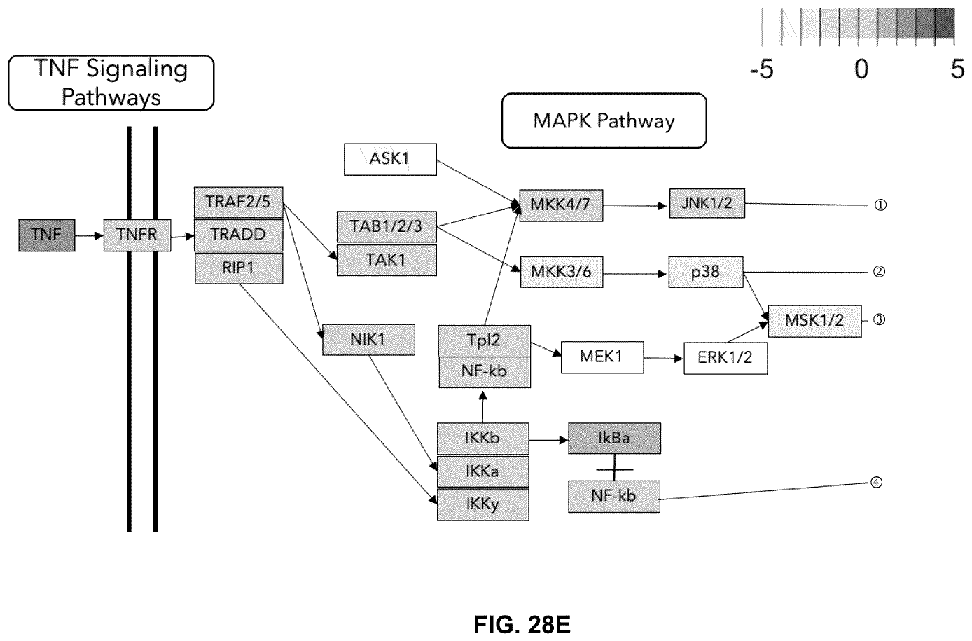

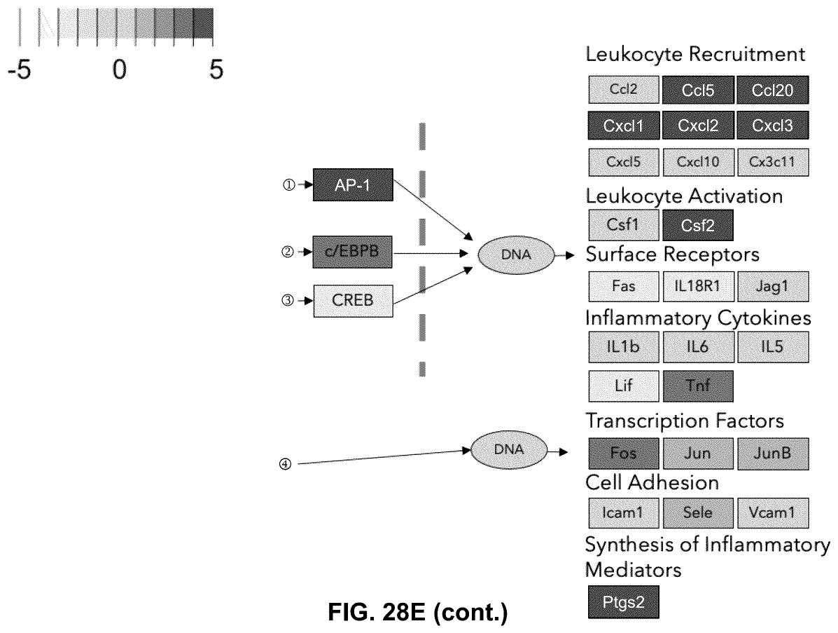

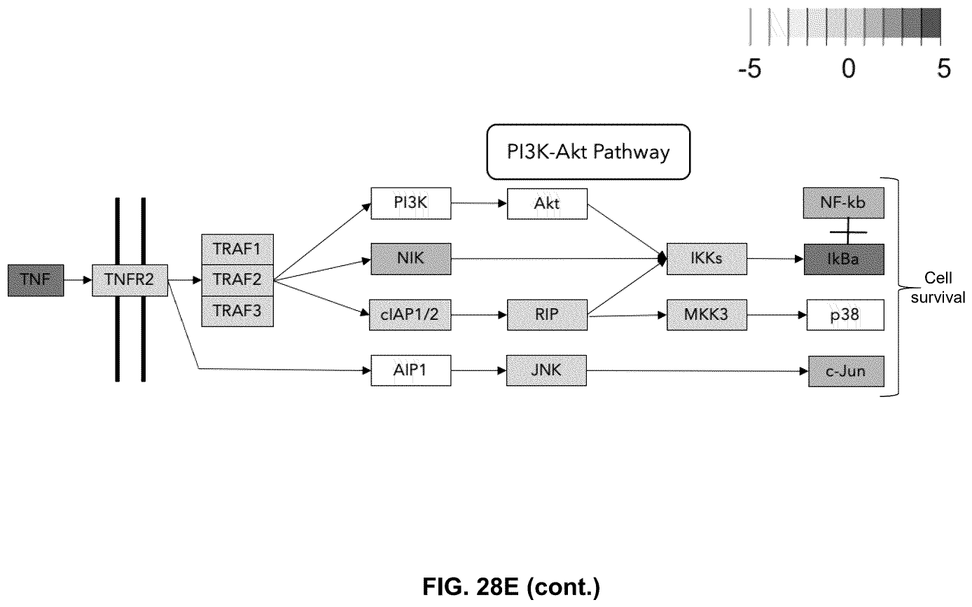

Further analysis revealed that treatment with Drug H led to altered expressed of AAV packaging genes such as Cap (FIG. 22A) and Rep (FIG. 22B). Additionally, the treatment resulted in changes in the host cell transcriptome (FIGS. 23A-23H). Pathway analysis of the differentially expressed genes revealed enrichment of apoptosis, viral, and signaling pathways (FIG. 24). Additionally, inflammatory response, defense response to virus pathway, and immediate early genes were dampened as compared to previously identified genes during AAV production after treatment with increasing concentrations of Drug H (FIGS. 25A-25G—from left to right: vehicle control, 5 μM, 10 μM, 20 μM, 40 μM at 24 hours (gray) and 48 hours (green); see also Chung, C H., et al., Mol Ther Methods Clin Dev. 28:272-283 (2023)). Furthermore, after treatment with Drug H, there was significant alteration of JAK/STAT signaling (FIG. 28A), IL-17 and NF-kB signaling pathways (FIG. 28B), P38 MAPK and PI3K-Akt signaling pathways (FIG. 28C), apoptosis pathways (FIG. 28D), and TNF signaling pathway (e.g., TNFAIP3 and FOS) (FIGS. 28E and 27A-27C). Additionally, the response of nucleosome assembly genes previously identified during AAV product was heightened after Drug H treatment (FIGS. 26A-26E).

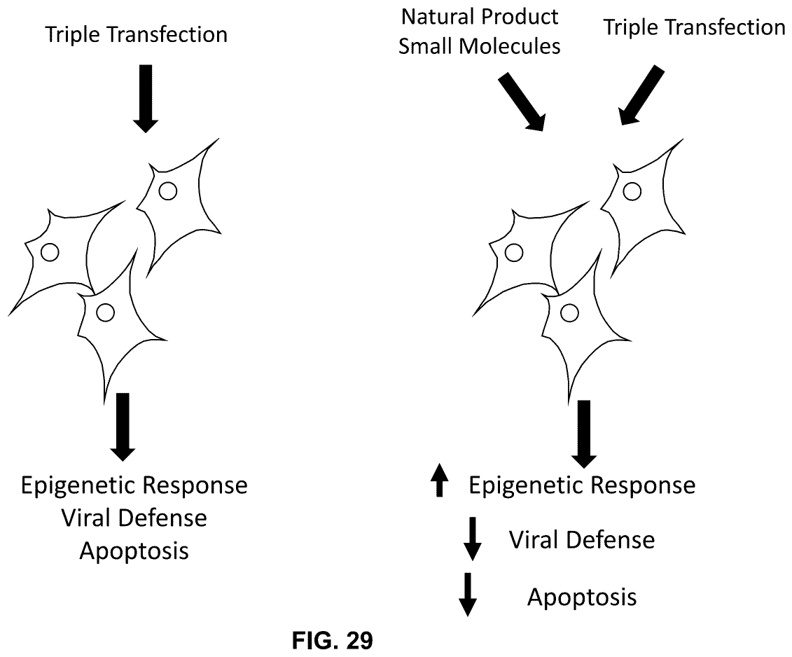

These results showed that many of the enriched pathways identified by Drug H treatment converged on apoptosis (e.g. FIG. 28D). Taken together, the data suggested that treatment of cells with Drug H during AAV production led to increased epigenetic response (e.g., enhancement of histone levels that increased metabolic activity to improve replication), reduced viral defense that allowed survival of producer cells, and reduced apoptosis through alteration of signaling pathways (e.g., NF-κB, TNF, MAPK, IL-7), which, without being bound by theory, may have allowed for more availability of resources for viral production. An exemplary scheme for a possible mode of action for Drug H on host cells during AAV production is shown in FIG. 29.

### Example 4—Inducible Rep System for AAV Production (Prophetic)

The inducible sequences identified in Example 1 were inserted upstream of Rep and used in a triple transfection test for the packaging of AAV-GFP. HEK293 cells were triple transfected with a helper plasmid that also expresses Cap, the GFP gene of interest plasmid, and a plasmid with either wild-type Rep or a version of Rep downstream of the identified splice sequence (e.g., SEQ ID NOs: 59-62).

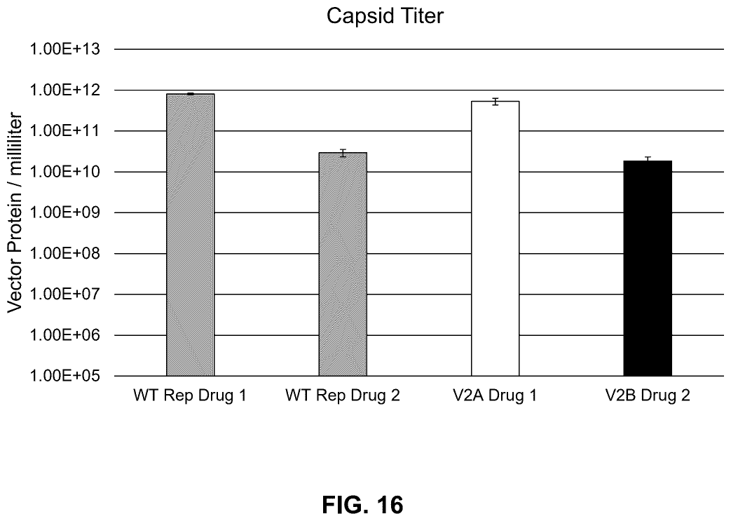

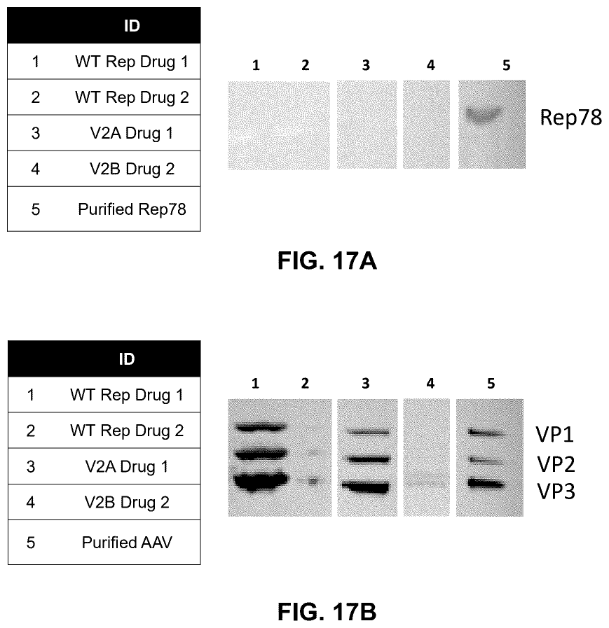

In this system, the ability of induced Rep to package AAV-GFP was observed by detecting genome titer (FIG. 15) and capsid titer (FIG. 16). Additionally, Rep protein (FIG. 17A) and VP1, VP2, and VP3 levels (FIG. 17B) were observed for both systems. The genome and capsid titer, along with the Rep and Cap protein levels, show that the inducible system was capable of packaging.

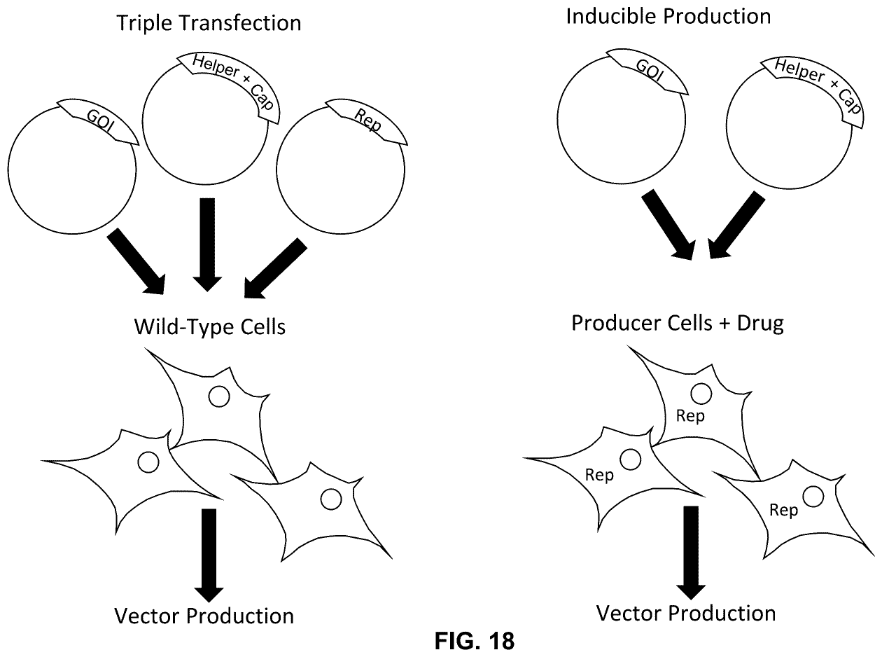

These results show that the tested inducible systems with Drug H or Drug K can be used to induce the expression of transgenes in a post-transcriptional manner. The system can include cell lines capable of expressing various genes necessary for AAV packaging, including Rep or Cap, which allow for increased vector production by increasing the number of cells actively generating AAV particles over transient triple transfection methods (See FIG. 18). The inducible system allows for finely tuned temporal control of expression to produce AAV vectors.

### Example 5—Expression of Vectorized Reporter Constructs In Vitro

### Small Molecule Drugs Tested

For in vitro dosing, Kinetin (MedChemExpress) (“Drug K”) or Homocarbonyltopsentin (MedChemExpress) (“Drug H”) were dissolved in 100% DMSO and this material was directly added to cells.

### AAV Vector Cloning and Production

The sequences of TRPV3 construct #3 (SEQ ID NO: 45) and LYPD3 construct #3 (SEQ ID NO: 54) were each placed directly downstream of the Rous Sarcoma Virus (RSV) promoter and upstream of enhanced GFP (eGFP). The RSV promoter, eGFP, and either TRPV3 construct #3 or LYPD3 construct #3 sequence were cloned between two AAV inverted terminal repeat sequences. Additionally, a vector was generated that contained just the RSV, eGFP and ITR sequences (Positive Control Construct). Recombinant AAV2 serotype vectors were produced with these constructs, purified, and their vector titers were determined by digital droplet polymerase chain reaction (ddPCR).

### Cell Culture and In Vitro AAV Vector Transduction

HEK293T human embryonic kidney cells, HepG2 human liver cells, U87 MG human glioblastoma cells, and Neuro2a mouse neuroblastoma cells (ATCC) were cultured in DMEM media containing 10% fetal bovine serum (Gibco) and 1% penicillin/streptomycin at 37 degrees Celsius with 5% carbon dioxide. Cells were plated into individual wells of a 48-well plate and cultured for 24-hours. After 24-hours of culture, AAV vectors containing either TRPV3 construct #3 and LYPD3 construct #3 were added to each well at a multiplicity of infection of 1e5 or 5e5 for viral transduction. 24-hours post transduction, cells were treated with DMSO, Kinetin, or Homocarbonyltopsentin, grown for 24-hours, washed with Dulbecco's phosphate buffered saline (DPBS) and imaged for eGFP expression.

### Fluorescent Microscopy

24 hours after drug treatment, cells were washed with DPBS and imaged using and EVOS M5000 (ThermoFisher) using identical settings for each well. 2 biological replicates were imaged for each condition.

### Methods and Results

To examine the effectiveness of Drug K or Drug H on AAV packaged splicing reporters, the plasmids containing TRPV3 Construct #3 or LYPD3 Construct #3 were cloned into a plasmid containing two inverted terminal repeat sequences (ITRs), the Rous Sarcoma Virus promoter (RSV), and the enhanced green fluorescent protein (eGFP) for rAAV vector production. rAAV2 vectors were generated, purified, and transduced into HEK293, Neuro2A, HepG2, or U87 MG cells at a multiplicity of infection of 1e5 vector genomes or 5e5 vector genomes per cell.

The test groups included cells transduced with no vector (Negative Control), AAV2 Positive Control Construct, AAV2 TRPV3 Construct #3, or AAV2 LYPD3 Construct #3. 24-hours after transduction, cells that received no vector were treated with dimethyl sulfoxide (vehicle), 300 micromolar Drug K, or 40 micromolar Drug H. Cells that were transduced with AAV2 Positive Control Construct were treated with dimethyl sulfoxide (vehicle), 150 micromolar Kinetin, 300 micromolar Kinetin, 20 micromolar Drug H, or 40 micromolar Drug H. Cells that were transduced with AAV2 TRPV3 Construct #3 were treated with dimethyl sulfoxide (vehicle), 150 micromolar Kinetin or 300 micromolar Kinetin. Cells that were transfected with LYPD3 Construct #3 were treated with dimethyl sulfoxide (vehicle), 20 micromolar Drug H, or 40 micromolar Drug H. 24-hours after treatment with the indicated small molecule, cells were monitored for GFP expression.

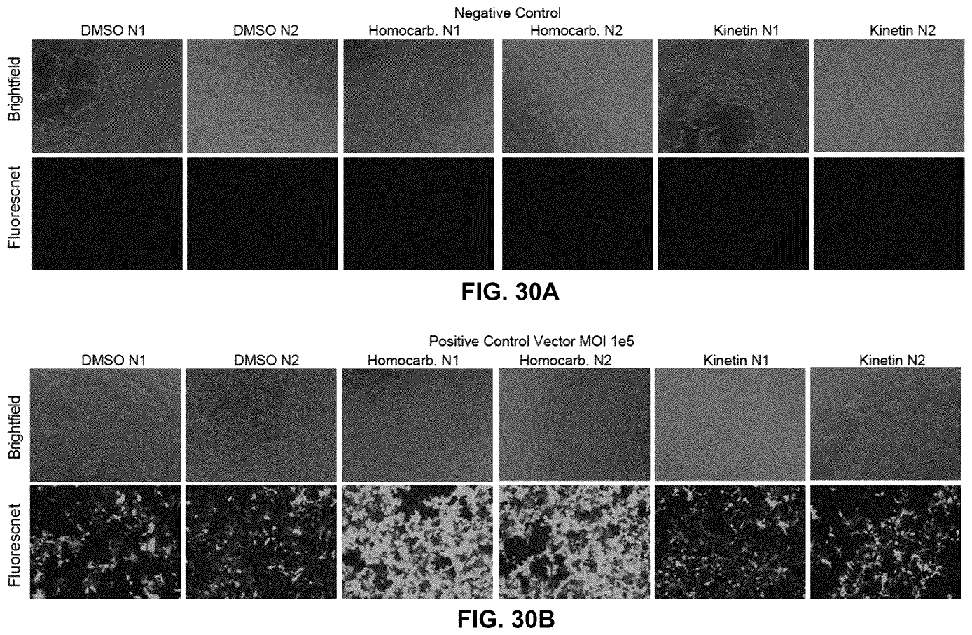

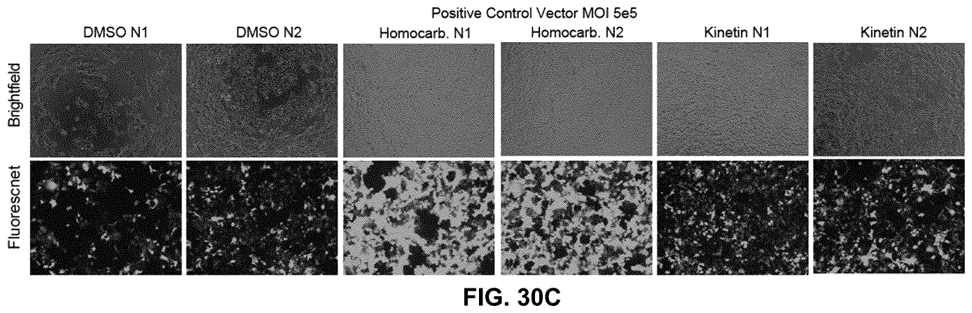

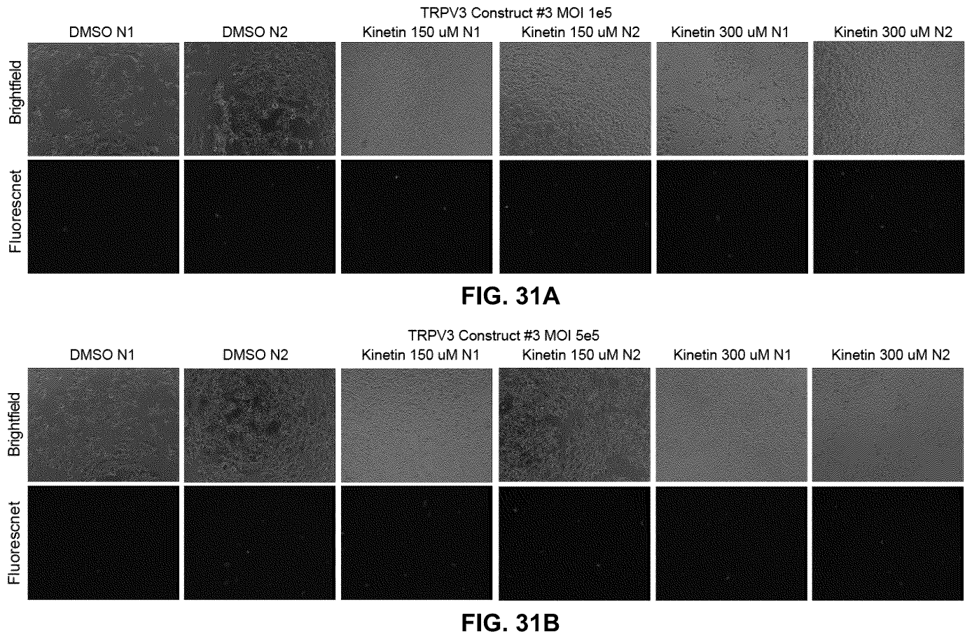

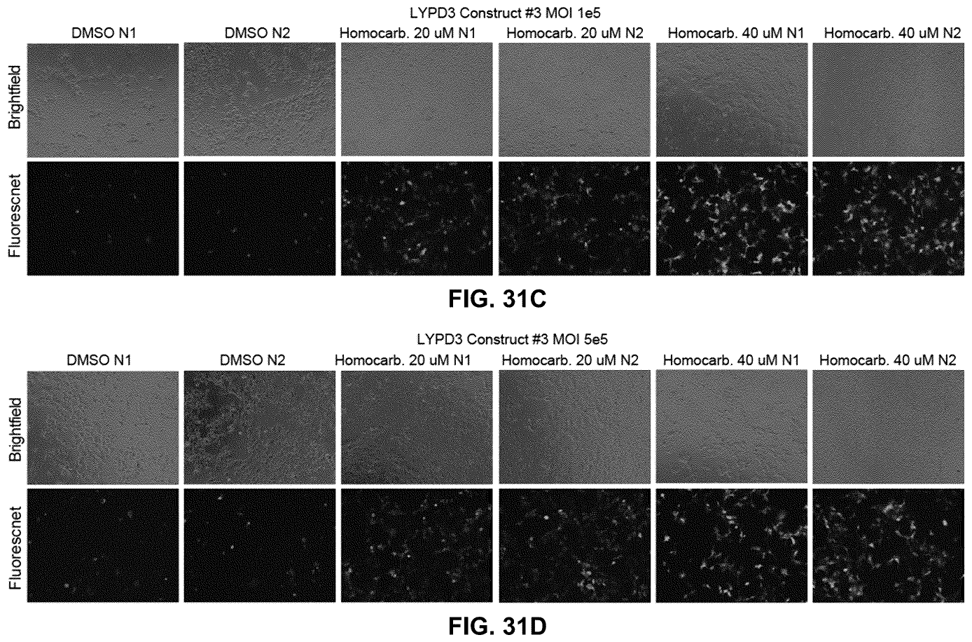

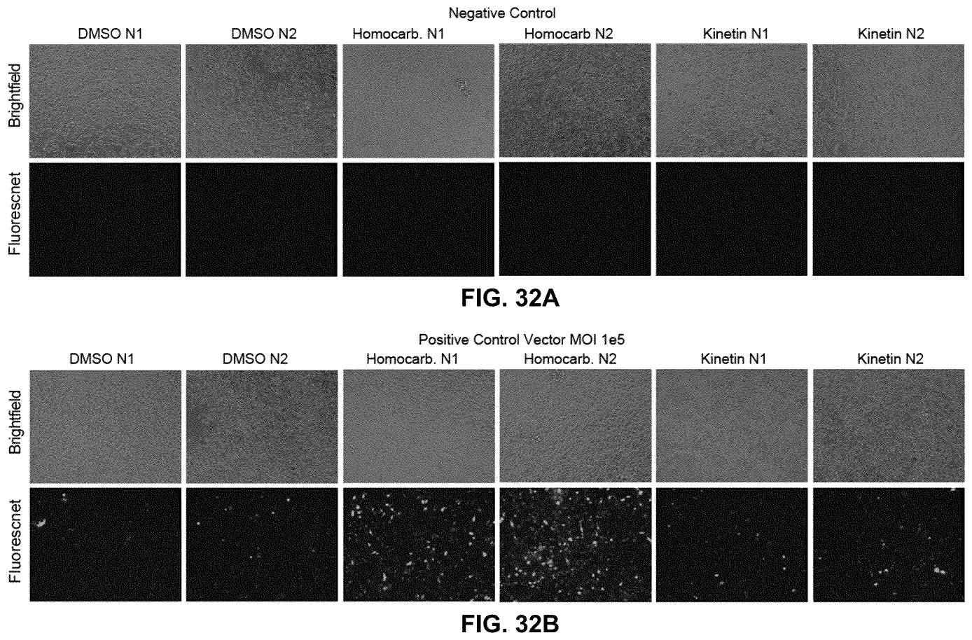

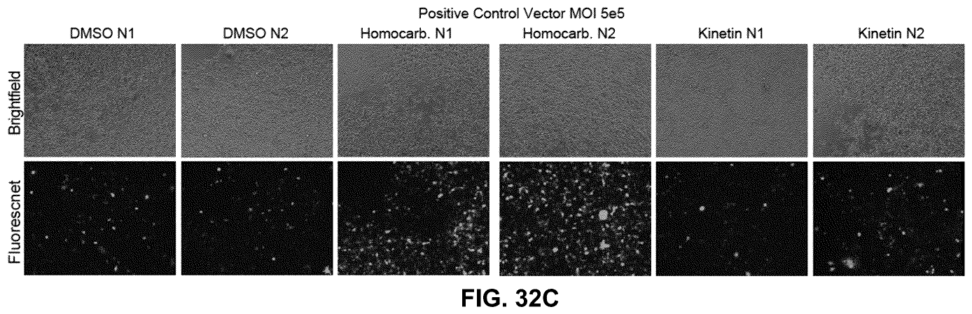

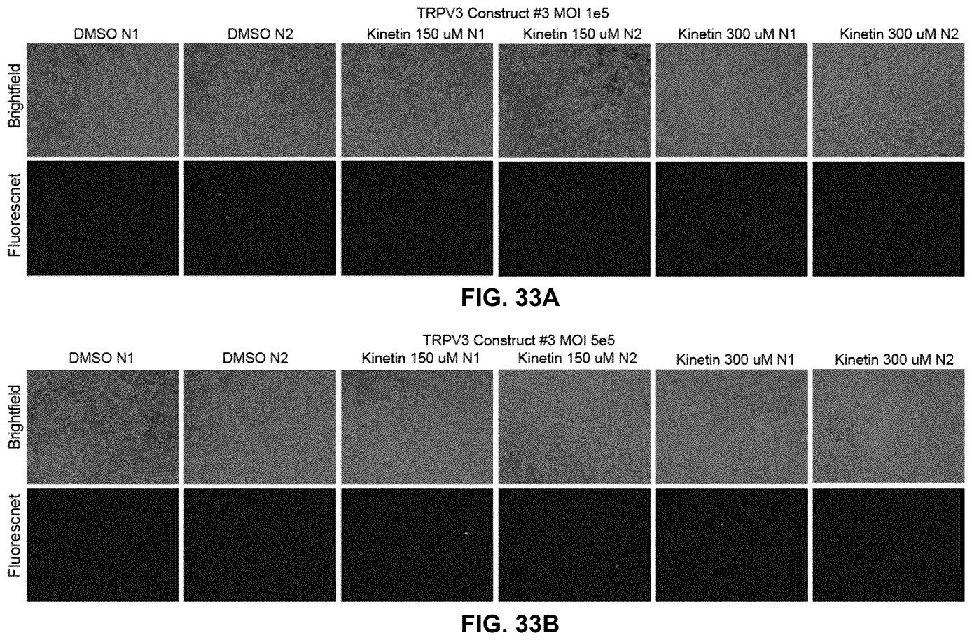

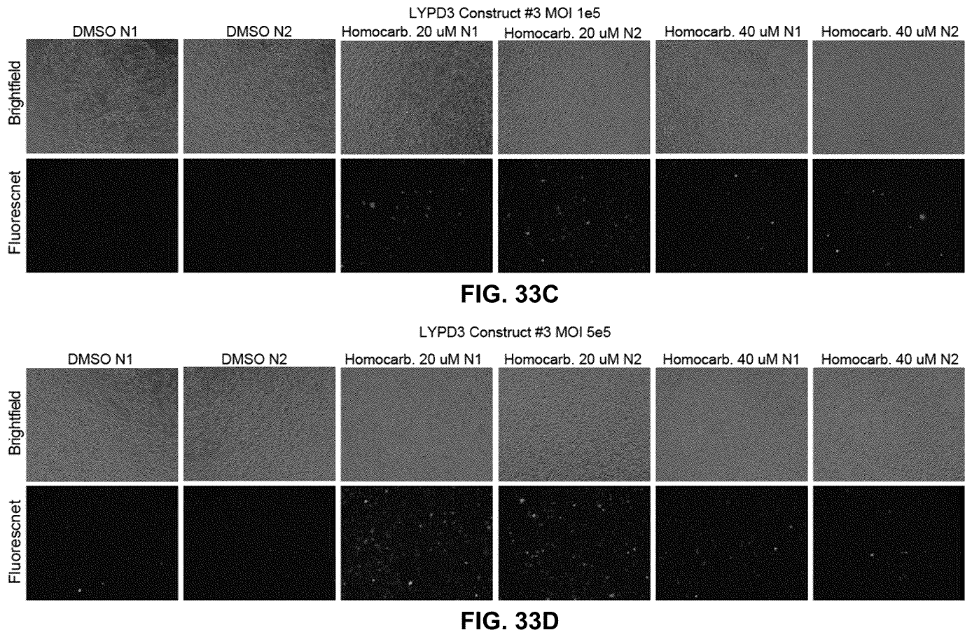

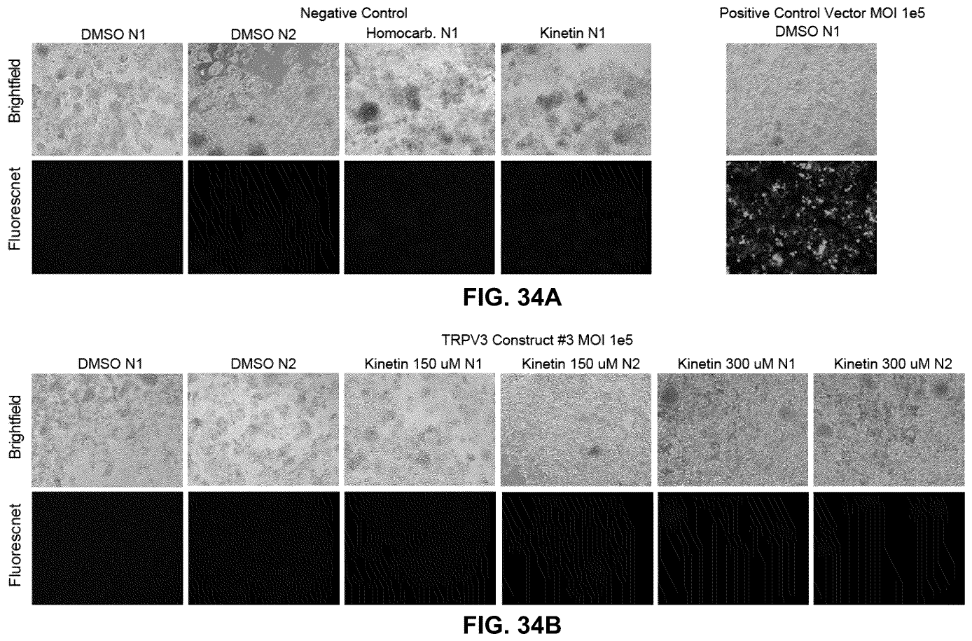

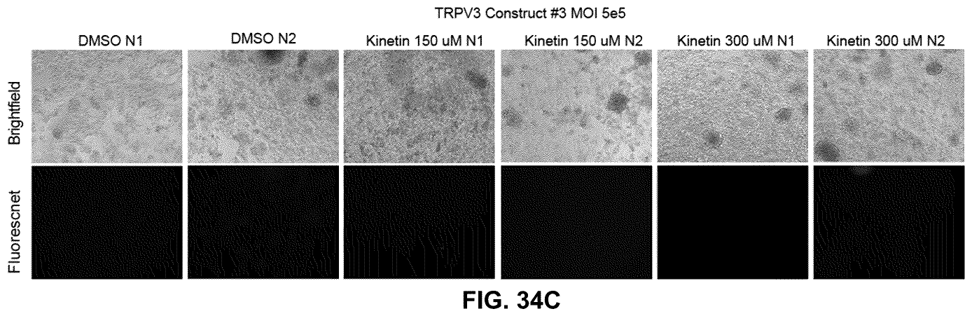

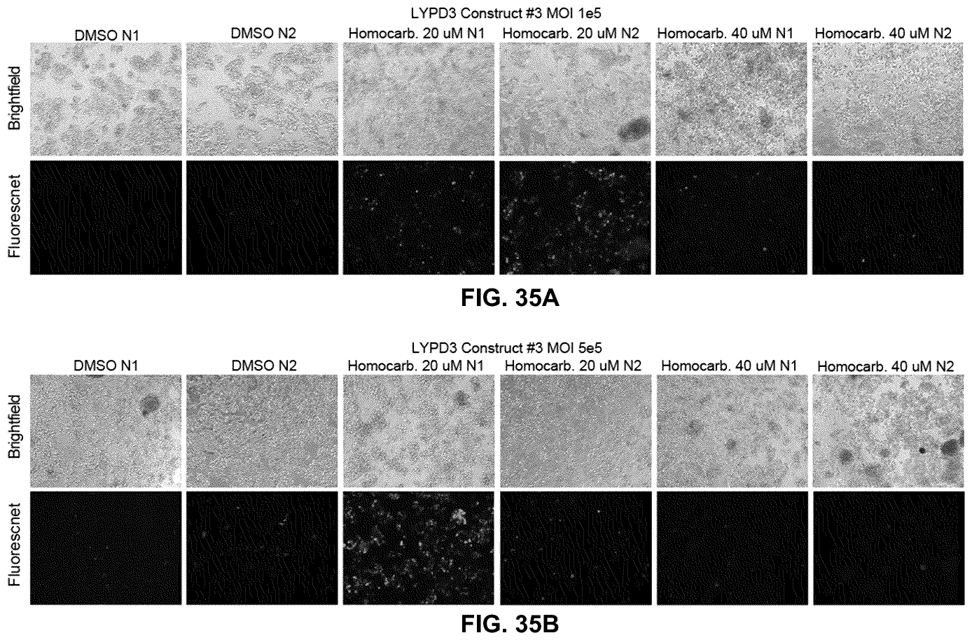

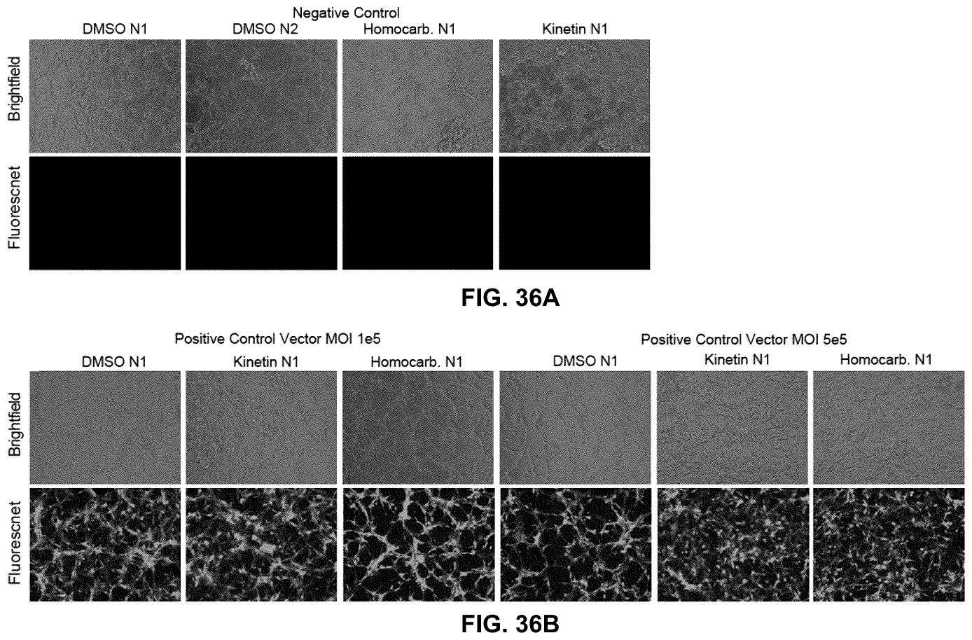

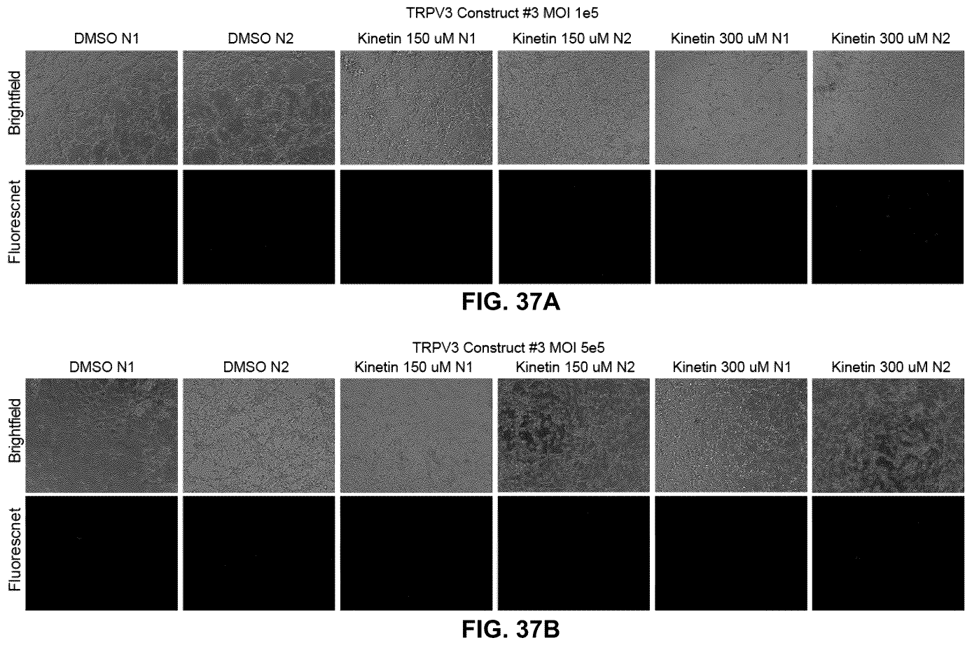

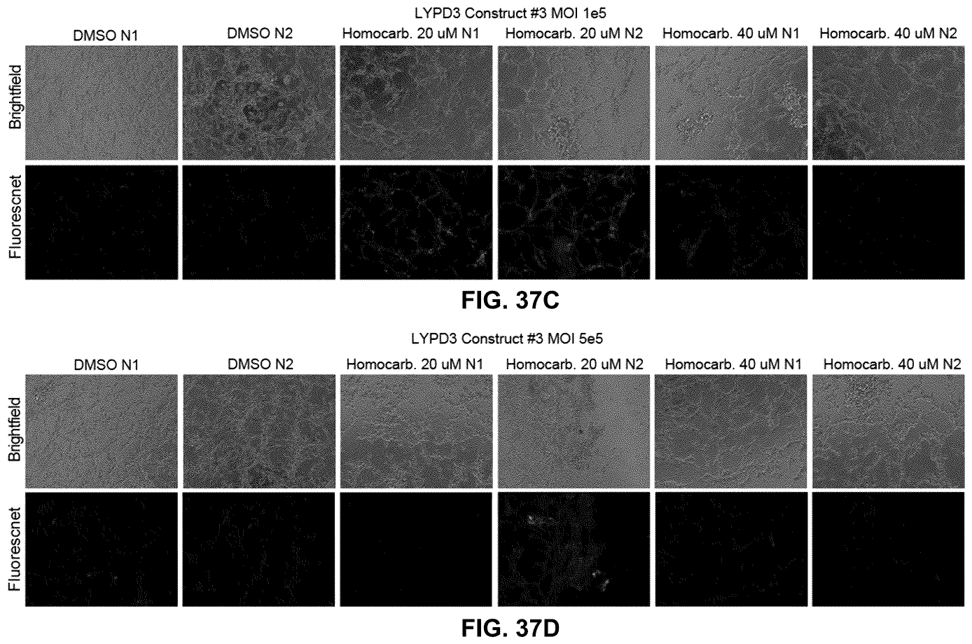

An increase in the expression of GFP was observed by fluorescent microscopy for all cell types (e.g., HEK293 cells and Neuro2A cells) after small molecule treatment in the cells that were transduced with TRPV3 Construct #3 or LYPD3 Construct #3 (FIGS. 30A-30C (controls), 31A-31C (TRPV3 Construct #3 and LYPD3 Construct #3 at 1e5 or 5e5), 32A-32C (controls), 33A-33D (TRPV3 Construct #3 and LYPD3 Construct #3 at 1e5 or 5e5), 34A-34C (TRPV3 Construct #3 at 1e5 or 5e5), 35A-35B (LYPD3 Construct #3 at 1e5 or 5e5), 36A-36B (controls), and 37A-37D (TRPV3 Construct #3 and LYPD3 Construct #3 at 1e5 or 5e5)), supporting that Drug K and Drug H can operate as modulators of alternative splicing of AAV vector splicing reporters in many cell lines.

### Example 6—Expression of Vectorized Reporter Construct In Vivo

### Small Molecule Drugs Tested

For in vivo dosing, Kinetin (“Drug K”) was first dissolved to 150 milligrams per milliliter in 100% DMSO and then vortexed and incubated at 80 degrees Celsius for 10 minutes with shaking. After shaking a final dosing solution was prepared of 60% PEG300, 5% Tween-80, 10% DMSO, and 25% phosphate-buffered saline. For in vivo dosing, Homocarbonyltopsentin (“Drug H”) was dissolved in 100% DMSO to make a 10 milligram per milliliter solution. The solution was vortexed and incubated at 50 degrees Celsius for 5 minutes with shaking. To make the final dosing solution, PEG300 was added at 30% of the desired final volume, followed by Tween-80 at 5% of the final volume, and then phosphate-buffered saline at 55% of the final volume. Each material was vortexed after addition. Both Kinetin and Homocarbonyltopsentin final dosing solutions were prepared the day of oral gavage.

### AAV Vector Cloning and Production

The sequences of TRPV3 construct #3 and LYPD3 construct #3 (same sequences as Example 5) were each placed directly downstream of the Rous Sarcoma Virus (RSV) promoter and upstream of enhanced GFP (eGFP). The RSV promoter, eGFP, and either TRPV3 construct #3 or LYPD3 construct #3 sequence were cloned between two AAV inverted terminal repeat sequences. Additionally, a vector was generated that contained just the RSV, eGFP and ITR sequences (Positive Control Construct). Recombinant AAV2 vectors were produced with these constructs, purified, and their vector titers were determined by digital droplet polymerase chain reaction (ddPCR).

### In Vivo Animal Study

Eight- to twelve-week-old female C57BL/6 mice at Charles River Laboratories were selected and randomized based on body weight for treatment with rAAV vector and the small molecules. TRPV3 construct #3 vector, LYPD3 construct #3 vector, or Positive Control vector was injected intravenously into a mouse at a dose of either 1e11 vector genomes or 1e12 vector genomes. Animals were housed and monitored for body weight changes for 4 weeks, 4 weeks post-injection, either Kinetin or Homocarbonyltopsentin were given by oral gavage. Kinetin was introduced at a concentration of either 70 or 140 milligrams per kilogram of body weight. Homocarbonyltopsentin was introduced at either 10 or 20 milligrams per kilograms of body weight. Twenty-four hours after small molecule introduction, animals were euthanized, and tissues were collected.

### Extraction for RNA and DNA Analyses

Whole hearts and livers were extracted, stored in RNAlater at 4 degrees Celsius for twenty-four hours, and then decanted and stored at −80 degrees Celsius.

### Extraction for Fluorescent Microscopy

Whole hearts and livers were extracted and then immersed in 4% paraformaldehyde overnight. After fixation, the samples were placed in 15% sucrose in phosphate-buffered saline for two hours, then transferred to 30% sucrose in phosphate-buffered saline for two hours, followed by freezing in Tissue Tec-OCT and stored at −80 degrees Celsius before sectioning. Each OCT block was sectioned once at 8-microns thick and mounted on Super-frost slides with VECTASHIELD Hardset Antifade Mounting Media with DAPI. Slides were then stored at −80 degrees Celsius until imaging.

### RNA and DNA Co-Extraction

From each mouse liver or heart, 7.5 to 10 milligrams of tissue were isolated with a razor blade and placed within a Precellys tube along with 6 Precellys 2.8 ceramic zirconium oxide beads and 300 microliters of DNA/RNA Shield (Zymo Research). The tubes were loaded into the Precellys 24 homogenizer and run two times with the settings 6500 rpm, 6 cycles, 50 second cycles, 15 second pause. The homogenates were then centrifuged at 10,000×g for 5 minutes at room temperature. 15 microliters of 20 milligram per milliliter Proteinase K was added and the tubes were vortexed and then incubated for 1 hour at 30 degrees Celsius with shaking at 1200 rotations per minute. 200 microliters of sample were then transferred to a ThermoFisher KingFisher 96 deep-well plate containing 500 microliters of Quick-DNA/RNA Magbead DNA/RNA Lysis buffer (Zymo Research). The Quick-DNA/RNA Magbead kit (Zymo Research) was used to extract the DNA and RNA according to the manufacturer's protocol with the use of the KingFisher Apex automated sample purification system. The concentration of DNA and RNA was then determined by the Invitrogen Qubit system.

### Vector DNA Quantification

Total extracted DNA was diluted to a concentration of 5 nanograms per microliter. Quantitative polymerase chain reaction (qPCR) was performed using the PrimeTime Gene Expression Master Mix (IDT) according to the manufacturer's protocol using a QuantStudio 5 Real-Time PCR system (Applied Biosystems). For quantification, a standard curve was generated using plasmid DNA of the construct used to produce the vector and vector expression was normalized to nanograms of input plasmid. Taqman probes (IDT) were used to detect vector DNA.

### Vector RNA Quantification

500 nanograms of total RNA was used as input to the High-Capacity cDNA Reverse Transcription Kit (Thermo) according to manufacturer's instructions. Total cDNA was diluted 1:10 and then used for quantitative reverse-transcription real-time polymerase reaction (qRT-PCR) using the PrimeTime Gene Expression Master Mix (IDT) according to the manufacturer's protocol using a QuantStudio 5 Real-Time PCR system (Applied Biosystems). Small molecule induced alternative splicing of either TRPV3 construct #3 or LYPD3 construct #3 was determined using two custom Taqman assays targeting the alternative exon sequence compared to the constitutive eGFP sequence. The fold change of induction was calculated using the delta-delta Ct method for the average Ct values from each animal in the no drug treatment group compared to the drug treatment group. Taqman probes (IDT) were used to quantify the RNA.

### Protein Extraction

Mouse liver lysate supernatant samples were prepared by homogenizing liver tissue samples in complete lysis buffer (Mesoscale Discovery Tris lysis buffer, ThermoScientific Halt protease and phosphatase inhibitor cocktail, ThermoScientific EDTA). Homogenization was performed for 3 rounds of 6,000 rpm using a Precellys 24 homogenizer (Bertin Technologies). The samples were then centrifuged at 14,000 rpm for 10 minutes at 4° C., and the supernatant was collected for GFP quantification analysis. Homogenized mouse liver lysate supernatant samples were analyzed for total protein concentration by DC assay (BioRad).

### Detection of Protein Expression

The concentration of GFP protein present in homogenized mouse liver lysate supernatant was quantified using a three-step (capture-analyte-detection) immunoassay specific to the protein. All analyses were performed on Gyrolab xPlore system (Gyros Protein Technologies) with Bioaffy 1000 CDs (Gyros Protein Technologies, method 1000-3W-001-A). The capture reagent consisted of biotinylated anti-GFP monoclonal antibody (Rockland Immunochemicals, 100 μg/mL), and the detection reagent consisted of a noncompeting Alexa Fluor 647 tagged anti-GFP polyclonal antibody (Novus Biologicals, 25 nM). A standard curve (0.12-500 ng/ml) was generated using a recombinant GFP protein (Novus Biologicals) diluted in lysate buffer solution consisting of 1:1 negative control mouse liver lysate supernatant and Rexxip AN (Gyros Protein Technologies). Mouse liver lysate supernatant samples were diluted 1:1 in Rexxip AN. The concentration of GFP protein in mouse liver lysate supernatant was determined by comparison of sample signal at 5% PMT to the standard curve signal fit to a five-parameter logistic curve using the Gyrolab Evaluator software (Gyros Protein Technologies).

### Methods and Results

To examine the effect of alternative splicing driven by Drug K or Drug H in vivo, rAAV2 packaged splicing reporters containing Positive Control Construct, TRPV3 Construct #3 or LYPD3 Construct #3 were injected intravenously into C57BL/6 mice at a concentration of either 1e11 or 1e12 vector genomes per animal. 4-weeks post injection, Drug K, Drug H, or vehicle were introduced to these animals by oral gavage at a dose of either 70 milligrams per kilogram or 140 milligrams per kilogram for Drug K or 10 milligrams per kilogram or 20 milligrams per kilogram for Drug H. Twenty-four hours after oral gavage, animals were euthanized, and the livers and hearts of the treated animals were extracted. From those tissues, RNA, DNA, and protein were extracted and analyzed for vector RNA or DNA expression, and GFP protein expression.

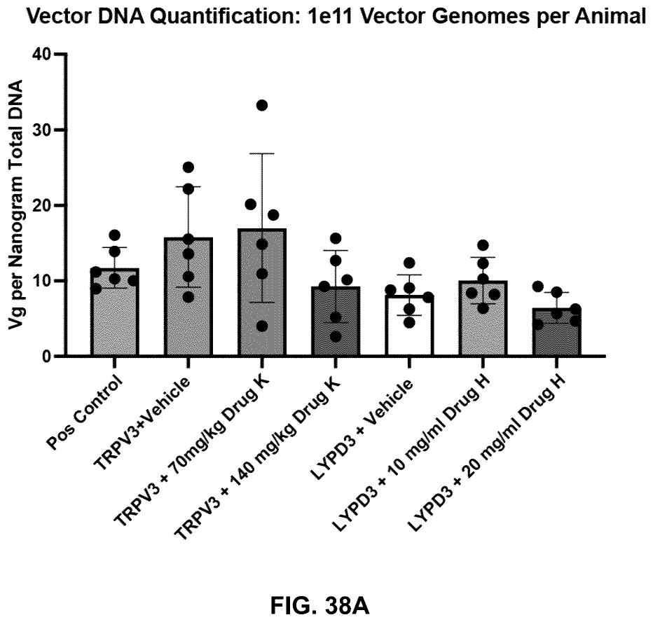

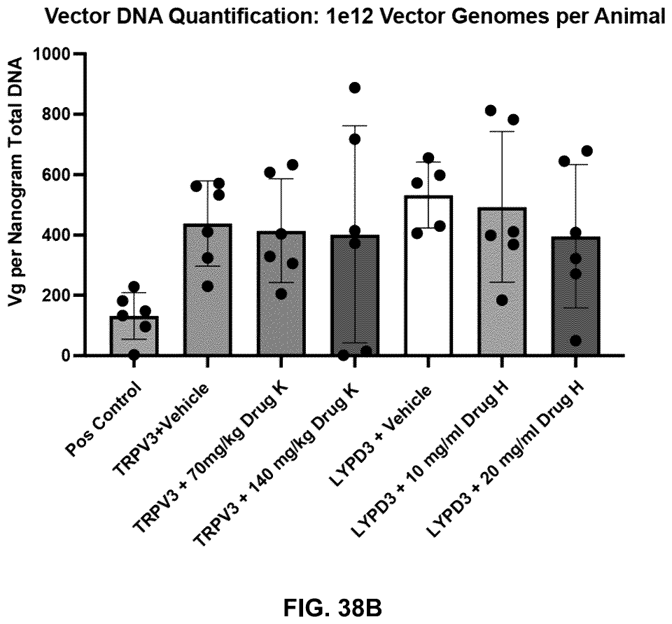

The total level of vector DNA within each animal in a treated group was not significantly different between animals that received Positive Control Construct, TRPV3 Construct #3 or LYPD3 Construct #3 (FIGS. 38A-38B).

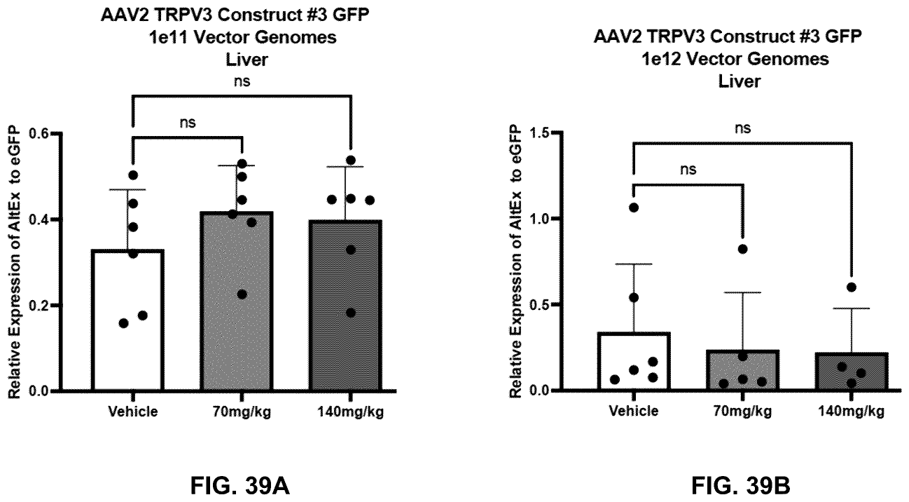

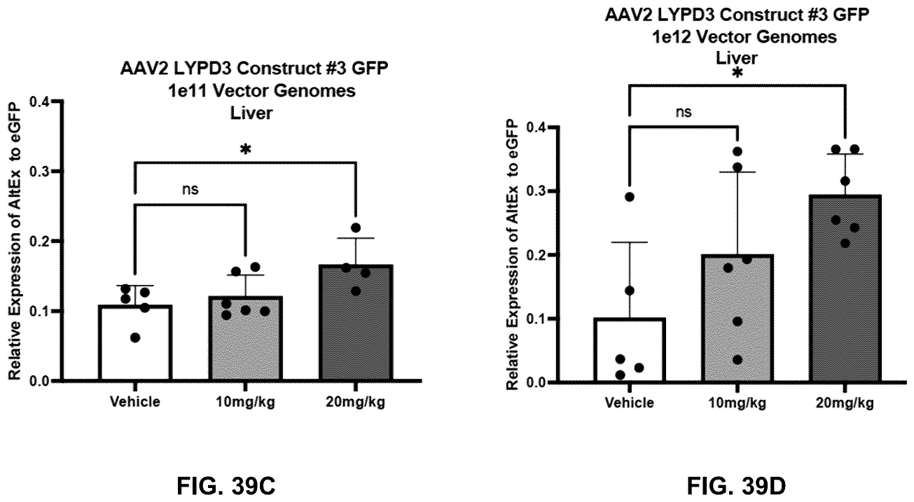

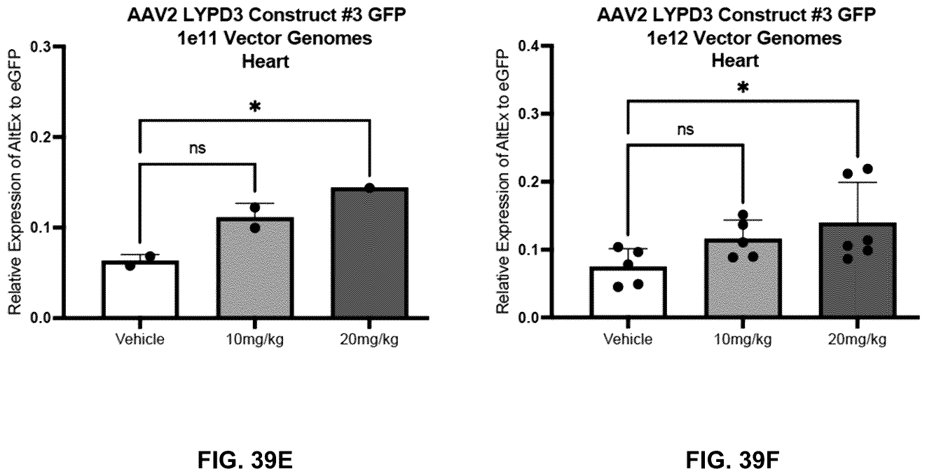

To examine the effect of alternative splicing driven by Drug K or Drug H, the RNA expression of the alternatively spliced exon was compared to the RNA expression of GFP in each drug condition. For animals that received TRPV3 Construct #3 at either vector genome dose, there was not a significant increase in the expression of the alternative exon upon treatment with 70 mg/kg or 140 mg/kg Drug K compared to the expression of the alternative exon upon treatment with vehicle in the liver (FIGS. 39A-39B). For animals that received LYPD3 Construct #3 at both vector dose, there was a significant increase in the in the expression of the alternative exon upon treatment with 10 mg/kg or 20 mg/kg Drug H compared to the expression of the alternative exon upon treatment with vehicle in the liver and heart (FIGS. 39C-39F). These results demonstrated that Drug H can be used to modulate the alternative splicing of a target transcript in animals, but Drug K was unable to modulate the splicing of the target transcript.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein.

All publications, patent applications, patents, and other references mentioned herein are incorporated by reference in their entirety.

TABLE 4
Sequences

| SEQ ID | | |
|---|---|---|
| NO: | Description | Sequence |
| 1 | TRPV3 genomic | ttttatccaagctgtgcttgtgatactgtctgtcttcttgtacttgtttgcctacaaagagtacctcgcctgcctcgtgctggccatg |
| | | gccctgggctgggcgaacatgctctactatacgcggggtttccagtccatgggcatgtacagcgtcatgatccagaaggtg |
| | | cgttgagagctgcgccggcgggagggccctcggaggatggccatttttcagatggggaaactgagatctggagaggaag |
| | | aggggtttaccaaggggcaggtcactgctctttctcccaaatgccatgcactccctttgctttcggccaggctttaagtgtcaa |
| | | gaccttaaatcccccaggcttgtggggtgctggggaggggtacagtttgtggaggaatgttctcacccctgggcagtggcgt |
| | | gctggtaaacatttaacgactgttctgtgtgtgtgtgtgtgtatgtgtgtgtgtgtaggtggaatatgtatattccatatatatacac |
| | | aaaagacttatcatacattttactatataaacgatgtgtatcacacaatttgcaaataatgaaaatataccatgcagtttattgtaaa |
| | | ttccacatagccagttgagtctcagaataatttgttgctttttttttttttttttggagaccaagtcttgctctgctgcccaggccaga |
| | | gtaaagtggtgcgatctcggctccctgcaacctctgcctcctgggttcaagcagttctcctgcctcagcctccagagtagctg |
| | | ggatcacaggcacgcaccaccatgttggctaatttttgtatttttagtagagacagggtttcagcatgttggccaggattgtcttg |
| | | aactcctgacctcaggtgatccacctgcctcggcctcccaaagtgctgggattacaggcatgaaccaccgcgccaggccta |
| | | tttgttgctttttattgaattctagtttttgttgtcaacttttggttgcaattgacaaaagagtgctatcagacatgaatgttggttgag |
| | | agttttctttataagcagtgattgaagtgaaacaattaagatttatgttgaactttacttgtttatcaatgatgtgagcaacttctttgc |
| | | tgaattggatactagtttgcaaataccagaataatattctgttgatatttttgtgctatacataatgtaaaggctgcagacagaaca |
| | | cattttaaaatttaaactggattaatagtttctccatcactgtcttaaatcatttagatctagatatcaacaaaacaatgagtcaatca |
| | | atctctgatttgtggcatttaccaattttcatgatgtaaatacagtagtccccccttattcttgggggagatgttctaagacccccc |
| | | atgaatgcctgaaactgcagatagtaccaaacattatataatatgctatgttttttcctatacatatacatctatgcaaataatgaaa |
| | | atatactatacaatacagtttattgtaaattccacttagccagttgagtcttagaataatttttgctttttttttttttttttgtttgtgactg |
| | | agtctcgctctgttgcccaggctggagtgcaatagtgcaatctcggctcactgcaacctctgcctcccgggctcaagcaattc |
| | | tcccgccccagcctcctgagtagctgggactacaggcatgcaccatgcccagctaatttttgtatttttagtagagatggggttt |
| | | cgccatgttggccaggctggtctccaactcttgacctcaggtgatccgtccgcctcggcctcccaaagtgctggggggtgg |
| | | gggggggaatcaacaaattattctaagactcaactggctatgtggaatttataaaatttattttatcaattaggcatagtgagatta |
| | | atggcaataactaacagaacaatgataacaatatactgtaataaaagtatgtgaatgttgtctctctcagaatattgtattgtgtgt |
| | | aatatttctgggtcatagttgaccacaggcaaccaaaactgcagaaagtgaaatcgaagagaagggcggactactgttctcc |
| | | tctcagggccaatttccagctaccaacattatgttactggacatagagttagggagaaacatgcagatccacatcgctctaga |
| | | attcctgccatatggctatgatagagacaagtaacctcaaaagcacacacaattgtaaatatagtgaaataactggacgggga |
| | | tgagttttgaacatttattgtctttgttttatttaattaaattaattaattaattaattttttgagacagagtctggctctgccccccaggc |
| | | tggagtgcagtggtgcgatctaggctcactgcaaccccttcctcctgggttcaagcaattctcccgcctcagcctctggagta |
| | | gctgggatcacaggtgtgtgccaccacacccagctaatttttgtatttttagtagagacggggtttcgccatgttggccaagct |
| | | ggcctccaactcctgacctcaggcgatccacccacctcagcctctcaaagtgttgggattacaggcgtgagtcactgcaccc |
| | | tggcctgtctttgttttaaatataacttattttatttaattgtaagtttatatggtttaattttttggcaatacaaataataatttctttaccc |
| | | ccagctttattgaggtacagctgacatatataattgtataaatttaagatgtacaatgtgaggatttgatataggtatatattgtgaa |
| | | atgatttccaatttccaattaacacacccatcacctcacatagttgccatttctttgtgtgtggtgagaacatttatgatctattctctt |
| | | agcaaatttctagtacacaacacggtattgttcactatagtcatcatgctgtccattagatccccagaacttattcatcttattcatt |
| | | gatttagttctgaataatggctgtgtttaacaaccagctcacaagcttcatgagaatttaacgatcagctcttgcgagctcttctg |
| | | agctggctccagcatgccactgcccccagcaagtgtgaggattccagtgtgagaccctatggtgaaaggtacagggtgaat |
| | | cacagggagaagagaacctggggctggagcagaaacagaaggcttcctggaggaggcaggatccagtgttgagccttg |
| | | gagcatcaggaggatttggctgggggaagggagagtggcccaagtgaagagacagttagggtggggggtgggagagg |
| | | gtgggaagcaggaagatcagggtgccgtgaatatgtgcttgcaccttacacttgaattatgtttatttccttataggtcattttgc |
| | | atgatgttctgaagttcttgtttgtatatatcgtgtttttgcttggatttggagtaggtaattgatttataaataatagtaactcccacat |
| | | ttataaacactttatacagatgtcaaaatggcaaagtgcatggcggccttggcaacaattagaccaactaccagtggggcact |
| | | tgccatgcttggcattttccaagcattacctttatatatatacatatatattttattatactttaagttctagggtacatgtgcacaaca |
| | | tgcaggtttgttacatatgtatacatgtgccatgttggtatgctgcacccattaactcgtcatttacattaggtatatctcctaatca |
| | | agcattacctttttacttctctcaacaacttgatcaagtgggaatcatcaccccgatcataaggtaactgaggcttagagactcc |
| | | aggattcaaaccaggccagccactcataatccaacttcaccatgtggtatatacctctggctttcccattcattgcttacgtggc |
| | | cctcacgacctaagataggttctgcaagtgttttcagccccattttaacagatgcggaaactaaagtccacagaaactcaactg |
| | | atggtctttgcaaagtaggagcaaactaataccctgaggtgatttcctttggatttcacatcacgttactctcacagtttgctttag |
| | | atacaactgtgagagcctctctggtctgctgggaccaggaaggagccccctcttaaaatctctccatttacaacagagggaa |
| | | cagcacgatctggttgtatttctaacctctgcatagagagatatcattttcccccacgtaccagttgcttttttttcttttcttcagtga |
| | | ccattgacatttgaaattaagtagctgtttccatctgtctggtaccttttctgggtcacaatttgcttatttatttgctccttcacctgct |
| | | tgtttattcatccatctatttactctctcatttaatttgctcatccacctgtctatccgtccatccatccatccatccatccatccatcca |
| | | tccatccaatcattcatccatcgatccatccattgatctatccatctatccatccatccatccatccatccatccatctaatcatcca |
| | | tccaatcatccatccatccatccatccatccatccatccatccagtcatccatccattcatccatttaatcatccatccatcgatca |
| | | atcggtccatccatccagctatccatccatccatccatccatctaatcattcatccatcaatccatccatccatccatccatccatc |
| | | catccatccatccatctatccatccatccaatcatccatccatccatcgatcaatcagtccatccatccatctaatcatccatctaa |
| | | tcatccatccatccatccatccatccatccagtcatccatccattcatccatttaatcatccatccatccatcgatcaattggtccat |
| | | ccatccatctatccatccatccatccatccatctaatcacccatccatccatccatccatccaatcatctatccatccatccatcta |
| | | atcatctatccatccatccatccatccatccatccaatcatccatccatccatccatccatccctccatctaatcatccatccctcc |
| | | atccatccctccatatccatccatccatccatccatccatccatccaaccattcagcagtgaacctctactgtggtgtgttggaa |
| | | agaagggaagtgtgagcctggtcttgctcacagggaactcaaaacagagcattaggaggagactcaggatcttacagcctt |
| | | gagggaggtgacccaggctagaggaagtcaggagctcaaagactagctggggagccagggacacacacatggattata |
| | | attagtggctctgcaaggctgcaagtgatcaagggcagactgcggggtaaaggaaggagagaagactctgggctttgttag |
| | | gtggaagggctttctgcaggaggccaggccagagaagccagcctgatgaggctttgagaggacctgaaactggagatgg |
| | | gatttaatggccagagagaaggaggcagcaggcttgtcatgtaggcaaatacacgcaagcttgtagagtaattaccaggca |
| | | gagagctgaggagagcaccctgttatgttcccttagccttggcctcgctgatcgagaagtgtcccaaagacaacaaggact |
| | | gcagctcctacggcagcttcagcgacgcagtgctggaactcttcaagctcaccataggcctgggtgacctgaacatccagc |
| | | agaactccaagtatcccattctctttctgttcctgctcatcacctatgtcatcctcacctttgttctcctcctcaacatgctcattgct |
| | | ctgatgggcgagactgtggagaacgtctccaaggagagcgaacgcatctggcgcctgc |
| 2 | STK31 genomic | aactctggtggtctccttacaatgagcttggaacgagatcttcttgatgctgagcccatgaaggaacttagcagcaagcgtcct |
| | | ttggtacgttctgaggttaatgggcagataattctgttaaaggtaagtctaacttcttcttacttgtgggagattcagcagcataaa |
| | | ggatagtggtgctgttacatttcaagaaagaaaaaacctcactgttagttttctaaagtgtataagttgactggcatgataattgt |
| | | gcttgtagtttggttgccagaactaattttcacttttaaagtttccctcaattgaacaatgagaacacttggacacagggcgggg |
| | | aacatcacccgtgggggcctgtcatggggtggggggcagggggagggatagcattaggagaaatacctaatgtaaatgat |
| | | gagttaatgggtgcagcaaaccaacatggcacatgtatacatatgtaacaaacctgcacgttgtgcacatgtaccctagaact |
| | | taaagtataataataaaaataagtatataaaagtcattaaataaagctttgtctcacaagaaaaaaaaataaagtttccctcatttc |
| | | cctaaatagaatatgtccttaataggatatactttcctttttttttgggtgaacttattatagtcatccaatatctttttaaaattcttgatt |
| | | ttcttacatttctccttgaaatctcagctgtgttatcagtacaatcttctactctttaatttcttcttctcattgatctgggaaactgttaa |
| | | tcatgataataagataattaatttggccttagttgctacacattctagaatgtgtagcaggctacaggtagaattgcactttcctttt |
| | | ccaaattatagcattcaaagaagatagaaaagtacttataaaattaaaaaataaaattagatttgatttaacttaaaataatgaatt |
| | | atgtttaaattggaatgatgtataattataatgagattttcatcttggaattacgagtgcctctttctttggtacaagggctattctgt |
| | | ggatgttgacacagaagccaaggtgattgagagagcagccacctaccatagagcttggagagaagctgaaggagactca |
| | | gggttactgccattgatattcctgtttttatgtaaggtaaagttcttatgctaatctcttgcagaaaccattttatcttgcagaaactat |
| | | tttatttaaaataaattttagctccaggttttagagcagcagttcattccccttaacataaaagaaaaattatcccatagaatcaatg |
| | | tatgctaaaaatatgttgaagaagtttttacttggagtcacattctctgttttgcttcttagtctgatcctatggcttatctgatggtcc |
| | | catactaccctagggcaaacctgaatgctgttcaagccaacatgcctttaaattcagaa |
| 3 | USP25 genomic | gtgtcacaacataatgaaagttgctcaagccaaactggaaatgataaaacctgaagaagtaaacttggaggaatatgaggta |
| | | atgtgctttcttagaggttaaactacagatttagggattctgtaattcactgatattcctttcgtttcagcccaactgaagttgaattt |
| | | gttatgaatgatgagtaaaaaaattaagtttccaagtcaatgctcaagtaattaataattttatatgtgattttaggattaacatgata |
| | | tgatagcatgtagaccaaaaagtttaaaatcaaaaaaggaagatttttggttttttatttcaatactaattctcaagtactttttaaat |
| | | gaacttttctagcttaaaaacagaataacaaatgcattgttaattttgaaatcaacttagaaattgctgttacataataccaacaag |
| | | taatatttatcaagaccctactatatgccaggcatttgtctaagccctttacatatattaactgatttaattcagacagtaacactgt |
| | | aagataggtagtgttcttattcccatttgacacacgagaaaataaggcatagagcagtgaagtaacctgcctaaggttttcaca |
| | | gccacaagtttacagcttggctaatagaacctacattcttaactgttgcactacactgtgcaactgtaccctgtgggctgtattttt |
| | | tccttcactttgattcatcagatatattttggggaagttaattcattttagtccctatatgccatatatgtttgtcatgagacagtgttgt |
| | | gtgtgagagagcacttacataggtacttgtacatgtgcttgtgggatttttttgtgcacaaaattattttttgtcaactttggctttaa |
| | | aatattctttattttaattgatgaattcatacacctagtatttcttcacatttttaaatttaatcttatgcattgtcttttttttaatttataagt |
| | | ctagtaaattttaagttgttttcttgggtgcctttgcttaatatttagttccatttacaaacttaattatggtactttagattccctaaata |
| | | attagtgtaattcactacacaaacagggaagaggaaagagagagaaaacactcattattgatgaaaaccttagctagtttggc |
| | | gtataaggtaactaccttaattcactaaagagtgtctctaaaaacaaaaataggaaataataaaactgcagcaaatatatagaa |
| | | ttgtgagcactgaaagcttaaaatttttgttgtcaaaagcaaggcaatgaaaataagcgaaaagtaaaatactggaaaagaag |
| | | aaacaaagacatcattatttgtagttttttttaccttgaaattcaaataaaatctacaactgatgtattagaactagtaagacagcttt |
| | | gcagcatgcaaacttgtgtcagacatcaattttattcccttttgccagtaaaaaaagtatgagaaagtataaaagtgagaccatt |
| | | tacaatagtatctgaaactatggagtatatagaaataaatttaacaaaaggtgcgtgtgacttctgtggggaaaattataaaactt |
| | | attgggagacattaacaaagacctaaataaatgggcatacatatggtatttggaaaacttgatattattaatatatagatatcactt |
| | | ttcccccaaatgctttataaattcagtgcaggctgtatgaaaattccagcaagatgttttatggagcttgataagccaactgaaa |
| | | aatgttacggaaatgcaaagatcccagtccacctcccatctctgacaagcaagttagtgaacagtataaagactttttaataaat |
| | | ggttctgggaaataaataaaatttgatttcttctttattccatatgcaaaaattcatttcaattgtgaaagactcctgtgtaaagctgt |
| | | ttagaagtcaacataggtagaagcccaaagacattcatttatccataagaggcactcactgaccagatatttttgaccaggata |
| | | tgtaatctagagccagagaaattcaaaatttgttttctcttgaccctgaggagcaagtcaagtcatcttttagctaataatgactcc |
| | | aaatctgtgccctccacattgattgagagctcttcccttgtccccaagattcgtgttcatggaacttgatttttaattgtgacttgatt |
| | | ctgcaatgcttccttcagtttgcttcaacagattggactcattatgttttttttcataaaatcttctcatattctaactgcagggctgtta |
| | | aatcatacaagtatttaaatgacttgtttttagattctgcatattcctcctatctttgtcttgctctaagttcagggttcagtaaaccat |
| | | agcctgcagaccagcttaccacctgtttttgtaaataaacttttattggaacatggccatttgtatacatattgtctgtggatacttt |
| | | cacactgcagttacagagttgagtaactgaagaagatgaggcttgcaaagtctaaaatattatctggccttttacagaaaaagt |
| | | ttgcctactcacaacttctaaggtgattctaatgacaacaaggaatgacagtttgaataccttccccagaaaagctaacttcata |
| | | cttgcaggacaaattttacctttatttgttatgaagatctactaatagcatgaaaattatgtcttgaaaattaaaattaagactggat |
| | | gtggtggctcatgcttataatcccagcacttttggaaatcaaggcacagggagttgtttgaggccaggaatttgagaccggcc |
| | | tggccaacatagcaagaccctgtctatacaaaacagattaagtaattaactggaactgggagcatgcacctgtaattcaagct |
| | | acttgggagacagaggtaggaggattgctggagcccagaaggtcaagtctgcagttagccgtgatcgtgccactgcactg |
| | | cagactgggcaactgagtgagaccctgtctccaaaaaaaaaaaaaaaagtttatattcaaggagaatatcgttggctaataac |
| | | tatgtttcttatttttgtttataatgtttatataaaagccttaacacttttgttatcagtattcactttccaatttatcctacattgaatttcag |
| | | ttatttttcttgagtcaattattataaagtaagctaagaaaatcttgactttggtatcaccttagaattatttagtcctattacatgccct |
| | | atagttgttttgggggtggggaggggcattgaaatgtgaactgcagttattcttttaccatatctcatgtaaataaactattcgcct |
| | | acacacttaaagtcaaatttaccacatatttgcgaagttaaaaggttaaataaatctaactttttttagtttcttaaaattatgtattaa |
| | | gtatgtgtccaagctttgattcatactcagcgtaatatttcattttcttccatcagttgatgtagatataataatagttcaataatatga |
| | | ttttatactgatgaatttttgagatgacctgattgattatcctgcctttatatttctgttatctgctagtaaaatctttattgtttaaaacat |
| | | ttcatatccatgcctaataatttttagatttaatgttgtattaataaatagtcttgtgtgccagaaagtaaagtcttgttttagttctgat |
| | | gtatcttcattttgaactctttatttctattaattcagatagtagcgaaacagtgcattactttttgtacagtttcatagtactttttaaat |
| | | cttactctgaacatttttaatattttctttcacctacaggagtggcatcaggattataggaaattcagggaaacaactatgtatctc |
| | | ataattgggctagaaaattttcaaagagaaag |
| 4 | GTF2B genomic | gatcgaacaaataatttattcaagcaagtatatgaacagaagagcctgaagggaagagctaatgatgctatagcttctgcttgt |
| | | ctctatattgcctgtagacaagaaggggttcctaggacatttaaaggtaagtttttagctttgtttttttgtgtttgtttgtttgtttgttt |
| | | gagatagggtctcactttgtcgcccaggccagagtgcagtggcgcaatcttgactcactgcaacctctgcctcccaggttca |
| | | agctatactcctgcctcagcctcctgagtagctgggattacaggcatgtgccaccacacctggttaacttttgtatttttagtaga |
| | | gacagggtttcgccatgttggccaggctggtctcaaacttctgacctcaggtgatctgcccactttggcctcccaaagtgcta |
| | | gaattaacaggcgtgaggcactgtgcccatcctaaaataacattttctacatcatattaaagaaacagaccaatgtcaggcag |
| | | ctttgacacttagaagctaagtcatctttattgtgactacagtaatccacttgagcatcatttgccaatgcctacttatttcattcctt |
| | | gacagcaaattcatagcatgttcttttaggctgttttcccatgtagactcattttagtccacgtaagcccactatgttcttttctagtg |
| | | agatcttttgatggactgttgctttgtgggctttcagaaggcctataatcgtaaaccaaagtaggccgtttatacatttggtttaca |
| | | tggaaacagtttccatttctagcactggtctctgtagatgagggagtcaagctttagaaagtatcagtgaaggccaggtatggt |
| | | gtctcacacctgtaatcccagaactttgggaggccgaggtggttgggtcatgaggccaggagttcaagaccagcctggcca |
| | | atatggtgaaaccccgtctctactaaaaatacaaaaattagccaggcgtggtggtgcgcgcctgtagtcccagctactcagg |
| | | aggctgaggcagaagaatcgcttgaaaccgggaggcagaggttgcagtgagccgagatcacgccactacactccagcct |
| | | gggcgacagagtgagacttcatctcaaaaaaaaaaaaaaaaaagttatcagttaagttttttctcttgaggtgtattgtttgcact |
| | | tttacacttcagttaagaactggtatttttcatttagtttgcagttacatgtgaatatgtgataatttgttcagtgttgctcactgacttc |
| | | cactaccaaagctcagtttagctgaagactatacttaaccctttattcatccagtgagtatttattttttatgttttcttacgtaaaagt |
| | | gaagaggtgatggcctttatcatcatcttttttgttgtttttataactaaaacaacttttgttaaatttcttctgaaagtgtgtggtggtt |
| | | gcatctgggaatagcagggtcagatattttatctcattgagctacagcccataatcgcagggatttcatgcacacatgacattgt |
| | | gttgcctaagaggcatcctttagcttgagtattttctttcaattccttggtaacactgcttacaacagattaatacataaacatcaaa |
| | | gaatttttcatttaaagaaatttagttgtatgaattaatccttagagcagttaatattttgaaagattaagacctaaatttagccaggc |
| | | acgatggctcacaccggtaatctcagcactttgtgaggccgaggcaggtagatcacgaggtcaggagttcaagaccagcct |
| | | ggccaagatggtgaaaccccatctctactaaaaatacaaaaaattagccgggtgtggtggcaggcgcctgtaatcccaacta |
| | | ctcgggggcaggagaatcgcttgaacccagggggcagaggttgcagtgagccaagatggtgtcactgcactccagtctg |
| | | gacgacagagtgaaatttcatctcaaaaaacaaaaaaactctaaattgaaagagtgaagaacattaggccaggcgcagtgg |
| | | ctcatgcctgtaatctcagcactttgggaggccgaggtaggaggatcacctgaggtcaggagttctagacaggcctggcca |
| | | acatggtgaaaccccatctctactaaaaatacaaaagttagccaggtgtggtggtgcacgcctgtagtcccagctactcggg |
| | | aggctgaggcaggagaatcacttgagcctaggaggtggaggttgcagtgagctgagatcatgccactacccttagtctggg |
| | | cgacagagtgagacttcatctcaaaaaaaaaggtgtgatgaacattaaagttaactgagaaatttgcagttaactctgtggga |
| | | agtatcactgagatgattagggtttcatctttaaggtatgattgaatgttttacaattagaatgtatattatgtttataattagaaatgg |
| | | tagattttaagatgaactatatggcttaagtgaattgatttatttaattttttttcttttagaaatatgtgccgtatcacgaatttctaaga |
| | | aagaaattggtcggtgttttaaacttattttgaaagcgctagaaaccagtgtggatttgattacaactggggacttcatgtccag |
| | | gttctgttccaacctttgtcttcctaaacaagtacagatggcagctacacatatagcccgtaaagctgtggaactggacttggtt |
| | | cctgggaggagccccatctctgtggcagcggcagctatttacatggcctcacaggcatcagctgaaaagaggacccaaaa |
| | | ag |
| 5 | LYPD3 genomic | atggaccccgccaggaaagcaggtgcccaggccatgatctggactgcaggctggctgctgctgctgctgcttcgcggagg |
| | | tgggtctgctgcccggagaggtggagggggtagagtgggggctcttagggaggaggctcctctggccccgtacagagaa |
| | | ggctgtgggaaggatccccacataagcagagggcactgtgggcaaagttcccccatcctaagcaagggaggcagaggg |
| | | aagggtcctccaccatgggccgtaggaagggttccccaccacggacaaggggagctgtgggcaaaattttcctatcctgaa |
| | | gctgcagaaagaagtcccctgttcctaggccgggcgcggtggttcatgcctgtaatctcagcactttgggaggccgaggca |
| | | ggcggatcatgaggtcaaacgatcgagaccagcctggccaacatggtgaaaccctgtctctactaaaaatacaaaaattag |
| | | ctgggcgcggtagcgcgagcctgtagtcccagctactcgggaggctgaggcaggagaatcgtttgaatccaggagggga |
| | | ggttgcagtgagccgagattgcgccactgcactccagcctggcgacagagcgagactccgtctcaaaaaaaaaaaaaag |
| | | aaaaaaagaaaaaaagaaaaaagtgttccctgtcctgggcagagaagctgtgggtagtgttctctcatttgagcagaaggg |
| | | ggctgtgggaagggatccctcactgagcacagtctactgtgggaaaagttctcccacacactgtgggaaaagttctctcattc |
| | | tgagcagaaaaccgtgaggaggtatccttcctaccccgggatgaggggcttcatggggggatcgcctacagtgggcata |
| | | cagggctgtcggaaggatgtgcccacagttggcatagtgggtgtgaaggaaaagggtccagtgattcccgtgcttccagat |
| | | ttgtggaaagcttttccctactcctgccctggaaaagtgtgtgcaataagactgtctaatcccgggacagagggcatgggaa |
| | | ggactatttctccttgggggtggacgctttagcaaggagggctcaggtctattcggctccgctcccccaggagcgcaggcc |
| | | ctggagtgctacagctgcgtgcagaaagcagatgacggatgctccccgaacaagatgaagacagtgaagtgcgcgccgg |
| | | gcgtggacgtctgcaccgaggccgtgggggcggtggagaccagtgagtggggacccccgggccacgcacaccactgc |
| | | aggctcctccggcccagccccgcctccttatagtcctttctggatctggccacgcccctttaacgcggccccacccctcccttt |
| | | cagcctggccgggaccctacgacacagccttgcaggccaggccttctctattaacaattacaagcgcgaggagcagagag |
| | | ttgagaactgggtggggcggacggggagagctcgggtttggaacgaggtcacgtcctatgcactttttctgctccaccaatg |
| | | agacctccccatccctgcctgtcaaccaggctgtctcctccgactcagctctttgaggttccgcccccgttccgtgaccccgc |
| | | tcctccctgcttcggtcgttctttccgctctgagcggctctttgggggcgtccatccgccaattcctgcctgtcggtcctgtggt |
| | | gttggcctatttgaggccccgcccctcccagaccctacccaactctgggccacgccccatctgagaccacgcccacagcta |
| | | atcccgcccttgtccctctccctagtccacggacaattctcgctggcagtgcggggttgcggttcgggactccccggcaaga |
| | | atgaccgcggcctggatcttcacgggcttctggcgttcatccagctgcagcaatgcgctcaggatcgctgcaacgccaagc |
| | | tcaacctcacctcgcgggcgctcgacccggcag |
| 6 | IK genomic | gtgagccgttctccaaccctttggcccccgatggccacgatgtggatgatcctcactccttccaccagtgagtattttgtgcta |
| | | ggaccatggaaatgagttgggcaatgaatagaaaagtggtggtgatggtgaaagaattctgaatgtcttgtcatcaaggatg |
| | | gtaaaaaattatacccgctggccctttatctctgtgagccttaactggatcaggccaagtgcataagggtattagaagggaaat |
| | | tcctgcaggaaaggtgttaagtttttttttttttttatacgtgttgtgtaattacacattatataggaattacttcttccaatagcaaatta |
| | | aaacataggaaataagggtaaaatcatctttttattttttttgagactgagtttcgctcttgttgctcaggctggaatgcagtggtg |
| | | cgatctcggctcaccacaacctccgcctcccgggttcaagcaattctcctgcctcagccttccgagtagctgggattacagg |
| | | catgttccactacgcccggctaattttgtatttttagtagagatggggtttctccatgttgatcaggctggtcttgaaatcccgact |
| | | tcaggtgatccacccgcctctgcctcccaaagtggtgggagtacaggcgtgagccaccgtgcctggcccccgataaaatc |
| | | atctttgataacatccccaattccagtccatttctttttttttttttttcttttcttttttttttttttttttttttgagacggagtctcactgtt |
| | | acccaggctggagtgcagtgatgcaatcttggctcactgcaacctctgcctcctgagttcaagcggttctcctgcctcagcct |
| | | cccaagtagctgggactacaggcatgtgccaccatgcacgactgatttttttttttgtatttttagtagagacagggtttcactgtg |
| | | ttcgtcaggctggtcttgaactcctggcctgaagtgatccacccgcctcagcctcctaaagtgctggcattacaggcgtgagc |
| | | caccgtgcccagcctcagtccttttcttagaagtaaccacagttaccttttttggcatatcttttcctcccgttgcccaggctgga |
| | | gttcaatggcattatttctgctcacagcaacctttgcctcctgggctcaagcaattcttctgcctcagtttccccagtagctggga |
| | | ttacaggcatgccccaccatgcttggctaatttttgtattagtagagacagggtttcgctatgttggccaggctggtcttgaactc |
| | | ctgacctcaaatgatctgcccgtctcggcggcccaaagtgctgggattacaggcgtgagccactgcgcctggccctcctact |
| | | cttttttaaatgcatttatacacatacatgcatatctgaggaaaacacaacattgctttgtgatttaaaatatttgagttaaatgatatt |
| | | atgctgtatatatcatttttgcaacttgcttctttttaaaccctgctcagtgtttctgagattatctttcagagcagtgacaagcattcc |
| | | ctccttggctaaactttagtcaggttccccaaagagtcccatttttcagcaggactcatccgtggcctgctgagcacagttttaa |
| | | caaagaaatcaaccaccgctccacccccatacctaaccaagttgcttttagtaattttctataaattctcacttgtccctgttgtatt |
| | | cggaattaagttcagtctctctactctattacaatggtcttgactcttgttacagtagtcttcaataaagtcttccttggggagaag |
| | | aggaacttgattagatatcaagttggattggatatcaagagtgggaaaactttctgtgaactgacttaggattcctttttaaaact |
| | | gaggtcagcaggccaaggaagagccctagtcagaagagagctttaaaggagtctgactttaatttgatcaagagaggagtc |
| | | tttgttagtgtaccaattaaaataatctcttgtaccattagtgctgtctttccctctttgggaaacaccatcatagtttagtcagtctc |
| | | cagctaataaacgcttagtctgtttcagactgattcttttcttttttttatttttttgagacagagtcttgctctttttgcccaggctgga |
| | | gtgtaatacctagatctcggctcactgcaaactccgcctcccaggttcaagcgattttcctgcctcagcctcctgagtagctag |
| | | gattacaggcgcccaccacaacgcccagctaatttttttgtatttttagtagagatgggatatcaccatgttggccatgctggtct |
| | | tgaactcctgacctcgggtgatccgcctgcgtcagcctcccaaagtgcggcgattataggtgtgagccactgcacccggcc |
| | | agactgattattttcaaggctataactgaattaaatttacaggccttcttatatacatatgtcagtaatggaccaggctaaatataa |
| | | ataaatggagttgctggctcataggatatatatacttaaattttaacaaatactgcaaaattgcccttcaaattggttatactggttt |
| | | acattctcatcagcattatatgagagagtccccttcccaaattctcactcacacttgatttatcaaactttctggtagttaacactct |
| | | gatgcattaaaagtggcatggcatatctggtcgggcgtggtggcttacacctataatcccagcactttgggaggctgaggtg |
| | | ggcagatcacctgaggtcagaagtttgggaccagcctggccaacacctctaataaaaatacaaaaattagctgggtgtgttg |
| | | gcgggcacctgtattcccagctacttgtgaggctgaggcaggagaattacttgaacccaggaggcagaggttgcagtggg |
| | | ctgagatcatgccactgcactccagccggggcaacagagggagactctgtcttaaaaaaaaaaaaaaaaagctgggtgcg |
| | | gtggctcatccctataatcccagcactttgggaggccgaggtgggtggatcacaaggtcaggagttcaagaccagcctggc |
| | | caatatgggtgacagtgagactccgtctcaaaaaaaaaaaaacagtgtcatatctttgttttaatttgtatttccctgatttctgtta |
| | | atttaatcatgatttttacttgttcctttttcatttcttattgagtcctaatatttaaaatctttgccttttcttctagatcaaaactcaccaa |
| | | tgaagacttcaggaaacttctcatgacccccagggctgcacctacctctgcaccaccttctaagtcacgtcaccatga |
| 7 | TEX 14 genomic | tggaggtgaaagaaatggcaaagaaagcagctactggacagctcacagtacctccttggcatcctcagagtagtctgacttt |
| | | agagagcgaggctgaaaatgagcccgacgccctgctgcagccccccattaggagcccagaaaacacggattggcagcg |
| | | agttattgagtatcatagggaaaatgatgagcccagaggaaatggcaagtttgacaagacgggcaacaatgactgtgacag |
| | | tgaccagcatggcagacagcccaggcttggaagcttcaccagtaagttcacttgcgggaatcaggataggtgaactcaatc |
| | | aagtatcagtcgggcgaggtggctcagcctgtaattccagcactttgggaggtgaggcgagcggatcacctgagcccagg |
| | | aatttgggaccagcctggtcaacatgatgaaacctggtctctacaaaaaaaaaaaaaaaaaaaaaaaaaacattagctggg |
| | | cgtggtggcacacgcctctagttccaggtacccaggaggctgaggtgggaggatcacttggacctgagaggtggaggttg |
| | | cagtgagctgtgatcatgaaactgcactccagcttgggtgacagtgagaccctgtctcaaaataaaaataaaagtaaaaaaa |
| | | agttatcagctctcaaaaaagtaaactttccagcatattttttttggtttggtcctctctcctttcctacttttctggaaaccttgaattc |
| | | caggggaagacagtatagccctatatctcattgcatttcccacaggcaaacagatccagacactgatcagtttttatattactga |
| | | aacaggtccctttgtccaaaggaagtaaaataatcaaatactgaccctggttttggcgaggaatcgatccttctttacctgggg |
| | | atagaggtaggccatcactgcctactgcccatgtctctgcattgtattctaagctgaaattaaattctcccagaatttatttagaat |
| | | gtattctaagctggcattaaatttttccagagagtctatgctggaggaacctagagacacatgacatttattgtcatcggtttata |
| | | gatttacaggttaaaaaaaatgaaggctgtatattttcagttgataatttgcttataatcagggcatggatattgtttcacagttgttt |
| | | ataagttaaagaaagatacatctaaacttgtcatttgtcttaggcagaaaatctattggcttgggtattatttgtatttagaatctgtt |
| | | tcttgtcaagcatcactctcgaatggggggcagatgatccagggctgcattttggggtcaggctaaactaacagtctctactc |
| | | tctggactgaacctaacgtatgggacatggccactcttggcagaatatgtatacatctcttctttcatgccacctttatcctcttcc |
| | | atgttgtctgaatatcactgaatgactttgaccctgactgtgttacaatcattaggtatcaggcacccatctcccagacaaaagg |
| | | agcaaccagagcatagtgaagccttccaagcaagttctgacacattggtggctgtagagaaatcttacagt |
| 8 | TRPV3 | ttttatccaagctgtgcttgtgatactgtctgtcttcttgtacttgtttgcctacaaagagtacctcgcctgcctcgtgctggccatg |
| | upstream exon | gccctgggctgggcgaacatgctctactatacgcggggtttccagtccatgggcatgtacagcgtcatgatccagaag |
| 9 | TRPV3 | gtgcgttgagagctgcgccggcgggagggccctcggaggatggccatttttcagatggggaaactgagatctggagagg |
| | upstream intron | aagaggggtttaccaaggggcaggtcactgctctttctcccaaatgccatgcactccctttgctttcggccaggctttaagtgt |
| | | caagaccttaaatcccccaggcttgtggggtgctggggaggggtacagtttgtggaggaatgttctcacccctgggcagtg |
| | | gcgtgctggtaaacatttaacgactgttctgtgtgtgtgtgtgtgtatgtgtgtgtgtgtaggtggaatatgtatattccatatatat |
| | | acacaaaagacttatcatacattttactatataaacgatgtgtatcacacaatttgcaaataatgaaaatataccatgcagtttatt |
| | | gtaaattccacatagccagttgagtctcagaataatttgttgctttttttttttttttttggagaccaagtcttgctctgctgcccaggc |
| | | cagagtaaagtggtgcgatctcggctccctgcaacctctgcctcctgggttcaagcagttctcctgcctcagcctccagagta |
| | | gctgggatcacaggcacgcaccaccatgttggctaatttttgtatttttagtagagacagggtttcagcatgttggccaggattg |
| | | tcttgaactcctgacctcaggtgatccacctgcctcggcctcccaaagtgctgggattacaggcatgaaccaccgcgccag |
| | | gcctatttgttgctttttattgaattctagtttttgttgtcaacttttggttgcaattgacaaaagagtgctatcagacatgaatgttggt |
| | | tgagagttttctttataagcagtgattgaagtgaaacaattaagatttatgttgaactttacttgtttatcaatgatgtgagcaacttc |
| | | tttgctgaattggatactagtttgcaaataccagaataatattctgttgatatttttgtgctatacataatgtaaaggctgcagacag |
| | | aacacattttaaaatttaaactggattaatagtttctccatcactgtcttaaatcatttagatctagatatcaacaaaacaatgagtc |
| | | aatcaatctctgatttgtggcatttaccaattttcatgatgtaaatacagtagtccccccttattcttgggggagatgttctaagacc |
| | | ccccatgaatgcctgaaactgcagatagtaccaaacattatataatatgctatgttttttcctatacatatacatctatgcaaataat |
| | | gaaaatatactatacaatacagtttattgtaaattccacttagccagttgagtcttagaataatttttgctttttttttttttttttgtttgtg |
| | | actgagtctcgctctgttgcccaggctggagtgcaatagtgcaatctcggctcactgcaacctctgcctcccgggctcaagc |
| | | aattctcccgccccagcctcctgagtagctgggactacaggcatgcaccatgcccagctaatttttgtatttttagtagagatgg |
| | | ggtttcgccatgttggccaggctggtctccaactcttgacctcaggtgatccgtccgcctcggcctcccaaagtgctggggg |
| | | gtgggggggggaatcaacaaattattctaagactcaactggctatgtggaatttataaaatttattttatcaattaggcatagtga |
| | | gattaatggcaataactaacagaacaatgataacaatatactgtaataaaagtatgtgaatgttgtctctctcagaatattgtattg |
| | | tgtgtaatatttctgggtcatagttgaccacaggcaaccaaaactgcagaaagtgaaatcgaagagaagggcggactactgt |
| | | tctcctctcagggccaatttccagctaccaacattatgttactggacatagagttagggagaaacatgcagatccacatcgctc |
| | | tagaattcctgccatatggctatgatagagacaagtaacctcaaaagcacacacaattgtaaatatagtgaaataactggacg |
| | | gggatgagttttgaacatttattgtctttgttttatttaattaaattaattaattaattaattttttgagacagagtctggctctgccccc |
| | | caggctggagtgcagtggtgcgatctaggctcactgcaaccccttcctcctgggttcaagcaattctcccgcctcagcctctg |
| | | gagtagctgggatcacaggtgtgtgccaccacacccagctaatttttgtatttttagtagagacggggtttcgccatgttggcc |
| | | aagctggcctccaactcctgacctcaggcgatccacccacctcagcctctcaaagtgttgggattacaggcgtgagtcactg |
| | | caccctggcctgtctttgttttaaatataacttattttatttaattgtaagtttatatggtttaattttttggcaatacaaataataatttctt |
| | | tacccccagctttattgaggtacagctgacatatataattgtataaatttaagatgtacaatgtgaggatttgatataggtatatatt |
| | | gtgaaatgatttccaatttccaattaacacacccatcacctcacatagttgccatttctttgtgtgtggtgagaacatttatgatcta |
| | | ttctcttagcaaatttctagtacacaacacggtattgttcactatagtcatcatgctgtccattagatccccagaacttattcatctta |
| | | ttcattgatttagttctgaataatggctgtgtttaacaaccagctcacaagcttcatgagaatttaacgatcagctcttgcgagctc |
| | | ttctgagctggctccagcatgccactgcccccagcaagtgtgaggattccagtgtgagaccctatggtgaaaggtacagggt |
| | | gaatcacagggagaagagaacctggggctggagcagaaacagaaggcttcctggaggaggcaggatccagtgttgagc |
| | | cttggagcatcaggaggatttggctgggggaagggagagtggcccaagtgaagagacagttagggtggggggtgggag |
| | | agggtgggaagcaggaagatcagggtgccgtgaatatgtgcttgcaccttacacttgaattatgtttatttccttatag |
| 10 | TRPV3 | gtcattttgcatgatgttctgaagttcttgtttgtatatatcgtgtttttgcttggatttggagtag |
| | alternatively | |
| | spliced exon | |
| 11 | TRPV3 | gtaattgatttataaataatagtaactcccacatttataaacactttatacagatgtcaaaatggcaaagtgcatggcggccttgg |
| | downstream | caacaattagaccaactaccagtggggcacttgccatgcttggcattttccaagcattacctttatatatatacatatatattttatt |
| | intron | atactttaagttctagggtacatgtgcacaacatgcaggtttgttacatatgtatacatgtgccatgttggtatgctgcacccatta |
| | | actcgtcatttacattaggtatatctcctaatcaagcattacctttttacttctctcaacaacttgatcaagtgggaatcatcacccc |
| | | gatcataaggtaactgaggcttagagactccaggattcaaaccaggccagccactcataatccaacttcaccatgtggtatat |
| | | acctctggctttcccattcattgcttacgtggccctcacgacctaagataggttctgcaagtgttttcagccccattttaacagat |
| | | gcggaaactaaagtccacagaaactcaactgatggtctttgcaaagtaggagcaaactaataccctgaggtgatttcctttgg |
| | | atttcacatcacgttactctcacagtttgctttagatacaactgtgagagcctctctggtctgctgggaccaggaaggagcccc |
| | | ctcttaaaatctctccatttacaacagagggaacagcacgatctggttgtatttctaacctctgcatagagagatatcattttccc |
| | | ccacgtaccagttgcttttttttcttttcttcagtgaccattgacatttgaaattaagtagctgtttccatctgtctggtaccttttctgg |
| | | gtcacaatttgcttatttatttgctccttcacctgcttgtttattcatccatctatttactctctcatttaatttgctcatccacctgtctatc |
| | | cgtccatccatccatccatccatccatccatccatccatccaatcattcatccatcgatccatccattgatctatccatctatccat |
| | | ccatccatccatccatccatccatctaatcatccatccaatcatccatccatccatccatccatccatccatccatccagtcatcc |
| | | atccattcatccatttaatcatccatccatcgatcaatcggtccatccatccagctatccatccatccatccatccatctaatcattc |
| | | atccatcaatccatccatccatccatccatccatccatccatccatccatctatccatccatccaatcatccatccatccatcgat |
| | | caatcagtccatccatccatctaatcatccatctaatcatccatccatccatccatccatccatccagtcatccatccattcatcca |
| | | tttaatcatccatccatccatcgatcaattggtccatccatccatctatccatccatccatccatccatctaatcacccatccatcc |
| | | atccatccatccaatcatctatccatccatccatctaatcatctatccatccatccatccatccatccatccaatcatccatccatc |
| | | catccatccatccctccatctaatcatccatccctccatccatccctccatatccatccatccatccatccatccatccatccaac |
| | | cattcagcagtgaacctctactgtggtgtgttggaaagaagggaagtgtgagcctggtcttgctcacagggaactcaaaaca |
| | | gagcattaggaggagactcaggatcttacagccttgagggaggtgacccaggctagaggaagtcaggagctcaaagact |
| | | agctggggagccagggacacacacatggattataattagtggctctgcaaggctgcaagtgatcaagggcagactgcggg |
| | | gtaaaggaaggagagaagactctgggctttgttaggtggaagggctttctgcaggaggccaggccagagaagccagcct |
| | | gatgaggctttgagaggacctgaaactggagatgggatttaatggccagagagaaggaggcagcaggcttgtcatgtagg |
| | | caaatacacgcaagcttgtagagtaattaccaggcagagagctgaggagagcaccctgttatgttcccttag |
| 12 | TRPV3 | ccttggcctcgctgatcgagaagtgtcccaaagacaacaaggactgcagctcctacggcagcttcagcgacgcagtgctg |
| | downstream | gaactcttcaagctcaccataggcctgggtgacctgaacatccagcagaactccaagtatcccattctctttctgttcctgctca |
| | exon | tcacctatgtcatcctcacctttgttctcctcctcaacatgctcattgctctgatgggcgagactgtggagaacgtctccaagga |
| | | gagcgaacgcatctggcgcctgc |
| 13 | STK31 upstream | aactctggtggtctccttacaatgagcttggaacgagatcttcttgatgctgagcccatgaaggaacttagcagcaagcgtcct |
| | exon | ttggtacgttctgaggttaatgggcagataattctgttaaag |
| 14 | STK31 upstream | gtaagtctaacttcttcttacttgtgggagattcagcagcataaaggatagtggtgctgttacatttcaagaaagaaaaaacctc |
| | intron | actgttagttttctaaagtgtataagttgactggcatgataattgtgcttgtagtttggttgccagaactaattttcacttttaaagttt |
| | | ccctcaattgaacaatgagaacacttggacacagggggggaacatcacccgtgggggcctgtcatggggtggggggca |
| | | gggggagggatagcattaggagaaatacctaatgtaaatgatgagttaatgggtgcagcaaaccaacatggcacatgtata |
| | | catatgtaacaaacctgcacgttgtgcacatgtaccctagaacttaaagtataataataaaaataagtatataaaagtcattaaat |
| | | aaagctttgtctcacaagaaaaaaaaataaagtttccctcatttccctaaatagaatatgtccttaataggatatactttcctttttttt |
| | | tgggtgaacttattatagtcatccaatatctttttaaaattcttgattttcttacatttctccttgaaatctcagctgtgttatcagtacaa |
| | | tcttctactctttaatttcttcttctcattgatctgggaaactgttaatcatgataataagataattaatttggccttagttgctacacatt |
| | | ctagaatgtgtagcaggctacaggtagaattgcactttccttttccaaattatagcattcaaagaagatagaaaagtacttataa |
| | | aattaaaaaataaaattagatttgatttaacttaaaataatgaattatgtttaaattggaatgatgtataattataatgagattttcatct |
| | | tggaattacgagtgcctctttctttggtacaag |
| 15 | STK31 | ggctattctgtggatgttgacacagaagccaaggtgattgagagagcagccacctaccatagagcttggagagaagctgaa |
| | alternatively | ggagactcagggttactgccattgatattcctgtttttatgtaag |
| | spliced exon | |
| 16 | STK31 | gtaaagttcttatgctaatctcttgcagaaaccattttatcttgcagaaactattttatttaaaataaattttagctccaggttttagag |
| | downstream | cagcagttcattccccttaacataaaagaaaaattatcccatagaatcaatgtatgctaaaaatatgttgaagaagtttttacttgg |
| | intron | agtcacattctctgttttgcttcttag |
| 17 | STK31 | tctgatcctatggcttatctgatggtcccatactaccctagggcaaacctgaatgctgttcaagccaacatgcctttaaattcag |
| | downstream | aa |
| | exon | |
| 18 | USP25 upstream | gtgtcacaacataatgaaagttgctcaagccaaactggaaatgataaaacctgaagaagtaaacttggaggaatatgag |
| | exon | |
| 19 | USP25 upstream | gtaatgtgctttcttagaggttaaactacagatttagggattctgtaattcactgatattcctttcgtttcagcccaactgaagttga |
| | intron | atttgttatgaatgatgagtaaaaaaattaagtttccaagtcaatgctcaagtaattaataattttatatgtgattttaggattaacat |
| | | gatatgatagcatgtagaccaaaaagtttaaaatcaaaaaaggaagatttttggttttttatttcaatactaattctcaagtacttttt |
| | | aaatgaacttttctagcttaaaaacagaataacaaatgcattgttaattttgaaatcaacttagaaattgctgttacataataccaa |
| | | caagtaatatttatcaagaccctactatatgccaggcatttgtctaagccctttacatatattaactgatttaattcagacagtaaca |
| | | ctgtaagataggtagtgttcttattcccatttgacacacgagaaaataaggcatagagcagtgaagtaacctgcctaaggttttc |
| | | acagccacaagtttacagcttggctaatagaacctacattcttaactgttgcactacactgtgcaactgtaccctgtgggctgta |
| | | ttttttccttcactttgattcatcagatatattttggggaagttaattcattttagtccctatatgccatatatgtttgtcatgagacagt |
| | | gttgtgtgtgagagagcacttacataggtacttgtacatgtgcttgtgggatttttttgtgcacaaaattattttttgtcaactttggc |
| | | tttaaaatattctttattttaattgatgaattcatacacctagtatttcttcacatttttaaatttaatcttatgcattgtcttttttttaatttat |
| | | aagtctagtaaattttaagttgttttcttgggtgcctttgcttaatatttagttccatttacaaacttaattatggtactttagattcccta |
| | | aataattagtgtaattcactacacaaacagggaagaggaaagagagagaaaacactcattattgatgaaaaccttagctagtt |
| | | tggcgtataaggtaactaccttaattcactaaagagtgtctctaaaaacaaaaataggaaataataaaactgcagcaaatatat |
| | | agaattgtgagcactgaaagcttaaaatttttgttgtcaaaagcaaggcaatgaaaataagcgaaaagtaaaatactggaaaa |
| | | gaagaaacaaagacatcattatttgtagttttttttaccttgaaattcaaataaaatctacaactgatgtattagaactagtaagac |
| | | agctttgcagcatgcaaacttgtgtcagacatcaattttattcccttttgccagtaaaaaaagtatgagaaagtataaaagtgag |
| | | accatttacaatagtatctgaaactatggagtatatagaaataaatttaacaaaaggtgcgtgtgacttctgtggggaaaattata |
| | | aaacttattgggagacattaacaaagacctaaataaatgggcatacatatggtatttggaaaacttgatattattaatatatagat |
| | | atcacttttcccccaaatgctttataaattcag |
| 20 | USP25 | tgcaggctgtatgaaaattccagcaagatgttttatggagcttgataagccaactgaaaaatgttacggaaatgcaaagatcc |
| | alternatively | cagtccacctcccatctctgacaagcaa |
| | spliced exon | |
| 21 | USP25 | gttagtgaacagtataaagactttttaataaatggttctgggaaataaataaaatttgatttcttctttattccatatgcaaaaattcat |
| | downstream | ttcaattgtgaaagactcctgtgtaaagctgtttagaagtcaacataggtagaagcccaaagacattcatttatccataagagg |
| | intron | cactcactgaccagatatttttgaccaggatatgtaatctagagccagagaaattcaaaatttgttttctcttgaccctgaggagc |
| | | aagtcaagtcatcttttagctaataatgactccaaatctgtgccctccacattgattgagagctcttcccttgtccccaagattcgt |
| | | gttcatggaacttgatttttaattgtgacttgattctgcaatgcttccttcagtttgcttcaacagattggactcattatgttttttttcat |
| | | aaaatcttctcatattctaactgcagggctgttaaatcatacaagtatttaaatgacttgtttttagattctgcatattcctcctatcttt |
| | | gtcttgctctaagttcagggttcagtaaaccatagcctgcagaccagcttaccacctgtttttgtaaataaacttttattggaacat |
| | | ggccatttgtatacatattgtctgtggatactttcacactgcagttacagagttgagtaactgaagaagatgaggcttgcaaagt |
| | | ctaaaatattatctggccttttacagaaaaagtttgcctactcacaacttctaaggtgattctaatgacaacaaggaatgacagtt |
| | | tgaataccttccccagaaaagctaacttcatacttgcaggacaaattttacctttatttgttatgaagatctactaatagcatgaaa |
| | | attatgtcttgaaaattaaaattaagactggatgtggtggctcatgcttataatcccagcacttttggaaatcaaggcacaggga |
| | | gttgtttgaggccaggaatttgagaccggcctggccaacatagcaagaccctgtctatacaaaacagattaagtaattaactg |
| | | gaactgggagcatgcacctgtaattcaagctacttgggagacagaggtaggaggattgctggagcccagaaggtcaagtct |
| | | gcagttagccgtgatcgtgccactgcactgcagactgggcaactgagtgagaccctgtctccaaaaaaaaaaaaaaaagttt |
| | | atattcaaggagaatatcgttggctaataactatgtttcttatttttgtttataatgtttatataaaagccttaacacttttgttatcagta |
| | | ttcactttccaatttatcctacattgaatttcagttatttttcttgagtcaattattataaagtaagctaagaaaatcttgactttggtatc |
| | | accttagaattatttagtcctattacatgccctatagttgttttgggggtggggaggggcattgaaatgtgaactgcagttattctt |
| | | ttaccatatctcatgtaaataaactattcgcctacacacttaaagtcaaatttaccacatatttgcgaagttaaaaggttaaataaa |
| | | tctaactttttttagtttcttaaaattatgtattaagtatgtgtccaagctttgattcatactcagcgtaatatttcattttcttccatcagtt |
| | | gatgtagatataataatagttcaataatatgattttatactgatgaatttttgagatgacctgattgattatcctgcctttatatttctgtt |
| | | atctgctagtaaaatctttattgtttaaaacatttcatatccatgcctaataatttttagatttaatgttgtattaataaatagtcttgtgt |
| | | gccagaaagtaaagtcttgttttagttctgatgtatcttcattttgaactctttatttctattaattcagatagtagcgaaacagtgcat |
| | | tactttttgtacagtttcatagtactttttaaatcttactctgaacatttttaatattttctttcacctacag |
| 22 | USP25 | gagtggcatcaggattataggaaattcagggaaacaactatgtatctcataattgggctagaaaattttcaaagagaaa |
| | downstream | |
| | exon | |
| 23 | GTF2B | gatcgaacaaataatttattcaagcaagtatatgaacagaagagcctgaagggaagagctaatgatgctatagcttctgcttgt |
| | upstream exon | ctctatattgcctgtagacaagaaggggttcctaggacatttaaag |
| 24 | GTF2B | gtaagtttttagctttgtttttttgtgtttgtttgtttgtttgtttgagatagggtctcactttgtcgcccaggccagagtgcagtggcg |
| | upstream intron | caatcttgactcactgcaacctctgcctcccaggttcaagctatactcctgcctcagcctcctgagtagctgggattacaggca |
| | | tgtgccaccacacctggttaacttttgtatttttagtagagacagggtttcgccatgttggccaggctggtctcaaacttctgacc |
| | | tcaggtgatctgcccactttggcctcccaaagtgctagaattaacaggcgtgaggcactgtgcccatcctaaaataacattttc |
| | | tacatcatattaaagaaacagaccaatgtcaggcagctttgacacttagaagctaagtcatctttattgtgactacagtaatcca |
| | | cttgagcatcatttgccaatgcctacttatttcattccttgacagcaaattcatagcatgttcttttaggctgttttcccatgtagact |
| | | cattttagtccacgtaagcccactatgttcttttctagtgagatcttttgatggactgttgctttgtgggctttcagaaggcctataat |
| | | cgtaaaccaaagtaggccgtttatacatttggtttacatggaaacagtttccatttctagcactggtctctgtagatgagggagtc |
| | | aagctttagaaagtatcagtgaaggccaggtatggtgtctcacacctgtaatcccagaactttgggaggccgaggtggttgg |
| | | gtcatgaggccaggagttcaagaccagcctggccaatatggtgaaaccccgtctctactaaaaatacaaaaattagccagg |
| | | cgtggtggtgcgcgcctgtagtcccagctactcaggaggctgaggcagaagaatcgcttgaaaccgggaggcagaggtt |
| | | gcagtgagccgagatcacgccactacactccagcctgggcgacagagtgagacttcatctcaaaaaaaaaaaaaaaaaag |
| | | ttatcagttaagttttttctcttgaggtgtattgtttgcacttttacacttcagttaagaactggtatttttcatttag |
| 25 | GTF2B | tttgcagttacatgtgaatatgtgataatttgttcagtgttgctcactgacttccactaccaaagctcagtttagctgaagactatac |
| | alternatively | ttaaccctttattcatcca |
| | spliced exon | |
| 26 | GTF2B | gtgagtatttattttttatgttttcttacgtaaaagtgaagaggtgatggcctttatcatcatcttttttgttgtttttataactaaaacaa |
| | downstream | cttttgttaaatttcttctgaaagtgtgtggtggttgcatctgggaatagcagggtcagatattttatctcattgagctacagcccat |
| | intron | aatcgcagggatttcatgcacacatgacattgtgttgcctaagaggcatcctttagcttgagtattttctttcaattccttggtaac |
| | | actgcttacaacagattaatacataaacatcaaagaatttttcatttaaagaaatttagttgtatgaattaatccttagagcagttaa |
| | | tattttgaaagattaagacctaaatttagccaggcacgatggctcacaccggtaatctcagcactttgtgaggccgaggcagg |
| | | tagatcacgaggtcaggagttcaagaccagcctggccaagatggtgaaaccccatctctactaaaaatacaaaaaattagcc |
| | | gggtgtggtggcaggcgcctgtaatcccaactactcgggtggcaggagaatcgcttgaacccagggggcagaggttgca |
| | | gtgagccaagatggtgtcactgcactccagtctggacgacagagtgaaatttcatctcaaaaaacaaaaaaactctaaattga |
| | | aagagtgaagaacattaggccaggcgcagtggctcatgcctgtaatctcagcactttgggaggccgaggtaggaggatca |
| | | cctgaggtcaggagttctagacaggcctggccaacatggtgaaaccccatctctactaaaaatacaaaagttagccaggtgt |
| | | ggtggtgcacgcctgtagtcccagctactcgggaggctgaggcaggagaatcacttgagcctaggaggtggaggttgcag |
| | | tgagctgagatcatgccactacccttagtctgggcgacagagtgagacttcatctcaaaaaaaaaggtgtgatgaacattaaa |
| | | gttaactgagaaatttgcagttaactctgtgggaagtatcactgagatgattagggtttcatctttaaggtatgattgaatgttttac |
| | | aattagaatgtatattatgtttataattagaaatggtagattttaagatgaactatatggcttaagtgaattgatttatttaattttttttct |
| | | tttag |
| 27 | GTF2B | aaatatgtgccgtatcacgaatttctaagaaagaaattggtcggtgttttaaacttattttgaaagcgctagaaaccagtgtggat |
| | downstream | ttgattacaactggggacttcatgtccaggttctgttccaacctttgtcttcctaaacaagtacagatggcagctacacatatagc |
| | exon | ccgtaaagctgtggaactggacttggttcctgggaggagccccatctctgtggcagcggcagctatttacatggcctcacag |
| | | gcatcagctgaaaagaggacccaaaaag |
| 28 | LYPD3 | atggaccccgccaggaaagcaggtgcccaggccatgatctggactgcaggctggctgctgctgctgctgcttcgcggag |
| | upstream exon | |
| 29 | LYPD3 | gtgggtctgctgcccggagaggtggagggggtagagtgggggctcttagggaggaggctcctctggccccgtacagaga |
| | upstream intron | aggctgtgggaaggatccccacataagcagagggcactgtgggcaaagttcccccatcctaagcaagggaggcagagg |
| | | gaagggtcctccaccatgggccgtaggaagggttccccaccacggacaaggggagctgtgggcaaaattttcctatcctg |
| | | aagctgcagaaagaagtcccctgttcctaggccgggcgcggtggttcatgcctgtaatctcagcactttgggaggccgagg |
| | | caggcggatcatgaggtcaaacgatcgagaccagcctggccaacatggtgaaaccctgtctctactaaaaatacaaaaatt |
| | | agctgggcgcggtagcgcgagcctgtagtcccagctactcgggaggctgaggcaggagaatcgtttgaatccaggaggg |
| | | gaggttgcagtgagccgagattgcgccactgcactccagcctggcgacagagcgagactccgtctcaaaaaaaaaaaaa |
| | | agaaaaaaagaaaaaaagaaaaaagtgttccctgtcctgggcagagaagctgtgggtagtgttctctcatttgagcagaag |
| | | ggggctgtgggaagggatccctcactgagcacagtctactgtgggaaaagttctcccacacactgtgggaaaagttctctca |
| | | ttctgagcagaaaaccgtgaggaggtatccttcctaccccgggatgaggggcttcatggcggggatcgcctacagtgggc |
| | | atacagggctgtcggaaggatgtgcccacagttggcatagtgggtgtgaaggaaaagggtccagtgattcccgtgcttcca |
| | | gatttgtggaaagcttttccctactcctgccctggaaaagtgtgtgcaataagactgtctaatcccgggacagagggcatggg |
| | | aaggactatttctccttgggggtggacgctttagcaaggagggctcaggtctattcggctccgctcccccag |
| 30 | LYPD3 | gagcgcaggccctggagtgctacagctgcgtgcagaaagcagatgacggatgctccccgaacaagatgaagacagtga |
| | alternatively | agtgcgcgccgggcgtggacgtctgcaccgaggccgtgggggcggtggagacca |
| | spliced exon | |
| 31 | LYPD3 | gtgagtggggacccccgggccacgcacaccactgcaggctcctccggcccagccccgcctccttatagtcctttctggatc |
| | downstream | tggccacgcccctttaacgcggccccacccctccctttcagcctggccgggaccctacgacacagccttgcaggccaggc |
| | intron | cttctctattaacaattacaagcgcgaggagcagagagttgagaactgggtggggcggacggggagagctcgggtttgga |
| | | acgaggtcacgtcctatgcactttttctgctccaccaatgagacctccccatccctgcctgtcaaccaggctgtctcctccgac |
| | | tcagctctttgaggttccgcccccgttccgtgaccccgctcctccctgcttcggtcgttctttccgctctgagcggctctttggg |
| | | ggcgtccatccgccaattcctgcctgtcggtcctgtggtgttggcctatttgaggccccgcccctcccagaccctacccaact |
| | | ctgggccacgccccatctgagaccacgcccacagctaatcccgcccttgtccctctccctag |
| 32 | LYPD3 | tccacggacaattctcgctggcagtgcggggttgcggttcgggactccccggcaagaatgaccgcggcctggatcttcacg |
| | downstream | ggcttctggcgttcatccagctgcagcaatgcgctcaggatcgctgcaacgccaagctcaacctcacctcgcgggcgctcg |
| | exon | acccggcag |
| 33 | IK upstream | gtgagccgttctccaaccctttggcccccgatggccacgatgtggatgatcctcactccttccacca |
| | exon | |
| 34 | IK upstream | gtgagtattttgtgctaggaccatggaaatgagttgggcaatgaatagaaaagtggtggtgatggtgaaagaattctgaatgt |
| | intron | cttgtcatcaaggatggtaaaaaattatacccgctggccctttatctctgtgagccttaactggatcaggccaagtgcataagg |
| | | gtattagaagggaaattcctgcaggaaaggtgttaagtttttttttttttttatacgtgttgtgtaattacacattatataggaattactt |
| | | cttccaatagcaaattaaaacataggaaataagggtaaaatcatctttttattttttttgagactgagtttcgctcttgttgctcag |
| 35 | IK alternatively | gctggaatgcagtggtgcgatctcggctcaccacaacctccgcctcccgggttcaagcaattctcctgcctcagccttccga |
| | spliced exon | gtagctgggattacaggcat |
| 36 | IK downstream | gttccactacgcccggctaattttgtatttttagtagagatggggtttctccatgttgatcaggctggtcttgaaatcccgacttca |
| | intron | ggtgatccacccgcctctgcctcccaaagtggtgggagtacaggcgtgagccaccgtgcctggcccccgataaaatcatct |
| | | ttgataacatccccaattccagtccatttctttttttttttcttttcttttttttctttctttttttttttttttgagacggagtctcactgttacc |
| | | caggctggagtgcagtgatgcaatcttggctcactgcaacctctgcctcctgagttcaagcggttctcctgcctcagcctccc |
| | | aagtagctgggactacaggcatgtgccaccatgcacgactgatttttttttttgtatttttagtagagacagggtttcactgtgttc |
| | | gtcaggctggtcttgaactcctggcctgaagtgatccacccgcctcagcctcctaaagtgctggcattacaggcgtgagcca |
| | | ccgtgcccagcctcagtccttttcttagaagtaaccacagttaccttttttggcatatcttttcctcccgttgcccaggctggagtt |
| | | caatggcattatttctgctcacagcaacctttgcctcctgggctcaagcaattcttctgcctcagtttccccagtagctgggatta |
| | | caggcatgccccaccatgcttggctaatttttgtattagtagagacagggtttcgctatgttggccaggctggtcttgaactcct |
| | | gacctcaaatgatctgcccgtctcggcggcccaaagtgctgggattacaggcgtgagccactgcgcctggccctcctactct |
| | | tttttaaatgcatttatacacatacatgcatatctgaggaaaacacaacattgctttgtgatttaaaatatttgagttaaatgatattat |
| | | gctgtatatatcatttttgcaacttgcttctttttaaaccctgctcagtgtttctgagattatctttcagagcagtgacaagcattccct |
| | | ccttggctaaactttagtcaggttccccaaagagtcccatttttcagcaggactcatccgtggcctgctgagcacagttttaaca |
| | | aagaaatcaaccaccgctccacccccatacctaaccaagttgcttttagtaattttctataaattctcacttgtccctgttgtattcg |
| | | gaattaagttcagtctctctactctattacaatggtcttgactcttgttacagtagtcttcaataaagtcttccttggggagaagag |
| | | gaacttgattagatatcaagttggattggatatcaagagtgggaaaactttctgtgaactgacttaggattcctttttaaaactga |
| | | ggtcagcaggccaaggaagagccctagtcagaagagagctttaaaggagtctgactttaatttgatcaagagaggagtcttt |
| | | gttagtgtaccaattaaaataatctcttgtaccattagtgctgtctttccctctttgggaaacaccatcatagtttagtcagtctcca |
| | | gctaataaacgcttagtctgtttcagactgattcttttcttttttttatttttttgagacagagtcttgctctttttgcccaggctggagt |
| | | gtaatacctagatctcggctcactgcaaactccgcctcccaggttcaagcgattttcctgcctcagcctcctgagtagctagga |
| | | ttacaggcgcccaccacaacgcccagctaatttttttgtatttttagtagagatgggatatcaccatgttggccatgctggtcttg |
| | | aactcctgacctcgggtgatccgcctgcgtcagcctcccaaagtgcggcgattataggtgtgagccactgcacccggccag |
| | | actgattattttcaaggctataactgaattaaatttacaggccttcttatatacatatgtcagtaatggaccaggctaaatataaata |
| | | aatggagttgctggctcataggatatatatacttaaattttaacaaatactgcaaaattgcccttcaaattggttatactggtttaca |
| | | ttctcatcagcattatatgagagagtccccttcccaaattctcactcacacttgatttatcaaactttctggtagttaacactctgat |
| | | gcattaaaagtggcatggcatatctggtcgggcgtggtggcttacacctataatcccagcactttgggaggctgaggtgggc |
| | | agatcacctgaggtcagaagtttgggaccagcctggccaacacctctaataaaaatacaaaaattagctgggtgtgttggcg |
| | | ggcacctgtattcccagctacttgtgaggctgaggcaggagaattacttgaacccaggaggcagaggttgcagtgggctga |
| | | gatcatgccactgcactccagccggggcaacagagggagactctgtcttaaaaaaaaaaaaaaaaagctgggtgcggtgg |
| | | ctcatccctataatcccagcactttgggaggccgaggtgggtggatcacaaggtcaggagttcaagaccagcctggccaat |
| | | atgggtgacagtgagactccgtctcaaaaaaaaaaaaacagtgtcatatctttgttttaatttgtatttccctgatttctgttaattta |
| | | atcatgatttttacttgttcctttttcatttcttattgagtcctaatatttaaaatctttgccttttcttctag |
| 37 | IK downstream | atcaaaactcaccaatgaagacttcaggaaacttctcatgacccccagggctgcacctacctctgcaccaccttctaagtcac |
| | exon | gtcaccatga |
| 38 | TEX14 upstream | tggaggtgaaagaaatggcaaagaaagcagctactggacagctcacagtacctccttggcatcctcagagtagtctgacttt |
| | exon | agagagcgaggctgaaaatgagcccgacgccctgctgcagccccccattaggagcccagaaaacacggattggcagcg |
| | | agttattgagtatcatagggaaaatgatgagcccagaggaaatggcaagtttgacaagacgggcaacaatgactgtgacag |
| | | tgaccagcatggcagacagcccaggcttggaagcttcacca |
| 39 | TEX14 upstream | gtaagttcacttgcgggaatcaggataggtgaactcaatcaagtatcagtcgggcgaggtggctcagcctgtaattccagca |
| | intron | ctttgggaggtgaggcgagcggatcacctgagcccaggaatttgggaccagcctggtcaacatgatgaaacctggtctcta |
| | | caaaaaaaaaaaaaaaaaaaaaaaaaacattagctgggcgtggtggcacacgcctctagttccaggtacccaggaggctg |
| | | aggtgggaggatcacttggacctgagaggtggaggttgcagtgagctgtgatcatgaaactgcactccagcttgggtgaca |
| | | gtgagaccctgtctcaaaataaaaataaaagtaaaaaaaagttatcagctctcaaaaaagtaaactttccagcatattttttttgg |
| | | tttggtcctctctcctttcctacttttctggaaaccttgaattccaggggaagacagtatagccctatatctcattgcatttcccaca |
| | | ggcaaacagatccagacactgatcagtttttatattactgaaacag |
| 40 | TEX14 | gtccctttgtccaaaggaagtaaaataatcaaatactgaccctggttttggcgaggaatcgatccttctttacctggggataga |
| | alternatively | g |
| | spliced exon | |
| 41 | TEX14 | gtaggccatcactgcctactgcccatgtctctgcattgtattctaagctgaaattaaattctcccagaatttatttagaatgtattct |
| | downstream | aagctggcattaaatttttccagagagtctatgctggaggaacctagagacacatgacatttattgtcatcggtttatagatttac |
| | intron | aggttaaaaaaaatgaaggctgtatattttcagttgataatttgcttataatcagggcatggatattgtttcacagttgtttataagtt |
| | | aaagaaagatacatctaaacttgtcatttgtcttaggcagaaaatctattggcttgggtattatttgtatttagaatctgtttcttgtc |
| | | aagcatcactctcgaatggggggcagatgatccagggctgcattttggggtcaggctaaactaacagtctctactctctgga |
| | | ctgaacctaacgtatgggacatggccactcttggcagaatatgtatacatctcttctttcatgccacctttatcctcttccatgttgt |
| | | ctgaatatcactgaatgactttgaccctgactgtgttacaatcattag |
| 42 | TEX14 | gtatcaggcacccatctcccagacaaaaggagcaaccagagcatagtgaagccttccaagcaagttctgacacattggtgg |
| | downstream | ctgtagagaaatcttacagt |
| | exon | |
| 43 | TRPV3 | ttttatccaagctgtgcttgtgatactgtctgtcttcttgtacttgtttgcctacaaagagtacctcgcctgcctcgtgctggccttg |
| | construct #1 | gccctgggctgggcgaacaagctctactatacgcggggtttccagtccatcggcttgtacagcgtcatcatccagaaggtgc |
| | (full) | gttgagagctgcgccggcgggaggggcctcggaggatggccatttttcagatggggaaactgagatctggagaggaaga |
| | | ggggtttaccaaggggcaggtcactgctctttctcccaaatgccatgcactccctttgctttcggccaggctttaagtgtcaag |
| | | accttaaatcccccaggcttgtggggtgctggggaggggtacagtttgtggaggaatgttctcacccctgggcagtggcgtg |
| | | ctggtaaacatttaacgactgttctgtgtgtgtgtgtgtgtatgtgtgtgtgtgtaggtggaatatgtatattccatatatatacaca |
| | | aaagacttatcatacattttactatataaacgatgtgtatcacacaatttgcaaataatgaaaatataccatgcagtttattgtaaat |
| | | tccacatagccagttgagtctcagaataatttgttgctttttttttttttttttggagaccaagtcttgctctgctgcccaggccagag |
| | | taaagtggtgcgatctcggctccctgcaacctctgcctcctgggttcaagcagttctcctgcctcagcctccagagtagctgg |
| | | gatcacaggcacgcaccaccatgttggctaatttttgtatttttagtagagacagggtttcagcatgttggccaggattgtcttga |
| | | actcctgacctcaggtgatccacctgcctcggcctcccaaagtgctgggattacaggcatgaaccaccgcgccaggcctatt |
| | | tgttgctttttattgaattctagtttttgttgtcaacttttggttgcaattgacaaaagagtgctatcagacatgaatgttggttgagag |
| | | ttttctttataagcagtgattgaagtgaaacaattaagatttatgttgaactttacttgtttatcaatgatgtgagcaacttctttgctg |
| | | aattggatactagtttgcaaataccagaataatattctgttgatatttttgtgctatacataatgtaaaggctgcagacagaacaca |
| | | ttttaaaatttaaactggattaatagtttctccatcactgtcttaaatcatttagatctagatatcaacaaaacaatgagtcaatcaat |
| | | ctctgatttgtggcatttaccaattttcatgatgtaaatacagtagtccccccttattcttgggggagatgttctaagaccccccat |
| | | gaatgcctgaaactgcagatagtaccaaacattatataatatgctatgttttttcctatacatatacatctatgcaaataatgaaaat |
| | | atactatacaatacagtttattgtaaattccacttagccagttgagtcttagaataatttttgctttttttttttttttttgtttgtgactgag |
| | | tctcgctctgttgcccaggctggagtgcaatagtgcaatctcggctcactgcaacctctgcctcccgggctcaagcaattctc |
| | | ccgccccagcctcctgagtagctgggactacaggcatgcaccatgcccagctaatttttgtatttttagtagagatggggtttc |
| | | gccatgttggccaggctggtctccaactcttgacctcaggtgatccgtccgcctcggcctcccaaagtgctggggggtggg |
| | | ggggggaatcaacaaattattctaagactcaactggctatgtggaatttataaaatttattttatcaattaggcatagtgagattaa |
| | | tggcaataactaacagaacaatgataacaatatactgtaataaaagtatgtgaatgttgtctctctcagaatattgtattgtgtgta |
| | | atatttctgggtcatagttgaccacaggcaaccaaaactgcagaaagtgaaatcgaagagaagggcggactactgttctcct |
| | | ctcagggccaatttccagctaccaacattatgttactggacatagagttagggagaaacatgcagatccacatcgctctagaa |
| | | ttcctgccatatggctatgatagagacaagtaacctcaaaagcacacacaattgtaaatatagtgaaataactggacggggat |
| | | gagttttgaacatttattgtctttgttttatttaattaaattaattaattaattaattttttgagacagagtctggctctgccccccaggct |
| | | ggagtgcagtggtgcgatctaggctcactgcaaccccttcctcctgggttcaagcaattctcccgcctcagcctctggagtag |
| | | ctgggatcacaggtgtgtgccaccacacccagctaatttttgtatttttagtagagacggggtttcgccatgttggccaagctg |
| | | gcctccaactcctgacctcaggcgatccacccacctcagcctctcaaagtgttgggattacaggcgtgagtcactgcaccct |
| | | ggcctgtctttgttttaaatataacttattttatttaattgtaagtttatatggtttaattttttggcaatacaaataataatttctttacccc |
| | | cagctttattgaggtacagctgacatatataattgtataaatttaagatgtacaatgtgaggatttgatataggtatatattgtgaaa |
| | | tgatttccaatttccaattaacacacccatcacctcacatagttgccatttctttgtgtgtggtgagaacatttatgatctattctctta |
| | | gcaaatttctagtacacaacacggtattgttcactatagtcatcatgctgtccattagatccccagaacttattcatcttattcattg |
| | | atttagttctgaataatggctgtgtttaacaaccagctcacaagcttcatgagaatttaacgatcagctcttgcgagctcttctga |
| | | gctggctccagcatgccactgcccccagcaagtgtgaggattccagtgtgagaccctatggtgaaaggtacagggtgaatc |
| | | acagggagaagagaacctggggctggagcagaaacagaaggcttcctggaggaggcaggatccagtgttgagccttgg |
| | | agcatcaggaggatttggctgggggaagggagagtggcccaagtgaagagacagttagggtgggggggggagaggg |
| | | tgggaagcaggaagatcagggtgccgtgaatatgtgcttgcaccttacacttgaattatgtttatttccttataggtcatttgcca |
| | | ccatgttctggagttcttgtttgtatatatcgtgtttttgcttggatttggagttggtaattgatttataaataatagtaactcccacatt |
| | | tataaacactttatacagatgtcaaaatggcaaagtgcatggcggccttggcaacaattagaccaactaccagtggggcactt |
| | | gccatgcttggcattttccaagcattacctttatatatatacatatatattttattatactttaagttctagggtacatgtgcacaacat |
| | | gcaggtttgttacatatgtatacatgtgccatgttggtatgctgcacccattaactcgtcatttacattaggtatatctcctaatcaa |
| | | gcattacctttttacttctctcaacaacttgatcaagtgggaatcatcaccccgatcataaggtaactgaggcttagagactcca |
| | | ggattcaaaccaggccagccactcataatccaacttcaccatgtggtatatacctctggctttcccattcattgcttacgtggcc |
| | | ctcacgacctaagataggttctgcaagtgttttcagccccattttaacagatgcggaaactaaagtccacagaaactcaactga |
| | | tggtctttgcaaagtaggagcaaactaataccctgaggtgatttcctttggatttcacatcacgttactctcacagtttgctttaga |
| | | tacaactgtgagagcctctctggtctgctgggaccaggaaggagccccctcttaaaatctctccatttacaacagagggaac |
| | | agcacgatctggttgtatttctaacctctgcatagagagatatcattttcccccacgtaccagttgcttttttttcttttcttcagtgac |
| | | cattgacatttgaaattaagtagctgtttccatctgtctggtaccttttctgggtcacaatttgcttatttatttgctccttcacctgctt |
| | | gtttattcatccatctatttactctctcatttaatttgctcatccacctgtctatccgtccatccatccatccatccatccatccatccat |
| | | ccatccaatcattcatccatcgatccatccattgatctatccatctatccatccatccatccatccatccatccatctaatcatccat |
| | | ccaatcatccatccatccatccatccatccatccatccatccagtcatccatccattcatccatttaatcatccatccatcgatcaa |
| | | tcggtccatccatccagctatccatccatccatccatccatctaatcattcatccatcaatccatccatccatccatccatccatcc |
| | | atccatccatccatctatccatccatccaatcatccatccatccatcgatcaatcagtccatccatccatctaatcatccatctaat |
| | | catccatccatccatccatccatccatccagtcatccatccattcatccatttaatcatccatccatccatcgatcaattggtccat |
| | | ccatccatctatccatccatccatccatccatctaatcacccatccatccatccatccatccaatcatctatccatccatccatcta |
| | | atcatctatccatccatccatccatccatccatccaatcatccatccatccatccatccatccctccatctaatcatccatccctcc |
| | | atccatccctccatatccatccatccatccatccatccatccatccaaccattcagcagtgaacctctactgtggtgtgttggaa |
| | | agaagggaagtgtgagcctggtcttgctcacagggaactcaaaacagagcattaggaggagactcaggatcttacagcctt |
| | | gagggaggtgacccaggctagaggaagtcaggagctcaaagactagctggggagccagggacacacacatggattata |
| | | attagtggctctgcaaggctgcaagtgatcaagggcagactgcggggtaaaggaaggagagaagactctgggctttgttag |
| | | gtggaagggctttctgcaggaggccaggccagagaagccagcctgatgaggctttgagaggacctgaaactggagatgg |
| | | gatttaatggccagagagaaggaggcagcaggcttgtcatgtaggcaaatacacgcaagcttgtagagtaattaccaggca |
| | | gagagctgaggagagcaccctgttatgttcccttagccttggcctcgcttatcgagaagtgtcccaaagacaacaaggactg |
| | | cagctcctacggcagcttcagcgacgcagtgctggaactcttcaagctcaccattggcctgggtgacctggacatccagca |
| | | gaactccaagtatcccattctctttctgttcctgctcatcacctatgtcatcctcacctttgttctcctcctcaacatgctcattgctct |
| | | tatgggcgagactgtggagaacgtctccaaggagagcgaacgcatctggcgcctgc |
| 44 | TRPV3 | ttttatccaagctgtgcttgtgatactgtctgtcttcttgtacttgtttgcctacaaagagtacctcgcctgcctcgtgctggccttg |
| | construct #2 | gccctgggctgggcgaacaagctctactatacgcggggtttccagtccatcggcttgtacagcgtcatcatccagaaggtgc |
| | (minimal v1) | gttgagagctgcgccggcgggaggggcctcggaggatggccatttttcagatggggaaactgagatctggagaggaaga |
| | Also referred to | ggggtttaccaaggggcagttagggtggggggtgggagaggggggaagcaggaagatcagggtgccgtgaatatgtg |
| | as V1B | cttgcaccttacacttgaattatgtttatttccttataggtcatttgccaccatgttctggagttcttgtttgtatatatcgtgtttttgctt |
| | | ggatttggagttggtaattgatttataaataatagtaactcccacatttataaacactttatacagatgtcaaaatggcaaagtgca |
| | | tggcggccttggcaacaattagaccagaaggaggcagcaggcttgtcatgtaggcaaatacacgcaagcttgtagagtaat |
| | | taccaggcagagagctgaggagagcaccctgttatgttcccttagccttggcctcgcttatcgagaagtgtcccaaagacaa |
| | | caaggactgcagctcctacggcagcttcagcgacgcagtgctggaactcttcaagctcaccattggcctgggtgacctgga |
| | | catccagcagaactccaagtatcccattctctttctgttcctgctcatcacctatgtcatcctcacctttgttctcctcctcaacatg |
| | | ctcattgctcttatgggcgagactgtggagaacgtctccaaggagagcgaacgcatctggcgcctgc |
| 45 | TRPV3 | tctactatacgcggggtttccagtccatcggcttgtacagcgtcatcatccagaaggtgcgttgagagctgcgccggcggga |
| | construct #3 | ggggcctcggaggatggccattttgtgaatatgtgcttgcaccttacacttgaattatgtttatttccttataggtcatttgccacc |
| | (minimal v2) | atgttctggagttcttgtttgtatatatcgtgtttttgcttggatttggagttggtaattgatttataaataatagtaactcccacatttat |
| | Also referred to | aaacactttatagtaattaccaggcagagagctgaggagagcaccctgttatgttcccttagccttggcctcgcttatcgagaa |
| | as V2B | gtgtcccaaagacaacaaggactgcagctcctacggcagcttcagcgacgcagtgctggaactct |
| 46 | STK31 construct | aactctggtggtctccttacaatcagcttggaacgagatcttcttgaagctgagcccttgaaggaacttagcagcaagcgtcct |
| | #1 (full) | ttggtacgttctgaggttattgggcagataattctgttaaaggtaagtctaacttcttcttacttgtgggagattcagcagcataaa |
| | | ggatagtggtgctgttacatttcaagaaagaaaaaacctcactgttagttttctaaagtgtataagttgactggcatgataattgt |
| | | gcttgtagtttggttgccagaactaattttcacttttaaagtttccctcaattgaacaatgagaacacttggacacagggcgggg |
| | | aacatcacccgtgggggcctgtcatggggtggggggcagggggagggatagcattaggagaaatacctaatgtaaatgat |
| | | gagttaatgggtgcagcaaaccaacatggcacatgtatacatatgtaacaaacctgcacgttgtgcacatgtaccctagaact |
| | | taaagtataataataaaaataagtatataaaagtcattaaataaagctttgtctcacaagaaaaaaaaataaagtttccctcatttc |
| | | cctaaatagaatatgtccttaataggatatactttcctttttttttgggtgaacttattatagtcatccaatatctttttaaaattcttgatt |
| | | ttcttacatttctccttgaaatctcagctgtgttatcagtacaatcttctactctttaatttcttcttctcattgatctgggaaactgttaa |
| | | tcatgataataagataattaatttggccttagttgctacacattctagaatgtgtagcaggctacaggtagaattgcactttcctttt |
| | | ccaaattatagcattcaaagaagatagaaaagtacttataaaattaaaaaataaaattagatttgatttaacttaaaataatgaatt |
| | | atgtttaaattggaatgatgtataattataatgagattttcatcttggaattacgagtgcctctttctttggtacaagggctattctgt |
| | | ggttgttgacacagaagccaaggtgattgagagagcagccacctaccatagagcttggagagaagctgaaggagactcag |
| | | ggttactgccattgatattcctgccaccatgaaaggtaaagttcttatgctaatctcttgcagaaaccattttatcttgcagaaact |
| | | attttatttaaaataaattttagctccaggttttagagcagcagttcattccccttaacataaaagaaaaattatcccatagaatcaa |
| | | tgtatgctaaaaatatgttgaagaagtttttacttggagtcacattctctgttttgcttcttagtctatcctatggcttatctgatggtc |
| | | ccatactaccctagggcaaacctgaatgctgttcaagccaacatgcctttaaattcagaa |
| 47 | STK31 construct | aactctggtggtctccttacaatcagcttggaacgagatcttcttgaagctgagcccttgaaggaacttagcagcaagcgtcct |
| | #2 (minimal v1) | ttggtacgttctgaggttattgggcagataattctgttaaaggtaagtctaacttcttcttacttgtgggagattcagcagcataaa |
| | | ggatagtggtgctgttacatttcaagaaagaaaaaacctcactgttagttttctaatttaacttaaaataatgaattatgtttaaattg |
| | | gaatgatgtataattataatgagattttcatcttggaattacgagtgcctctttctttggtacaagggctattctgtggttgttgacac |
| | | agaagccaaggtgattgagagagcagccacctaccatagagcttggagagaagctgaaggagactcagggttactgccat |
| | | tgatattcctgccaccatgaaaggtaaagttcttatgctaatctcttgcagaaaccattttatcttgcagaaactattttatttaaaat |
| | | aaattttagctccaggttttagagcagcagttcattccccttaacataaaagaaaaattatcccatagaatcaatgtatgctaaaa |
| | | atatgttgaagaagtttttacttggagtcacattctctgttttgcttcttagtctatcctatggcttatctgatggtcccatactaccct |
| | | agggcaaacctgaatgctgttcaagccaacatgcctttaaattcagaa |
| 48 | USP25 construct | gtgtcacaacatattgaaagttgctcaagccaaactggaattgataaaacctgaagaagtaaacttggaggaatatcaggtaa |
| | #1 (full) | tgtgctttcttagaggttaaactacagatttagggattctgtaattcactgatattcctttcgtttcagcccaactgaagttgaatttg |
| | | ttatgaatgatgagtaaaaaaattaagtttccaagtcaatgctcaagtaattaataattttatatgtgattttaggattaacatgatat |
| | | gatagcatgtagaccaaaaagtttaaaatcaaaaaaggaagatttttggttttttatttcaatactaattctcaagtactttttaaatg |
| | | aacttttctagcttaaaaacagaataacaaatgcattgttaattttgaaatcaacttagaaattgctgttacataataccaacaagt |
| | | aatatttatcaagaccctactatatgccaggcatttgtctaagccctttacatatattaactgatttaattcagacagtaacactgta |
| | | agataggtagtgttcttattcccatttgacacacgagaaaataaggcatagagcagtgaagtaacctgcctaaggttttcacag |
| | | ccacaagtttacagcttggctaatagaacctacattcttaactgttgcactacactgtgcaactgtaccctgtgggctgtatttttt |
| | | ccttcactttgattcatcagatatattttggggaagttaattcattttagtccctatatgccatatatgtttgtcatgagacagtgttgt |
| | | gtgtgagagagcacttacataggtacttgtacatgtgcttgtgggatttttttgtgcacaaaattattttttgtcaactttggctttaa |
| | | aatattctttattttaattgatgaattcatacacctagtatttcttcacatttttaaatttaatcttatgcattgtcttttttttaatttataagt |
| | | ctagtaaattttaagttgttttcttgggtgcctttgcttaatatttagttccatttacaaacttaattatggtactttagattccctaaata |
| | | attagtgtaattcactacacaaacagggaagaggaaagagagagaaaacactcattattgatgaaaaccttagctagtttggc |
| | | gtataaggtaactaccttaattcactaaagagtgtctctaaaaacaaaaataggaaataataaaactgcagcaaatatatagaa |
| | | ttgtgagcactgaaagcttaaaatttttgttgtcaaaagcaaggcaatgaaaataagcgaaaagtaaaatactggaaaagaag |
| | | aaacaaagacatcattatttgtagttttttttaccttgaaattcaaataaaatctacaactgatgtattagaactagtaagacagcttt |
| | | gcagcatgcaaacttgtgtcagacatcaattttattcccttttgccagtaaaaaaagtatgagaaagtataaaagtgagaccatt |
| | | tacaatagtatctgaaactatggagtatatagaaataaatttaacaaaaggtgcgtgtgacttctgtggggaaaattataaaactt |
| | | attgggagacattaacaaagacctaaataaatgggcatacatatggtatttggaaaacttgatattattaatatatagatatcactt |
| | | ttcccccaaatgctttataaattcagtgcaggctgtatgaaaattccagcaagatgttttatcgagcttgataagccaactgaaa |
| | | aatcttacggaattgccaccatgccagtccacctcccatctctgacaagcaagttagtgaacagtataaagactttttaataaat |
| | | ggttctgggaaataaataaaatttgatttcttctttattccatatgcaaaaattcatttcaattgtgaaagactcctgtgtaaagctgt |
| | | ttagaagtcaacataggtagaagcccaaagacattcatttatccataagaggcactcactgaccagatatttttgaccaggata |
| | | tgtaatctagagccagagaaattcaaaatttgttttctcttgaccctgaggagcaagtcaagtcatcttttagctaataatgactcc |
| | | aaatctgtgccctccacattgattgagagctcttcccttgtccccaagattcgtgttcatggaacttgatttttaattgtgacttgatt |
| | | ctgcaatgcttccttcagtttgcttcaacagattggactcattatgttttttttcataaaatcttctcatattctaactgcagggctgtta |
| | | aatcatacaagtatttaaatgacttgtttttagattctgcatattcctcctatctttgtcttgctctaagttcagggttcagtaaaccat |
| | | agcctgcagaccagcttaccacctgtttttgtaaataaacttttattggaacatggccatttgtatacatattgtctgtggatacttt |
| | | cacactgcagttacagagttgagtaactgaagaagatgaggcttgcaaagtctaaaatattatctggccttttacagaaaaagt |
| | | ttgcctactcacaacttctaaggtgattctaatgacaacaaggaatgacagtttgaataccttccccagaaaagctaacttcata |
| | | cttgcaggacaaattttacctttatttgttatgaagatctactaatagcatgaaaattatgtcttgaaaattaaaattaagactggat |
| | | gtggtggctcatgcttataatcccagcacttttggaaatcaaggcacagggagttgtttgaggccaggaatttgagaccggcc |
| | | tggccaacatagcaagaccctgtctatacaaaacagattaagtaattaactggaactgggagcatgcacctgtaattcaagct |
| | | acttgggagacagaggtaggaggattgctggagcccagaaggtcaagtctgcagttagccgtgatcgtgccactgcactg |
| | | cagactgggcaactgagtgagaccctgtctccaaaaaaaaaaaaaaaagtttatattcaaggagaatatcgttggctaataac |
| | | tatgtttcttatttttgtttataatgtttatataaaagccttaacacttttgttatcagtattcactttccaatttatcctacattgaatttcag |
| | | ttatttttcttgagtcaattattataaagtaagctaagaaaatcttgactttggtatcaccttagaattatttagtcctattacatgccct |
| | | atagttgttttgggggtggggggggcattgaaatgtgaactgcagttattcttttaccatatctcatgtaaataaactattcgcct |
| | | acacacttaaagtcaaatttaccacatatttgcgaagttaaaaggttaaataaatctaactttttttagtttcttaaaattatgtattaa |
| | | gtatgtgtccaagctttgattcatactcagcgtaatatttcattttcttccatcagttgatgtagatataataatagttcaataatatga |
| | | ttttatactgatgaatttttgagatgacctgattgattatcctgcctttatatttctgttatctgctagtaaaatctttattgtttaaaacat |
| | | ttcatatccatgcctaataatttttagatttaatgttgtattaataaatagtcttgtgtgccagaaagtaaagtcttgttttagttctgat |
| | | gtatcttcattttgaactctttatttctattaattcagatagtagcgaaacagtgcattactttttgtacagtttcatagtactttttaaat |
| | | cttactctgaacatttttaatattttctttcacctacaggagtggcatcaggattataggaaattcagggaaacaactatgtatctc |
| | | aaaattgggccagaaaattttcaaagagaaa |
| 49 | USP25 construct | gtgtcacaacatattgaaagttgctcaagccaaactggaattgataaaacctgaagaagtaaacttggaggaatatcaggtaa |
| | #2 (minimal v1) | tgtgctttcttagaggttaaactacagatttagggattctgtaattcactgatattcctttcgtttcagcccaactgaagttgaatttg |
| | | ttatgaaaacaaagacctaaataaatgggcatacatatggtatttggaaaacttgatattattaatatatagatatcacttttcccc |
| | | caaatgctttataaattcagtgcaggctgtatgaaaattccagcaagatgttttatcgagcttgataagccaactgaaaaatctta |
| | | cggaattgccaccatgccagtccacctcccatctctgacaagcaagttagtgaacagtataaagactttttaataaatggttctg |
| | | ggaaataaataaaatttgatttcttctttattccatatgcaaaaattcatttcaattgtgaattcagatagtagcgaaacagtgcatt |
| | | actttttgtacagtttcatagtactttttaaatcttactctgaacatttttaatattttctttcacctacaggagtggcatcaggattata |
| | | ggaaattcagggaaacaactatgtatctcaaaattgggccagaaaattttcaaagagaaa |
| 50 | GTF2B | gatcgaacaaataatttattcaagcaagtataagaacagaagagcctgaagggaagagctattgatcctatagcttctgcttgt |
| | construct #1 | ctctatattgcctgtagacaagaaggggttcctaggacatttaaaggtaagtttttagctttgtttttttgtgtttgtttgtttgtttgttt |
| | (full) | gagatagggtctcactttgtcgcccaggccagagtgcagtggcgcaatcttgactcactgcaacctctgcctcccaggttca |
| | | agctatactcctgcctcagcctcctgagtagctgggattacaggcatgtgccaccacacctggttaacttttgtatttttagtaga |
| | | gacagggtttcgccatgttggccaggctggtctcaaacttctgacctcaggtgatctgcccactttggcctcccaaagtgcta |
| | | gaattaacaggcgtgaggcactgtgcccatcctaaaataacattttctacatcatattaaagaaacagaccaatgtcaggcag |
| | | ctttgacacttagaagctaagtcatctttattgtgactacagtaatccacttgagcatcatttgccaatgcctacttatttcattcctt |
| | | gacagcaaattcatagcatgttcttttaggctgttttcccatgtagactcattttagtccacgtaagcccactatgttcttttctagtg |
| | | agatcttttgatggactgttgctttgtgggctttcagaaggcctataatcgtaaaccaaagtaggccgtttatacatttggtttaca |
| | | tggaaacagtttccatttctagcactggtctctgtagatgagggagtcaagctttagaaagtatcagtgaaggccaggtatggt |
| | | gtctcacacctgtaatcccagaactttgggaggccgaggtggttgggtcatgaggccaggagttcaagaccagcctggcca |
| | | atatggtgaaaccccgtctctactaaaaatacaaaaattagccaggcgtggtggtgcgcgcctgtagtcccagctactcagg |
| | | aggctgaggcagaagaatcgcttgaaaccgggaggcagaggttgcagtgagccgagatcacgccactacactccagcct |
| | | gggcgacagagtgagacttcatctcaaaaaaaaaaaaaaaaaagttatcagttaagttttttctcttgaggtgtattgtttgcact |
| | | tttacacttcagttaagaactggtatttttcatttagtttgcagttacttgtgaatatctgataatttgttcagtgttgctcactgacttc |
| | | cactaccaaagctcagtttagctgaagacgccaccatggcctttattcatccagtgagtatttattttttatgttttcttacgtaaaa |
| | | gtgaagaggtgatggcctttatcatcatcttttttgttgtttttataactaaaacaacttttgttaaatttcttctgaaagtgtgtggtgg |
| | | ttgcatctgggaatagcagggtcagatattttatctcattgagctacagcccataatcgcagggatttcatgcacacatgacatt |
| | | gtgttgcctaagaggcatcctttagcttgagtattttctttcaattccttggtaacactgcttacaacagattaatacataaacatca |
| | | aagaatttttcatttaaagaaatttagttgtatgaattaatccttagagcagttaatattttgaaagattaagacctaaatttagccag |
| | | gcacgatggctcacaccggtaatctcagcactttgtgaggccgaggcaggtagatcacgaggtcaggagttcaagaccag |
| | | cctggccaagatggtgaaaccccatctctactaaaaatacaaaaaattagccgggtgtggtggcaggcgcctgtaatcccaa |
| | | ctactcgggtggcaggagaatcgcttgaacccagggggcagaggttgcagtgagccaagatggtgtcactgcactccagt |
| | | ctggacgacagagtgaaatttcatctcaaaaaacaaaaaaactctaaattgaaagagtgaagaacattaggccaggcgcag |
| | | tggctcatgcctgtaatctcagcactttgggaggccgaggtaggaggatcacctgaggtcaggagttctagacaggcctgg |
| | | ccaacatggtgaaaccccatctctactaaaaatacaaaagttagccaggtgtggtggtgcacgcctgtagtcccagctactc |
| | | gggaggctgaggcaggagaatcacttgagcctaggaggtggaggttgcagtgagctgagatcatgccactacccttagtct |
| | | gggcgacagagtgagacttcatctcaaaaaaaaaggtgtgatgaacattaaagttaactgagaaatttgcagttaactctgtg |
| | | ggaagtatcactgagatgattagggtttcatctttaaggtatgattgaatgttttacaattagaatgtatattatgtttataattagaa |
| | | atggtagattttaagatgaactatatggcttaagtgaattgatttatttaattttttttttttagaaatatgtgccgtatcacgaatttct |
| | | aagaaagaaattggtcggtgttttaaacttatttagaaagcgcttgaaaccagtgtggattttattacaactggggacttcatgtc |
| | | caggttctgttccaacctttgtcttcctaaacaagtacagatggcagctacacatattgcccgtaaagctgtggaactggacttg |
| | | gttcctgggaggagccccatctctgtggcagcggcagctatttacatggcctcacaggcatcagctgaaaagaggacccaa |
| | | aaag |
| 51 | GTF2B | gatcgaacaaataatttattcaagcaagtataagaacagaagagcctgaagggaagagctattgatcctatagcttctgcttgt |
| | construct #2 | ctctatattgcctgtagacaagaaggggttcctaggacatttaaaggtaagtttttagctttgtttttttgtgtttgtttgtttgtttgttt |
| | (minimal v1) | gagatagggtctcactttgtcgcccaggccagagtgcagtggcgcaatcttgactcaaaaaaaaaaaaaaaaaagttatcag |
| | | ttaagttttttctcttgaggtgtattgtttgcacttttacacttcagttaagaactggtatttttcatttagtttgcagttacttgtgaatat |
| | | ctgataatttgttcagtgttgctcactgacttccactaccaaagctcagtttagctgaagacgccgccaccatggcctttattcat |
| | | ccagtgagtatttattttttatgttttcttacgtaaaagtgaagaggtgatggcctttatcatcatcttttttgttgtttttataactaaaa |
| | | caacttttgttaacaattagaatgtatattatgtttataattagaaatggtagattttaagatgaactatatggcttaagtgaattgatt |
| | | tatttaattttttttcttttagaaatatgtgccgtatcacgaatttctaagaaagaaattggtcggtgttttaaacttatttagaaagcg |
| | | cttgaaaccagtgtggattttattacaactggggacttcatgtccaggttctgttccaacctttgtcttcctaaacaagtacagatg |
| | | gcagctacacatattgcccgtaaagctgtggaactggacttggttcctgggaggagccccatctctgtggcagcggcagcta |
| | | tttacatggcctcacaggcatcagctgaaaagaggacccaaaaag |
| 52 | LYPD3 | gaccccgccaggaaagcaggtgcccaggccttgatctggactgcaggctggctgctgctgctgctgcttcgcggaggtgg |
| | construct #1 | gtctgctgcccggagaggtggagggggtagagtgggggctcttagggaggaggctcctctggccccgtacagagaagg |
| | (full) | ctgtgggaaggatccccacataagcagagggcactgtgggcaaagttcccccatcctaagcaagggaggcagagggaa |
| | | gggtcctccaccatgggccgtaggaagggttccccaccacggacaaggggagctgtgggcaaaattttcctatcctgaag |
| | | ctgcagaaagaagtcccctgttcctaggccgggcgcggtggttcatgcctgtaatctcagcactttgggaggccgaggcag |
| | | gcggatcatgaggtcaaacgatcgagaccagcctggccaacatggtgaaaccctgtctctactaaaaatacaaaaattagct |
| | | gggcgcggtagcgcgagcctgtagtcccagctactcgggaggctgaggcaggagaatcgtttgaatccaggaggggag |
| | | gttgcagtgagccgagattgcgccactgcactccagcctggcgacagagcgagactccgtctcaaaaaaaaaaaaaagaa |
| | | aaaaagaaaaaaagaaaaaagtgttccctgtcctgggcagagaagctgtgggtagtgttctctcatttgagcagaaggggg |
| | | ctgtgggaagggatccctcactgagcacagtctactgtgggaaaagttctcccacacactgtgggaaaagttctctcattctg |
| | | agcagaaaaccgtgaggaggtatccttcctaccccgggatgaggggcttcatggcggggatcgcctacagtgggcataca |
| | | gggctgtcggaaggatgtgcccacagttggcatagtgggtgtgaaggaaaagggtccagtgattcccgtgcttccagatttg |
| | | tggaaagcttttccctactcctgccctggaaaagtgtgtgcaataagactgtctaatcccgggacagagggcatgggaagga |
| | | ctatttctccttgggggtggacgctttagcaaggagggctcaggtctattcggctccgctcccccaggagcgcaggccctgg |
| | | agtgctacagctgcgtgcagagccaccatggcggatcctccccgaacaagatcaagacagttaagtgcgcgccgggcgt |
| | | ggacgtctgcaccgaggccgtgggggcggtggagaccagtgagtggggacccccgggccacgcacaccactgcaggc |
| | | tcctccggcccagccccgcctccttatagtcctttctggatctggccacgcccctttaacgcggccccacccctccctttcagc |
| | | ctggccgggaccctacgacacagccttgcaggccaggccttctctattaacaattacaagcgcgaggagcagagagttga |
| | | gaactgggtggggcggacggggagagctcgggtttggaacgaggtcacgtcctatgcactttttctgctccaccaatgaga |
| | | cctccccatccctgcctgtcaaccaggctgtctcctccgactcagctctttgaggttccgcccccgttccgtgaccccgctcct |
| | | ccctgcttcggtcgttctttccgctctgagcggctctttgggggcgtccatccgccaattcctgcctgtcggtcctgtggtgttg |
| | | gcctatttgaggccccgcccctcccagaccctacccaactctgggccacgccccatctgagaccacgcccacagctaatcc |
| | | cgcccttgtccctctccctagtccacggacaattctcgctggcagtgcggggttgcggttcgggactccccggcaagaatga |
| | | ccgcggcctggatcttcacgggcttctggcgttcatccagctgcagcattgcgctcaggatcgctgcaacgccaagctcaac |
| | | ctcacctcgcgggcgctcgacccggcag |
| 53 | LYPD3 | gaccccgccaggaaagcaggtgcccaggccttgatctggactgcaggctggctgctgctgctgctgcttcgcggaggtgg |
| | construct #2 | gtctgctgcccggagaggtggagggggtagagtgggggctcttagggaggaggctcctctggccccgtacagagaagg |
| | (minimal v1) | ctgtgggaaggatccccatgtctaatcccgggacagagggcatgggaaggactatttctccttgggggtggacgctttagca |
| | Also referred to | aggagggctcaggtctattcggctccgctcccccaggagcgcaggccctggagtgctacagctgcgtgcagagccaccat |
| | as VIA | ggcggatcctccccgaacaagatcaagacagttaagtgcgcgccgggcgtggacgtctgcaccgaggccgtgggggcg |
| | | gtggagaccagtgagtggggacccccgggccacgcacaccactgcaggctcctccggcccagccccgcctccttatagt |
| | | cctttctggatctggccacgcccctttaacgctatttgaggccccgcccctcccagaccctacccaactctgggccacgccc |
| | | catctgagaccacgcccacagctaatcccgcccttgtccctctccctagtccacggacaattctcgctggcagtgcggggtt |
| | | gcggttcgggactccccggcaagaatgaccgcggcctggatcttcacgggcttctggcgttcatccagctgcagcattgcg |
| | | ctcaggatcgctgcaacgccaagctcaacctcacctcgcgggcgctcgacccggcag |
| 54 | LYPD3 | gctgctgctgctgctgcttcgcggaggtgggtctgctgcccggagaggtggagggggtagagtgggggctcttaggtgga |
| | construct #3 | cgctttagcaaggagggctcaggtctattcggctccgctcccccaggagcgcaggccctggagtgctacagctgcgtgca |
| | (minimal v2) | gagccaccatggcggatcctccccgaacaagatcaagacagttaagtgcgcgccgggcgtggacgtctgcaccgaggcc |
| | Also referred to | gtgggggcggtggagaccagtgagtggggacccccgggccacgcacaccactgcaggctcctccggcccccatctgag |
| | as V2A | accacgcccacagctaatcccgcccttgtccctctccctagtccacggacaattctcgctggcagtgcggggttgcggttcg |
| | | ggactccccggcaaga |
| 55 | IK construct #1 | gtgagccgttctccaaccctttggcccccgttggccacgatctggaagatcctcactccttccaccagtgagtattttgtgctag |
| | (full) | gaccatggaaatgagttgggcaatgaatagaaaagtggtggtgatggtgaaagaattctgaatgtcttgtcatcaaggatggt |
| | | aaaaaattatacccgctggccctttatctctgtgagccttaactggatcaggccaagtgcataagggtattagaagggaaattc |
| | | ctgcaggaaaggtgttaagtttttttttttttttatacgtgttgtgtaattacacattatataggaattacttcttccaatagcaaattaa |
| | | aacataggaaataagggtaaaatcatctttttattttttttgagactgagtttcgctcttgttgctcaggctggaatgcagtggtgc |
| | | gatctcggctcacgccaccatgggcctcccgggttcaagcaattctcctgcctcagccttccgagtagctgggattacaggc |
| | | atgttccactacgcccggctaattttgtatttttagtagagatggggtttctccatgttgatcaggctggtcttgaaatcccgactt |
| | | caggtgatccacccgcctctgcctcccaaagtggtgggagtacaggcgtgagccaccgtgcctggcccccgataaaatca |
| | | tctttgataacatccccaattccagtccatttctttttttttttcttttcttttttttctttctttttttttttttttgagacggagtctcactgtta |
| | | cccaggctggagtgcagtgatgcaatcttggctcactgcaacctctgcctcctgagttcaagcggttctcctgcctcagcctc |
| | | ccaagtagctgggactacaggcatgtgccaccatgcacgactgatttttttttttgtatttttagtagagacagggtttcactgtgt |
| | | tcgtcaggctggtcttgaactcctggcctgaagtgatccacccgcctcagcctcctaaagtgctggcattacaggcgtgagc |
| | | caccgtgcccagcctcagtccttttcttagaagtaaccacagttaccttttttggcatatcttttcctcccgttgcccaggctgga |
| | | gttcaatggcattatttctgctcacagcaacctttgcctcctgggctcaagcaattcttctgcctcagtttccccagtagctggga |
| | | ttacaggcatgccccaccatgcttggctaatttttgtattagtagagacagggtttcgctatgttggccaggctggtcttgaactc |
| | | ctgacctcaaatgatctgcccgtctcggcggcccaaagtgctgggattacaggcgtgagccactgcgcctggccctcctact |
| | | cttttttaaatgcatttatacacatacatgcatatctgaggaaaacacaacattgctttgtgatttaaaatatttgagttaaatgatatt |
| | | atgctgtatatatcatttttgcaacttgcttctttttaaaccctgctcagtgtttctgagattatctttcagagcagtgacaagcattcc |
| | | ctccttggctaaactttagtcaggttccccaaagagtcccatttttcagcaggactcatccgtggcctgctgagcacagttttaa |
| | | caaagaaatcaaccaccgctccacccccatacctaaccaagttgcttttagtaattttctataaattctcacttgtccctgttgtatt |
| | | cggaattaagttcagtctctctactctattacaatggtcttgactcttgttacagtagtcttcaataaagtcttccttggggagaag |
| | | aggaacttgattagatatcaagttggattggatatcaagagtgggaaaactttctgtgaactgacttaggattcctttttaaaact |
| | | gaggtcagcaggccaaggaagagccctagtcagaagagagctttaaaggagtctgactttaatttgatcaagagaggagtc |
| | | tttgttagtgtaccaattaaaataatctcttgtaccattagtgctgtctttccctctttgggaaacaccatcatagtttagtcagtctc |
| | | cagctaataaacgcttagtctgtttcagactgattcttttcttttttttatttttttgagacagagtcttgctctttttgcccaggctgga |
| | | gtgtaatacctagatctcggctcactgcaaactccgcctcccaggttcaagcgattttcctgcctcagcctcctgagtagctag |
| | | gattacaggcgcccaccacaacgcccagctaatttttttgtatttttagtagagatgggatatcaccatgttggccatgctggtct |
| | | tgaactcctgacctcgggtgatccgcctgcgtcagcctcccaaagtgcggcgattataggtgtgagccactgcacccggcc |
| | | agactgattattttcaaggctataactgaattaaatttacaggccttcttatatacatatgtcagtaatggaccaggctaaatataa |
| | | ataaatggagttgctggctcataggatatatatacttaaattttaacaaatactgcaaaattgcccttcaaattggttatactggttt |
| | | acattctcatcagcattatatgagagagtccccttcccaaattctcactcacacttgatttatcaaactttctggtagttaacactct |
| | | gatgcattaaaagtggcatggcatatctggtcgggcgtggtggcttacacctataatcccagcactttgggaggctgaggtg |
| | | ggcagatcacctgaggtcagaagtttgggaccagcctggccaacacctctaataaaaatacaaaaattagctgggtgtgttg |
| | | gcgggcacctgtattcccagctacttgtgaggctgaggcaggagaattacttgaacccaggaggcagaggttgcagtggg |
| | | ctgagatcatgccactgcactccagccggggcaacagagggagactctgtcttaaaaaaaaaaaaaaaaagctgggtgcg |
| | | gtggctcatccctataatcccagcactttgggaggccgaggtgggtggatcacaaggtcaggagttcaagaccagcctggc |
| | | caatatgggtgacagtgagactccgtctcaaaaaaaaaaaaacagtgtcatatctttgttttaatttgtatttccctgatttctgtta |
| | | atttaatcatgatttttacttgttcctttttcatttcttattgagtcctaatatttaaaatctttgccttttcttctagatcaaaactcaccatt |
| | | gaagacttcaggaaacttctcagacccccagggctgcacctacctctgcaccaccttcaagtcacgtcaccatta |
| 56 | IK construct #2 | gtgagccgttctccaaccctttggcccccgttggccacgatctggaagatcctcactccttccaccagtgagtattttgtgctag |
| | (minimal v1) | gaccatggaaatgagttgggcaatgaatagaaaagtggtggtgatggtgaaagaattctgaatgtcttgtcatcaaggatgg |
| | | ataggaattacttcttccaatagcaaattaaaacataggaaataagggtaaaatcatctttttattttttttgagactgagtttcgctc |
| | | ttgttgctcaggctggaatgcagtggtgcgatctcggctcacgccaccatgggcctcccgggttcaagcaattctcctgcctc |
| | | agccttccgagtagctgggattacaggcatgttccactacgcccggctaattttgtatttttagtagagatggggtttctccatgt |
| | | tgatcaggctggtcttgaaatcccgacttcaggtgatccacccggtatttccctgatttctgttaatttaatcatgatttttacttgtt |
| | | cctttttcatttcttattgagtcctaatatttaaaatctttgccttttcttctagatcaaaactcaccattgaagacttcaggaaacttct |
| | | cagacccccagggctgcacctacctctgcaccaccttcaagtcacgtcaccatta |
| 57 | TEX14 construct | tggaggtgaaagaaatggcaaagaaagcagctactggacagctcacagtacctccttggcatcctcagagtagtctgacttt |
| | #1 (full) | agagagcgaggctgaaaatgagcccgacgccctgctgcagccccccattaggagcccagaaaacacggattggcagcg |
| | | agttattgagtatcatagggaaaatgatgagcccagaggaaatggcaagtttgacaagacgggcaacaatgactgtgacag |
| | | tgaccagcatggcagacagcccaggcttggaagcttcaccagtaagttcacttgcgggaatcaggataggtgaactcaatc |
| | | aagtatcagtcgggcgaggtggctcagcctgtaattccagcactttgggaggtgaggcgagcggatcacctgagcccagg |
| | | aatttgggaccagcctggtcaacatgatgaaacctggtctctacaaaaaaaaaaaaaaaaaaaaaaaaaacattagctggg |
| | | cgtggtggcacacgcctctagttccaggtacccaggaggctgaggtgggaggatcacttggacctgagaggtggaggttg |
| | | cagtgagctgtgatcatgaaactgcactccagcttgggtgacagtgagaccctgtctcaaaataaaaataaaagtaaaaaaa |
| | | agttatcagctctcaaaaaagtaaactttccagcatattttttttggtttggtcctctctcctttcctacttttctggaaaccttgaattc |
| | | caggggaagacagtatagccctatatctcattgcatttcccacaggcaaacagatccagacactgatcagtttttatattactga |
| | | aacaggtccctttgtccaaaggaagtaaaataatcaaatactgaccctggttttggcgaggaatcgatccttgccaccatggg |
| | | gatagaggtaggccatcactgcctactgcccatgtctctgcattgtattctaagctgaaattaaattctcccagaatttatttagaa |
| | | tgtattctaagctggcattaaatttttccagagagtctatgctggaggaacctagagacacatgacatttattgtcatcggtttata |
| | | gatttacaggttaaaaaaaatgaaggctgtatattttcagttgataatttgcttataatcagggcatggatattgtttcacagttgttt |
| | | ataagttaaagaaagatacatctaaacttgtcatttgtcttaggcagaaaatctattggcttgggtattatttgtatttagaatctgtt |
| | | tcttgtcaagcatcactctcgaatggggggcagatgatccagggctgcattttggggtcaggctaaactaacagtctctactc |
| | | tctggactgaacctaacgtatgggacatggccactcttggcagaatatgtatacatctcttctttcatgccacctttatcctcttcc |
| | | atgttgtctgaatatcactgaatgactttgaccctgactgtgttacaatcattaggtatcaggcacccatctcccagacaaaagg |
| | | agcaaccagagcagtgaagccttccaagcaagttctgacacattggtggctgtagagaaatcttacagt |
| 58 | TEX14 construct | tggaggtgaaagaaatggcaaagaaagcagctactggacagctcacagtacctccttggcatcctcagagtagtctgacttt |
| | #2 (minimal v1) | agagagcgaggctgaaaatgagcccgacgccctgctgcagccccccattaggagcccagaaaacacggattggcagcg |
| | | agttattgagtatcatagggaaaatgatgagcccagaggaaatggcaagtttgacaagacgggcaacaatgactgtgacag |
| | | tgaccagcatggcagacagcccaggcttggaagcttcaccagtaagttcacttgcgggaatcaggataggtgaactcaatc |
| | | aagtatcagtcgggcgaggtggctcagcctgtaattccagcactttgggaggtgaggcgaccttgaattccaggggaagac |
| | | agtatagccctatatctcattgcatttcccacaggcaaacagatccagacactgatcagtttttatattactgaaacaggtcccttt |
| | | gtccaaaggaagtaaaataatcaaatactgaccctggttttggcgaggaatcgatccttgccaccatggggatagaggtagg |
| | | ccatcactgcctactgcccatgtctctgcattgtattctaagctgaaattaaattctcccagaatttatttagaatgtattctaagct |
| | | ggcattagaatatgtatacatctcttctttcatgccacctttatcctcttccatgttgtctgaatatcactgaatgactttgaccctga |
| | | ctgtgttacaatcattaggtatcaggcacccatctcccagacaaaaggagcaaccagagcagtgaagccttccaagcaagtt |
| | | ctgacacattggtggctgtagagaaatcttacagt |
| 59 | Rep Construct | caattgtcgcgaaataaaatatctttattttcattacatctgtgtgttggttttttgtgtgaatcgatagtactaacatacgctctccat |
| | #1 | caaaacaaaacgaaacaaaacaaactagcaaaataggctgtccccagtgcaagtgcaggtgccagaacatttctctggcct |
| | | aactggccggtacctgagctcgctagcctcgagcctgcaggcgacatgaagaatctgcttagggttaggcgttttgcgctgc |
| | | ttcgcgttgtacgggccagatatacgcgtatctgaggggactagggtgtgtttaggcgaaaagcggggcttcggttgtacgc |
| | | ggttaggagtcccctcaggatatagtagtttcgcttttgcatagggagggggattgcgccttttccaaggcagccctgggtttg |
| | | cgcagggacgcggctgctctgggcgtggttccgggaaacgcagcggcgccgaccctgggtctcgcacattcttcacgtcc |
| | | gttcgcagcgtcacccggatcttcgccgctacccttgtgggccccccggcgacgcttcctgctccgcccctaagtcgggaa |
| | | ggttccttgcggttcgcggcgtgccggacgtgacaaacggaagccgcacgtctcactagtaccctcgcagacggacagcg |
| | | ccagggagcaatggcagcgcgccgaccgcgatgggctgtggccaatagcggctgctcagcagggcgcgccgagagca |
| | | gcggccgggaaggggcggtgcgggaggcggggtgtggggcggtagtgtgggccctgttcctgcccgcgcggtgttccg |
| | | cattctgcaagcctccggagcgcacgtcggcagtcggctccctcgttgaccgaatcaccgacctctctccccagggggatc |
| | | caccggagcttaccgaccccgccaggaaagcaggtgcccaggccttgatctggactgcaggctggctgctgctgctgctg |
| | | cttcgcggaggtgggtctgctgcccggagaggtggagggggtagagtgggggctcttagggaggaggctcctctggccc |
| | | cgtacagagaaggctgtgggaaggatccccatgtctaatcccgggacagagggcatgggaaggactatttctccttgggg |
| | | gtggacgctttagcaaggagggctcaggtctattcggctccgctcccccaggagcgcaggccctggagtgctacagctgc |
| | | gtgcagagccaccatggcggatcctccccgaacaagatcaagacagttaagtgcgcgccgggcgtggacgtctgcaccg |
| | | aggccgtgggggcggtggagaccagtgagtggggacccccgggccacgcacaccactgcaggctcctccggcccagc |
| | | cccgcctccttatagtcctttctggatctggccacgcccctttaacgctatttgaggccccgcccctcccagaccctacccaac |
| | | tctgggccacgccccatctgagaccacgcccacagctaatcccgcccttgtccctctccctagtccacggacaattctcgct |
| | | ggcagtgcggggttgcggttcgggactccccggcaagattgaccgcggcctggatcttcacgggcttctggcgttcatcca |
| | | gctgcagcattgcgctcaggatcgctgcaacgccaagctcaacctcacctcgcgggcgctcgacccggcaggtgccggg |
| | | gttttacgagattgtgattaaggtccccagcgaccttgacgagcatctgcccggcatttctgacagctttgtgaactgggtggc |
| | | cgagaaggaatgggagttgccgccagattctgacatggatctgaatctgattgagcaggcacccctgaccgtggccgaga |
| | | agctgcagcgcgactttctgacggaatggcgccgtgtgagtaaggccccggaggcccttttctttgtgcaatttgagaaggg |
| | | agagagctacttccacatgcacgtgctcgtggaaaccaccggggtgaaatctatggttttgggacgtttcctgagtcagattc |
| | | gcgaaaaactgattcagagaatttaccgcgggatcgagccgactttgccaaactggttcgcggtcacaaagaccagaaatg |
| | | gcgccggaggcgggaacaaggtggtggatgagtgctacatccccaattacttgctccccaaaacccagcctgagctccag |
| | | tgggcgtggactaatatggaacagtatttaagcgcctgtttgaatctcacggagcgtaaacggttggtggcgcagcatctgac |
| | | gcacgtgtcgcagacgcaggagcagaacaaagagaatcagaatcccaattctgatgcgccggtgatcagatcaaaaactt |
| | | cagccaggtacatggagctggtcgggtggctcgtggacaaggggattacctcggagaagcagtggattcaggaggacca |
| | | ggcctcatacatctccttcaatgcggcctccaactcgcggtcccaaatcaaggctgccttggacaatgcgggaaagattatg |
| | | agcctgactaaaaccgcccccgactacctggtgggccagcagcccgtggaggacatttccagcaatcggatttataaaattt |
| | | tggaactaaacgggtacgatccccaatatgcggcttccgtctttctgggatgggccacgaaaaagttcggcaagaggaaca |
| | | ccatctggctgtttgggcctgcaactaccggaaagaccaacatcgcggaggccatagcccacactgtgcccttctacgggt |
| | | gcgtaaactggaccaatgagaactttcccttcaacgactgtgtcgacaagatggtgatctggtgggaggaggggaagatga |
| | | ccgccaaggtcgtggagtcggccaaagccattctcggaggaagcaaggtgcgcgtggaccagaaatgcaagtcctcggc |
| | | ccagatagacccgactcccgtgatcgtcacctccaacaccaacatgtgcgccgtgattgacgggaactcaacgaccttcga |
| | | acaccagcagccgttgcaagaccggatgttcaaatttgaactcacccgccgtctggatcatgactttgggaaggtcaccaag |
| | | caggaagtcaaagactttttccggtgggcaaaggatcacgtggttgaggtggagcatgagttctacgtcaaaaagggtgga |
| | | gccaagaaaagacccgcccccagtgacgcagatataagtgagcccaaacgggtgcgcgagtcagttgcgcagccatcg |
| | | acgtcagacgcggaggcttcgatcaactacgcagacaggtaccaaaacaaatgttctcgtcacgtgggcatgaatctgatg |
| | | ctgtttccctgcagacaatgcgagagaatgaatcagaattcaaatatctgcttcactcacggacagaaagactgtttagagtgc |
| | | tttcccgtgtcagaatctcaacccgtttctgtcgtcaaaaaggcgtatcagaaactgtgctacattcatcatatcatgggaaagg |
| | | tgccagacgcttgcactgcctgcgatctggtcaatgtggatttggatgactgcatctttgaacaataaaggtaaacttgttaatc |
| | | aataaaccgtttaattcgtttcagttgaacttttctctgctaactttctttctttctagtttggacttccctccaggtaaccggtcggg |
| | | gcaacatgactacgtccggcgttccatttggcatgacactacgaccaacacgatctcggttgtctcggcgcactccgtacagt |
| | | agggatcgtctacctccttttgagacagaaacccgcgctaccatactggaggatcatccgctgctgcccgaatgtaacacttt |
| | | gacaatgcacaacgtgagttacgtgcgaggacttccctgcagtgtgggatttacgctgattcaggaatgggttgttccctggg |
| | | atatggttctaacgcgggaggagcttgtaatcctgaggaagtgtatgcacgtgtgcctgtgttgtgccaacattgacatcatga |
| | | cgagcatgatgatccacggttacgagtcctgggctctccactgtcattgttccagtcccggttccctgcagtgtatagccggc |
| | | gggcaggttttggccagctggtttaggatggtggtggatggcgccatgtttaatcagaggtttatatggtatcgggaggtggt |
| | | gaattacaacatgccaaaagaggtaatgtttatgtccagcgtgtttatgaggggtcgccacttaatctacctgcgcttgtggtat |
| | | gatggccacgtgggttctgtggtccccgccatgagctttggatacagcgccttgcactgtgggattttgaacaatattgtggtg |
| | | ctgtgctgcagttactgtgctgatttaagtgagatcagggtgcgctgctgtgcccggaggacaaggcgccttatgctgcggg |
| | | cggtgcgaatcatcgctgaggagactaccgccatgttgtattcctgcaggacggagcggcggcggcagcagtttattcgcg |
| | | cgctgctgcagcaccaccgccctatcctgatgcacgattatgactctacccccatgtaggttgtttgacccctcccccgtgcct |
| | | tccttgaccctggaaggtgccactcccactgtcctttcctaataaaatgaggaaattgcatcgcattgtctgagtaggtgtcatt |
| | | ctattctggggggtggggggggcaggacagcaagggggatccctgcaggcgcaggcggtcagcatcgccatggagac |
| | | caacacccttcccaccgccactcccccttcctctcagggtccctgtcccctccagtgaatcccagaaggactctggagagttc |
| | | tgagcagggggcggcactctggcctctgattggtccaaggaaggctggggggcaggacgggaggcgaaaaccctgga |
| | | atattcccgacctggcagcctcatcgagctcggtgattggctcagaagggaaaaggcgggtctccgtgacgacttataaaa |
| | | gcccaggggcaagcggtccggataacggctacctgaggagctgctgcgacagtccactacctttttcgaggtgactcccgt |
| | | tgtcccaaggcttcccagagcgaacctgtgcggctgcaggcaccggcgcgtcgagtttccggcgtccggaaggaccgag |
| | | ctcttctcgctgatccgtgttccgtttccgcccccaatctctcgttgccgacgcagggaaccggcggcaaccatggccagtc |
| | | gggaagaggagcagcgcgaaaccacccccgagcgcggacgcggtgcggcgcgacgtcccccaaccatggaggacgt |
| | | gtcgtccccgtccccgtcgccgccgcctccccgggcgcccccaaaaaagcggatgaggcggcgtatcgagtccgagga |
| | | cgaggaggactcatcacaagacgcgctggtgccgcgcacacccagcccgcggccatcgacctcggcggcggatttggc |
| | | cattgcgcccaagaagaaaaagaagcgcccttctcccaagcccgagcgcccgccatcaccagaggtaatcgtggacagc |
| | | gaggaagaaagagaagatgtggcgctacaaatggtgggtttcagcaaccctccggtgctaatcaagcatggcaaaggag |
| | | gtaagcgcacagtgcggcggctgaatgaagatgacccagtggcgcgtggtatgcggacgcaagaggaagaggaagag |
| | | cccagcgaagcggaaagtgaaattacggtgatgaacccgctgagtgtgccgatcgtgtctgcgtgggagaagggcatgg |
| | | aggctgcgcgggcgctgatggacaagtaccacgtggataacgatctaaaggcgaacttcaaactactgcctgaccaagtg |
| | | gaagctctggcagccgtatgcaagacctggctgaacgaggagcaccgcgggttgcagctgaccttcaccagcaacaaga |
| | | cctttgtgacgatgatggggcgattcctgcaggcgtacctgcagtcgtttgcagaggtgacctacaagcatcacgagcccac |
| | | gggctgcgcgttgtggctgcaccgctgcgctgagatcgaaggcgagcttaagtgtctacacggaagcattatgataaataa |
| | | ggagcacgtgattgaaatggatgtgacgagcgaatttgggcagcgcgcgctgaaggagcagtctagcaaggccaagatc |
| | | gtgaagaatcggtggggccgaaatgtggtgcagatttccaacaccgacgcaaggtgctgcgtgcacgacgcggcctgtcc |
| | | ggccaatcagttttccggcaagtcttgcggcatgttcttctctgaaggcgcaaaggctcaggtggcttttaagcagatcaagg |
| | | cttttatgcaggcgctgtatcctaacgcccagaccgggcacggtcaccttttgatgccactacggtgcgagtgcaactcaaa |
| | | gcctgggcacgcgccctttttgggaaggcagctaccaaagttgactccgttcgccctgagcaacgcggaggacctggacg |
| | | cggatctgatctccgacaagagcgtgctggccagcgtgcaccacccggcgctgatagtgttccagtgctgcaaccctgtgt |
| | | atcgcaactcgcgggcgcagggcggaggccccaactgcgacttcaagatatcggcgcccgacctgctaaacgcgttggt |
| | | gatggtgcgcagcctgtggagtgaaaacttcaccgagctgccgcggatggttgtgcctgagtttaagtggagcactaaaca |
| | | ccagtatcgcaacgtgtccctgccagtggcgcatagcgatgcgcggcagaacccctttgatttttaaacggcgcagacggc |
| | | aagtaaataatcacccgagagtgtacaaataaaagatcttgccacaaaatagttacaggaccaagcgagcgtgagagtcca |
| | | aggcgtgcgcagtcgttgacgctctagaccgtgcaaaaggagagcctgtaagcgggcactcttccgtggtctggtggataa |
| | | attcgcaagggtatcatggcggacgacccgggttcgaaccccggatcgggccgtccgccgtgatccatgcggttaccgcc |
| | | cgcgtgtcgaacccaggtgtgcgacgtcagacaacgggggagcgctcctttttggcttccttccaggcgcggcggctgctg |
| | | cgctcttttttcggccgcggccgc |
| 60 | Rep Construct | caattgagggtggaggtcctgtattagaggtcacgtgagtgttttgcgacattttgcgacaccatgtggtcacgctgggtattta |
| | #2 | agcccgagtgagcacgcagggctctccattttgaagcgggaggtttgaacgcgcagccgccgaccccgccaggaaagc |
| | | aggtgcccaggccttgatctggactgcaggctggctgctgctgctgctgcttcgcggaggtgggtctgctgcccggagagg |
| | | tggagggggtagagtgggggctcttagggaggaggctcctctggccccgtacagagaaggctgtgggaaggatccccat |
| | | gtctaatcccgggacagagggcatgggaaggactatttctccttgggggtggacgctttagcaaggagggctcaggtctatt |
| | | cggctccgctcccccaggagcgcaggccctggagtgctacagctgcgtgcagagccaccatggcggatcctccccgaac |
| | | aagatcaagacagttaagtgcgcgccgggcgtggacgtctgcaccgaggccgtgggggcggtggagaccagtgagtgg |
| | | ggacccccgggccacgcacaccactgcaggctcctccggcccagccccgcctccttatagtcctttctggatctggccacg |
| | | cccctttaacgctatttgaggccccgcccctcccagaccctacccaactctgggccacgccccatctgagaccacgcccac |
| | | agctaatcccgcccttgtccctctccctagtccacggacaattctcgctggcagtgcggggttgcggttcgggactccccgg |
| | | caagattgaccgcggcctggatcttcacgggcttctggcgttcatccagctgcagcattgcgctcaggatcgctgcaacgcc |
| | | aagctcaacctcacctcgcgggcgctcgacccggcaggtgccggggttttacgagattgtgattaaggtccccagcgacct |
| | | tgacgagcatctgcccggcatttctgacagctttgtgaactgggtggccgagaaggaatgggagttgccgccagattctgac |
| | | atggatctgaatctgattgagcaggcacccctgaccgtggccgagaagctgcagcgcgactttctgacggaatggcgccgt |
| | | gtgagtaaggccccggaggcccttttctttgtgcaatttgagaagggagagagctacttccacatgcacgtgctcgtggaaa |
| | | ccaccggggtgaaatctatggttttgggacgtttcctgagtcagattcgcgaaaaactgattcagagaatttaccgcgggatc |
| | | gagccgactttgccaaactggttcgcggtcacaaagaccagaaatggcgccggagggggaacaaggtggtggatgagt |
| | | gctacatccccaattacttgctccccaaaacccagcctgagctccagtgggcgtggactaatatggaacagtatttaagcgcc |
| | | tgtttgaatctcacggagcgtaaacggttggtggcgcagcatctgacgcacgtgtcgcagacgcaggagcagaacaaaga |
| | | gaatcagaatcccaattctgatgcgccggtgatcagatcaaaaacttcagccaggtacatggagctggtcgggtggctcgtg |
| | | gacaaggggattacctcggagaagcagtggattcaggaggaccaggcctcatacatctccttcaatgcggcctccaactcg |
| | | cggtcccaaatcaaggctgccttggacaatgcgggaaagattatgagcctgactaaaaccgcccccgactacctggtggg |
| | | ccagcagcccgtggaggacatttccagcaatcggatttataaaattttggaactaaacgggtacgatccccaatatgcggctt |
| | | ccgtctttctgggatgggccacgaaaaagttcggcaagaggaacaccatctggctgtttgggcctgcaactaccggaaaga |
| | | ccaacatcgcggaggccatagcccacactgtgcccttctacgggtgcgtaaactggaccaatgagaactttcccttcaacga |
| | | ctgtgtcgacaagatggtgatctggtgggaggaggggaagatgaccgccaaggtcgtggagtcggccaaagccattctcg |
| | | gaggaagcaaggtgcgcgtggaccagaaatgcaagtcctcggcccagatagacccgactcccgtgatcgtcacctccaa |
| | | caccaacatgtgcgccgtgattgacgggaactcaacgaccttcgaacaccagcagccgttgcaagaccggatgttcaaattt |
| | | gaactcacccgccgtctggatcatgactttgggaaggtcaccaagcaggaagtcaaagactttttccggtgggcaaaggat |
| | | cacgtggttgaggtggagcatgagttctacgtcaaaaagggtggagccaagaaaagacccgcccccagtgacgcagatat |
| | | aagtgagcccaaacgggtgcgcgagtcagttgcgcagccatcgacgtcagacgcggaggcttcgatcaactacgcagac |
| | | aggtaccaaaacaaatgttctcgtcacgtgggcatgaatctgatgctgtttccctgcagacaatgcgagagaatgaatcagaa |
| | | ttcaaatatctgcttcactcacggacagaaagactgtttagagtgctttcccgtgtcagaatctcaacccgtttctgtcgtcaaaa |
| | | aggcgtatcagaaactgtgctacattcatcatatcatgggaaaggtgccagacgcttgcactgcctgcgatctggtcaatgtg |
| | | gatttggatgactgcatctttgaacaataaaggtaaacttgttaatcaataaaccgtttaattcgtttcagttgaacttttctctgcta |
| | | actttctttctttctagtttggacttccctccaggtaaccggtcggggcaacatgactacgtccggcgttccatttggcatgacac |
| | | tacgaccaacacgatctcggttgtctcggcgcactccgtacagtagggatcgtctacctccttttgagacagaaacccgcgct |
| | | accatactggaggatcatccgctgctgcccgaatgtaacactttgacaatgcacaacgtgagttacgtgcgaggacttccctg |
| | | cagtgtgggatttacgctgattcaggaatgggttgttccctgggatatggttctaacgcgggaggagcttgtaatcctgagga |
| | | agtgtatgcacgtgtgcctgtgttgtgccaacattgacatcatgacgagcatgatgatccacggttacgagtcctgggctctcc |
| | | actgtcattgttccagtcccggttccctgcagtgtatagccggcgggcaggttttggccagctggtttaggatggtggtggatg |
| | | gcgccatgtttaatcagaggtttatatggtatcgggaggtggtgaattacaacatgccaaaagaggtaatgtttatgtccagcg |
| | | tgtttatgaggggtcgccacttaatctacctgcgcttgtggtatgatggccacgtgggttctgtggtccccgccatgagctttgg |
| | | atacagcgccttgcactgtgggattttgaacaatattgtggtgctgtgctgcagttactgtgctgatttaagtgagatcagggtg |
| | | cgctgctgtgcccggaggacaaggcgccttatgctgcgggcggtgcgaatcatcgctgaggagactaccgccatgttgtat |
| | | tcctgcaggacggagcggcggcggcagcagtttattcgcgcgctgctgcagcaccaccgccctatcctgatgcacgattat |
| | | gactctacccccatgtaggttgtttgacccctcccccgtgccttccttgaccctggaaggtgccactcccactgtcctttcctaa |
| | | taaaatgaggaaattgcatcgcattgtctgagtaggtgtcattctattctggggggtggggggggcaggacagcaagggg |
| | | gatccctgcaggcgcaggcggtcagcatcgccatggagaccaacacccttcccaccgccactcccccttcctctcagggtc |
| | | cctgtcccctccagtgaatcccagaaggactctggagagttctgagcagggggcggcactctggcctctgattggtccaag |
| | | gaaggctggggggcaggacgggaggcgaaaaccctggaatattcccgacctggcagcctcatcgagctcggtgattggc |
| | | tcagaagggaaaagggggtctccgtgacgacttataaaagcccaggggcaagcggtccggataacggctacctgagga |
| | | gctgctgcgacagtccactacctttttcgaggtgactcccgttgtcccaaggcttcccagagcgaacctgtgcggctgcagg |
| | | caccggcgcgtcgagtttccggcgtccggaaggaccgagctcttctcgctgatccgtgttccgtttccgcccccaatctctcg |
| | | ttgccgacgcagggaaccggcggcaaccatggccagtcgggaagaggagcagcgcgaaaccacccccgagcgcgga |
| | | cgcggtgcggcgcgacgtcccccaaccatggaggacgtgtcgtccccgtccccgtcgccgccgcctccccgggcgccc |
| | | ccaaaaaagcggatgaggcggcgtatcgagtccgaggacgaggaggactcatcacaagacgcgctggtgccgcgcaca |
| | | cccagcccgcggccatcgacctcggcggcggatttggccattgcgcccaagaagaaaaagaagcgcccttctcccaagc |
| | | ccgagcgcccgccatcaccagaggtaatcgtggacagcgaggaagaaagagaagatgtggcgctacaaatggtgggttt |
| | | cagcaaccctccggtgctaatcaagcatggcaaaggaggtaagcgcacagtgcggcggctgaatgaagatgacccagtg |
| | | gcgcgtggtatgcggacgcaagaggaagaggaagagcccagcgaagcggaaagtgaaattacggtgatgaacccgctg |
| | | agtgtgccgatcgtgtctgcgtgggagaagggcatggaggctgcgcgggcgctgatggacaagtaccacgtggataacg |
| | | atctaaaggcgaacttcaaactactgcctgaccaagtggaagctctggcagccgtatgcaagacctggctgaacgaggag |
| | | caccgcgggttgcagctgaccttcaccagcaacaagacctttgtgacgatgatggggcgattcctgcaggcgtacctgcag |
| | | tcgtttgcagaggtgacctacaagcatcacgagcccacgggctgcgcgttgtggctgcaccgctgcgctgagatcgaagg |
| | | cgagcttaagtgtctacacggaagcattatgataaataaggagcacgtgattgaaatggatgtgacgagcgaatttgggcag |
| | | cgcgcgctgaaggagcagtctagcaaggccaagatcgtgaagaatcggtggggccgaaatgtggtgcagatttccaaca |
| | | ccgacgcaaggtgctgcgtgcacgacgcggcctgtccggccaatcagttttccggcaagtcttgcggcatgttcttctctga |
| | | aggcgcaaaggctcaggtggcttttaagcagatcaaggcttttatgcaggcgctgtatcctaacgcccagaccgggcacgg |
| | | tcaccttttgatgccactacggtgcgagtgcaactcaaagcctgggcacgcgccctttttgggaaggcagctaccaaagttg |
| | | actccgttcgccctgagcaacgcggaggacctggacgcggatctgatctccgacaagagcgtgctggccagcgtgcacc |
| | | acccggcgctgatagtgttccagtgctgcaaccctgtgtatcgcaactcgcgggcgcagggcggaggccccaactgcgac |
| | | ttcaagatatcggcgcccgacctgctaaacgcgttggtgatggtgcgcagcctgtggagtgaaaacttcaccgagctgccg |
| | | cggatggttgtgcctgagtttaagtggagcactaaacaccagtatcgcaacgtgtccctgccagtggcgcatagcgatgcgc |
| | | ggcagaacccctttgatttttaaacggcgcagacggcaagtaaataatcacccgagagtgtacaaataaaagatcttgccac |
| | | aaaatagttacaggaccaagcgagcgtgagagtccaaggcgtgcgcagtcgttgacgctctagaccgtgcaaaaggaga |
| | | gcctgtaagcgggcactcttccgtggtctggtggataaattcgcaagggtatcatggcggacgacccgggttcgaaccccg |
| | | gatcgggccgtccgccgtgatccatgcggttaccgcccgcgtgtcgaacccaggtgtgcgacgtcagacaacgggggag |
| | | cgctcctttttggcttccttccaggcgcggggctgctgcgctcttttttcggccggcggccgc |
| 61 | Rep Construct | caattgtcgcgaaataaaatatctttattttcattacatctgtgtgttggttttttgtgtgaatcgatagtactaacatacgctctccat |
| | #3 | caaaacaaaacgaaacaaaacaaactagcaaaataggctgtccccagtgcaagtgcaggtgccagaacatttctctggcct |
| | | aactggccggtacctgagctcgctagcctcgagcctgcaggcgacatgaagaatctgcttagggttaggcgttttgcgctgc |
| | | ttcgcgttgtacgggccagatatacgcgtatctgaggggactagggtgtgtttaggcgaaaagcggggcttcggttgtacgc |
| | | ggttaggagtcccctcaggatatagtagtttcgcttttgcatagggagggggattgcgccttttccaaggcagccctgggtttg |
| | | cgcagggacgcggctgctctgggcgtggttccgggaaacgcagcggcgccgaccctgggtctcgcacattcttcacgtcc |
| | | gttcgcagcgtcacccggatcttcgccgctacccttgtgggccccccggcgacgcttcctgctccgcccctaagtcgggaa |
| | | ggttccttgcggttcgcggcgtgccggacgtgacaaacggaagccgcacgtctcactagtaccctcgcagacggacagcg |
| | | ccagggagcaatggcagcgcgccgaccgcgatgggctgtggccaatagcggctgctcagcagggcgcgccgagagca |
| | | gcggccgggaaggggcggtgcgggaggggggtgtggggcggtagtgtgggccctgttcctgcccgcgcggtgttccg |
| | | cattctgcaagcctccggagcgcacgtcggcagtcggctccctcgttgaccgaatcaccgacctctctccccagggggatc |
| | | caccggagcttaccttttatccaagctgtgcttgtgatactgtctgtcttcttgtacttgtttgcctacaaagagtacctcgcctgc |
| | | ctcgtgctggccttggccctgggctgggcgaacaagctctactatacgcggggtttccagtccatcggcttgtacagcgtcat |
| | | catccagaaggtgcgttgagagctgcgccggcgggaggggcctcggaggatggccatttttcagatggggaaactgagat |
| | | ctggagaggaagaggggtttaccaaggggcagttagggtggggggtgggagagggtgggaagcaggaagatcagggt |
| | | gccgtgaatatgtgcttgcaccttacacttgaattatgtttatttccttataggtcatttgccaccatgttctggagttcttgtttgtat |
| | | atatcgtgtttttgcttggatttggagttggtaattgatttataaataatagtaactcccacatttataaacactttatacagatgtcaa |
| | | aatggcaaagtgcatggcggccttggcaacaattagaccagaaggaggcagcaggcttgtcatgtaggcaaatacacgca |
| | | agcttgtagagtaattaccaggcagagagctgaggagagcaccctgttatgttcccttagccttggcctcgcttatcgagaag |
| | | tgtcccaaagacaacaaggactgcagctcctacggcagcttcagcgacgcagtgctggaactcttcaagctcaccattggc |
| | | ctgggtgacctggacatccagcagaactccaagtatcccattctctttctgttcctgctcatcacctttgtcatcctcacctttgttc |
| | | tcctcctcaacaagctcattgctcttatcggcgagactgtggagaacgtctccaaggagagcgaacgcatctggcgcctgcg |
| | | tgccggggttttacgagattgtgattaaggtccccagcgaccttgacgagcatctgcccggcatttctgacagctttgtgaact |
| | | gggtggccgagaaggaatgggagttgccgccagattctgacatggatctgaatctgattgagcaggcacccctgaccgtg |
| | | gccgagaagctgcagcgcgactttctgacggaatggcgccgtgtgagtaaggccccggaggcccttttctttgtgcaatttg |
| | | agaagggagagagctacttccacatgcacgtgctcgtggaaaccaccggggtgaaatctatggttttgggacgtttcctgag |
| | | tcagattcgcgaaaaactgattcagagaatttaccgcgggatcgagccgactttgccaaactggttcgcggtcacaaagacc |
| | | agaaatggcgccggaggcgggaacaaggtggtggatgagtgctacatccccaattacttgctccccaaaacccagcctga |
| | | gctccagtgggcgtggactaatatggaacagtatttaagcgcctgtttgaatctcacggagcgtaaacggttggtggcgcag |
| | | catctgacgcacgtgtcgcagacgcaggagcagaacaaagagaatcagaatcccaattctgatgcgccggtgatcagatc |
| | | aaaaacttcagccaggtacatggagctggtcgggtggctcgtggacaaggggattacctcggagaagcagtggattcagg |
| | | aggaccaggcctcatacatctccttcaatgcggcctccaactcgcggtcccaaatcaaggctgccttggacaatgcgggaa |
| | | agattatgagcctgactaaaaccgcccccgactacctggtgggccagcagcccgtggaggacatttccagcaatcggattt |
| | | ataaaattttggaactaaacgggtacgatccccaatatgcggcttccgtctttctgggatgggccacgaaaaagttcggcaag |
| | | aggaacaccatctggctgtttgggcctgcaactaccggaaagaccaacatcgcggaggccatagcccacactgtgcccttc |
| | | tacgggtgcgtaaactggaccaatgagaactttcccttcaacgactgtgtcgacaagatggtgatctggtgggaggagggg |
| | | aagatgaccgccaaggtcgtggagtcggccaaagccattctcggaggaagcaaggtgcgcgtggaccagaaatgcaagt |
| | | cctcggcccagatagacccgactcccgtgatcgtcacctccaacaccaacatgtgcgccgtgattgacgggaactcaacga |
| | | ccttcgaacaccagcagccgttgcaagaccggatgttcaaatttgaactcacccgccgtctggatcatgactttgggaaggtc |
| | | accaagcaggaagtcaaagactttttccgggggcaaaggatcacgtggttgaggtggagcatgagttctacgtcaaaaag |
| | | ggtggagccaagaaaagacccgcccccagtgacgcagatataagtgagcccaaacgggtgcgcgagtcagttgcgcag |
| | | ccatcgacgtcagacgcggaggcttcgatcaactacgcagacaggtaccaaaacaaatgttctcgtcacgtgggcatgaat |
| | | ctgatgctgtttccctgcagacaatgcgagagaatgaatcagaattcaaatatctgcttcactcacggacagaaagactgttta |
| | | gagtgctttcccgtgtcagaatctcaacccgtttctgtcgtcaaaaaggcgtatcagaaactgtgctacattcatcatatcatgg |
| | | gaaaggtgccagacgcttgcactgcctgcgatctggtcaatgtggatttggatgactgcatctttgaacaataaaggtaaactt |
| | | gttaatcaataaaccgtttaattcgtttcagttgaacttttctctgctaactttctttctttctagtttggacttccctccaggtaaccgg |
| | | tcggggcaacatgactacgtccggcgttccatttggcatgacactacgaccaacacgatctcggttgtctcggcgcactccg |
| | | tacagtagggatcgtctacctccttttgagacagaaacccgcgctaccatactggaggatcatccgctgctgcccgaatgtaa |
| | | cactttgacaatgcacaacgtgagttacgtgcgaggacttccctgcagtgtgggatttacgctgattcaggaatgggttgttcc |
| | | ctgggatatggttctaacgcgggaggagcttgtaatcctgaggaagtgtatgcacgtgtgcctgtgttgtgccaacattgacat |
| | | catgacgagcatgatgatccacggttacgagtcctgggctctccactgtcattgttccagtcccggttccctgcagtgtatagc |
| | | cggcgggcaggttttggccagctggtttaggatggtggtggatggcgccatgtttaatcagaggtttatatggtatcgggagg |
| | | tggtgaattacaacatgccaaaagaggtaatgtttatgtccagcgtgtttatgaggggtcgccacttaatctacctgcgcttgtg |
| | | gtatgatggccacgtgggttctgtggtccccgccatgagctttggatacagcgccttgcactgtgggattttgaacaatattgt |
| | | ggtgctgtgctgcagttactgtgctgatttaagtgagatcagggtgcgctgctgtgcccggaggacaaggcgccttatgctg |
| | | cgggcggtgcgaatcatcgctgaggagactaccgccatgttgtattcctgcaggacggagcggcggcggcagcagtttatt |
| | | cgcgcgctgctgcagcaccaccgccctatcctgatgcacgattatgactctacccccatgtaggttgtttgacccctcccccg |
| | | tgccttccttgaccctggaaggtgccactcccactgtcctttcctaataaaatgaggaaattgcatcgcattgtctgagtaggtg |
| | | tcattctattctggggggtggggggggcaggacagcaagggggatccctgcaggcgcaggcggtcagcatcgccatgg |
| | | agaccaacacccttcccaccgccactcccccttcctctcagggtccctgtcccctccagtgaatcccagaaggactctggag |
| | | agttctgagcagggggcggcactctggcctctgattggtccaaggaaggctggggggcaggacgggaggcgaaaaccc |
| | | tggaatattcccgacctggcagcctcatcgagctcggtgattggctcagaagggaaaaggcgggtctccgtgacgacttata |
| | | aaagcccaggggcaagcggtccggataacggctacctgaggagctgctgcgacagtccactacctttttcgaggtgactcc |
| | | cgttgtcccaaggcttcccagagcgaacctgtgcggctgcaggcaccggcgcgtcgagtttccggcgtccggaaggacc |
| | | gagctcttctcgctgatccgtgttccgtttccgcccccaatctctcgttgccgacgcagggaaccggcggcaaccatggcca |
| | | gtcgggaagaggagcagcgcgaaaccacccccgagcgcggacgcggtgcggcgcgacgtcccccaaccatggagga |
| | | cgtgtcgtccccgtccccgtcgccgccgcctccccgggcgcccccaaaaaagcggatgaggcggcgtatcgagtccgag |
| | | gacgaggaggactcatcacaagacgcgctggtgccgcgcacacccagcccgcggccatcgacctcggcggcggatttg |
| | | gccattgcgcccaagaagaaaaagaagcgcccttctcccaagcccgagcgcccgccatcaccagaggtaatcgtggaca |
| | | gcgaggaagaaagagaagatgtggcgctacaaatggtgggtttcagcaaccctccggtgctaatcaagcatggcaaagga |
| | | ggtaagcgcacagtgcggcggctgaatgaagatgacccagtggcgcgtggtatgcggacgcaagaggaagaggaaga |
| | | gcccagcgaagcggaaagtgaaattacggtgatgaacccgctgagtgtgccgatcgtgtctgcgtgggagaagggcatg |
| | | gaggctgcgcgggcgctgatggacaagtaccacgtggataacgatctaaaggcgaacttcaaactactgcctgaccaagt |
| | | ggaagctctggcagccgtatgcaagacctggctgaacgaggagcaccgcgggttgcagctgaccttcaccagcaacaag |
| | | acctttgtgacgatgatggggcgattcctgcaggcgtacctgcagtcgtttgcagaggtgacctacaagcatcacgagccca |
| | | cgggctgcgcgttgtggctgcaccgctgcgctgagatcgaaggcgagcttaagtgtctacacggaagcattatgataaata |
| | | aggagcacgtgattgaaatggatgtgacgagcgaatttgggcagcgcgcgctgaaggagcagtctagcaaggccaagat |
| | | cgtgaagaatcggtggggccgaaatgtggtgcagatttccaacaccgacgcaaggtgctgcgtgcacgacgcggcctgtc |
| | | cggccaatcagttttccggcaagtcttgcggcatgttcttctctgaaggcgcaaaggctcaggtggcttttaagcagatcaag |
| | | gcttttatgcaggcgctgtatcctaacgcccagaccgggcacggtcaccttttgatgccactacggtgcgagtgcaactcaa |
| | | agcctgggcacgcgccctttttgggaaggcagctaccaaagttgactccgttcgccctgagcaacgcggaggacctggac |
| | | gcggatctgatctccgacaagagcgtgctggccagcgtgcaccacccggcgctgatagtgttccagtgctgcaaccctgtg |
| | | tatcgcaactcgcgggcgcagggcggaggccccaactgcgacttcaagatatcggcgcccgacctgctaaacgcgttggt |
| | | gatggtgcgcagcctgtggagtgaaaacttcaccgagctgccgcggatggttgtgcctgagtttaagtggagcactaaaca |
| | | ccagtatcgcaacgtgtccctgccagtggcgcatagcgatgcgcggcagaacccctttgatttttaaacggcgcagacggc |
| | | aagtaaataatcacccgagagtgtacaaataaaagatcttgccacaaaatagttacaggaccaagcgagcgtgagagtcca |
| | | aggcgtgcgcagtcgttgacgctctagaccgtgcaaaaggagagcctgtaagcgggcactcttccgtggtctggtggataa |
| | | attcgcaagggtatcatggcggacgacccgggttcgaaccccggatcgggccgtccgccgtgatccatgcggttaccgcc |
| | | cgcgtgtcgaacccaggtgtgcgacgtcagacaacgggggagcgctcctttttggcttccttccaggcgcggcggctgctg |
| | | cgctcttttttcggccgcggccgc |
| 62 | Rep Construct | caattgagggtggaggtcctgtattagaggtcacgtgagtgttttgcgacattttgcgacaccatgtggtcacgctgggtattta |
| | #4 | agcccgagtgagcacgcagggctctccattttgaagcgggaggtttgaacgcgcagccgccttttatccaagctgtgcttgt |
| | | gatactgtctgtcttcttgtacttgtttgcctacaaagagtacctcgcctgcctcgtgctggccttggccctgggctgggcgaac |
| | | aagctctactatacgcggggtttccagtccatcggcttgtacagcgtcatcatccagaaggtgcgttgagagctgcgccggc |
| | | gggaggggcctcggaggatggccatttttcagatggggaaactgagatctggagaggaagaggggtttaccaaggggca |
| | | gttagggtggggggtgggagagggtgggaagcaggaagatcagggtgccgtgaatatgtgcttgcaccttacacttgaatt |
| | | atgtttatttccttataggtcatttgccaccatgttctggagttcttgtttgtatatatcgtgtttttgcttggatttggagttggtaattg |
| | | atttataaataatagtaactcccacatttataaacactttatacagatgtcaaaatggcaaagtgcatggcggccttggcaacaa |
| | | ttagaccagaaggaggcagcaggcttgtcatgtaggcaaatacacgcaagcttgtagagtaattaccaggcagagagctga |
| | | ggagagcaccctgttatgttcccttagccttggcctcgcttatcgagaagtgtcccaaagacaacaaggactgcagctcctac |
| | | ggcagcttcagcgacgcagtgctggaactcttcaagctcaccattggcctgggtgacctggacatccagcagaactccaag |
| | | tatcccattctctttctgttcctgctcatcacctttgtcatcctcacctttgttctcctcctcaacaagctcattgctcttatcggcgag |
| | | actgtggagaacgtctccaaggagagcgaacgcatctggcgcctgcgtgccggggttttacgagattgtgattaaggtccc |
| | | cagcgaccttgacgagcatctgcccggcatttctgacagctttgtgaactgggtggccgagaaggaatgggagttgccgcc |
| | | agattctgacatggatctgaatctgattgagcaggcacccctgaccgtggccgagaagctgcagcgcgactttctgacgga |
| | | atggcgccgtgtgagtaaggccccggaggcccttttctttgtgcaatttgagaagggagagagctacttccacatgcacgtg |
| | | ctcgtggaaaccaccggggtgaaatctatggttttgggacgtttcctgagtcagattcgcgaaaaactgattcagagaatttac |
| | | cgcgggatcgagccgactttgccaaactggttcgcggtcacaaagaccagaaatggcgccggaggcgggaacaaggtg |
| | | gtggatgagtgctacatccccaattacttgctccccaaaacccagcctgagctccagtgggcgtggactaatatggaacagt |
| | | atttaagcgcctgtttgaatctcacggagcgtaaacggttggtggcgcagcatctgacgcacgtgtcgcagacgcaggagc |
| | | agaacaaagagaatcagaatcccaattctgatgcgccggtgatcagatcaaaaacttcagccaggtacatggagctggtcg |
| | | ggtggctcgtggacaaggggattacctcggagaagcagtggattcaggaggaccaggcctcatacatctccttcaatgcgg |
| | | cctccaactcgcggtcccaaatcaaggctgccttggacaatgcgggaaagattatgagcctgactaaaaccgcccccgact |
| | | acctggtgggccagcagcccgtggaggacatttccagcaatcggatttataaaattttggaactaaacgggtacgatcccca |
| | | atatgcggcttccgtctttctgggatgggccacgaaaaagttcggcaagaggaacaccatctggctgtttgggcctgcaact |
| | | accggaaagaccaacatcgcggaggccatagcccacactgtgcccttctacgggtgcgtaaactggaccaatgagaacttt |
| | | cccttcaacgactgtgtcgacaagatggtgatctggtgggaggaggggaagatgaccgccaaggtcgtggagtcggcca |
| | | aagccattctcggaggaagcaaggtgcgcgtggaccagaaatgcaagtcctcggcccagatagacccgactcccgtgatc |
| | | gtcacctccaacaccaacatgtgcgccgtgattgacgggaactcaacgaccttcgaacaccagcagccgttgcaagaccg |
| | | gatgttcaaatttgaactcacccgccgtctggatcatgactttgggaaggtcaccaagcaggaagtcaaagactttttccggtg |
| | | ggcaaaggatcacgtggttgaggtggagcatgagttctacgtcaaaaagggtggagccaagaaaagacccgcccccagt |
| | | gacgcagatataagtgagcccaaacgggtgcgcgagtcagttgcgcagccatcgacgtcagacgcggaggcttcgatca |
| | | actacgcagacaggtaccaaaacaaatgttctcgtcacgtgggcatgaatctgatgctgtttccctgcagacaatgcgagag |
| | | aatgaatcagaattcaaatatctgcttcactcacggacagaaagactgtttagagtgctttcccgtgtcagaatctcaacccgtt |
| | | tctgtcgtcaaaaaggcgtatcagaaactgtgctacattcatcatatcatgggaaaggtgccagacgcttgcactgcctgcga |
| | | tctggtcaatgtggatttggatgactgcatctttgaacaataaaggtaaacttgttaatcaataaaccgtttaattcgtttcagttga |
| | | acttttctctgctaactttctttctttctagtttggacttccctccaggtaaccggtcggggcaacatgactacgtccggcgttcca |
| | | tttggcatgacactacgaccaacacgatctcggttgtctcggcgcactccgtacagtagggatcgtctacctccttttgagaca |
| | | gaaacccgcgctaccatactggaggatcatccgctgctgcccgaatgtaacactttgacaatgcacaacgtgagttacgtgc |
| | | gaggacttccctgcagtgtgggatttacgctgattcaggaatgggttgttccctgggatatggttctaacgcgggaggagctt |
| | | gtaatcctgaggaagtgtatgcacgtgtgcctgtgttgtgccaacattgacatcatgacgagcatgatgatccacggttacga |
| | | gtcctgggctctccactgtcattgttccagtcccggttccctgcagtgtatagccggcgggcaggttttggccagctggtttag |
| | | gatggtggtggatggcgccatgtttaatcagaggtttatatggtatcgggaggtggtgaattacaacatgccaaaagaggtaa |
| | | tgtttatgtccagcgtgtttatgaggggtcgccacttaatctacctgcgcttgtggtatgatggccacgtgggttctgtggtcccc |
| | | gccatgagctttggatacagcgccttgcactgtgggattttgaacaatattgtggtgctgtgctgcagttactgtgctgatttaag |
| | | tgagatcagggtgcgctgctgtgcccggaggacaaggcgccttatgctgcgggcggtgcgaatcatcgctgaggagacta |
| | | ccgccatgttgtattcctgcaggacggagcggggggcagcagtttattcgcgcgctgctgcagcaccaccgccctatcc |
| | | tgatgcacgattatgactctacccccatgtaggttgtttgacccctcccccgtgccttccttgaccctggaaggtgccactccc |
| | | actgtcctttcctaataaaatgaggaaattgcatcgcattgtctgagtaggtgtcattctattctggggggtggggggggcag |
| | | gacagcaagggggatccctgcaggcgcaggcggtcagcatcgccatggagaccaacacccttcccaccgccactcccc |
| | | cttcctctcagggtccctgtcccctccagtgaatcccagaaggactctggagagttctgagcagggggcggcactctggcct |
| | | ctgattggtccaaggaaggctggggggcaggacgggaggcgaaaaccctggaatattcccgacctggcagcctcatcga |
| | | gctcggtgattggctcagaagggaaaaggcgggtctccgtgacgacttataaaagcccaggggcaagcggtccggataa |
| | | cggctacctgaggagctgctgcgacagtccactacctttttcgaggtgactcccgttgtcccaaggcttcccagagcgaacc |
| | | tgtgcggctgcaggcaccggcgcgtcgagtttccggcgtccggaaggaccgagctcttctcgctgatccgtgttccgtttcc |
| | | gcccccaatctctcgttgccgacgcagggaaccggcggcaaccatggccagtcgggaagaggagcagcgcgaaacca |
| | | cccccgagcgcggacgcggtgcggcgcgacgtcccccaaccatggaggacgtgtcgtccccgtccccgtcgccgccgc |
| | | ctccccgggcgcccccaaaaaagcggatgaggcggcgtatcgagtccgaggacgaggaggactcatcacaagacgcg |
| | | ctggtgccgcgcacacccagcccgcggccatcgacctcggcggcggatttggccattgcgcccaagaagaaaaagaag |
| | | cgcccttctcccaagcccgagcgcccgccatcaccagaggtaatcgtggacagcgaggaagaaagagaagatgtggcg |
| | | ctacaaatggtgggtttcagcaaccctccggtgctaatcaagcatggcaaaggaggtaagcgcacagtgcggcggctgaa |
| | | tgaagatgacccagtggcgcgtggtatgcggacgcaagaggaagaggaagagcccagcgaagcggaaagtgaaattac |
| | | ggtgatgaacccgctgagtgtgccgatcgtgtctgcgtgggagaagggcatggaggctgcgcgggcgctgatggacaag |
| | | taccacgtggataacgatctaaaggcgaacttcaaactactgcctgaccaagtggaagctctggcagccgtatgcaagacct |
| | | ggctgaacgaggagcaccgcgggttgcagctgaccttcaccagcaacaagacctttgtgacgatgatggggcgattcctg |
| | | caggcgtacctgcagtcgtttgcagaggtgacctacaagcatcacgagcccacgggctgcgcgttgtggctgcaccgctg |
| | | cgctgagatcgaaggcgagcttaagtgtctacacggaagcattatgataaataaggagcacgtgattgaaatggatgtgacg |
| | | agcgaatttgggcagcgcgcgctgaaggagcagtctagcaaggccaagatcgtgaagaatcggtggggccgaaatgtgg |
| | | tgcagatttccaacaccgacgcaaggtgctgcgtgcacgacgcggcctgtccggccaatcagttttccggcaagtcttgcg |
| | | gcatgttcttctctgaaggcgcaaaggctcaggtggcttttaagcagatcaaggcttttatgcaggcgctgtatcctaacgccc |
| | | agaccgggcacggtcaccttttgatgccactacggtgcgagtgcaactcaaagcctgggcacgcgccctttttgggaaggc |
| | | agctaccaaagttgactccgttcgccctgagcaacgcggaggacctggacgcggatctgatctccgacaagagcgtgctg |
| | | gccagcgtgcaccacccggcgctgatagtgttccagtgctgcaaccctgtgtatcgcaactcgcgggcgcagggcggag |
| | | gccccaactgcgacttcaagatatcggcgcccgacctgctaaacgcgttggtgatggtgcgcagcctgtggagtgaaaact |
| | | tcaccgagctgccgcggatggttgtgcctgagtttaagtggagcactaaacaccagtatcgcaacgtgtccctgccagtggc |
| | | gcatagcgatgcgcggcagaacccctttgatttttaaacggcgcagacggcaagtaaataatcacccgagagtgtacaaat |
| | | aaaagatcttgccacaaaatagttacaggaccaagcgagcgtgagagtccaaggcgtgcgcagtcgttgacgctctagac |
| | | cgtgcaaaaggagagcctgtaagcgggcactcttccgtggtctggtggataaattcgcaagggtatcatggcggacgaccc |
| | | gggttcgaaccccggatcgggccgtccgccgtgatccatgcggttaccgcccgcgtgtcgaacccaggtgtgcgacgtca |
| | | gacaacgggggagcgctcctttttggcttccttccaggcgcggcggctgctgcgctcttttttcggccggcggccgc |
| 63 | iRFP Construct | tcgcgaaataaaatatctttattttcattacatctgtgtgttggttttttgtgtgaatcgatagtactaacatacgctctccatcaaaa |
| | #1 | caaaacgaaacaaaacaaactagcaaaataggctgtccccagtgcaagtgcaggtgccagaacatttctctggcctaactg |
| | | gccggtacctgagctcgctagcctcgagcctgcaggcgacaattgcatgaagaatctgcttagggttaggcgttttgcgctg |
| | | cttcgcgttgtacgggccagatatacgcgtatctgaggggactagggtgtgtttaggcgaaaagcggggcttcggttgtacg |
| | | cggttaggagtcccctcaggatatagtagtttcgcttttgcatagggagggggaaatgtagtcttatgcaatacacttgtagtct |
| | | tgcaacatggtaacgatgagttagcaacatgccttacaaggagagaaaaagcaccgtgcatgccgattggtggaagtaagg |
| | | tggtacgatcgtgccttattaggaaggcaacagacaggtctgacatggattggacgaaccactgaattgcgcattgcagaga |
| | | taattgtatttaagtgcctagctcgatacaataaacgccatttgaccattcaccacattggtgtgcacctccaaggccacagatc |
| | | cgtttaaacaagcttggcaatccggtactgttggtaaagccaccatggcggatcctccccgaacaagatcaagacagttaag |
| | | tgcgcgccgggcgtggacgtctgcaccgaggccgtgggggcggtggagaccatccacggacaattctcgctggcagtg |
| | | cggggttgcggttcgggactccccggcaagagcggaaggatccgtcgccaggcagcctgacctcttgacctgcgacgat |
| | | gagccgatccatatccccggtgccatccaaccgcatggactgctgctcgccctcgccgccgacatgacgatcgttgccggc |
| | | agcgacaaccttcccgaactcaccggactggcgatcggcgccctgatcggccgctctgcggccgatgtcttcgactcgga |
| | | gacgcacaaccgtctgacgatcgccttggccgagcccggggcggccgtcggagcaccgatcactgtcggcttcacgatg |
| | | cgaaaggacgcaggcttcatcggctcctggcatcgccatgatcagctcatcttcctcgagctcgagcctccccagcgggac |
| | | gtcgccgagccgcaggcgttcttccgccgcaccaacagcgccatccgccgcctgcaggccgccgaaaccttggaaagc |
| | | gcctgcgccgccgcggcgcaagaggtgcggaagattaccggcttcgatcgggtgatgatctatcgcttcgcctccgacttc |
| | | agcggcgaagtgatcgcagaggatcggtgcgccgaggtcgagtcaaaactaggcctgcactatcctgcctcaaccgtgcc |
| | | ggcgcaggcccgtcggctctataccatcaacccggtacggatcattcccgatatcaattatcggccggtgccggtcacccc |
| | | agacctcaatccggtcaccgggcggccgattgatcttagcttcgccatcctgcgcagcgtctcgcccgtccatctggaattca |
| | | tgcgcaacataggcatgcacggcacgatgtcgatctcgattttgcgcggcgagcgactgtggggattgatcgtttgccatca |
| | | ccgaacgccgtactacgtcgatctcgatggccgccaagcctgcgagctagtcgcccaggttctggcctggcagatcggcg |
| | | tgatggaagagtgagggccc |
| 64 | iRFP Construct | gacggatcgggagatctcccgatcccctatggtcgactctcagtacaatctgctctgatgccgcatagttaagccagtatctg |
| | #2 | ctccctgcttgtgtgttggaggtcgctgagtagtgcgcgagcaaaatttaagctacaacaaggcaaggcttgaccgacaatt |
| | | gcatgaagaatctgcttagggttaggcgttttgcgctgcttcgcgaaataaaatatctttattttcattacatctgtgtgttggtttttt |
| | | gtgtgaatcgatagtactaacatacgctctccatcaaaacaaaacgaaacaaaacaaactagcaaaataggctgtccccagt |
| | | gcaagtgcaggtgccagaacatttctctggcctaactggccggtacctgagctcgctagcctcgagcctgcaggcgacaat |
| | | tgcatgaagaatctgcttagggttaggcgttttgcgctgcttcgcgttgtacgggccagatatacgcgtatctgaggggacta |
| | | gggtgtgtttaggcgaaaagcggggcttcggttgtacgcggttaggagtcccctcaggatatagtagtttcgcttttgcatagg |
| | | gagggggaaatgtagtcttatgcaatacacttgtagtcttgcaacatggtaacgatgagttagcaacatgccttacaaggaga |
| | | gaaaaagcaccgtgcatgccgattggtggaagtaaggtggtacgatcgtgccttattaggaaggcaacagacaggtctgac |
| | | atggattggacgaaccactgaattgcgcattgcagagataattgtatttaagtgcctagctcgatacaataaacgccatttgac |
| | | cattcaccacattggtgtgcacctccaaggccacagatccgtttaaacaagcttggcaatccggtactgttggtaaagctgct |
| | | gctgctgctgcttcgcggaggtgggtctgctgcccggagaggtggagggggtagagtgggggctcttaggtggacgcttt |
| | | agcaaggagggctcaggtctattcggctccgctcccccaggagcgcaggccctggagtgctacagctgcgtgcagagcc |
| | | accatggcggatcctccccgaacaagatcaagacagttaagtgcgcgccgggcgtggacgtctgcaccgaggccgtggg |
| | | ggcggtggagaccagtgagtggggacccccgggccacgcacaccactgcaggctcctccggcccccatctgagaccac |
| | | gcccacagctaatcccgcccttgtccctctccctagtccacggacaattctcgctggcagtgcggggttgcggttcgggact |
| | | ccccggcaagagtggcggaaggatccgtcgccaggcagcctgacctcttgacctgcgacgatgagccgatccatatcccc |
| | | ggtgccatccaaccgcatggactgctgctcgccctcgccgccgacatgacgatcgttgccggcagcgacaaccttcccga |
| | | actcaccggactggcgatcggcgccctgatcggccgctctgcggccgatgtcttcgactcggagacgcacaaccgtctga |
| | | cgatcgccttggccgagcccggggcggccgtcggagcaccgatcactgtcggcttcacgatgcgaaaggacgcaggctt |
| | | catcggctcctggcatcgccatgatcagctcatcttcctcgagctcgagcctccccagcgggacgtcgccgagccgcagg |
| | | cgttcttccgccgcaccaacagcgccatccgccgcctgcaggccgccgaaaccttggaaagcgcctgcgccgccgcggc |
| | | gcaagaggtgcggaagattaccggcttcgatcgggtgatgatctatcgcttcgcctccgacttcagcggcgaagtgatcgc |
| | | agaggatcggtgcgccgaggtcgagtcaaaactaggcctgcactatcctgcctcaaccgtgccggcgcaggcccgtcgg |
| | | ctctataccatcaacccggtacggatcattcccgatatcaattatcggccggtgccggtcaccccagacctcaatccggtcac |
| | | cgggcggccgattgatcttagcttcgccatcctgcgcagcgtctcgcccgtccatctggaattcatgcgcaacataggcatg |
| | | cacggcacgatgtcgatctcgattttgcgcggcgagcgactgtggggattgatcgtttgccatcaccgaacgccgtactacg |
| | | tcgatctcgatggccgccaagcctgcaagagggtcgccgagaggctggccactcagatcggcgtgatggaagagtgagg |
| | | gcccgtttaaacccgctgatcagcctcgactgtgccttctagttgccagccatctgttgtttgcccctcccccgtgccttccttga |
| | | ccctggaaggtgccactcccactgtcctttcctaataaaatgaggaaattgcatcgcattgtctgagtaggtgtcattctattctg |
| | | gggggtggggggggcaggacagcaagggggaggattgggaagacaatagcaggcatgctggggatgcggtgggctc |
| | | tatggcttctgaggcggaaagaaccagctggggctctagggggtatccccacgcgccctgtagcggcgcattaagcgcgg |
| | | cgggtgtggtggttacgcgcagcgtgaccgctacacttgccagcgccctagcgcccgctcctttcgctttcttcccttcctttct |
| | | cgccacgttcgccggctttccccgtcaagctctaaatcggggcatccctttagggttccgatttagtgctttacggcacctcga |
| | | ccccaaaaaacttgattagggtgatggttcacgtagtgggccatcgccctgatagacggtttttcgccctttgacgttggagtc |
| | | cacgttctttaatagtggactcttgttccaaactggaacaacactcaaccctatctcggtctattcttttgatttataagggattttg |
| | | gggatttcggcctattggttaaaaaatgagctgatttaacaaaaatttaacgcgaattaattctgtggaatgtgtgtcagttaggg |
| | | tgtggaaagtccccaggctccccaggcaggcagaagtatgcaaagcatgcatctcaattagtcagcaaccaggtgtggaa |
| | | agtccccaggctccccagcaggcagaagtatgcaaagcatgcatctcaattagtcagcaaccatagtcccgcccctaactc |
| | | cgcccatcccgcccctaactccgcccagttccgcccattctccgccccatggctgactaattttttttatttatgcagaggccga |
| | | ggccgcctctgcctctgagctattccagaagtagtgaggaggcttttttggaggcctaggcttttgcaaaaagctcccgggag |
| | | cttgtatatccattttcggatctgatcagcacgtgatgaaaaagcctgaactcaccgcgacgtctgtcgagaagtttctgatcga |
| | | aaagttcgacagcgtctccgacctgatgcagctctcggagggcgaagaatctcgtgctttcagcttcgatgtaggagggcgt |
| | | ggatatgtcctgcgggtaaatagctgcgccgatggtttctacaaagatcgttatgtttatcggcactttgcatcggccgcgctc |
| | | ccgattccggaagtgcttgacattggggaattcagcgagagcctgacctattgcatctcccgccgtgcacagggtgtcacgt |
| | | tgcaagacctgcctgaaaccgaactgcccgctgttctgcagccggtcgcggaggccatggatgcgatcgctgcggccgat |
| | | cttagccagacgagcgggttcggcccattcggaccgcaaggaatcggtcaatacactacatggcgtgatttcatatgcgcg |
| | | attgctgatccccatgtgtatcactggcaaactgtgatggacgacaccgtcagtgcgtccgtcgcgcaggctctcgatgagct |
| | | gatgctttgggccgaggactgccccgaagtccggcacctcgtgcacgcggatttcggctccaacaatgtcctgacggacaa |
| | | tggccgcataacagcggtcattgactggagcgaggcgatgttcggggattcccaatacgaggtcgccaacatcttcttctgg |
| | | aggccgtggttggcttgtatggagcagcagacgcgctacttcgagcggaggcatccggagcttgcaggatcgccgcggct |
| | | ccgggcgtatatgctccgcattggtcttgaccaactctatcagagcttggttgacggcaatttcgatgatgcagcttgggcgca |
| | | gggtcgatgcgacgcaatcgtccgatccggagccgggactgtcgggcgtacacaaatcgcccgcagaagcgcggccgt |
| | | ctggaccgatggctgtgtagaagtactcgccgatagtggaaaccgacgccccagcactcgtccgagggcaaaggaatag |
| | | cacgtgctacgagatttcgattccaccgccgccttctatgaaaggttgggcttcggaatcgttttccgggacgccggctggat |
| | | gatcctccagcgcggggatctcatgctggagttcttcgcccaccccaacttgtttattgcagcttataatggttacaaataaagc |
| | | aatagcatcacaaatttcacaaataaagcatttttttcactgcattctagttgtggtttgtccaaactcatcaatgtatcttatcatgt |
| | | ctgtataccgtcgacctctagctagagcttggcgtaatcatggtcatagctgtttcctgtgtgaaattgttatccgctcacaattcc |
| | | acacaacatacgagccggaagcataaagtgtaaagcctggggtgcctaatgagtgagctaactcacattaattgcgttgcgc |
| | | tcactgcccgctttccagtcgggaaacctgtcgtgccagctgcattaatgaatcggccaacgcgcggggagaggcggtttg |
| | | cgtattgggcgctcttccgcttcctcgctcactgactcgctgcgctcggtcgttcggctgcggcgagcggtatcagctcactc |
| | | aaaggcggtaatacggttatccacagaatcaggggataacgcaggaaagaacatgtgagcaaaaggccagcaaaaggc |
| | | caggaaccgtaaaaaggccgcgttgctggcgtttttccataggctccgcccccctgacgagcatcacaaaaatcgacgctc |
| | | aagtcagaggtggcgaaacccgacaggactataaagataccaggcgtttccccctggaagctccctcgtgcgctctcctgtt |
| | | ccgaccctgccgcttaccggatacctgtccgcctttctcccttcgggaagcgtggcgctttctcaatgctcacgctgtaggtat |
| | | ctcagttcggtgtaggtcgttcgctccaagctgggctgtgtgcacgaaccccccgttcagcccgaccgctgcgccttatccg |
| | | gtaactatcgtcttgagtccaacccggtaagacacgacttatcgccactggcagcagccactggtaacaggattagcagag |
| | | cgaggtatgtaggcggtgctacagagttcttgaagtggtggcctaactacggctacactagaaggacagtatttggtatctgc |
| | | gctctgctgaagccagttaccttcggaaaaagagttggtagctcttgatccggcaaacaaaccaccgctggtagcggtggttt |
| | | ttttgtttgcaagcagcagattacgcgcagaaaaaaaggatctcaagaagatcctttgatcttttctacggggtctgacgctca |
| | | gtggaacgaaaactcacgttaagggattttggtcatgagattatcaaaaaggatcttcacctagatccttttaaattaaaaatga |
| | | agttttaaatcaatctaaagtatatatgagtaaacttggtctgacagttaccaatgcttaatcagtgaggcacctatctcagcgat |
| | | ctgtctatttcgttcatccatagttgcctgactccccgtgtgtagataactacgatacgggagggcttaccatctggccccagt |
| | | gctgcaatgataccgcgagacccacgctcaccggctccagatttatcagcaataaaccagccagccggaagggccgagc |
| | | gcagaagtggtcctgcaactttatccgcctccatccagtctattaattgttgccgggaagctagagtaagtagttcgccagtta |
| | | atagtttgcgcaacgttgttgccattgctacaggcatcgtggtgtcacgctcgtcgtttggtatggcttcattcagctccggttcc |
| | | caacgatcaaggcgagttacatgatcccccatgttgtgcaaaaaagcggttagctccttcggtcctccgatcgttgtcagaag |
| | | taagttggccgcagtgttatcactcatggttatggcagcactgcataattctcttactgtcatgccatccgtaagatgcttttctgt |
| | | gactggtgagtactcaaccaagtcattctgagaatagtgtatgcggcgaccgagttgctcttgcccggcgtcaatacgggat |
| | | aataccgcgccacatagcagaactttaaaagtgctcatcattggaaaacgttcttcggggcgaaaactctcaaggatcttacc |
| | | gctgttgagatccagttcgatgtaacccactcgtgcacccaactgatcttcagcatcttttactttcaccagcgtttctgggtgag |
| | | caaaaacaggaaggcaaaatgccgcaaaaaagggaataagggcgacacggaaatgttgaatactcatactcttcctttttca |
| | | atattattgaagcatttatcagggttattgtctcatgagcggatacatatttgaatgtatttagaaaaataaacaaataggggttcc |
| | | gcgcacatttccccgaaaagtgccacctgacgtc |
| 65 | iRFP Construct | tcgcgaaataaaatatctttattttcattacatctgtgtgttggttttttgtgtgaatcgatagtactaacatacgctctccatcaaaa |
| | #3 | caaaacgaaacaaaacaaactagcaaaataggctgtccccagtgcaagtgcaggtgccagaacatttctctggcctaactg |
| | | gccggtacctgagctcgctagcctcgagcctgcaggcgacaattgcatgaagaatctgcttagggttaggcgttttgcgctg |
| | | cttcgcgttgtacgggccagatatacgcgtatctgaggggactagggtgtgtttaggcgaaaagcggggcttcggttgtacg |
| | | cggttaggagtcccctcaggatatagtagtttcgcttttgcatagggagggggaaatgtagtcttatgcaatacacttgtagtct |
| | | tgcaacatggtaacgatgagttagcaacatgccttacaaggagagaaaaagcaccgtgcatgccgattggtggaagtaagg |
| | | tggtacgatcgtgccttattaggaaggcaacagacaggtctgacatggattggacgaaccactgaattgcgcattgcagaga |
| | | taattgtatttaagtgcctagctcgatacaataaacgccatttgaccattcaccacattggtgtgcacctccaaggccacagatc |
| | | cgtttaaacaagcttggcaatccggtactgttggtaaagccaccatggcggaaggatccgtcgccaggcagcctgacctctt |
| | | gacctgcgacgatgagccgatccatatccccggtgccatccaaccgcatggactgctgctcgccctcgccgccgacatga |
| | | cgatcgttgccggcagcgacaaccttcccgaactcaccggactggcgatcggcgccctgatcggccgctctgcggccgat |
| | | gtcttcgactcggagacgcacaaccgtctgacgatcgccttggccgagcccggggggccgtcggagcaccgatcactgt |
| | | cggcttcacgatgcgaaaggacgcaggcttcatcggctcctggcatcgccatgatcagctcatcttcctcgagctcgagcct |
| | | ccccagcgggacgtcgccgagccgcaggcgttcttccgccgcaccaacagcgccatccgccgcctgcaggccgccgaa |
| | | accttggaaagcgcctgcgccgccgcggcgcaagaggtgcggaagattaccggcttcgatcgggtgatgatctatcgcttc |
| | | gcctccgacttcagcggcgaagtgatcgcagaggatcggtgcgccgaggtcgagtcaaaactaggcctgcactatcctgc |
| | | ctcaaccgtgccggcgcaggcccgtcggctctataccatcaacccggtacggatcattcccgatatcaattatcggccggtg |
| | | ccggtcaccccagacctcaatccggtcaccgggcggccgattgatcttagcttcgccatcctgcgcagcgtctcgcccgtc |
| | | catctggaattcatgcgcaacataggcatgcacggcacgatgtcgatctcgattttgcgcggcgagcgactgtggggattga |
| | | tcgtttgccatcaccgaacgccgtactacgtcgatctcgatggccgccaagcctgcaagagggtcgccgagaggctggcc |
| | | actcagatcggcgtgatggaagagtgagggccc |
| 66 | iRFP Construct | tcgcgaaataaaatatctttattttcattacatctgtgtgttggttttttgtgtgaatcgatagtactaacatacgctctccatcaaaa |
| | #4 | caaaacgaaacaaaacaaactagcaaaataggctgtccccagtgcaagtgcaggtgccagaacatttctctggcctaactg |
| | | gccggtacctgagctcgctagcctcgagcctgcaggcgacaattgcatgaagaatctgcttagggttaggcgttttgcgctg |
| | | cttcgcgttgtacgggccagatatacgcgtatctgaggggactagggtgtgtttaggcgaaaagcggggcttcggttgtacg |
| | | cggttaggagtcccctcaggatatagtagtttcgcttttgcatagggagggggaaatgtagtcttatgcaatacacttgtagtct |
| | | tgcaacatggtaacgatgagttagcaacatgccttacaaggagagaaaaagcaccgtgcatgccgattggtggaagtaagg |
| | | tggtacgatcgtgccttattaggaaggcaacagacaggtctgacatggattggacgaaccactgaattgcgcattgcagaga |
| | | taattgtatttaagtgcctagctcgatacaataaacgccatttgaccattcaccacattggtgtgcacctccaaggccacagatc |
| | | cgtttaaacaagcttggcaatccggtactgttggtaaatctactatacgcggggtttccagtccatcggcttgtacagcgtcatc |
| | | atccagaaggtgcgttgagagctgcgccggcgggaggggcctcggaggatggccattttgtgaatatgtgcttgcacctta |
| | | cacttgaattatgtttatttccttataggtcatttgccaccatgttctggagttcttgtttgtatatatcgtgtttttgcttggatttggag |
| | | ttggtaattgatttataaataatagtaactcccacatttataaacactttatagtaattaccaggcagagagctgaggagagcac |
| | | cctgttatgttcccttagccttggcctcgcttatcgagaagtgtcccaaagacaacaaggactgcagctcctacggcagcttca |
| | | gcgacgcagtgctggaactctgtggcggaaggatccgtcgccaggcagcctgacctcttgacctgcgacgatgagccgat |
| | | ccatatccccggtgccatccaaccgcatggactgctgctcgccctcgccgccgacatgacgatcgttgccggcagcgaca |
| | | accttcccgaactcaccggactggcgatcggcgccctgatcggccgctctgcggccgatgtcttcgactcggagacgcac |
| | | aaccgtctgacgatcgccttggccgagcccggggcggccgtcggagcaccgatcactgtcggcttcacgatgcgaaagg |
| | | acgcaggcttcatcggctcctggcatcgccatgatcagctcatcttcctcgagctcgagcctccccagcgggacgtcgccg |
| | | agccgcaggcgttcttccgccgcaccaacagcgccatccgccgcctgcaggccgccgaaaccttggaaagcgcctgcgc |
| | | cgccgcggcgcaagaggtgcggaagattaccggcttcgatcgggtgatgatctatcgcttcgcctccgacttcagcggcga |
| | | agtgatcgcagaggatcggtgcgccgaggtcgagtcaaaactaggcctgcactatcctgcctcaaccgtgccggcgcagg |
| | | cccgtcggctctataccatcaacccggtacggatcattcccgatatcaattatcggccggtgccggtcaccccagacctcaat |
| | | ccggtcaccgggcggccgattgatcttagcttcgccatcctgcgcagcgtctcgcccgtccatctggaattcatgcgcaaca |
| | | taggcatgcacggcacgatgtcgatctcgattttgcgcggcgagcgactgtggggattgatcgtttgccatcaccgaacgcc |
| | | gtactacgtcgatctcgatggccgccaagcctgcgagctagtcgcccaggttctggcctggcagatcggcgtgatggaaga |
| | | gtgagggccc |

What is claimed is:

1. An expression cassette comprising from 5′ to 3′: (i) promoter, (ii) an upstream exon, (iii) an upstream intron, (iv) an alternatively spliced exon comprising a translation initiation sequence, (v) a downstream intron, (vi) a downstream exon, and (vii) a heterologous nucleic acid sequence; wherein the upstream intron, the alternatively spliced exon, and the downstream intron each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

2. The expression cassette of claim 1, wherein the upstream exon and the downstream exon each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene.

3. The expression cassette of claim 1, wherein the upstream exon and the downstream exon each comprise a portion of a gene, which is different from the gene for the portion of the upstream intron, the alternatively spliced exon, and the downstream intron.

4. The expression cassette of claim 1, wherein the upstream exon and the downstream exon comprise synthetic exons.

5. The expression cassette of any one of claims 1-4, wherein the alternatively spliced exon is regulatable by a splicing modulator.

6. The expression cassette of claim 5, wherein the splicing modulator is Kinetin or Homocarbonyltopsentin.

7. The expression cassette of any one of claims 1-6, wherein the gene is TRPV3, STK31, or USP25; and the splicing modulator is Kinetin.

8. The expression cassette of any one of claims 1-6, wherein the gene is GTF2B, LYPD3, IK, or TEX14; and the splicing modulator is Homocarbonyltopsentin.

9. The expression cassette of any one of claims 5-8, wherein the splicing modulator promotes inclusion of the alternatively spliced exon thereby driving expression of the heterologous nucleic acid sequence.

10. The expression cassette of any one of claims 1-9, wherein the alternatively spliced exon further comprises a kozak sequence.

11. The expression cassette of any one of claims 1-9, wherein the promoter is selected from the group consisting of a CBA promoter, a smCBA promoter, a CMV promoter, an EF-1a (Elongation Factor 1a) promoter, a RSV (Rous Sarcoma Virus) promoter, an Ubiquitin (UbC) promoter, a CAG promoter, a SV40 (simian vacuolating virus 40) promoter, a PGK (phosphoglycerate kinase) promoter, a human beta actin promoter, a H1 (human polymerase III RNA) promoter, a human U6 small nuclear promoter, tetracycline responsive element, and any combination thereof.

12. The expression cassette of any one of the previous claims, further comprising an enhancer.

13. The expression cassette of claim 12, wherein the enhancer is selected from the group consisting of a CMV enhancer, a SV40 enhancer, and any combination thereof.

14. The expression cassette of any one of claims 1-13, which further comprises a cleavage site between the downstream exon and the heterologous nucleic acid sequence.

15. The expression cassette of claim 14, wherein the cleavage site is selected from the group consisting of a furin cleavage site, a foot-and-mouth disease virus 18 2A peptide (F2A), an equine rhinitis A virus 2A peptide (E2A), a porcine teschovirus-1 2A peptide (P2A), a Thosea asigna virus 2A peptide (T2A), and any combination thereof.

16. The expression cassette of any one of the claims 1-15, which further comprises a leader sequence between the downstream exon and the heterologous nucleic acid sequence.

17. The expression cassette of claim 16, wherein the leader sequence is selected from the group consisting of a human opticin (hOPT) leader sequence, an interleukin-2 (IL-2) leader sequence, an interleukin-12 (IL-12) leader sequence, an interleukin-6 (IL-6) leader sequence, a human insulin leader sequence, a human serum albumin (HSA) leader sequence, and any combination thereof.

18. The expression cassette of any one of the previous claims, wherein the combined length of the upstream exon, the upstream intron, the alternatively spliced exon, the downstream intron, and the downstream exon is between about 300 basepairs to about 2100 basepairs.

19. The expression cassette of any one of the previous claims, wherein the length of the heterologous nucleic acid sequence is less than about 4100 basepairs, less than about 4000 basepairs, or less than about 3900 basepairs.

20. The expression cassette of any one of the previous claims, wherein the heterologous nucleic acid sequence encodes a protein.

21. The expression cassette of claim 20, wherein the protein is a therapeutic protein.

22. The expression cassette of any one of the previous claims, wherein the upstream exon comprises a sequence selected from the group consisting of SEQ ID NO: 8, 13, 18, 23, 28, 33, and 38.

23. The expression cassette of any one of the previous claims, wherein the alternatively spliced exon comprises a sequence selected from the group consisting of SEQ ID NO: 10, 15, 20, 25, 30, 35, and 40.

24. The expression cassette of any one of the previous claims, wherein the downstream exon comprises a sequence selected from the group consisting of SEQ ID NO: 12, 17, 22, 27, 32, 37, and 42.

25. The expression cassette of any one of the previous claims, comprising a sequence selected from the group consisting of SEQ ID NO: 8-42.

26. The expression cassette of any one of the previous claims, comprising a sequence selected from the group consisting of SEQ ID NO: 1-8 or 43-66.

27. The expression cassette of any one of the previous claims, further comprising an untranslated region (UTR), a microRNA binding site, a polyA (pA) sequence, an intron sequence, or any combination thereof.

28. The expression cassette any one of the previous claims, wherein the expression cassette comprises a poly (A) (pA) sequence.

29. The expression cassette of claim 27 or claim 28, wherein the pA sequence is a synthetic pA sequence, a bovine growth hormone (bGH) pA sequence, or a human growth hormone (hGH) pA sequence.

30. A delivery vector comprising the expression cassette of any one of claims 1-26.

31. The delivery vector of claim 30, wherein the expression cassette is flanked by inverted terminal repeat (ITR) sequences.

32. The delivery vector of claim 31, wherein the delivery vector is selected from the group consisting of a viral vector, a plasmid, a lipid, a protein particle, a bacterial vector, a lysosome, a virus-like particle, a polymeric particle, an exosome, and a vault particle.

33. The delivery vector of claim 31 or claim 32, wherein the delivery vector is a viral vector.

34. The delivery vector of claim 33, wherein the viral vector is a lentiviral vector, an adenoviral vector, an adeno-associated viral (AAV) vector, or a retroviral vector.

35. The delivery vector of any one of claims 32-34, wherein the viral vector is an adeno-associated viral (AAV) vector.

36. The delivery vector of any one of claims 31-35, which is a recombinant AAV (rAAV) vector comprising an AAV serotype selected from the group consisting of AAV1, AAV2, AAV3, AAV4, AAV5, AAV6, AAV7, AAV8, AAVrh8, AAV9, AAV10, AAVrh10, AAV11, and AAV12.

37. A cell comprising the expression cassette of any one of claims 1-30, or the delivery vector of any one of claims 31-36.

38. A pharmaceutical composition comprising the expression cassette of any one of claims 1-30, the delivery vector of any one of claims 31-36, or the cell of claim 37.

39. The pharmaceutical composition of claim 38, further comprising a pharmaceutically acceptable excipient.

40. A kit comprising i) the expression cassette of any one of claims 1-30, the delivery vector of any one of claims 31-36, or the pharmaceutical composition of claim 38 or claim 39; and (ii) optionally instructions for use.

41. The kit of claim 40, further comprising (iii) a splicing modulator molecule that promotes the inclusion of the alternatively spliced exon.

42. The kit of claim 41, wherein the splicing modulator molecule is kinetin.

43. The kit of claim 42, wherein the splicing modulator molecule is homocarbonyltopsentin.

44. A method of regulating expression of a protein in a mammalian cell comprising administering to the cell the expression cassette of any one of claims 1-30, the delivery vector of any one of claims 31-36, or the pharmaceutical composition of claim 38 or claim 39, followed by administering the splicing modulator molecule that promotes the inclusion of the alternatively spliced exon.

45. A method of expressing a protein in a subject comprising administering to the subject the expression cassette of any one of claims 1-30, the delivery vector of any one of claims 31-36, the cell of claim 37, or the pharmaceutical composition of claim 38 or claim 39, followed by administering the splicing modulator molecule that promotes the inclusion of the alternatively spliced exon.

46. A method of treating a disease in a mammal comprising administering to a subject the expression cassette of any one of claims 1-30, the delivery vector of any one of claims 31-36, the cell of claim 37, or the pharmaceutical composition of claim 38 or claim 39, followed by administering the splicing modulator molecule that promotes the inclusion of the alternatively spliced exon.

47. The methods of any one of claims 44-46, wherein the administration of the expression cassette, delivery vector, or cell is systemic or tissue specific.

48. The methods of any one of claims 44-47, wherein the administration of the molecule that promotes the inclusion of the alternatively spliced exon is systemic.

49. The method of any one of claims 44-48, wherein the molecule that promotes the inclusion of the alternatively spliced exon is administered at least 1 day after administration of the expression cassette.

50. The method of any one of claims 44-49, further comprising administering a second dose of the molecule that promotes the inclusion of the alternatively spliced exon.

51. A method of regulating transgene expression post-transcriptionally, comprising administering to a cell or subject (a) a nucleic acid construct comprising (i) an upstream exon, (ii) an upstream intron, (iii) an alternatively spliced exon comprising a translation initiation sequence, (iv) a downstream intron, (v) a downstream exon, and (vi) a heterologous nucleic acid sequence; wherein the upstream intron, the alternatively spliced exon, and the downstream intron each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene; and (b) a splicing modulator, wherein the splicing modulator is Kinetin or Homocarbonyltopsentin.

52. A method of producing an adeno-associated viral (AAV) vector comprising:
(a) introducing a first plasmid comprising an adenovirus helper gene and a second plasmid comprising a heterologous nucleic acid sequence into a host cell, wherein the host cell comprises an AAV capsid (Cap) gene and a nucleic acid construct comprising (i) an upstream exon, (ii) an upstream intron, (iii) an alternatively spliced exon comprising a translation initiation sequence, (iv) a downstream intron, (v) a downstream exon, and (vi) an AAV replication (Rep) gene; wherein the upstream intron, the alternatively spliced exon, and the downstream intron each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene; and
(b) culturing the host cell in the presence of a splicing modulator to produce AAV particles.

53. A method of producing an adeno-associated viral (AAV) vector comprising:
(a) introducing a first plasmid comprising an adenovirus helper gene and a second plasmid comprising a heterologous nucleic acid sequence into a host cell, wherein the host cell comprises an AAV replication (Rep) gene and a nucleic acid construct comprising (i) an upstream exon, (ii) an upstream intron, (iii) an alternatively spliced exon comprising a translation initiation sequence, (iv) a downstream intron, (v) a downstream exon, and (vi) an AAV capsid (Cap) gene; wherein the upstream intron, the alternatively spliced exon, and the downstream intron each comprise a portion of a gene selected from the group consisting of a Transient Receptor Potential Cation Channel Subfamily V Member 3 (TRPV3) gene, a Serine/Threonine Kinase 31 (STK31) gene, an Ubiquitin Specific Peptidase 25 (USP25) gene, a IK gene, a Testis Expressed 14, Intercellular Bridge Forming Factor (TEX14) gene, a General Transcription Factor IIB (GTF2B) gene, and a LY6/PLAUR Domain Containing 3 (LYPD3) gene; and
(b) culturing the host cell in the presence of a splicing modulator to produce AAV particles.

54. The method of claim 52 or 53, further comprising (c) harvesting the AAV particles from the cultured host cell.

55. The method of claim 54, further comprising (d) purifying the harvested AAV particles.

56. The method of claim 52 or 53, wherein the host cell is a HEK293 cell.

57. The method of any one of claims 52 to 56, wherein the splicing modulator is Kinetin or Homocarbonyltopsentin.

58. The method of any one of claims 52 to 57, wherein the adenovirus helper gene is selected from the group consisting of an Early 4 (E4) gene, an Early 2A (E2A) gene, and a viral associated (VA) gene.

59. The method of any one of claims 52 to 58, wherein the nucleic acid construct is stably integrated into the host cell genome.

60. The method of any one of claims 52 to 58, wherein the nucleic acid construct comprises a sequence selected from the group consisting of SEQ ID NO: 59-62.

61. A method of producing an AAV particle comprising contacting a host cell comprising rep genes, cap genes, and adenoviral helper genes with a topsentin alkaloid or derivative thereof.

62. The method of claim 61, wherein topsentin alkaloid or derivative thereof is selected from the group consisting of Homocarbonyltopsentin, Topsentin, Bromotopsentin, Bromodeoxytopsentin, Deoxytopsentin, and any combination thereof.

63. The method of claim 62, wherein the method: i) reduces one or more viral response genes, ii) increases one or more immediate early genes, iii) increases a NF-κB signaling pathway, iv) increases a TNF signaling pathway, v) increases a IL-17 signaling pathway, vi) inhibits apoptosis, vii) inhibits viral defense and/or vii) increases histone levels.

64. The method of any one of claims 61-63, wherein the apoptosis is inhibited by altering a signaling pathway selected from the group consisting of a NF-κB signaling pathway, a TNF signaling pathway, a IL-17 signaling pathway, a JAK/STAT signaling pathway, a P38 MAPK signaling pathway, a PI3K-Akt signaling pathway, or any combination thereof.

65. The method of any one of claims 61-65, wherein the increased histone levels result in increased metabolic activity.

66. The method of any one of claims 61-65, wherein the increased histone levels result in increased host cell replication.

67. The method of any one of claims 61-66, wherein the host cell comprises an AAV capsid (Cap) gene.

68. The method of any one of claims 61-67, wherein the host cell comprises an AAV replication (Rep) gene.

69. The method of any one of claims 61-68, wherein the host cell is a HEK293 cell.

70. The method of any one of claims 61 to 69, wherein the topsentin alkaloid or derivative thereof is Homocarbonyltopsentin.

71. The method of claim 70, wherein the host cell is contacted with about 5 μM to about 40 μM of Homocarbonyltopsentin.

72. The method of any one of claims 61 to 71, wherein the host cell is contacted with the topsentin alkaloid or derivative thereof for at least about 24 hours, 48 hours, or 72 hours.

73. The method of any one of claims 61 to 72, further comprising harvesting the AAV particle.

74. The method of claim 73, further comprising purifying the harvested AAV particle.


SEQUENCE LISTING (66 sequences)

SEQ ID NO: 1 - DNA, 5786 bp, Homo sapiens
  source 1..5786: mol_type = genomic DNA; note = TRPV3 genomic
ttttatccaagctgtgcttgtgatactgtctgtcttcttgtacttgtttgcctacaaagagtacctcgcctgcctcgtgctggccatggccctgggctgggcgaacatgctctactatacgcggggtttccagtccatgggcatgtacagcgtcatgatccagaaggtgcgttgagagctgcgccggcgggagggccctcggaggatggccatttttcagatggggaaactgagatctggagaggaagaggggtttaccaaggggcaggtcactgctctttctcccaaatgccatgcactccctttgctttcggccaggctttaagtgtcaagaccttaaatcccccaggcttgtggggtgctggggaggggtacagtttgtggaggaatgttctcacccctgggcagtggcgtgctggtaaacatttaacgactgttctgtgtgtgtgtgtgtgtatgtgtgtgtgtgtaggtggaatatgtatattccatatatatacacaaaagacttatcatacattttactatataaacgatgtgtatcacacaatttgcaaataatgaaaatataccatgcagtttattgtaaattccacatagccagttgagtctcagaataatttgttgctttttttttttttttttggagaccaagtcttgctctgctgcccaggccagagtaaagtggtgcgatctcggctccctgcaacctctgcctcctgggttcaagcagttctcctgcctcagcctccagagtagctgggatcacaggcacgcaccaccatgttggctaatttttgtatttttagtagagacagggtttcagcatgttggccaggattgtcttgaactcctgacctcaggtgatccacctgcctcggcctcccaaagtgctgggattacaggcatgaaccaccgcgccaggcctatttgttgctttttattgaattctagtttttgttgtcaacttttggttgcaattgacaaaagagtgctatcagacatgaatgttggttgagagttttctttataagcagtgattgaagtgaaacaattaagatttatgttgaactttacttgtttatcaatgatgtgagcaacttctttgctgaattggatactagtttgcaaataccagaataatattctgttgatatttttgtgctatacataatgtaaaggctgcagacagaacacattttaaaatttaaactggattaatagtttctccatcactgtcttaaatcatttagatctagatatcaacaaaacaatgagtcaatcaatctctgatttgtggcatttaccaattttcatgatgtaaatacagtagtccccccttattcttgggggagatgttctaagaccccccatgaatgcctgaaactgcagatagtaccaaacattatataatatgctatgttttttcctatacatatacatctatgcaaataatgaaaatatactatacaatacagtttattgtaaattccacttagccagttgagtcttagaataatttttgctttttttttttttttttgtttgtgactgagtctcgctctgttgcccaggctggagtgcaatagtgcaatctcggctcactgcaacctctgcctcccgggctcaagcaattctcccgccccagcctcctgagtagctgggactacaggcatgcaccatgcccagctaatttttgtatttttagtagagatggggtttcgccatgttggccaggctggtctccaactcttgacctcaggtgatccgtccgcctcggcctcccaaagtgctggggggtgggggggggaatcaacaaattattctaagactcaactggctatgtggaatttataaaatttattttatcaattaggcatagtgagattaatggcaataactaacagaacaatgataacaatatactgtaataaaagtatgtgaatgttgtctctctcagaatattgtattgtgtgtaatatttctgggtcatagttgaccacaggcaaccaaaactgcagaaagtgaaatcgaagagaagggcggactactgttctcctctcagggccaatttccagctaccaacattatgttactggacatagagttagggagaaacatgcagatccacatcgctctagaattcctgccatatggctatgatagagacaagtaacctcaaaagcacacacaattgtaaatatagtgaaataactggacggggatgagttttgaacatttattgtctttgttttatttaattaaattaattaattaattaattttttgagacagagtctggctctgccccccaggctggagtgcagtggtgcgatctaggctcactgcaaccccttcctcctgggttcaagcaattctcccgcctcagcctctggagtagctgggatcacaggtgtgtgccaccacacccagctaatttttgtatttttagtagagacggggtttcgccatgttggccaagctggcctccaactcctgacctcaggcgatccacccacctcagcctctcaaagtgttgggattacaggcgtgagtcactgcaccctggcctgtctttgttttaaatataacttattttatttaattgtaagtttatatggtttaattttttggcaatacaaataataatttctttacccccagctttattgaggtacagctgacatatataattgtataaatttaagatgtacaatgtgaggatttgatataggtatatattgtgaaatgatttccaatttccaattaacacacccatcacctcacatagttgccatttctttgtgtgtggtgagaacatttatgatctattctcttagcaaatttctagtacacaacacggtattgttcactatagtcatcatgctgtccattagatccccagaacttattcatcttattcattgatttagttctgaataatggctgtgtttaacaaccagctcacaagcttcatgagaatttaacgatcagctcttgcgagctcttctgagctggctccagcatgccactgcccccagcaagtgtgaggattccagtgtgagaccctatggtgaaaggtacagggtgaatcacagggagaagagaacctggggctggagcagaaacagaaggcttcctggaggaggcaggatccagtgttgagccttggagcatcaggaggatttggctgggggaagggagagtggcccaagtgaagagacagttagggtggggggtgggagagggtgggaagcaggaagatcagggtgccgtgaatatgtgcttgcaccttacacttgaattatgtttatttccttataggtcattttgcatgatgttctgaagttcttgtttgtatatatcgtgtttttgcttggatttggagtaggtaattgatttataaataatagtaactcccacatttataaacactttatacagatgtcaaaatggcaaagtgcatggcggccttggcaacaattagaccaactaccagtggggcacttgccatgcttggcattttccaagcattacctttatatatatacatatatattttattatactttaagttctagggtacatgtgcacaacatgcaggtttgttacatatgtatacatgtgccatgttggtatgctgcacccattaactcgtcatttacattaggtatatctcctaatcaagcattacctttttacttctctcaacaacttgatcaagtgggaatcatcaccccgatcataaggtaactgaggcttagagactccaggattcaaaccaggccagccactcataatccaacttcaccatgtggtatatacctctggctttcccattcattgcttacgtggccctcacgacctaagataggttctgcaagtgttttcagccccattttaacagatgcggaaactaaagtccacagaaactcaactgatggtctttgcaaagtaggagcaaactaataccctgaggtgatttcctttggatttcacatcacgttactctcacagtttgctttagatacaactgtgagagcctctctggtctgctgggaccaggaaggagccccctcttaaaatctctccatttacaacagagggaacagcacgatctggttgtatttctaacctctgcatagagagatatcattttcccccacgtaccagttgcttttttttcttttcttcagtgaccattgacatttgaaattaagtagctgtttccatctgtctggtaccttttctgggtcacaatttgcttatttatttgctccttcacctgcttgtttattcatccatctatttactctctcatttaatttgctcatccacctgtctatccgtccatccatccatccatccatccatccatccatccatccaatcattcatccatcgatccatccattgatctatccatctatccatccatccatccatccatccatccatctaatcatccatccaatcatccatccatccatccatccatccatccatccatccagtcatccatccattcatccatttaatcatccatccatcgatcaatcggtccatccatccagctatccatccatccatccatccatctaatcattcatccatcaatccatccatccatccatccatccatccatccatccatccatctatccatccatccaatcatccatccatccatcgatcaatcagtccatccatccatctaatcatccatctaatcatccatccatccatccatccatccatccagtcatccatccattcatccatttaatcatccatccatccatcgatcaattggtccatccatccatctatccatccatccatccatccatctaatcacccatccatccatccatccatccaatcatctatccatccatccatctaatcatctatccatccatccatccatccatccatccaatcatccatccatccatccatccatccctccatctaatcatccatccctccatccatccctccatatccatccatccatccatccatccatccatccaaccattcagcagtgaacctctactgtggtgtgttggaaagaagggaagtgtgagcctggtcttgctcacagggaactcaaaacagagcattaggaggagactcaggatcttacagccttgagggaggtgacccaggctagaggaagtcaggagctcaaagactagctggggagccagggacacacacatggattataattagtggctctgcaaggctgcaagtgatcaagggcagactgcggggtaaaggaaggagagaagactctgggctttgttaggtggaagggctttctgcaggaggccaggccagagaagccagcctgatgaggctttgagaggacctgaaactggagatgggatttaatggccagagagaaggaggcagcaggcttgtcatgtaggcaaatacacgcaagcttgtagagtaattaccaggcagagagctgaggagagcaccctgttatgttcccttagccttggcctcgctgatcgagaagtgtcccaaagacaacaaggactgcagctcctacggcagcttcagcgacgcagtgctggaactcttcaagctcaccataggcctgggtgacctgaacatccagcagaactccaagtatcccattctctttctgttcctgctcatcacctatgtcatcctcacctttgttctcctcctcaacatgctcattgctctgatgggcgagactgtggagaacgtctccaaggagagcgaacgcatctggcgcctgc

SEQ ID NO: 2 - DNA, 1447 bp, Homo sapiens
  source 1..1447: mol_type = genomic DNA; note = STK31 genomic
aactctggtggtctccttacaatgagcttggaacgagatcttcttgatgctgagcccatgaaggaacttagcagcaagcgtcctttggtacgttctgaggttaatgggcagataattctgttaaaggtaagtctaacttcttcttacttgtgggagattcagcagcataaaggatagtggtgctgttacatttcaagaaagaaaaaacctcactgttagttttctaaagtgtataagttgactggcatgataattgtgcttgtagtttggttgccagaactaattttcacttttaaagtttccctcaattgaacaatgagaacacttggacacagggcggggaacatcacccgtgggggcctgtcatggggtggggggcagggggagggatagcattaggagaaatacctaatgtaaatgatgagttaatgggtgcagcaaaccaacatggcacatgtatacatatgtaacaaacctgcacgttgtgcacatgtaccctagaacttaaagtataataataaaaataagtatataaaagtcattaaataaagctttgtctcacaagaaaaaaaaataaagtttccctcatttccctaaatagaatatgtccttaataggatatactttcctttttttttgggtgaacttattatagtcatccaatatctttttaaaattcttgattttcttacatttctccttgaaatctcagctgtgttatcagtacaatcttctactctttaatttcttcttctcattgatctgggaaactgttaatcatgataataagataattaatttggccttagttgctacacattctagaatgtgtagcaggctacaggtagaattgcactttccttttccaaattatagcattcaaagaagatagaaaagtacttataaaattaaaaaataaaattagatttgatttaacttaaaataatgaattatgtttaaattggaatgatgtataattataatgagattttcatcttggaattacgagtgcctctttctttggtacaagggctattctgtggatgttgacacagaagccaaggtgattgagagagcagccacctaccatagagcttggagagaagctgaaggagactcagggttactgccattgatattcctgtttttatgtaaggtaaagttcttatgctaatctcttgcagaaaccattttatcttgcagaaactattttatttaaaataaattttagctccaggttttagagcagcagttcattccccttaacataaaagaaaaattatcccatagaatcaatgtatgctaaaaatatgttgaagaagtttttacttggagtcacattctctgttttgcttcttagtctgatcctatggcttatctgatggtcccatactaccctagggcaaacctgaatgctgttcaagccaacatgcctttaaattcagaa

SEQ ID NO: 3 - DNA, 3882 bp, Homo sapiens
  source 1..3882: mol_type = genomic DNA; note = USP25 genomic
gtgtcacaacataatgaaagttgctcaagccaaactggaaatgataaaacctgaagaagtaaacttggaggaatatgaggtaatgtgctttcttagaggttaaactacagatttagggattctgtaattcactgatattcctttcgtttcagcccaactgaagttgaatttgttatgaatgatgagtaaaaaaattaagtttccaagtcaatgctcaagtaattaataattttatatgtgattttaggattaacatgatatgatagcatgtagaccaaaaagtttaaaatcaaaaaaggaagatttttggttttttatttcaatactaattctcaagtactttttaaatgaacttttctagcttaaaaacagaataacaaatgcattgttaattttgaaatcaacttagaaattgctgttacataataccaacaagtaatatttatcaagaccctactatatgccaggcatttgtctaagccctttacatatattaactgatttaattcagacagtaacactgtaagataggtagtgttcttattcccatttgacacacgagaaaataaggcatagagcagtgaagtaacctgcctaaggttttcacagccacaagtttacagcttggctaatagaacctacattcttaactgttgcactacactgtgcaactgtaccctgtgggctgtattttttccttcactttgattcatcagatatattttggggaagttaattcattttagtccctatatgccatatatgtttgtcatgagacagtgttgtgtgtgagagagcacttacataggtacttgtacatgtgcttgtgggatttttttgtgcacaaaattattttttgtcaactttggctttaaaatattctttattttaattgatgaattcatacacctagtatttcttcacatttttaaatttaatcttatgcattgtcttttttttaatttataagtctagtaaattttaagttgttttcttgggtgcctttgcttaatatttagttccatttacaaacttaattatggtactttagattccctaaataattagtgtaattcactacacaaacagggaagaggaaagagagagaaaacactcattattgatgaaaaccttagctagtttggcgtataaggtaactaccttaattcactaaagagtgtctctaaaaacaaaaataggaaataataaaactgcagcaaatatatagaattgtgagcactgaaagcttaaaatttttgttgtcaaaagcaaggcaatgaaaataagcgaaaagtaaaatactggaaaagaagaaacaaagacatcattatttgtagttttttttaccttgaaattcaaataaaatctacaactgatgtattagaactagtaagacagctttgcagcatgcaaacttgtgtcagacatcaattttattcccttttgccagtaaaaaaagtatgagaaagtataaaagtgagaccatttacaatagtatctgaaactatggagtatatagaaataaatttaacaaaaggtgcgtgtgacttctgtggggaaaattataaaacttattgggagacattaacaaagacctaaataaatgggcatacatatggtatttggaaaacttgatattattaatatatagatatcacttttcccccaaatgctttataaattcagtgcaggctgtatgaaaattccagcaagatgttttatggagcttgataagccaactgaaaaatgttacggaaatgcaaagatcccagtccacctcccatctctgacaagcaagttagtgaacagtataaagactttttaataaatggttctgggaaataaataaaatttgatttcttctttattccatatgcaaaaattcatttcaattgtgaaagactcctgtgtaaagctgtttagaagtcaacataggtagaagcccaaagacattcatttatccataagaggcactcactgaccagatatttttgaccaggatatgtaatctagagccagagaaattcaaaatttgttttctcttgaccctgaggagcaagtcaagtcatcttttagctaataatgactccaaatctgtgccctccacattgattgagagctcttcccttgtccccaagattcgtgttcatggaacttgatttttaattgtgacttgattctgcaatgcttccttcagtttgcttcaacagattggactcattatgttttttttcataaaatcttctcatattctaactgcagggctgttaaatcatacaagtatttaaatgacttgtttttagattctgcatattcctcctatctttgtcttgctctaagttcagggttcagtaaaccatagcctgcagaccagcttaccacctgtttttgtaaataaacttttattggaacatggccatttgtatacatattgtctgtggatactttcacactgcagttacagagttgagtaactgaagaagatgaggcttgcaaagtctaaaatattatctggccttttacagaaaaagtttgcctactcacaacttctaaggtgattctaatgacaacaaggaatgacagtttgaataccttccccagaaaagctaacttcatacttgcaggacaaattttacctttatttgttatgaagatctactaatagcatgaaaattatgtcttgaaaattaaaattaagactggatgtggtggctcatgcttataatcccagcacttttggaaatcaaggcacagggagttgtttgaggccaggaatttgagaccggcctggccaacatagcaagaccctgtctatacaaaacagattaagtaattaactggaactgggagcatgcacctgtaattcaagctacttgggagacagaggtaggaggattgctggagcccagaaggtcaagtctgcagttagccgtgatcgtgccactgcactgcagactgggcaactgagtgagaccctgtctccaaaaaaaaaaaaaaaagtttatattcaaggagaatatcgttggctaataactatgtttcttatttttgtttataatgtttatataaaagccttaacacttttgttatcagtattcactttccaatttatcctacattgaatttcagttatttttcttgagtcaattattataaagtaagctaagaaaatcttgactttggtatcaccttagaattatttagtcctattacatgccctatagttgttttgggggtggggaggggcattgaaatgtgaactgcagttattcttttaccatatctcatgtaaataaactattcgcctacacacttaaagtcaaatttaccacatatttgcgaagttaaaaggttaaataaatctaactttttttagtttcttaaaattatgtattaagtatgtgtccaagctttgattcatactcagcgtaatatttcattttcttccatcagttgatgtagatataataatagttcaataatatgattttatactgatgaatttttgagatgacctgattgattatcctgcctttatatttctgttatctgctagtaaaatctttattgtttaaaacatttcatatccatgcctaataatttttagatttaatgttgtattaataaatagtcttgtgtgccagaaagtaaagtcttgttttagttctgatgtatcttcattttgaactctttatttctattaattcagatagtagcgaaacagtgcattactttttgtacagtttcatagtactttttaaatcttactctgaacatttttaatattttctttcacctacaggagtggcatcaggattataggaaattcagggaaacaactatgtatctcataattgggctagaaaattttcaaagagaaag

SEQ ID NO: 4 - DNA, 2806 bp, Homo sapiens
  source 1..2806: mol_type = genomic DNA; note = GTF2B genomic
gatcgaacaaataatttattcaagcaagtatatgaacagaagagcctgaagggaagagctaatgatgctatagcttctgcttgtctctatattgcctgtagacaagaaggggttcctaggacatttaaaggtaagtttttagctttgtttttttgtgtttgtttgtttgtttgtttgagatagggtctcactttgtcgcccaggccagagtgcagtggcgcaatcttgactcactgcaacctctgcctcccaggttcaagctatactcctgcctcagcctcctgagtagctgggattacaggcatgtgccaccacacctggttaacttttgtatttttagtagagacagggtttcgccatgttggccaggctggtctcaaacttctgacctcaggtgatctgcccactttggcctcccaaagtgctagaattaacaggcgtgaggcactgtgcccatcctaaaataacattttctacatcatattaaagaaacagaccaatgtcaggcagctttgacacttagaagctaagtcatctttattgtgactacagtaatccacttgagcatcatttgccaatgcctacttatttcattccttgacagcaaattcatagcatgttcttttaggctgttttcccatgtagactcattttagtccacgtaagcccactatgttcttttctagtgagatcttttgatggactgttgctttgtgggctttcagaaggcctataatcgtaaaccaaagtaggccgtttatacatttggtttacatggaaacagtttccatttctagcactggtctctgtagatgagggagtcaagctttagaaagtatcagtgaaggccaggtatggtgtctcacacctgtaatcccagaactttgggaggccgaggtggttgggtcatgaggccaggagttcaagaccagcctggccaatatggtgaaaccccgtctctactaaaaatacaaaaattagccaggcgtggtggtgcgcgcctgtagtcccagctactcaggaggctgaggcagaagaatcgcttgaaaccgggaggcagaggttgcagtgagccgagatcacgccactacactccagcctgggcgacagagtgagacttcatctcaaaaaaaaaaaaaaaaaagttatcagttaagttttttctcttgaggtgtattgtttgcacttttacacttcagttaagaactggtatttttcatttagtttgcagttacatgtgaatatgtgataatttgttcagtgttgctcactgacttccactaccaaagctcagtttagctgaagactatacttaaccctttattcatccagtgagtatttattttttatgttttcttacgtaaaagtgaagaggtgatggcctttatcatcatcttttttgttgtttttataactaaaacaacttttgttaaatttcttctgaaagtgtgtggtggttgcatctgggaatagcagggtcagatattttatctcattgagctacagcccataatcgcagggatttcatgcacacatgacattgtgttgcctaagaggcatcctttagcttgagtattttctttcaattccttggtaacactgcttacaacagattaatacataaacatcaaagaatttttcatttaaagaaatttagttgtatgaattaatccttagagcagttaatattttgaaagattaagacctaaatttagccaggcacgatggctcacaccggtaatctcagcactttgtgaggccgaggcaggtagatcacgaggtcaggagttcaagaccagcctggccaagatggtgaaaccccatctctactaaaaatacaaaaaattagccgggtgtggtggcaggcgcctgtaatcccaactactcgggtggcaggagaatcgcttgaacccagggggcagaggttgcagtgagccaagatggtgtcactgcactccagtctggacgacagagtgaaatttcatctcaaaaaacaaaaaaactctaaattgaaagagtgaagaacattaggccaggcgcagtggctcatgcctgtaatctcagcactttgggaggccgaggtaggaggatcacctgaggtcaggagttctagacaggcctggccaacatggtgaaaccccatctctactaaaaatacaaaagttagccaggtgtggtggtgcacgcctgtagtcccagctactcgggaggctgaggcaggagaatcacttgagcctaggaggtggaggttgcagtgagctgagatcatgccactacccttagtctgggcgacagagtgagacttcatctcaaaaaaaaaggtgtgatgaacattaaagttaactgagaaatttgcagttaactctgtgggaagtatcactgagatgattagggtttcatctttaaggtatgattgaatgttttacaattagaatgtatattatgtttataattagaaatggtagattttaagatgaactatatggcttaagtgaattgatttatttaattttttttcttttagaaatatgtgccgtatcacgaatttctaagaaagaaattggtcggtgttttaaacttattttgaaagcgctagaaaccagtgtggatttgattacaactggggacttcatgtccaggttctgttccaacctttgtcttcctaaacaagtacagatggcagctacacatatagcccgtaaagctgtggaactggacttggttcctgggaggagccccatctctgtggcagcggcagctatttacatggcctcacaggcatcagctgaaaagaggacccaaaaag

SEQ ID NO: 5 - DNA, 1973 bp, Homo sapiens
  source 1..1973: mol_type = genomic DNA
atggaccccgccaggaaagcaggtgcccaggccatgatctggactgcaggctggctgctgctgctgctgcttcgcggaggtgggtctgctgcccggagaggtggagggggtagagtgggggctcttagggaggaggctcctctggccccgtacagagaaggctgtgggaaggatccccacataagcagagggcactgtgggcaaagttcccccatcctaagcaagggaggcagagggaagggtcctccaccatgggccgtaggaagggttccccaccacggacaaggggagctgtgggcaaaattttcctatcctgaagctgcagaaagaagtcccctgttcctaggccgggcgcggtggttcatgcctgtaatctcagcactttgggaggccgaggcaggcggatcatgaggtcaaacgatcgagaccagcctggccaacatggtgaaaccctgtctctactaaaaatacaaaaattagctgggcgcggtagcgcgagcctgtagtcccagctactcgggaggctgaggcaggagaatcgtttgaatccaggaggggaggttgcagtgagccgagattgcgccactgcactccagcctggcgacagagcgagactccgtctcaaaaaaaaaaaaaagaaaaaaagaaaaaaagaaaaaagtgttccctgtcctgggcagagaagctgtgggtagtgttctctcatttgagcagaagggggctgtgggaagggatccctcactgagcacagtctactgtgggaaaagttctcccacacactgtgggaaaagttctctcattctgagcagaaaaccgtgaggaggtatccttcctaccccgggatgaggggcttcatggcggggatcgcctacagtgggcatacagggctgtcggaaggatgtgcccacagttggcatagtgggtgtgaaggaaaagggtccagtgattcccgtgcttccagatttgtggaaagcttttccctactcctgccctggaaaagtgtgtgcaataagactgtctaatcccgggacagagggcatgggaaggactatttctccttgggggtggacgctttagcaaggagggctcaggtctattcggctccgctcccccaggagcgcaggccctggagtgctacagctgcgtgcagaaagcagatgacggatgctccccgaacaagatgaagacagtgaagtgcgcgccgggcgtggacgtctgcaccgaggccgtgggggcggtggagaccagtgagtggggacccccgggccacgcacaccactgcaggctcctccggcccagccccgcctccttatagtcctttctggatctggccacgcccctttaacgcggccccacccctccctttcagcctggccgggaccctacgacacagccttgcaggccaggccttctctattaacaattacaagcgcgaggagcagagagttgagaactgggtggggcggacggggagagctcgggtttggaacgaggtcacgtcctatgcactttttctgctccaccaatgagacctccccatccctgcctgtcaaccaggctgtctcctccgactcagctctttgaggttccgcccccgttccgtgaccccgctcctccctgcttcggtcgttctttccgctctgagcggctctttgggggcgtccatccgccaattcctgcctgtcggtcctgtggtgttggcctatttgaggccccgcccctcccagaccctacccaactctgggccacgccccatctgagaccacgcccacagctaatcccgcccttgtccctctccctagtccacggacaattctcgctggcagtgcggggttgcggttcgggactccccggcaagaatgaccgcggcctggatcttcacgggcttctggcgttcatccagctgcagcaatgcgctcaggatcgctgcaacgccaagctcaacctcacctcgcgggcgctcgacccggcag

SEQ ID NO: 6 - DNA, 3336 bp, Homo sapiens
  source 1..3336: mol_type = genomic DNA
gtgagccgttctccaaccctttggcccccgatggccacgatgtggatgatcctcactccttccaccagtgagtattttgtgctaggaccatggaaatgagttgggcaatgaatagaaaagtggtggtgatggtgaaagaattctgaatgtcttgtcatcaaggatggtaaaaaattatacccgctggccctttatctctgtgagccttaactggatcaggccaagtgcataagggtattagaagggaaattcctgcaggaaaggtgttaagtttttttttttttttatacgtgttgtgtaattacacattatataggaattacttcttccaatagcaaattaaaacataggaaataagggtaaaatcatctttttattttttttgagactgagtttcgctcttgttgctcaggctggaatgcagtggtgcgatctcggctcaccacaacctccgcctcccgggttcaagcaattctcctgcctcagccttccgagtagctgggattacaggcatgttccactacgcccggctaattttgtatttttagtagagatggggtttctccatgttgatcaggctggtcttgaaatcccgacttcaggtgatccacccgcctctgcctcccaaagtggtgggagtacaggcgtgagccaccgtgcctggcccccgataaaatcatctttgataacatccccaattccagtccatttctttttttttttcttttcttttcttttctttctttttttttttttttgagacggagtctcactgttacccaggctggagtgcagtgatgcaatcttggctcactgcaacctctgcctcctgagttcaagcggttctcctgcctcagcctcccaagtagctgggactacaggcatgtgccaccatgcacgactgatttttttttttgtatttttagtagagacagggtttcactgtgttcgtcaggctggtcttgaactcctggcctgaagtgatccacccgcctcagcctcctaaagtgctggcattacaggcgtgagccaccgtgcccagcctcagtccttttcttagaagtaaccacagttaccttttttggcatatcttttcctcccgttgcccaggctggagttcaatggcattatttctgctcacagcaacctttgcctcctgggctcaagcaattcttctgcctcagtttccccagtagctgggattacaggcatgccccaccatgcttggctaatttttgtattagtagagacagggtttcgctatgttggccaggctggtcttgaactcctgacctcaaatgatctgcccgtctcggcggcccaaagtgctgggattacaggcgtgagccactgcgcctggccctcctactcttttttaaatgcatttatacacatacatgcatatctgaggaaaacacaacattgctttgtgatttaaaatatttgagttaaatgatattatgctgtatatatcatttttgcaacttgcttctttttaaaccctgctcagtgtttctgagattatctttcagagcagtgacaagcattccctccttggctaaactttagtcaggttccccaaagagtcccatttttcagcaggactcatccgtggcctgctgagcacagttttaacaaagaaatcaaccaccgctccacccccatacctaaccaagttgcttttagtaattttctataaattctcacttgtccctgttgtattcggaattaagttcagtctctctactctattacaatggtcttgactcttgttacagtagtcttcaataaagtcttccttggggagaagaggaacttgattagatatcaagttggattggatatcaagagtgggaaaactttctgtgaactgacttaggattcctttttaaaactgaggtcagcaggccaaggaagagccctagtcagaagagagctttaaaggagtctgactttaatttgatcaagagaggagtctttgttagtgtaccaattaaaataatctcttgtaccattagtgctgtctttccctctttgggaaacaccatcatagtttagtcagtctccagctaataaacgcttagtctgtttcagactgattcttttcttttttttatttttttgagacagagtcttgctctttttgcccaggctggagtgtaatacctagatctcggctcactgcaaactccgcctcccaggttcaagcgattttcctgcctcagcctcctgagtagctaggattacaggcgcccaccacaacgcccagctaatttttttgtatttttagtagagatgggatatcaccatgttggccatgctggtcttgaactcctgacctcgggtgatccgcctgcgtcagcctcccaaagtgcggcgattataggtgtgagccactgcacccggccagactgattattttcaaggctataactgaattaaatttacaggccttcttatatacatatgtcagtaatggaccaggctaaatataaataaatggagttgctggctcataggatatatatacttaaattttaacaaatactgcaaaattgcccttcaaattggttatactggtttacattctcatcagcattatatgagagagtccccttcccaaattctcactcacacttgatttatcaaactttctggtagttaacactctgatgcattaaaagtggcatggcatatctggtcgggcgtggtggcttacacctataatcccagcactttgggaggctgaggtgggcagatcacctgaggtcagaagtttgggaccagcctggccaacacctctaataaaaatacaaaaattagctgggtgtgttggcgggcacctgtattcccagctacttgtgaggctgaggcaggagaattacttgaacccaggaggcagaggttgcagtgggctgagatcatgccactgcactccagccggggcaacagagggagactctgtcttaaaaaaaaaaaaaaaaagctgggtgcggtggctcatccctataatcccagcactttgggaggccgaggtgggtggatcacaaggtcaggagttcaagaccagcctggccaatatgggtgacagtgagactccgtctcaaaaaaaaaaaaacagtgtcatatctttgttttaatttgtatttccctgatttctgttaatttaatcatgatttttacttgttcctttttcatttcttattgagtcctaatatttaaaatctttgccttttcttctagatcaaaactcaccaatgaagacttcaggaaacttctcatgacccccagggctgcacctacctctgcaccaccttctaagtcacgtcaccatga

SEQ ID NO: 7 - DNA, 1583 bp, Homo sapiens
  source 1..1583: mol_type = genomic DNA; note = TEX 14 genomic
tggaggtgaaagaaatggcaaagaaagcagctactggacagctcacagtacctccttggcatcctcagagtagtctgactttagagagcgaggctgaaaatgagcccgacgccctgctgcagccccccattaggagcccagaaaacacggattggcagcgagttattgagtatcatagggaaaatgatgagcccagaggaaatggcaagtttgacaagacgggcaacaatgactgtgacagtgaccagcatggcagacagcccaggcttggaagcttcaccagtaagttcacttgcgggaatcaggataggtgaactcaatcaagtatcagtcgggcgaggtggctcagcctgtaattccagcactttgggaggtgaggcgagcggatcacctgagcccaggaatttgggaccagcctggtcaacatgatgaaacctggtctctacaaaaaaaaaaaaaaaaaaaaaaaaaacattagctgggcgtggtggcacacgcctctagttccaggtacccaggaggctgaggtgggaggatcacttggacctgagaggtggaggttgcagtgagctgtgatcatgaaactgcactccagcttgggtgacagtgagaccctgtctcaaaataaaaataaaagtaaaaaaaagttatcagctctcaaaaaagtaaactttccagcatattttttttggtttggtcctctctcctttcctacttttctggaaaccttgaattccaggggaagacagtatagccctatatctcattgcatttcccacaggcaaacagatccagacactgatcagtttttatattactgaaacaggtccctttgtccaaaggaagtaaaataatcaaatactgaccctggttttggcgaggaatcgatccttctttacctggggatagaggtaggccatcactgcctactgcccatgtctctgcattgtattctaagctgaaattaaattctcccagaatttatttagaatgtattctaagctggcattaaatttttccagagagtctatgctggaggaacctagagacacatgacatttattgtcatcggtttatagatttacaggttaaaaaaaatgaaggctgtatattttcagttgataatttgcttataatcagggcatggatattgtttcacagttgtttataagttaaagaaagatacatctaaacttgtcatttgtcttaggcagaaaatctattggcttgggtattatttgtatttagaatctgtttcttgtcaagcatcactctcgaatgggtgggcagatgatccagggctgcattttggggtcaggctaaactaacagtctctactctctggactgaacctaacgtatgggacatggccactcttggcagaatatgtatacatctcttctttcatgccacctttatcctcttccatgttgtctgaatatcactgaatgactttgaccctgactgtgttacaatcattaggtatcaggcacccatctcccagacaaaaggagcaaccagagcatagtgaagccttccaagcaagttctgacacattggtggctgtagagaaatcttacagt

SEQ ID NO: 8 - DNA, 166 bp, Synthetic construct
  source 1..166: mol_type = other DNA; note = TRPV3 upstream exon
ttttatccaagctgtgcttgtgatactgtctgtcttcttgtacttgtttgcctacaaagagtacctcgcctgcctcgtgctggccatggccctgggctgggcgaacatgctctactatacgcggggtttccagtccatgggcatgtacagcgtcatgatccagaag

SEQ ID NO: 9 - DNA, 3158 bp, Synthetic construct
  source 1..3158: mol_type = other DNA; note = TRPV3 upstream intron
gtgcgttgagagctgcgccggcgggagggccctcggaggatggccatttttcagatggggaaactgagatctggagaggaagaggggtttaccaaggggcaggtcactgctctttctcccaaatgccatgcactccctttgctttcggccaggctttaagtgtcaagaccttaaatcccccaggcttgtggggtgctggggaggggtacagtttgtggaggaatgttctcacccctgggcagtggcgtgctggtaaacatttaacgactgttctgtgtgtgtgtgtgtgtatgtgtgtgtgtgtaggtggaatatgtatattccatatatatacacaaaagacttatcatacattttactatataaacgatgtgtatcacacaatttgcaaataatgaaaatataccatgcagtttattgtaaattccacatagccagttgagtctcagaataatttgttgctttttttttttttttttggagaccaagtcttgctctgctgcccaggccagagtaaagtggtgcgatctcggctccctgcaacctctgcctcctgggttcaagcagttctcctgcctcagcctccagagtagctgggatcacaggcacgcaccaccatgttggctaatttttgtatttttagtagagacagggtttcagcatgttggccaggattgtcttgaactcctgacctcaggtgatccacctgcctcggcctcccaaagtgctgggattacaggcatgaaccaccgcgccaggcctatttgttgctttttattgaattctagtttttgttgtcaacttttggttgcaattgacaaaagagtgctatcagacatgaatgttggttgagagttttctttataagcagtgattgaagtgaaacaattaagatttatgttgaactttacttgtttatcaatgatgtgagcaacttctttgctgaattggatactagtttgcaaataccagaataatattctgttgatatttttgtgctatacataatgtaaaggctgcagacagaacacattttaaaatttaaactggattaatagtttctccatcactgtcttaaatcatttagatctagatatcaacaaaacaatgagtcaatcaatctctgatttgtggcatttaccaattttcatgatgtaaatacagtagtccccccttattcttgggggagatgttctaagaccccccatgaatgcctgaaactgcagatagtaccaaacattatataatatgctatgttttttcctatacatatacatctatgcaaataatgaaaatatactatacaatacagtttattgtaaattccacttagccagttgagtcttagaataatttttgctttttttttttttttttgtttgtgactgagtctcgctctgttgcccaggctggagtgcaatagtgcaatctcggctcactgcaacctctgcctcccgggctcaagcaattctcccgccccagcctcctgagtagctgggactacaggcatgcaccatgcccagctaatttttgtatttttagtagagatggggtttcgccatgttggccaggctggtctccaactcttgacctcaggtgatccgtccgcctcggcctcccaaagtgctggggggtgggggggggaatcaacaaattattctaagactcaactggctatgtggaatttataaaatttattttatcaattaggcatagtgagattaatggcaataactaacagaacaatgataacaatatactgtaataaaagtatgtgaatgttgtctctctcagaatattgtattgtgtgtaatatttctgggtcatagttgaccacaggcaaccaaaactgcagaaagtgaaatcgaagagaagggcggactactgttctcctctcagggccaatttccagctaccaacattatgttactggacatagagttagggagaaacatgcagatccacatcgctctagaattcctgccatatggctatgatagagacaagtaacctcaaaagcacacacaattgtaaatatagtgaaataactggacggggatgagttttgaacatttattgtctttgttttatttaattaaattaattaattaattaattttttgagacagagtctggctctgccccccaggctggagtgcagtggtgcgatctaggctcactgcaaccccttcctcctgggttcaagcaattctcccgcctcagcctctggagtagctgggatcacaggtgtgtgccaccacacccagctaatttttgtatttttagtagagacggggtttcgccatgttggccaagctggcctccaactcctgacctcaggcgatccacccacctcagcctctcaaagtgttgggattacaggcgtgagtcactgcaccctggcctgtctttgttttaaatataacttattttatttaattgtaagtttatatggtttaattttttggcaatacaaataataatttctttacccccagctttattgaggtacagctgacatatataattgtataaatttaagatgtacaatgtgaggatttgatataggtatatattgtgaaatgatttccaatttccaattaacacacccatcacctcacatagttgccatttctttgtgtgtggtgagaacatttatgatctattctcttagcaaatttctagtacacaacacggtattgttcactatagtcatcatgctgtccattagatccccagaacttattcatcttattcattgatttagttctgaataatggctgtgtttaacaaccagctcacaagcttcatgagaatttaacgatcagctcttgcgagctcttctgagctggctccagcatgccactgcccccagcaagtgtgaggattccagtgtgagaccctatggtgaaaggtacagggtgaatcacagggagaagagaacctggggctggagcagaaacagaaggcttcctggaggaggcaggatccagtgttgagccttggagcatcaggaggatttggctgggggaagggagagtggcccaagtgaagagacagttagggtggggggtgggagagggtgggaagcaggaagatcagggtgccgtgaatatgtgcttgcaccttacacttgaattatgtttatttccttatag

SEQ ID NO: 10 - DNA, 67 bp, Synthetic construct
  source 1..67: mol_type = other DNA; note = TRPV3 alternatively spliced exon
gtcattttgcatgatgttctgaagttcttgtttgtatatatcgtgtttttgcttggatttggagtag

SEQ ID NO: 11 - DNA, 2122 bp, Synthetic construct
  source 1..2122: mol_type = other DNA; note = TRPV3 downstream intron
gtaattgatttataaataatagtaactcccacatttataaacactttatacagatgtcaaaatggcaaagtgcatggcggccttggcaacaattagaccaactaccagtggggcacttgccatgcttggcattttccaagcattacctttatatatatacatatatattttattatactttaagttctagggtacatgtgcacaacatgcaggtttgttacatatgtatacatgtgccatgttggtatgctgcacccattaactcgtcatttacattaggtatatctcctaatcaagcattacctttttacttctctcaacaacttgatcaagtgggaatcatcaccccgatcataaggtaactgaggcttagagactccaggattcaaaccaggccagccactcataatccaacttcaccatgtggtatatacctctggctttcccattcattgcttacgtggccctcacgacctaagataggttctgcaagtgttttcagccccattttaacagatgcggaaactaaagtccacagaaactcaactgatggtctttgcaaagtaggagcaaactaataccctgaggtgatttcctttggatttcacatcacgttactctcacagtttgctttagatacaactgtgagagcctctctggtctgctgggaccaggaaggagccccctcttaaaatctctccatttacaacagagggaacagcacgatctggttgtatttctaacctctgcatagagagatatcattttcccccacgtaccagttgcttttttttcttttcttcagtgaccattgacatttgaaattaagtagctgtttccatctgtctggtaccttttctgggtcacaatttgcttatttatttgctccttcacctgcttgtttattcatccatctatttactctctcatttaatttgctcatccacctgtctatccgtccatccatccatccatccatccatccatccatccatccaatcattcatccatcgatccatccattgatctatccatctatccatccatccatccatccatccatccatctaatcatccatccaatcatccatccatccatccatccatccatccatccatccagtcatccatccattcatccatttaatcatccatccatcgatcaatcggtccatccatccagctatccatccatccatccatccatctaatcattcatccatcaatccatccatccatccatccatccatccatccatccatccatctatccatccatccaatcatccatccatccatcgatcaatcagtccatccatccatctaatcatccatctaatcatccatccatccatccatccatccatccagtcatccatccattcatccatttaatcatccatccatccatcgatcaattggtccatccatccatctatccatccatccatccatccatctaatcacccatccatccatccatccatccaatcatctatccatccatccatctaatcatctatccatccatccatccatccatccatccaatcatccatccatccatccatccatccctccatctaatcatccatccctccatccatccctccatatccatccatccatccatccatccatccatccaaccattcagcagtgaacctctactgtggtgtgttggaaagaagggaagtgtgagcctggtcttgctcacagggaactcaaaacagagcattaggaggagactcaggatcttacagccttgagggaggtgacccaggctagaggaagtcaggagctcaaagactagctggggagccagggacacacacatggattataattagtggctctgcaaggctgcaagtgatcaagggcagactgcggggtaaaggaaggagagaagactctgggctttgttaggtggaagggctttctgcaggaggccaggccagagaagccagcctgatgaggctttgagaggacctgaaactggagatgggatttaatggccagagagaaggaggcagcaggcttgtcatgtaggcaaatacacgcaagcttgtagagtaattaccaggcagagagctgaggagagcaccctgttatgttcccttag

SEQ ID NO: 12 - DNA, 273 bp, Synthetic construct
  source 1..273: mol_type = other DNA; note = TRPV3 downstream exon
ccttggcctcgctgatcgagaagtgtcccaaagacaacaaggactgcagctcctacggcagcttcagcgacgcagtgctggaactcttcaagctcaccataggcctgggtgacctgaacatccagcagaactccaagtatcccattctctttctgttcctgctcatcacctatgtcatcctcacctttgttctcctcctcaacatgctcattgctctgatgggcgagactgtggagaacgtctccaaggagagcgaacgcatctggcgcctgc

SEQ ID NO: 13 - DNA, 126 bp, Synthetic construct
  source 1..126: mol_type = other DNA; note = STK31 upstream exon
aactctggtggtctccttacaatgagcttggaacgagatcttcttgatgctgagcccatgaaggaacttagcagcaagcgtcctttggtacgttctgaggttaatgggcagataattctgttaaag

SEQ ID NO: 14 - DNA, 904 bp, Synthetic construct
  source 1..904: mol_type = other DNA; note = STK31 upstream intron
gtaagtctaacttcttcttacttgtgggagattcagcagcataaaggatagtggtgctgttacatttcaagaaagaaaaaacctcactgttagttttctaaagtgtataagttgactggcatgataattgtgcttgtagtttggttgccagaactaattttcacttttaaagtttccctcaattgaacaatgagaacacttggacacagggcggggaacatcacccgtgggggcctgtcatggggtggggggcagggggagggatagcattaggagaaatacctaatgtaaatgatgagttaatgggtgcagcaaaccaacatggcacatgtatacatatgtaacaaacctgcacgttgtgcacatgtaccctagaacttaaagtataataataaaaataagtatataaaagtcattaaataaagctttgtctcacaagaaaaaaaaataaagtttccctcatttccctaaatagaatatgtccttaataggatatactttcctttttttttgggtgaacttattatagtcatccaatatctttttaaaattcttgattttcttacatttctccttgaaatctcagctgtgttatcagtacaatcttctactctttaatttcttcttctcattgatctgggaaactgttaatcatgataataagataattaatttggccttagttgctacacattctagaatgtgtagcaggctacaggtagaattgcactttccttttccaaattatagcattcaaagaagatagaaaagtacttataaaattaaaaaataaaattagatttgatttaacttaaaataatgaattatgtttaaattggaatgatgtataattataatgagattttcatcttggaattacgagtgcctctttctttggtacaag

SEQ ID NO: 15 - DNA, 126 bp, Synthetic construct
  source 1..126: mol_type = other DNA; note = STK31 alternatively spliced exon
ggctattctgtggatgttgacacagaagccaaggtgattgagagagcagccacctaccatagagcttggagagaagctgaaggagactcagggttactgccattgatattcctgtttttatgtaag

SEQ ID NO: 16 - DNA, 204 bp, Synthetic construct
  source 1..204: mol_type = other DNA; note = STK31 downstream intron
gtaaagttcttatgctaatctcttgcagaaaccattttatcttgcagaaactattttatttaaaataaattttagctccaggttttagagcagcagttcattccccttaacataaaagaaaaattatcccatagaatcaatgtatgctaaaaatatgttgaagaagtttttacttggagtcacattctctgttttgcttcttag

SEQ ID NO: 17 - DNA, 87 bp, Synthetic construct
  source 1..87: mol_type = other DNA; note = STK31 downstream exon
tctgatcctatggcttatctgatggtcccatactaccctagggcaaacctgaatgctgttcaagccaacatgcctttaaattcagaa

SEQ ID NO: 18 - DNA, 79 bp, Synthetic construct
  source 1..79: mol_type = other DNA; note = USP25 upstream exon
gtgtcacaacataatgaaagttgctcaagccaaactggaaatgataaaacctgaagaagtaaacttggaggaatatgag

SEQ ID NO: 19 - DNA, 1607 bp, Synthetic construct
  source 1..1607: mol_type = other DNA; note = USP25 upstream intron
gtaatgtgctttcttagaggttaaactacagatttagggattctgtaattcactgatattcctttcgtttcagcccaactgaagttgaatttgttatgaatgatgagtaaaaaaattaagtttccaagtcaatgctcaagtaattaataattttatatgtgattttaggattaacatgatatgatagcatgtagaccaaaaagtttaaaatcaaaaaaggaagatttttggttttttatttcaatactaattctcaagtactttttaaatgaacttttctagcttaaaaacagaataacaaatgcattgttaattttgaaatcaacttagaaattgctgttacataataccaacaagtaatatttatcaagaccctactatatgccaggcatttgtctaagccctttacatatattaactgatttaattcagacagtaacactgtaagataggtagtgttcttattcccatttgacacacgagaaaataaggcatagagcagtgaagtaacctgcctaaggttttcacagccacaagtttacagcttggctaatagaacctacattcttaactgttgcactacactgtgcaactgtaccctgtgggctgtattttttccttcactttgattcatcagatatattttggggaagttaattcattttagtccctatatgccatatatgtttgtcatgagacagtgttgtgtgtgagagagcacttacataggtacttgtacatgtgcttgtgggatttttttgtgcacaaaattattttttgtcaactttggctttaaaatattctttattttaattgatgaattcatacacctagtatttcttcacatttttaaatttaatcttatgcattgtcttttttttaatttataagtctagtaaattttaagttgttttcttgggtgcctttgcttaatatttagttccatttacaaacttaattatggtactttagattccctaaataattagtgtaattcactacacaaacagggaagaggaaagagagagaaaacactcattattgatgaaaaccttagctagtttggcgtataaggtaactaccttaattcactaaagagtgtctctaaaaacaaaaataggaaataataaaactgcagcaaatatatagaattgtgagcactgaaagcttaaaatttttgttgtcaaaagcaaggcaatgaaaataagcgaaaagtaaaatactggaaaagaagaaacaaagacatcattatttgtagttttttttaccttgaaattcaaataaaatctacaactgatgtattagaactagtaagacagctttgcagcatgcaaacttgtgtcagacatcaattttattcccttttgccagtaaaaaaagtatgagaaagtataaaagtgagaccatttacaatagtatctgaaactatggagtatatagaaataaatttaacaaaaggtgcgtgtgacttctgtggggaaaattataaaacttattgggagacattaacaaagacctaaataaatgggcatacatatggtatttggaaaacttgatattattaatatatagatatcacttttcccccaaatgctttataaattcag

SEQ ID NO: 20 - DNA, 111 bp, Synthetic construct
  source 1..111: mol_type = other DNA; note = USP25 alternatively spliced exon
tgcaggctgtatgaaaattccagcaagatgttttatggagcttgataagccaactgaaaaatgttacggaaatgcaaagatcccagtccacctcccatctctgacaagcaa

SEQ ID NO: 21 - DNA, 2005 bp, Synthetic construct
  source 1..2005: mol_type = other DNA; note = USP25 downstream intron
gttagtgaacagtataaagactttttaataaatggttctgggaaataaataaaatttgatttcttctttattccatatgcaaaaattcatttcaattgtgaaagactcctgtgtaaagctgtttagaagtcaacataggtagaagcccaaagacattcatttatccataagaggcactcactgaccagatatttttgaccaggatatgtaatctagagccagagaaattcaaaatttgttttctcttgaccctgaggagcaagtcaagtcatcttttagctaataatgactccaaatctgtgccctccacattgattgagagctcttcccttgtccccaagattcgtgttcatggaacttgatttttaattgtgacttgattctgcaatgcttccttcagtttgcttcaacagattggactcattatgttttttttcataaaatcttctcatattctaactgcagggctgttaaatcatacaagtatttaaatgacttgtttttagattctgcatattcctcctatctttgtcttgctctaagttcagggttcagtaaaccatagcctgcagaccagcttaccacctgtttttgtaaataaacttttattggaacatggccatttgtatacatattgtctgtggatactttcacactgcagttacagagttgagtaactgaagaagatgaggcttgcaaagtctaaaatattatctggccttttacagaaaaagtttgcctactcacaacttctaaggtgattctaatgacaacaaggaatgacagtttgaataccttccccagaaaagctaacttcatacttgcaggacaaattttacctttatttgttatgaagatctactaatagcatgaaaattatgtcttgaaaattaaaattaagactggatgtggtggctcatgcttataatcccagcacttttggaaatcaaggcacagggagttgtttgaggccaggaatttgagaccggcctggccaacatagcaagaccctgtctatacaaaacagattaagtaattaactggaactgggagcatgcacctgtaattcaagctacttgggagacagaggtaggaggattgctggagcccagaaggtcaagtctgcagttagccgtgatcgtgccactgcactgcagactgggcaactgagtgagaccctgtctccaaaaaaaaaaaaaaaagtttatattcaaggagaatatcgttggctaataactatgtttcttatttttgtttataatgtttatataaaagccttaacacttttgttatcagtattcactttccaatttatcctacattgaatttcagttatttttcttgagtcaattattataaagtaagctaagaaaatcttgactttggtatcaccttagaattatttagtcctattacatgccctatagttgttttgggggtggggaggggcattgaaatgtgaactgcagttattcttttaccatatctcatgtaaataaactattcgcctacacacttaaagtcaaatttaccacatatttgcgaagttaaaaggttaaataaatctaactttttttagtttcttaaaattatgtattaagtatgtgtccaagctttgattcatactcagcgtaatatttcattttcttccatcagttgatgtagatataataatagttcaataatatgattttatactgatgaatttttgagatgacctgattgattatcctgcctttatatttctgttatctgctagtaaaatctttattgtttaaaacatttcatatccatgcctaataatttttagatttaatgttgtattaataaatagtcttgtgtgccagaaagtaaagtcttgttttagttctgatgtatcttcattttgaactctttatttctattaattcagatagtagcgaaacagtgcattactttttgtacagtttcatagtactttttaaatcttactctgaacatttttaatattttctttcacctacag

SEQ ID NO: 22 - DNA, 79 bp, Synthetic construct
  source 1..79: mol_type = other DNA; note = USP25 downstream exon
gagtggcatcaggattataggaaattcagggaaacaactatgtatctcataattgggctagaaaattttcaaagagaaa

SEQ ID NO: 23 - DNA, 130 bp, Synthetic construct
  source 1..130: mol_type = other DNA; note = GTF2B upstream exon
gatcgaacaaataatttattcaagcaagtatatgaacagaagagcctgaagggaagagctaatgatgctatagcttctgcttgtctctatattgcctgtagacaagaaggggttcctaggacatttaaag

SEQ ID NO: 24 - DNA, 1094 bp, Synthetic construct
  source 1..1094: mol_type = other DNA; note = GTF2B upstream intron
gtaagtttttagctttgtttttttgtgtttgtttgtttgtttgtttgagatagggtctcactttgtcgcccaggccagagtgcagtggcgcaatcttgactcactgcaacctctgcctcccaggttcaagctatactcctgcctcagcctcctgagtagctgggattacaggcatgtgccaccacacctggttaacttttgtatttttagtagagacagggtttcgccatgttggccaggctggtctcaaacttctgacctcaggtgatctgcccactttggcctcccaaagtgctagaattaacaggcgtgaggcactgtgcccatcctaaaataacattttctacatcatattaaagaaacagaccaatgtcaggcagctttgacacttagaagctaagtcatctttattgtgactacagtaatccacttgagcatcatttgccaatgcctacttatttcattccttgacagcaaattcatagcatgttcttttaggctgttttcccatgtagactcattttagtccacgtaagcccactatgttcttttctagtgagatcttttgatggactgttgctttgtgggctttcagaaggcctataatcgtaaaccaaagtaggccgtttatacatttggtttacatggaaacagtttccatttctagcactggtctctgtagatgagggagtcaagctttagaaagtatcagtgaaggccaggtatggtgtctcacacctgtaatcccagaactttgggaggccgaggtggttgggtcatgaggccaggagttcaagaccagcctggccaatatggtgaaaccccgtctctactaaaaatacaaaaattagccaggcgtggtggtgcgcgcctgtagtcccagctactcaggaggctgaggcagaagaatcgcttgaaaccgggaggcagaggttgcagtgagccgagatcacgccactacactccagcctgggcgacagagtgagacttcatctcaaaaaaaaaaaaaaaaaagttatcagttaagttttttctcttgaggtgtattgtttgcacttttacacttcagttaagaactggtatttttcatttag

SEQ ID NO: 25 - DNA, 107 bp, Synthetic construct
  source 1..107: mol_type = other DNA; note = GTF2B alternatively spliced exon
tttgcagttacatgtgaatatgtgataatttgttcagtgttgctcactgacttccactaccaaagctcagtttagctgaagactatacttaaccctttattcatcca

SEQ ID NO: 26 - DNA, 1193 bp, Synthetic construct
  source 1..1193: mol_type = other DNA; note = GTF2B downstream intron
gtgagtatttattttttatgttttcttacgtaaaagtgaagaggtgatggcctttatcatcatcttttttgttgtttttataactaaaacaacttttgttaaatttcttctgaaagtgtgtggtggttgcatctgggaatagcagggtcagatattttatctcattgagctacagcccataatcgcagggatttcatgcacacatgacattgtgttgcctaagaggcatcctttagcttgagtattttctttcaattccttggtaacactgcttacaacagattaatacataaacatcaaagaatttttcatttaaagaaatttagttgtatgaattaatccttagagcagttaatattttgaaagattaagacctaaatttagccaggcacgatggctcacaccggtaatctcagcactttgtgaggccgaggcaggtagatcacgaggtcaggagttcaagaccagcctggccaagatggtgaaaccccatctctactaaaaatacaaaaaattagccgggtgtggtggcaggcgcctgtaatcccaactactcgggtggcaggagaatcgcttgaacccagggggcagaggttgcagtgagccaagatggtgtcactgcactccagtctggacgacagagtgaaatttcatctcaaaaaacaaaaaaactctaaattgaaagagtgaagaacattaggccaggcgcagtggctcatgcctgtaatctcagcactttgggaggccgaggtaggaggatcacctgaggtcaggagttctagacaggcctggccaacatggtgaaaccccatctctactaaaaatacaaaagttagccaggtgtggtggtgcacgcctgtagtcccagctactcgggaggctgaggcaggagaatcacttgagcctaggaggtggaggttgcagtgagctgagatcatgccactacccttagtctgggcgacagagtgagacttcatctcaaaaaaaaaggtgtgatgaacattaaagttaactgagaaatttgcagttaactctgtgggaagtatcactgagatgattagggtttcatctttaaggtatgattgaatgttttacaattagaatgtatattatgtttataattagaaatggtagattttaagatgaactatatggcttaagtgaattgatttatttaattttttttcttttag

SEQ ID NO: 27 - DNA, 282 bp, Synthetic construct
  source 1..282: mol_type = other DNA; note = GTF2B downstream exon
aaatatgtgccgtatcacgaatttctaagaaagaaattggtcggtgttttaaacttattttgaaagcgctagaaaccagtgtggatttgattacaactggggacttcatgtccaggttctgttccaacctttgtcttcctaaacaagtacagatggcagctacacatatagcccgtaaagctgtggaactggacttggttcctgggaggagccccatctctgtggcagcggcagctatttacatggcctcacaggcatcagctgaaaagaggacccaaaaag

SEQ ID NO: 28 - DNA, 79 bp, Synthetic construct
  source 1..79: mol_type = other DNA; note = LYPD3 upstream exon
atggaccccgccaggaaagcaggtgcccaggccatgatctggactgcaggctggctgctgctgctgctgcttcgcggag

SEQ ID NO: 29 - DNA, 1036 bp, Synthetic construct
  source 1..1036: mol_type = other DNA; note = LYPD3 upstream intron
gtgggtctgctgcccggagaggtggagggggtagagtgggggctcttagggaggaggctcctctggccccgtacagagaaggctgtgggaaggatccccacataagcagagggcactgtgggcaaagttcccccatcctaagcaagggaggcagagggaagggtcctccaccatgggccgtaggaagggttccccaccacggacaaggggagctgtgggcaaaattttcctatcctgaagctgcagaaagaagtcccctgttcctaggccgggcgcggtggttcatgcctgtaatctcagcactttgggaggccgaggcaggcggatcatgaggtcaaacgatcgagaccagcctggccaacatggtgaaaccctgtctctactaaaaatacaaaaattagctgggcgcggtagcgcgagcctgtagtcccagctactcgggaggctgaggcaggagaatcgtttgaatccaggaggggaggttgcagtgagccgagattgcgccactgcactccagcctggcgacagagcgagactccgtctcaaaaaaaaaaaaaagaaaaaaagaaaaaaagaaaaaagtgttccctgtcctgggcagagaagctgtgggtagtgttctctcatttgagcagaagggggctgtgggaagggatccctcactgagcacagtctactgtgggaaaagttctcccacacactgtgggaaaagttctctcattctgagcagaaaaccgtgaggaggtatccttcctaccccgggatgaggggcttcatggcggggatcgcctacagtgggcatacagggctgtcggaaggatgtgcccacagttggcatagtgggtgtgaaggaaaagggtccagtgattcccgtgcttccagatttgtggaaagcttttccctactcctgccctggaaaagtgtgtgcaataagactgtctaatcccgggacagagggcatgggaaggactatttctccttgggggtggacgctttagcaaggagggctcaggtctattcggctccgctcccccag

SEQ ID NO: 30 - DNA, 132 bp, Synthetic construct
  source 1..132: mol_type = other DNA; note = LYPD3 alternatively spliced exon
gagcgcaggccctggagtgctacagctgcgtgcagaaagcagatgacggatgctccccgaacaagatgaagacagtgaagtgcgcgccgggcgtggacgtctgcaccgaggccgtgggggcggtggagacca

SEQ ID NO: 31 - DNA, 555 bp, Synthetic construct
  source 1..555: mol_type = other DNA; note = LYPD3 downstream intron
gtgagtggggacccccgggccacgcacaccactgcaggctcctccggcccagccccgcctccttatagtcctttctggatctggccacgcccctttaacgcggccccacccctccctttcagcctggccgggaccctacgacacagccttgcaggccaggccttctctattaacaattacaagcgcgaggagcagagagttgagaactgggtggggcggacggggagagctcgggtttggaacgaggtcacgtcctatgcactttttctgctccaccaatgagacctccccatccctgcctgtcaaccaggctgtctcctccgactcagctctttgaggttccgcccccgttccgtgaccccgctcctccctgcttcggtcgttctttccgctctgagcggctctttgggggcgtccatccgccaattcctgcctgtcggtcctgtggtgttggcctatttgaggccccgcccctcccagaccctacccaactctgggccacgccccatctgagaccacgcccacagctaatcccgcccttgtccctctccctag

SEQ ID NO: 32 - DNA, 171 bp, Synthetic construct
  source 1..171: mol_type = other DNA; note = LYPD3 downstream exon
tccacggacaattctcgctggcagtgcggggttgcggttcgggactccccggcaagaatgaccgcggcctggatcttcacgggcttctggcgttcatccagctgcagcaatgcgctcaggatcgctgcaacgccaagctcaacctcacctcgcgggcgctcgacccggcag

SEQ ID NO: 33 - DNA, 67 bp, Synthetic construct
  source 1..67: mol_type = other DNA; note = IK upstream exon
gtgagccgttctccaaccctttggcccccgatggccacgatgtggatgatcctcactccttccacca

SEQ ID NO: 34 - DNA, 345 bp, Synthetic construct
  source 1..345: mol_type = other DNA; note = IK upstream intron
gtgagtattttgtgctaggaccatggaaatgagttgggcaatgaatagaaaagtggtggtgatggtgaaagaattctgaatgtcttgtcatcaaggatggtaaaaaattatacccgctggccctttatctctgtgagccttaactggatcaggccaagtgcataagggtattagaagggaaattcctgcaggaaaggtgttaagtttttttttttttttatacgtgttgtgtaattacacattatataggaattacttcttccaatagcaaattaaaacataggaaataagggtaaaatcatctttttattttttttgagactgagtttcgctcttgttgctcag

SEQ ID NO: 35 - DNA, 102 bp, Synthetic construct
  source 1..102: mol_type = other DNA; note = IK alternatively spliced exon
gctggaatgcagtggtgcgatctcggctcaccacaacctccgcctcccgggttcaagcaattctcctgcctcagccttccgagtagctgggattacaggcat

SEQ ID NO: 36 - DNA, 2729 bp, Synthetic construct
  source 1..2729: mol_type = other DNA; note = IK downstream intron
gttccactacgcccggctaattttgtatttttagtagagatggggtttctccatgttgatcaggctggtcttgaaatcccgacttcaggtgatccacccgcctctgcctcccaaagtggtgggagtacaggcgtgagccaccgtgcctggcccccgataaaatcatctttgataacatccccaattccagtccatttctttttttttttcttttcttttcttttctttctttttttttttttttgagacggagtctcactgttacccaggctggagtgcagtgatgcaatcttggctcactgcaacctctgcctcctgagttcaagcggttctcctgcctcagcctcccaagtagctgggactacaggcatgtgccaccatgcacgactgatttttttttttgtatttttagtagagacagggtttcactgtgttcgtcaggctggtcttgaactcctggcctgaagtgatccacccgcctcagcctcctaaagtgctggcattacaggcgtgagccaccgtgcccagcctcagtccttttcttagaagtaaccacagttaccttttttggcatatcttttcctcccgttgcccaggctggagttcaatggcattatttctgctcacagcaacctttgcctcctgggctcaagcaattcttctgcctcagtttccccagtagctgggattacaggcatgccccaccatgcttggctaatttttgtattagtagagacagggtttcgctatgttggccaggctggtcttgaactcctgacctcaaatgatctgcccgtctcggcggcccaaagtgctgggattacaggcgtgagccactgcgcctggccctcctactcttttttaaatgcatttatacacatacatgcatatctgaggaaaacacaacattgctttgtgatttaaaatatttgagttaaatgatattatgctgtatatatcatttttgcaacttgcttctttttaaaccctgctcagtgtttctgagattatctttcagagcagtgacaagcattccctccttggctaaactttagtcaggttccccaaagagtcccatttttcagcaggactcatccgtggcctgctgagcacagttttaacaaagaaatcaaccaccgctccacccccatacctaaccaagttgcttttagtaattttctataaattctcacttgtccctgttgtattcggaattaagttcagtctctctactctattacaatggtcttgactcttgttacagtagtcttcaataaagtcttccttggggagaagaggaacttgattagatatcaagttggattggatatcaagagtgggaaaactttctgtgaactgacttaggattcctttttaaaactgaggtcagcaggccaaggaagagccctagtcagaagagagctttaaaggagtctgactttaatttgatcaagagaggagtctttgttagtgtaccaattaaaataatctcttgtaccattagtgctgtctttccctctttgggaaacaccatcatagtttagtcagtctccagctaataaacgcttagtctgtttcagactgattcttttcttttttttatttttttgagacagagtcttgctctttttgcccaggctggagtgtaatacctagatctcggctcactgcaaactccgcctcccaggttcaagcgattttcctgcctcagcctcctgagtagctaggattacaggcgcccaccacaacgcccagctaatttttttgtatttttagtagagatgggatatcaccatgttggccatgctggtcttgaactcctgacctcgggtgatccgcctgcgtcagcctcccaaagtgcggcgattataggtgtgagccactgcacccggccagactgattattttcaaggctataactgaattaaatttacaggccttcttatatacatatgtcagtaatggaccaggctaaatataaataaatggagttgctggctcataggatatatatacttaaattttaacaaatactgcaaaattgcccttcaaattggttatactggtttacattctcatcagcattatatgagagagtccccttcccaaattctcactcacacttgatttatcaaactttctggtagttaacactctgatgcattaaaagtggcatggcatatctggtcgggcgtggtggcttacacctataatcccagcactttgggaggctgaggtgggcagatcacctgaggtcagaagtttgggaccagcctggccaacacctctaataaaaatacaaaaattagctgggtgtgttggcgggcacctgtattcccagctacttgtgaggctgaggcaggagaattacttgaacccaggaggcagaggttgcagtgggctgagatcatgccactgcactccagccggggcaacagagggagactctgtcttaaaaaaaaaaaaaaaaagctgggtgcggtggctcatccctataatcccagcactttgggaggccgaggtgggtggatcacaaggtcaggagttcaagaccagcctggccaatatgggtgacagtgagactccgtctcaaaaaaaaaaaaacagtgtcatatctttgttttaatttgtatttccctgatttctgttaatttaatcatgatttttacttgttcctttttcatttcttattgagtcctaatatttaaaatctttgccttttcttctag

SEQ ID NO: 37 - DNA, 93 bp, Synthetic construct
  source 1..93: mol_type = other DNA; note = IK downstream exon
atcaaaactcaccaatgaagacttcaggaaacttctcatgacccccagggctgcacctacctctgcaccaccttctaagtcacgtcaccatga

SEQ ID NO: 38 - DNA, 282 bp, Synthetic construct
  source 1..282: mol_type = other DNA; note = TEX14 upstream exon
tggaggtgaaagaaatggcaaagaaagcagctactggacagctcacagtacctccttggcatcctcagagtagtctgactttagagagcgaggctgaaaatgagcccgacgccctgctgcagccccccattaggagcccagaaaacacggattggcagcgagttattgagtatcatagggaaaatgatgagcccagaggaaatggcaagtttgacaagacgggcaacaatgactgtgacagtgaccagcatggcagacagcccaggcttggaagcttcacca

SEQ ID NO: 39 - DNA, 544 bp, Synthetic construct
  source 1..544: mol_type = other DNA; note = TEX14 upstream intron
gtaagttcacttgcgggaatcaggataggtgaactcaatcaagtatcagtcgggcgaggtggctcagcctgtaattccagcactttgggaggtgaggcgagcggatcacctgagcccaggaatttgggaccagcctggtcaacatgatgaaacctggtctctacaaaaaaaaaaaaaaaaaaaaaaaaaacattagctgggcgtggtggcacacgcctctagttccaggtacccaggaggctgaggtgggaggatcacttggacctgagaggtggaggttgcagtgagctgtgatcatgaaactgcactccagcttgggtgacagtgagaccctgtctcaaaataaaaataaaagtaaaaaaaagttatcagctctcaaaaaagtaaactttccagcatattttttttggtttggtcctctctcctttcctacttttctggaaaccttgaattccaggggaagacagtatagccctatatctcattgcatttcccacaggcaaacagatccagacactgatcagtttttatattactgaaacag

SEQ ID NO: 40 - DNA, 85 bp, Synthetic construct
  source 1..85: mol_type = other DNA; note = TEX14 alternatively spliced exon
gtccctttgtccaaaggaagtaaaataatcaaatactgaccctggttttggcgaggaatcgatccttctttacctggggatagag

SEQ ID NO: 41 - DNA, 571 bp, Synthetic construct
  source 1..571: mol_type = other DNA; note = TEX14 downstream intron
gtaggccatcactgcctactgcccatgtctctgcattgtattctaagctgaaattaaattctcccagaatttatttagaatgtattctaagctggcattaaatttttccagagagtctatgctggaggaacctagagacacatgacatttattgtcatcggtttatagatttacaggttaaaaaaaatgaaggctgtatattttcagttgataatttgcttataatcagggcatggatattgtttcacagttgtttataagttaaagaaagatacatctaaacttgtcatttgtcttaggcagaaaatctattggcttgggtattatttgtatttagaatctgtttcttgtcaagcatcactctcgaatgggtgggcagatgatccagggctgcattttggggtcaggctaaactaacagtctctactctctggactgaacctaacgtatgggacatggccactcttggcagaatatgtatacatctcttctttcatgccacctttatcctcttccatgttgtctgaatatcactgaatgactttgaccctgactgtgttacaatcattag

SEQ ID NO: 42 - DNA, 101 bp, Synthetic construct
  source 1..101: mol_type = other DNA; note = TEX14 downstream exon
gtatcaggcacccatctcccagacaaaaggagcaaccagagcatagtgaagccttccaagcaagttctgacacattggtggctgtagagaaatcttacagt

SEQ ID NO: 43 - DNA, 5786 bp, Synthetic construct
  source 1..5786: mol_type = other DNA; note = TRPV3 construct #1 (full)
ttttatccaagctgtgcttgtgatactgtctgtcttcttgtacttgtttgcctacaaagagtacctcgcctgcctcgtgctggccttggccctgggctgggcgaacaagctctactatacgcggggtttccagtccatcggcttgtacagcgtcatcatccagaaggtgcgttgagagctgcgccggcgggaggggcctcggaggatggccatttttcagatggggaaactgagatctggagaggaagaggggtttaccaaggggcaggtcactgctctttctcccaaatgccatgcactccctttgctttcggccaggctttaagtgtcaagaccttaaatcccccaggcttgtggggtgctggggaggggtacagtttgtggaggaatgttctcacccctgggcagtggcgtgctggtaaacatttaacgactgttctgtgtgtgtgtgtgtgtatgtgtgtgtgtgtaggtggaatatgtatattccatatatatacacaaaagacttatcatacattttactatataaacgatgtgtatcacacaatttgcaaataatgaaaatataccatgcagtttattgtaaattccacatagccagttgagtctcagaataatttgttgctttttttttttttttttggagaccaagtcttgctctgctgcccaggccagagtaaagtggtgcgatctcggctccctgcaacctctgcctcctgggttcaagcagttctcctgcctcagcctccagagtagctgggatcacaggcacgcaccaccatgttggctaatttttgtatttttagtagagacagggtttcagcatgttggccaggattgtcttgaactcctgacctcaggtgatccacctgcctcggcctcccaaagtgctgggattacaggcatgaaccaccgcgccaggcctatttgttgctttttattgaattctagtttttgttgtcaacttttggttgcaattgacaaaagagtgctatcagacatgaatgttggttgagagttttctttataagcagtgattgaagtgaaacaattaagatttatgttgaactttacttgtttatcaatgatgtgagcaacttctttgctgaattggatactagtttgcaaataccagaataatattctgttgatatttttgtgctatacataatgtaaaggctgcagacagaacacattttaaaatttaaactggattaatagtttctccatcactgtcttaaatcatttagatctagatatcaacaaaacaatgagtcaatcaatctctgatttgtggcatttaccaattttcatgatgtaaatacagtagtccccccttattcttgggggagatgttctaagaccccccatgaatgcctgaaactgcagatagtaccaaacattatataatatgctatgttttttcctatacatatacatctatgcaaataatgaaaatatactatacaatacagtttattgtaaattccacttagccagttgagtcttagaataatttttgctttttttttttttttttgtttgtgactgagtctcgctctgttgcccaggctggagtgcaatagtgcaatctcggctcactgcaacctctgcctcccgggctcaagcaattctcccgccccagcctcctgagtagctgggactacaggcatgcaccatgcccagctaatttttgtatttttagtagagatggggtttcgccatgttggccaggctggtctccaactcttgacctcaggtgatccgtccgcctcggcctcccaaagtgctggggggtgggggggggaatcaacaaattattctaagactcaactggctatgtggaatttataaaatttattttatcaattaggcatagtgagattaatggcaataactaacagaacaatgataacaatatactgtaataaaagtatgtgaatgttgtctctctcagaatattgtattgtgtgtaatatttctgggtcatagttgaccacaggcaaccaaaactgcagaaagtgaaatcgaagagaagggcggactactgttctcctctcagggccaatttccagctaccaacattatgttactggacatagagttagggagaaacatgcagatccacatcgctctagaattcctgccatatggctatgatagagacaagtaacctcaaaagcacacacaattgtaaatatagtgaaataactggacggggatgagttttgaacatttattgtctttgttttatttaattaaattaattaattaattaattttttgagacagagtctggctctgccccccaggctggagtgcagtggtgcgatctaggctcactgcaaccccttcctcctgggttcaagcaattctcccgcctcagcctctggagtagctgggatcacaggtgtgtgccaccacacccagctaatttttgtatttttagtagagacggggtttcgccatgttggccaagctggcctccaactcctgacctcaggcgatccacccacctcagcctctcaaagtgttgggattacaggcgtgagtcactgcaccctggcctgtctttgttttaaatataacttattttatttaattgtaagtttatatggtttaattttttggcaatacaaataataatttctttacccccagctttattgaggtacagctgacatatataattgtataaatttaagatgtacaatgtgaggatttgatataggtatatattgtgaaatgatttccaatttccaattaacacacccatcacctcacatagttgccatttctttgtgtgtggtgagaacatttatgatctattctcttagcaaatttctagtacacaacacggtattgttcactatagtcatcatgctgtccattagatccccagaacttattcatcttattcattgatttagttctgaataatggctgtgtttaacaaccagctcacaagcttcatgagaatttaacgatcagctcttgcgagctcttctgagctggctccagcatgccactgcccccagcaagtgtgaggattccagtgtgagaccctatggtgaaaggtacagggtgaatcacagggagaagagaacctggggctggagcagaaacagaaggcttcctggaggaggcaggatccagtgttgagccttggagcatcaggaggatttggctgggggaagggagagtggcccaagtgaagagacagttagggtggggggtgggagagggtgggaagcaggaagatcagggtgccgtgaatatgtgcttgcaccttacacttgaattatgtttatttccttataggtcatttgccaccatgttctggagttcttgtttgtatatatcgtgtttttgcttggatttggagttggtaattgatttataaataatagtaactcccacatttataaacactttatacagatgtcaaaatggcaaagtgcatggcggccttggcaacaattagaccaactaccagtggggcacttgccatgcttggcattttccaagcattacctttatatatatacatatatattttattatactttaagttctagggtacatgtgcacaacatgcaggtttgttacatatgtatacatgtgccatgttggtatgctgcacccattaactcgtcatttacattaggtatatctcctaatcaagcattacctttttacttctctcaacaacttgatcaagtgggaatcatcaccccgatcataaggtaactgaggcttagagactccaggattcaaaccaggccagccactcataatccaacttcaccatgtggtatatacctctggctttcccattcattgcttacgtggccctcacgacctaagataggttctgcaagtgttttcagccccattttaacagatgcggaaactaaagtccacagaaactcaactgatggtctttgcaaagtaggagcaaactaataccctgaggtgatttcctttggatttcacatcacgttactctcacagtttgctttagatacaactgtgagagcctctctggtctgctgggaccaggaaggagccccctcttaaaatctctccatttacaacagagggaacagcacgatctggttgtatttctaacctctgcatagagagatatcattttcccccacgtaccagttgcttttttttcttttcttcagtgaccattgacatttgaaattaagtagctgtttccatctgtctggtaccttttctgggtcacaatttgcttatttatttgctccttcacctgcttgtttattcatccatctatttactctctcatttaatttgctcatccacctgtctatccgtccatccatccatccatccatccatccatccatccatccaatcattcatccatcgatccatccattgatctatccatctatccatccatccatccatccatccatccatctaatcatccatccaatcatccatccatccatccatccatccatccatccatccagtcatccatccattcatccatttaatcatccatccatcgatcaatcggtccatccatccagctatccatccatccatccatccatctaatcattcatccatcaatccatccatccatccatccatccatccatccatccatccatctatccatccatccaatcatccatccatccatcgatcaatcagtccatccatccatctaatcatccatctaatcatccatccatccatccatccatccatccagtcatccatccattcatccatttaatcatccatccatccatcgatcaattggtccatccatccatctatccatccatccatccatccatctaatcacccatccatccatccatccatccaatcatctatccatccatccatctaatcatctatccatccatccatccatccatccatccaatcatccatccatccatccatccatccctccatctaatcatccatccctccatccatccctccatatccatccatccatccatccatccatccatccaaccattcagcagtgaacctctactgtggtgtgttggaaagaagggaagtgtgagcctggtcttgctcacagggaactcaaaacagagcattaggaggagactcaggatcttacagccttgagggaggtgacccaggctagaggaagtcaggagctcaaagactagctggggagccagggacacacacatggattataattagtggctctgcaaggctgcaagtgatcaagggcagactgcggggtaaaggaaggagagaagactctgggctttgttaggtggaagggctttctgcaggaggccaggccagagaagccagcctgatgaggctttgagaggacctgaaactggagatgggatttaatggccagagagaaggaggcagcaggcttgtcatgtaggcaaatacacgcaagcttgtagagtaattaccaggcagagagctgaggagagcaccctgttatgttcccttagccttggcctcgcttatcgagaagtgtcccaaagacaacaaggactgcagctcctacggcagcttcagcgacgcagtgctggaactcttcaagctcaccattggcctgggtgacctggacatccagcagaactccaagtatcccattctctttctgttcctgctcatcacctatgtcatcctcacctttgttctcctcctcaacatgctcattgctcttatgggcgagactgtggagaacgtctccaaggagagcgaacgcatctggcgcctgc

SEQ ID NO: 44 - DNA, 906 bp, Synthetic construct
  source 1..906: mol_type = other DNA; note = TRPV3 construct #2 (minimal v1) Also referred to as V1B
ttttatccaagctgtgcttgtgatactgtctgtcttcttgtacttgtttgcctacaaagagtacctcgcctgcctcgtgctggccttggccctgggctgggcgaacaagctctactatacgcggggtttccagtccatcggcttgtacagcgtcatcatccagaaggtgcgttgagagctgcgccggcgggaggggcctcggaggatggccatttttcagatggggaaactgagatctggagaggaagaggggtttaccaaggggcagttagggtggggggtgggagagggtgggaagcaggaagatcagggtgccgtgaatatgtgcttgcaccttacacttgaattatgtttatttccttataggtcatttgccaccatgttctggagttcttgtttgtatatatcgtgtttttgcttggatttggagttggtaattgatttataaataatagtaactcccacatttataaacactttatacagatgtcaaaatggcaaagtgcatggcggccttggcaacaattagaccagaaggaggcagcaggcttgtcatgtaggcaaatacacgcaagcttgtagagtaattaccaggcagagagctgaggagagcaccctgttatgttcccttagccttggcctcgcttatcgagaagtgtcccaaagacaacaaggactgcagctcctacggcagcttcagcgacgcagtgctggaactcttcaagctcaccattggcctgggtgacctggacatccagcagaactccaagtatcccattctctttctgttcctgctcatcacctatgtcatcctcacctttgttctcctcctcaacatgctcattgctcttatgggcgagactgtggagaacgtctccaaggagagcgaacgcatctggcgcctgc

SEQ ID NO: 45 - DNA, 410 bp, Synthetic construct
  source 1..410: mol_type = other DNA; note = TRPV3 construct #3 (minimal v2) Also referred to as V2B
tctactatacgcggggtttccagtccatcggcttgtacagcgtcatcatccagaaggtgcgttgagagctgcgccggcgggaggggcctcggaggatggccattttgtgaatatgtgcttgcaccttacacttgaattatgtttatttccttataggtcatttgccaccatgttctggagttcttgtttgtatatatcgtgtttttgcttggatttggagttggtaattgatttataaataatagtaactcccacatttataaacactttatagtaattaccaggcagagagctgaggagagcaccctgttatgttcccttagccttggcctcgcttatcgagaagtgtcccaaagacaacaaggactgcagctcctacggcagcttcagcgacgcagtgctggaactct

SEQ ID NO: 46 - DNA, 1446 bp, Synthetic construct
  source 1..1446: mol_type = other DNA; note = STK31 construct #1 (full)
aactctggtggtctccttacaatcagcttggaacgagatcttcttgaagctgagcccttgaaggaacttagcagcaagcgtcctttggtacgttctgaggttattgggcagataattctgttaaaggtaagtctaacttcttcttacttgtgggagattcagcagcataaaggatagtggtgctgttacatttcaagaaagaaaaaacctcactgttagttttctaaagtgtataagttgactggcatgataattgtgcttgtagtttggttgccagaactaattttcacttttaaagtttccctcaattgaacaatgagaacacttggacacagggcggggaacatcacccgtgggggcctgtcatggggtggggggcagggggagggatagcattaggagaaatacctaatgtaaatgatgagttaatgggtgcagcaaaccaacatggcacatgtatacatatgtaacaaacctgcacgttgtgcacatgtaccctagaacttaaagtataataataaaaataagtatataaaagtcattaaataaagctttgtctcacaagaaaaaaaaataaagtttccctcatttccctaaatagaatatgtccttaataggatatactttcctttttttttgggtgaacttattatagtcatccaatatctttttaaaattcttgattttcttacatttctccttgaaatctcagctgtgttatcagtacaatcttctactctttaatttcttcttctcattgatctgggaaactgttaatcatgataataagataattaatttggccttagttgctacacattctagaatgtgtagcaggctacaggtagaattgcactttccttttccaaattatagcattcaaagaagatagaaaagtacttataaaattaaaaaataaaattagatttgatttaacttaaaataatgaattatgtttaaattggaatgatgtataattataatgagattttcatcttggaattacgagtgcctctttctttggtacaagggctattctgtggttgttgacacagaagccaaggtgattgagagagcagccacctaccatagagcttggagagaagctgaaggagactcagggttactgccattgatattcctgccaccatgaaaggtaaagttcttatgctaatctcttgcagaaaccattttatcttgcagaaactattttatttaaaataaattttagctccaggttttagagcagcagttcattccccttaacataaaagaaaaattatcccatagaatcaatgtatgctaaaaatatgttgaagaagtttttacttggagtcacattctctgttttgcttcttagtctatcctatggcttatctgatggtcccatactaccctagggcaaacctgaatgctgttcaagccaacatgcctttaaattcagaa

SEQ ID NO: 47 - DNA, 742 bp, Synthetic construct
  source 1..742: mol_type = other DNA; note = STK31 construct #2 (minimal v1)
aactctggtggtctccttacaatcagcttggaacgagatcttcttgaagctgagcccttgaaggaacttagcagcaagcgtcctttggtacgttctgaggttattgggcagataattctgttaaaggtaagtctaacttcttcttacttgtgggagattcagcagcataaaggatagtggtgctgttacatttcaagaaagaaaaaacctcactgttagttttctaatttaacttaaaataatgaattatgtttaaattggaatgatgtataattataatgagattttcatcttggaattacgagtgcctctttctttggtacaagggctattctgtggttgttgacacagaagccaaggtgattgagagagcagccacctaccatagagcttggagagaagctgaaggagactcagggttactgccattgatattcctgccaccatgaaaggtaaagttcttatgctaatctcttgcagaaaccattttatcttgcagaaactattttatttaaaataaattttagctccaggttttagagcagcagttcattccccttaacataaaagaaaaattatcccatagaatcaatgtatgctaaaaatatgttgaagaagtttttacttggagtcacattctctgttttgcttcttagtctatcctatggcttatctgatggtcccatactaccctagggcaaacctgaatgctgttcaagccaacatgcctttaaattcagaa

SEQ ID NO: 48 - DNA, 3881 bp, Synthetic construct
  source 1..3881: mol_type = other DNA; note = USP25 construct #1 (full)
gtgtcacaacatattgaaagttgctcaagccaaactggaattgataaaacctgaagaagtaaacttggaggaatatcaggtaatgtgctttcttagaggttaaactacagatttagggattctgtaattcactgatattcctttcgtttcagcccaactgaagttgaatttgttatgaatgatgagtaaaaaaattaagtttccaagtcaatgctcaagtaattaataattttatatgtgattttaggattaacatgatatgatagcatgtagaccaaaaagtttaaaatcaaaaaaggaagatttttggttttttatttcaatactaattctcaagtactttttaaatgaacttttctagcttaaaaacagaataacaaatgcattgttaattttgaaatcaacttagaaattgctgttacataataccaacaagtaatatttatcaagaccctactatatgccaggcatttgtctaagccctttacatatattaactgatttaattcagacagtaacactgtaagataggtagtgttcttattcccatttgacacacgagaaaataaggcatagagcagtgaagtaacctgcctaaggttttcacagccacaagtttacagcttggctaatagaacctacattcttaactgttgcactacactgtgcaactgtaccctgtgggctgtattttttccttcactttgattcatcagatatattttggggaagttaattcattttagtccctatatgccatatatgtttgtcatgagacagtgttgtgtgtgagagagcacttacataggtacttgtacatgtgcttgtgggatttttttgtgcacaaaattattttttgtcaactttggctttaaaatattctttattttaattgatgaattcatacacctagtatttcttcacatttttaaatttaatcttatgcattgtcttttttttaatttataagtctagtaaattttaagttgttttcttgggtgcctttgcttaatatttagttccatttacaaacttaattatggtactttagattccctaaataattagtgtaattcactacacaaacagggaagaggaaagagagagaaaacactcattattgatgaaaaccttagctagtttggcgtataaggtaactaccttaattcactaaagagtgtctctaaaaacaaaaataggaaataataaaactgcagcaaatatatagaattgtgagcactgaaagcttaaaatttttgttgtcaaaagcaaggcaatgaaaataagcgaaaagtaaaatactggaaaagaagaaacaaagacatcattatttgtagttttttttaccttgaaattcaaataaaatctacaactgatgtattagaactagtaagacagctttgcagcatgcaaacttgtgtcagacatcaattttattcccttttgccagtaaaaaaagtatgagaaagtataaaagtgagaccatttacaatagtatctgaaactatggagtatatagaaataaatttaacaaaaggtgcgtgtgacttctgtggggaaaattataaaacttattgggagacattaacaaagacctaaataaatgggcatacatatggtatttggaaaacttgatattattaatatatagatatcacttttcccccaaatgctttataaattcagtgcaggctgtatgaaaattccagcaagatgttttatcgagcttgataagccaactgaaaaatcttacggaattgccaccatgccagtccacctcccatctctgacaagcaagttagtgaacagtataaagactttttaataaatggttctgggaaataaataaaatttgatttcttctttattccatatgcaaaaattcatttcaattgtgaaagactcctgtgtaaagctgtttagaagtcaacataggtagaagcccaaagacattcatttatccataagaggcactcactgaccagatatttttgaccaggatatgtaatctagagccagagaaattcaaaatttgttttctcttgaccctgaggagcaagtcaagtcatcttttagctaataatgactccaaatctgtgccctccacattgattgagagctcttcccttgtccccaagattcgtgttcatggaacttgatttttaattgtgacttgattctgcaatgcttccttcagtttgcttcaacagattggactcattatgttttttttcataaaatcttctcatattctaactgcagggctgttaaatcatacaagtatttaaatgacttgtttttagattctgcatattcctcctatctttgtcttgctctaagttcagggttcagtaaaccatagcctgcagaccagcttaccacctgtttttgtaaataaacttttattggaacatggccatttgtatacatattgtctgtggatactttcacactgcagttacagagttgagtaactgaagaagatgaggcttgcaaagtctaaaatattatctggccttttacagaaaaagtttgcctactcacaacttctaaggtgattctaatgacaacaaggaatgacagtttgaataccttccccagaaaagctaacttcatacttgcaggacaaattttacctttatttgttatgaagatctactaatagcatgaaaattatgtcttgaaaattaaaattaagactggatgtggtggctcatgcttataatcccagcacttttggaaatcaaggcacagggagttgtttgaggccaggaatttgagaccggcctggccaacatagcaagaccctgtctatacaaaacagattaagtaattaactggaactgggagcatgcacctgtaattcaagctacttgggagacagaggtaggaggattgctggagcccagaaggtcaagtctgcagttagccgtgatcgtgccactgcactgcagactgggcaactgagtgagaccctgtctccaaaaaaaaaaaaaaaagtttatattcaaggagaatatcgttggctaataactatgtttcttatttttgtttataatgtttatataaaagccttaacacttttgttatcagtattcactttccaatttatcctacattgaatttcagttatttttcttgagtcaattattataaagtaagctaagaaaatcttgactttggtatcaccttagaattatttagtcctattacatgccctatagttgttttgggggtggggaggggcattgaaatgtgaactgcagttattcttttaccatatctcatgtaaataaactattcgcctacacacttaaagtcaaatttaccacatatttgcgaagttaaaaggttaaataaatctaactttttttagtttcttaaaattatgtattaagtatgtgtccaagctttgattcatactcagcgtaatatttcattttcttccatcagttgatgtagatataataatagttcaataatatgattttatactgatgaatttttgagatgacctgattgattatcctgcctttatatttctgttatctgctagtaaaatctttattgtttaaaacatttcatatccatgcctaataatttttagatttaatgttgtattaataaatagtcttgtgtgccagaaagtaaagtcttgttttagttctgatgtatcttcattttgaactctttatttctattaattcagatagtagcgaaacagtgcattactttttgtacagtttcatagtactttttaaatcttactctgaacatttttaatattttctttcacctacaggagtggcatcaggattataggaaattcagggaaacaactatgtatctcaaaattgggccagaaaattttcaaagagaaa

SEQ ID NO: 49 - DNA, 669 bp, Synthetic construct
  source 1..669: mol_type = other DNA; note = USP25 construct #2 (minimal v1)
gtgtcacaacatattgaaagttgctcaagccaaactggaattgataaaacctgaagaagtaaacttggaggaatatcaggtaatgtgctttcttagaggttaaactacagatttagggattctgtaattcactgatattcctttcgtttcagcccaactgaagttgaatttgttatgaaaacaaagacctaaataaatgggcatacatatggtatttggaaaacttgatattattaatatatagatatcacttttcccccaaatgctttataaattcagtgcaggctgtatgaaaattccagcaagatgttttatcgagcttgataagccaactgaaaaatcttacggaattgccaccatgccagtccacctcccatctctgacaagcaagttagtgaacagtataaagactttttaataaatggttctgggaaataaataaaatttgatttcttctttattccatatgcaaaaattcatttcaattgtgaattcagatagtagcgaaacagtgcattactttttgtacagtttcatagtactttttaaatcttactctgaacatttttaatattttctttcacctacaggagtggcatcaggattataggaaattcagggaaacaactatgtatctcaaaattgggccagaaaattttcaaagagaaa

SEQ ID NO: 50 - DNA, 2806 bp, Synthetic construct
  source 1..2806: mol_type = other DNA; note = GTF2B construct #1 (full)
gatcgaacaaataatttattcaagcaagtataagaacagaagagcctgaagggaagagctattgatcctatagcttctgcttgtctctatattgcctgtagacaagaaggggttcctaggacatttaaaggtaagtttttagctttgtttttttgtgtttgtttgtttgtttgtttgagatagggtctcactttgtcgcccaggccagagtgcagtggcgcaatcttgactcactgcaacctctgcctcccaggttcaagctatactcctgcctcagcctcctgagtagctgggattacaggcatgtgccaccacacctggttaacttttgtatttttagtagagacagggtttcgccatgttggccaggctggtctcaaacttctgacctcaggtgatctgcccactttggcctcccaaagtgctagaattaacaggcgtgaggcactgtgcccatcctaaaataacattttctacatcatattaaagaaacagaccaatgtcaggcagctttgacacttagaagctaagtcatctttattgtgactacagtaatccacttgagcatcatttgccaatgcctacttatttcattccttgacagcaaattcatagcatgttcttttaggctgttttcccatgtagactcattttagtccacgtaagcccactatgttcttttctagtgagatcttttgatggactgttgctttgtgggctttcagaaggcctataatcgtaaaccaaagtaggccgtttatacatttggtttacatggaaacagtttccatttctagcactggtctctgtagatgagggagtcaagctttagaaagtatcagtgaaggccaggtatggtgtctcacacctgtaatcccagaactttgggaggccgaggtggttgggtcatgaggccaggagttcaagaccagcctggccaatatggtgaaaccccgtctctactaaaaatacaaaaattagccaggcgtggtggtgcgcgcctgtagtcccagctactcaggaggctgaggcagaagaatcgcttgaaaccgggaggcagaggttgcagtgagccgagatcacgccactacactccagcctgggcgacagagtgagacttcatctcaaaaaaaaaaaaaaaaaagttatcagttaagttttttctcttgaggtgtattgtttgcacttttacacttcagttaagaactggtatttttcatttagtttgcagttacttgtgaatatctgataatttgttcagtgttgctcactgacttccactaccaaagctcagtttagctgaagacgccaccatggcctttattcatccagtgagtatttattttttatgttttcttacgtaaaagtgaagaggtgatggcctttatcatcatcttttttgttgtttttataactaaaacaacttttgttaaatttcttctgaaagtgtgtggtggttgcatctgggaatagcagggtcagatattttatctcattgagctacagcccataatcgcagggatttcatgcacacatgacattgtgttgcctaagaggcatcctttagcttgagtattttctttcaattccttggtaacactgcttacaacagattaatacataaacatcaaagaatttttcatttaaagaaatttagttgtatgaattaatccttagagcagttaatattttgaaagattaagacctaaatttagccaggcacgatggctcacaccggtaatctcagcactttgtgaggccgaggcaggtagatcacgaggtcaggagttcaagaccagcctggccaagatggtgaaaccccatctctactaaaaatacaaaaaattagccgggtgtggtggcaggcgcctgtaatcccaactactcgggtggcaggagaatcgcttgaacccagggggcagaggttgcagtgagccaagatggtgtcactgcactccagtctggacgacagagtgaaatttcatctcaaaaaacaaaaaaactctaaattgaaagagtgaagaacattaggccaggcgcagtggctcatgcctgtaatctcagcactttgggaggccgaggtaggaggatcacctgaggtcaggagttctagacaggcctggccaacatggtgaaaccccatctctactaaaaatacaaaagttagccaggtgtggtggtgcacgcctgtagtcccagctactcgggaggctgaggcaggagaatcacttgagcctaggaggtggaggttgcagtgagctgagatcatgccactacccttagtctgggcgacagagtgagacttcatctcaaaaaaaaaggtgtgatgaacattaaagttaactgagaaatttgcagttaactctgtgggaagtatcactgagatgattagggtttcatctttaaggtatgattgaatgttttacaattagaatgtatattatgtttataattagaaatggtagattttaagatgaactatatggcttaagtgaattgatttatttaattttttttcttttagaaatatgtgccgtatcacgaatttctaagaaagaaattggtcggtgttttaaacttatttagaaagcgcttgaaaccagtgtggattttattacaactggggacttcatgtccaggttctgttccaacctttgtcttcctaaacaagtacagatggcagctacacatattgcccgtaaagctgtggaactggacttggttcctgggaggagccccatctctgtggcagcggcagctatttacatggcctcacaggcatcagctgaaaagaggacccaaaaag

SEQ ID NO: 51 - DNA, 923 bp, Synthetic construct
  source 1..923: mol_type = other DNA; note = GTF2B construct #2 (minimal v1)
gatcgaacaaataatttattcaagcaagtataagaacagaagagcctgaagggaagagctattgatcctatagcttctgcttgtctctatattgcctgtagacaagaaggggttcctaggacatttaaaggtaagtttttagctttgtttttttgtgtttgtttgtttgtttgtttgagatagggtctcactttgtcgcccaggccagagtgcagtggcgcaatcttgactcaaaaaaaaaaaaaaaaaagttatcagttaagttttttctcttgaggtgtattgtttgcacttttacacttcagttaagaactggtatttttcatttagtttgcagttacttgtgaatatctgataatttgttcagtgttgctcactgacttccactaccaaagctcagtttagctgaagacgccgccaccatggcctttattcatccagtgagtatttattttttatgttttcttacgtaaaagtgaagaggtgatggcctttatcatcatcttttttgttgtttttataactaaaacaacttttgttaacaattagaatgtatattatgtttataattagaaatggtagattttaagatgaactatatggcttaagtgaattgatttatttaattttttttcttttagaaatatgtgccgtatcacgaatttctaagaaagaaattggtcggtgttttaaacttatttagaaagcgcttgaaaccagtgtggattttattacaactggggacttcatgtccaggttctgttccaacctttgtcttcctaaacaagtacagatggcagctacacatattgcccgtaaagctgtggaactggacttggttcctgggaggagccccatctctgtggcagcggcagctatttacatggcctcacaggcatcagctgaaaagaggacccaaaaag

SEQ ID NO: 52 - DNA, 1970 bp, Synthetic construct
  source 1..1970: mol_type = other DNA; note = LYPD3 construct #1 (full)
gaccccgccaggaaagcaggtgcccaggccttgatctggactgcaggctggctgctgctgctgctgcttcgcggaggtgggtctgctgcccggagaggtggagggggtagagtgggggctcttagggaggaggctcctctggccccgtacagagaaggctgtgggaaggatccccacataagcagagggcactgtgggcaaagttcccccatcctaagcaagggaggcagagggaagggtcctccaccatgggccgtaggaagggttccccaccacggacaaggggagctgtgggcaaaattttcctatcctgaagctgcagaaagaagtcccctgttcctaggccgggcgcggtggttcatgcctgtaatctcagcactttgggaggccgaggcaggcggatcatgaggtcaaacgatcgagaccagcctggccaacatggtgaaaccctgtctctactaaaaatacaaaaattagctgggcgcggtagcgcgagcctgtagtcccagctactcgggaggctgaggcaggagaatcgtttgaatccaggaggggaggttgcagtgagccgagattgcgccactgcactccagcctggcgacagagcgagactccgtctcaaaaaaaaaaaaaagaaaaaaagaaaaaaagaaaaaagtgttccctgtcctgggcagagaagctgtgggtagtgttctctcatttgagcagaagggggctgtgggaagggatccctcactgagcacagtctactgtgggaaaagttctcccacacactgtgggaaaagttctctcattctgagcagaaaaccgtgaggaggtatccttcctaccccgggatgaggggcttcatggcggggatcgcctacagtgggcatacagggctgtcggaaggatgtgcccacagttggcatagtgggtgtgaaggaaaagggtccagtgattcccgtgcttccagatttgtggaaagcttttccctactcctgccctggaaaagtgtgtgcaataagactgtctaatcccgggacagagggcatgggaaggactatttctccttgggggtggacgctttagcaaggagggctcaggtctattcggctccgctcccccaggagcgcaggccctggagtgctacagctgcgtgcagagccaccatggcggatcctccccgaacaagatcaagacagttaagtgcgcgccgggcgtggacgtctgcaccgaggccgtgggggcggtggagaccagtgagtggggacccccgggccacgcacaccactgcaggctcctccggcccagccccgcctccttatagtcctttctggatctggccacgcccctttaacgcggccccacccctccctttcagcctggccgggaccctacgacacagccttgcaggccaggccttctctattaacaattacaagcgcgaggagcagagagttgagaactgggtggggcggacggggagagctcgggtttggaacgaggtcacgtcctatgcactttttctgctccaccaatgagacctccccatccctgcctgtcaaccaggctgtctcctccgactcagctctttgaggttccgcccccgttccgtgaccccgctcctccctgcttcggtcgttctttccgctctgagcggctctttgggggcgtccatccgccaattcctgcctgtcggtcctgtggtgttggcctatttgaggccccgcccctcccagaccctacccaactctgggccacgccccatctgagaccacgcccacagctaatcccgcccttgtccctctccctagtccacggacaattctcgctggcagtgcggggttgcggttcgggactccccggcaagaatgaccgcggcctggatcttcacgggcttctggcgttcatccagctgcagcattgcgctcaggatcgctgcaacgccaagctcaacctcacctcgcgggcgctcgacccggcag

SEQ ID NO: 53 - DNA, 779 bp, Synthetic construct
  source 1..779: mol_type = other DNA; note = LYPD3 construct #2 (minimal v1) Also referred to as V1A
gaccccgccaggaaagcaggtgcccaggccttgatctggactgcaggctggctgctgctgctgctgcttcgcggaggtgggtctgctgcccggagaggtggagggggtagagtgggggctcttagggaggaggctcctctggccccgtacagagaaggctgtgggaaggatccccatgtctaatcccgggacagagggcatgggaaggactatttctccttgggggtggacgctttagcaaggagggctcaggtctattcggctccgctcccccaggagcgcaggccctggagtgctacagctgcgtgcagagccaccatggcggatcctccccgaacaagatcaagacagttaagtgcgcgccgggcgtggacgtctgcaccgaggccgtgggggcggtggagaccagtgagtggggacccccgggccacgcacaccactgcaggctcctccggcccagccccgcctccttatagtcctttctggatctggccacgcccctttaacgctatttgaggccccgcccctcccagaccctacccaactctgggccacgccccatctgagaccacgcccacagctaatcccgcccttgtccctctccctagtccacggacaattctcgctggcagtgcggggttgcggttcgggactccccggcaagaatgaccgcggcctggatcttcacgggcttctggcgttcatccagctgcagcattgcgctcaggatcgctgcaacgccaagctcaacctcacctcgcgggcgctcgacccggcag

SEQ ID NO: 54 - DNA, 415 bp, Synthetic construct
  source 1..415: mol_type = other DNA; note = LYPD3 construct #3 (minimal v2) Also referred to as V2A
gctgctgctgctgctgcttcgcggaggtgggtctgctgcccggagaggtggagggggtagagtgggggctcttaggtggacgctttagcaaggagggctcaggtctattcggctccgctcccccaggagcgcaggccctggagtgctacagctgcgtgcagagccaccatggcggatcctccccgaacaagatcaagacagttaagtgcgcgccgggcgtggacgtctgcaccgaggccgtgggggcggtggagaccagtgagtggggacccccgggccacgcacaccactgcaggctcctccggcccccatctgagaccacgcccacagctaatcccgcccttgtccctctccctagtccacggacaattctcgctggcagtgcggggttgcggttcgggactccccggcaaga

SEQ ID NO: 55 - DNA, 3334 bp, Synthetic construct
  source 1..3334: mol_type = other DNA; note = IK construct #1 (full)
gtgagccgttctccaaccctttggcccccgttggccacgatctggaagatcctcactccttccaccagtgagtattttgtgctaggaccatggaaatgagttgggcaatgaatagaaaagtggtggtgatggtgaaagaattctgaatgtcttgtcatcaaggatggtaaaaaattatacccgctggccctttatctctgtgagccttaactggatcaggccaagtgcataagggtattagaagggaaattcctgcaggaaaggtgttaagtttttttttttttttatacgtgttgtgtaattacacattatataggaattacttcttccaatagcaaattaaaacataggaaataagggtaaaatcatctttttattttttttgagactgagtttcgctcttgttgctcaggctggaatgcagtggtgcgatctcggctcacgccaccatgggcctcccgggttcaagcaattctcctgcctcagccttccgagtagctgggattacaggcatgttccactacgcccggctaattttgtatttttagtagagatggggtttctccatgttgatcaggctggtcttgaaatcccgacttcaggtgatccacccgcctctgcctcccaaagtggtgggagtacaggcgtgagccaccgtgcctggcccccgataaaatcatctttgataacatccccaattccagtccatttctttttttttttcttttcttttcttttctttctttttttttttttttgagacggagtctcactgttacccaggctggagtgcagtgatgcaatcttggctcactgcaacctctgcctcctgagttcaagcggttctcctgcctcagcctcccaagtagctgggactacaggcatgtgccaccatgcacgactgatttttttttttgtatttttagtagagacagggtttcactgtgttcgtcaggctggtcttgaactcctggcctgaagtgatccacccgcctcagcctcctaaagtgctggcattacaggcgtgagccaccgtgcccagcctcagtccttttcttagaagtaaccacagttaccttttttggcatatcttttcctcccgttgcccaggctggagttcaatggcattatttctgctcacagcaacctttgcctcctgggctcaagcaattcttctgcctcagtttccccagtagctgggattacaggcatgccccaccatgcttggctaatttttgtattagtagagacagggtttcgctatgttggccaggctggtcttgaactcctgacctcaaatgatctgcccgtctcggcggcccaaagtgctgggattacaggcgtgagccactgcgcctggccctcctactcttttttaaatgcatttatacacatacatgcatatctgaggaaaacacaacattgctttgtgatttaaaatatttgagttaaatgatattatgctgtatatatcatttttgcaacttgcttctttttaaaccctgctcagtgtttctgagattatctttcagagcagtgacaagcattccctccttggctaaactttagtcaggttccccaaagagtcccatttttcagcaggactcatccgtggcctgctgagcacagttttaacaaagaaatcaaccaccgctccacccccatacctaaccaagttgcttttagtaattttctataaattctcacttgtccctgttgtattcggaattaagttcagtctctctactctattacaatggtcttgactcttgttacagtagtcttcaataaagtcttccttggggagaagaggaacttgattagatatcaagttggattggatatcaagagtgggaaaactttctgtgaactgacttaggattcctttttaaaactgaggtcagcaggccaaggaagagccctagtcagaagagagctttaaaggagtctgactttaatttgatcaagagaggagtctttgttagtgtaccaattaaaataatctcttgtaccattagtgctgtctttccctctttgggaaacaccatcatagtttagtcagtctccagctaataaacgcttagtctgtttcagactgattcttttcttttttttatttttttgagacagagtcttgctctttttgcccaggctggagtgtaatacctagatctcggctcactgcaaactccgcctcccaggttcaagcgattttcctgcctcagcctcctgagtagctaggattacaggcgcccaccacaacgcccagctaatttttttgtatttttagtagagatgggatatcaccatgttggccatgctggtcttgaactcctgacctcgggtgatccgcctgcgtcagcctcccaaagtgcggcgattataggtgtgagccactgcacccggccagactgattattttcaaggctataactgaattaaatttacaggccttcttatatacatatgtcagtaatggaccaggctaaatataaataaatggagttgctggctcataggatatatatacttaaattttaacaaatactgcaaaattgcccttcaaattggttatactggtttacattctcatcagcattatatgagagagtccccttcccaaattctcactcacacttgatttatcaaactttctggtagttaacactctgatgcattaaaagtggcatggcatatctggtcgggcgtggtggcttacacctataatcccagcactttgggaggctgaggtgggcagatcacctgaggtcagaagtttgggaccagcctggccaacacctctaataaaaatacaaaaattagctgggtgtgttggcgggcacctgtattcccagctacttgtgaggctgaggcaggagaattacttgaacccaggaggcagaggttgcagtgggctgagatcatgccactgcactccagccggggcaacagagggagactctgtcttaaaaaaaaaaaaaaaaagctgggtgcggtggctcatccctataatcccagcactttgggaggccgaggtgggtggatcacaaggtcaggagttcaagaccagcctggccaatatgggtgacagtgagactccgtctcaaaaaaaaaaaaacagtgtcatatctttgttttaatttgtatttccctgatttctgttaatttaatcatgatttttacttgttcctttttcatttcttattgagtcctaatatttaaaatctttgccttttcttctagatcaaaactcaccattgaagacttcaggaaacttctcagacccccagggctgcacctacctctgcaccaccttcaagtcacgtcaccatta

SEQ ID NO: 56 - DNA, 660 bp, Synthetic construct
  source 1..660: mol_type = other DNA; note = IK construct #2 (minimal v1)
gtgagccgttctccaaccctttggcccccgttggccacgatctggaagatcctcactccttccaccagtgagtattttgtgctaggaccatggaaatgagttgggcaatgaatagaaaagtggtggtgatggtgaaagaattctgaatgtcttgtcatcaaggatggataggaattacttcttccaatagcaaattaaaacataggaaataagggtaaaatcatctttttattttttttgagactgagtttcgctcttgttgctcaggctggaatgcagtggtgcgatctcggctcacgccaccatgggcctcccgggttcaagcaattctcctgcctcagccttccgagtagctgggattacaggcatgttccactacgcccggctaattttgtatttttagtagagatggggtttctccatgttgatcaggctggtcttgaaatcccgacttcaggtgatccacccggtatttccctgatttctgttaatttaatcatgatttttacttgttcctttttcatttcttattgagtcctaatatttaaaatctttgccttttcttctagatcaaaactcaccattgaagacttcaggaaacttctcagacccccagggctgcacctacctctgcaccaccttcaagtcacgtcaccatta

SEQ ID NO: 57 - DNA, 1581 bp, Synthetic construct
  source 1..1581: mol_type = other DNA; note = TEX14 construct #1 (full)
tggaggtgaaagaaatggcaaagaaagcagctactggacagctcacagtacctccttggcatcctcagagtagtctgactttagagagcgaggctgaaaatgagcccgacgccctgctgcagccccccattaggagcccagaaaacacggattggcagcgagttattgagtatcatagggaaaatgatgagcccagaggaaatggcaagtttgacaagacgggcaacaatgactgtgacagtgaccagcatggcagacagcccaggcttggaagcttcaccagtaagttcacttgcgggaatcaggataggtgaactcaatcaagtatcagtcgggcgaggtggctcagcctgtaattccagcactttgggaggtgaggcgagcggatcacctgagcccaggaatttgggaccagcctggtcaacatgatgaaacctggtctctacaaaaaaaaaaaaaaaaaaaaaaaaaacattagctgggcgtggtggcacacgcctctagttccaggtacccaggaggctgaggtgggaggatcacttggacctgagaggtggaggttgcagtgagctgtgatcatgaaactgcactccagcttgggtgacagtgagaccctgtctcaaaataaaaataaaagtaaaaaaaagttatcagctctcaaaaaagtaaactttccagcatattttttttggtttggtcctctctcctttcctacttttctggaaaccttgaattccaggggaagacagtatagccctatatctcattgcatttcccacaggcaaacagatccagacactgatcagtttttatattactgaaacaggtccctttgtccaaaggaagtaaaataatcaaatactgaccctggttttggcgaggaatcgatccttgccaccatggggatagaggtaggccatcactgcctactgcccatgtctctgcattgtattctaagctgaaattaaattctcccagaatttatttagaatgtattctaagctggcattaaatttttccagagagtctatgctggaggaacctagagacacatgacatttattgtcatcggtttatagatttacaggttaaaaaaaatgaaggctgtatattttcagttgataatttgcttataatcagggcatggatattgtttcacagttgtttataagttaaagaaagatacatctaaacttgtcatttgtcttaggcagaaaatctattggcttgggtattatttgtatttagaatctgtttcttgtcaagcatcactctcgaatgggtgggcagatgatccagggctgcattttggggtcaggctaaactaacagtctctactctctggactgaacctaacgtatgggacatggccactcttggcagaatatgtatacatctcttctttcatgccacctttatcctcttccatgttgtctgaatatcactgaatgactttgaccctgactgtgttacaatcattaggtatcaggcacccatctcccagacaaaaggagcaaccagagcagtgaagccttccaagcaagttctgacacattggtggctgtagagaaatcttacagt

SEQ ID NO: 58 - DNA, 866 bp, Synthetic construct
  source 1..866: mol_type = other DNA; note = TEX14 construct #2 (minimal v1)
tggaggtgaaagaaatggcaaagaaagcagctactggacagctcacagtacctccttggcatcctcagagtagtctgactttagagagcgaggctgaaaatgagcccgacgccctgctgcagccccccattaggagcccagaaaacacggattggcagcgagttattgagtatcatagggaaaatgatgagcccagaggaaatggcaagtttgacaagacgggcaacaatgactgtgacagtgaccagcatggcagacagcccaggcttggaagcttcaccagtaagttcacttgcgggaatcaggataggtgaactcaatcaagtatcagtcgggcgaggtggctcagcctgtaattccagcactttgggaggtgaggcgaccttgaattccaggggaagacagtatagccctatatctcattgcatttcccacaggcaaacagatccagacactgatcagtttttatattactgaaacaggtccctttgtccaaaggaagtaaaataatcaaatactgaccctggttttggcgaggaatcgatccttgccaccatggggatagaggtaggccatcactgcctactgcccatgtctctgcattgtattctaagctgaaattaaattctcccagaatttatttagaatgtattctaagctggcattagaatatgtatacatctcttctttcatgccacctttatcctcttccatgttgtctgaatatcactgaatgactttgaccctgactgtgttacaatcattaggtatcaggcacccatctcccagacaaaaggagcaaccagagcagtgaagccttccaagcaagttctgacacattggtggctgtagagaaatcttacagt

SEQ ID NO: 59 - DNA, 7163 bp, Synthetic construct
  source 1..7163: mol_type = other DNA; note = Rep Construct #1
caattgtcgcgaaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcgctagcctcgagcctgcaggcgacatgaagaatctgcttagggttaggcgttttgcgctgcttcgcgttgtacgggccagatatacgcgtatctgaggggactagggtgtgtttaggcgaaaagcggggcttcggttgtacgcggttaggagtcccctcaggatatagtagtttcgcttttgcatagggagggggattgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccgaccccgccaggaaagcaggtgcccaggccttgatctggactgcaggctggctgctgctgctgctgcttcgcggaggtgggtctgctgcccggagaggtggagggggtagagtgggggctcttagggaggaggctcctctggccccgtacagagaaggctgtgggaaggatccccatgtctaatcccgggacagagggcatgggaaggactatttctccttgggggtggacgctttagcaaggagggctcaggtctattcggctccgctcccccaggagcgcaggccctggagtgctacagctgcgtgcagagccaccatggcggatcctccccgaacaagatcaagacagttaagtgcgcgccgggcgtggacgtctgcaccgaggccgtgggggcggtggagaccagtgagtggggacccccgggccacgcacaccactgcaggctcctccggcccagccccgcctccttatagtcctttctggatctggccacgcccctttaacgctatttgaggccccgcccctcccagaccctacccaactctgggccacgccccatctgagaccacgcccacagctaatcccgcccttgtccctctccctagtccacggacaattctcgctggcagtgcggggttgcggttcgggactccccggcaagattgaccgcggcctggatcttcacgggcttctggcgttcatccagctgcagcattgcgctcaggatcgctgcaacgccaagctcaacctcacctcgcgggcgctcgacccggcaggtgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatctatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggattcaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccggaaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgagttctacgtcaaaaagggtggagccaagaaaagacccgcccccagtgacgcagatataagtgagcccaaacgggtgcgcgagtcagttgcgcagccatcgacgtcagacgcggaggcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaaggtaaacttgttaatcaataaaccgtttaattcgtttcagttgaacttttctctgctaactttctttctttctagtttggacttccctccaggtaaccggtcggggcaacatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggacttccctgcagtgtgggatttacgctgattcaggaatgggttgttccctgggatatggttctaacgcgggaggagcttgtaatcctgaggaagtgtatgcacgtgtgcctgtgttgtgccaacattgacatcatgacgagcatgatgatccacggttacgagtcctgggctctccactgtcattgttccagtcccggttccctgcagtgtatagccggcgggcaggttttggccagctggtttaggatggtggtggatggcgccatgtttaatcagaggtttatatggtatcgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagactaccgccatgttgtattcctgcaggacggagcggcggcggcagcagtttattcgcgcgctgctgcagcaccaccgccctatcctgatgcacgattatgactctacccccatgtaggttgtttgacccctcccccgtgccttccttgaccctggaaggtgccactcccactgtcctttcctaataaaatgaggaaattgcatcgcattgtctgagtaggtgtcattctattctggggggtggggtggggcaggacagcaagggggatccctgcaggcgcaggcggtcagcatcgccatggagaccaacacccttcccaccgccactcccccttcctctcagggtccctgtcccctccagtgaatcccagaaggactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctacctgaggagctgctgcgacagtccactacctttttcgaggtgactcccgttgtcccaaggcttcccagagcgaacctgtgcggctgcaggcaccggcgcgtcgagtttccggcgtccggaaggaccgagctcttctcgctgatccgtgttccgtttccgcccccaatctctcgttgccgacgcagggaaccggcggcaaccatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaggactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaaccctccggtgctaatcaagcatggcaaaggaggtaagcgcacagtgcggcggctgaatgaagatgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgggcgctgatggacaagtaccacgtggataacgatctaaaggcgaacttcaaactactgcctgaccaagtggaagctctggcagccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaatttgggcagcgcgcgctgaaggagcagtctagcaaggccaagatcgtgaagaatcggtggggccgaaatgtggtgcagatttccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgggcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagtaaataatcacccgagagtgtacaaataaaagatcttgccacaaaatagttacaggaccaagcgagcgtgagagtccaaggcgtgcgcagtcgttgacgctctagaccgtgcaaaaggagagcctgtaagcgggcactcttccgtggtctggtggataaattcgcaagggtatcatggcggacgacccgggttcgaaccccggatcgggccgtccgccgtgatccatgcggttaccgcccgcgtgtcgaacccaggtgtgcgacgtcagacaacgggggagcgctcctttttggcttccttccaggcgcggcggctgctgcgctcttttttcggccgcggccgc

SEQ ID NO: 60 - DNA, 6400 bp, Synthetic construct
  source 1..6400: mol_type = other DNA; note = Rep Construct #2
caattgagggtggaggtcctgtattagaggtcacgtgagtgttttgcgacattttgcgacaccatgtggtcacgctgggtatttaagcccgagtgagcacgcagggctctccattttgaagcgggaggtttgaacgcgcagccgccgaccccgccaggaaagcaggtgcccaggccttgatctggactgcaggctggctgctgctgctgctgcttcgcggaggtgggtctgctgcccggagaggtggagggggtagagtgggggctcttagggaggaggctcctctggccccgtacagagaaggctgtgggaaggatccccatgtctaatcccgggacagagggcatgggaaggactatttctccttgggggtggacgctttagcaaggagggctcaggtctattcggctccgctcccccaggagcgcaggccctggagtgctacagctgcgtgcagagccaccatggcggatcctccccgaacaagatcaagacagttaagtgcgcgccgggcgtggacgtctgcaccgaggccgtgggggcggtggagaccagtgagtggggacccccgggccacgcacaccactgcaggctcctccggcccagccccgcctccttatagtcctttctggatctggccacgcccctttaacgctatttgaggccccgcccctcccagaccctacccaactctgggccacgccccatctgagaccacgcccacagctaatcccgcccttgtccctctccctagtccacggacaattctcgctggcagtgcggggttgcggttcgggactccccggcaagattgaccgcggcctggatcttcacgggcttctggcgttcatccagctgcagcattgcgctcaggatcgctgcaacgccaagctcaacctcacctcgcgggcgctcgacccggcaggtgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatctatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggattcaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccggaaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgagttctacgtcaaaaagggtggagccaagaaaagacccgcccccagtgacgcagatataagtgagcccaaacgggtgcgcgagtcagttgcgcagccatcgacgtcagacgcggaggcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaaggtaaacttgttaatcaataaaccgtttaattcgtttcagttgaacttttctctgctaactttctttctttctagtttggacttccctccaggtaaccggtcggggcaacatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggacttccctgcagtgtgggatttacgctgattcaggaatgggttgttccctgggatatggttctaacgcgggaggagcttgtaatcctgaggaagtgtatgcacgtgtgcctgtgttgtgccaacattgacatcatgacgagcatgatgatccacggttacgagtcctgggctctccactgtcattgttccagtcccggttccctgcagtgtatagccggcgggcaggttttggccagctggtttaggatggtggtggatggcgccatgtttaatcagaggtttatatggtatcgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagactaccgccatgttgtattcctgcaggacggagcggcggcggcagcagtttattcgcgcgctgctgcagcaccaccgccctatcctgatgcacgattatgactctacccccatgtaggttgtttgacccctcccccgtgccttccttgaccctggaaggtgccactcccactgtcctttcctaataaaatgaggaaattgcatcgcattgtctgagtaggtgtcattctattctggggggtggggtggggcaggacagcaagggggatccctgcaggcgcaggcggtcagcatcgccatggagaccaacacccttcccaccgccactcccccttcctctcagggtccctgtcccctccagtgaatcccagaaggactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctacctgaggagctgctgcgacagtccactacctttttcgaggtgactcccgttgtcccaaggcttcccagagcgaacctgtgcggctgcaggcaccggcgcgtcgagtttccggcgtccggaaggaccgagctcttctcgctgatccgtgttccgtttccgcccccaatctctcgttgccgacgcagggaaccggcggcaaccatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaggactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaaccctccggtgctaatcaagcatggcaaaggaggtaagcgcacagtgcggcggctgaatgaagatgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgggcgctgatggacaagtaccacgtggataacgatctaaaggcgaacttcaaactactgcctgaccaagtggaagctctggcagccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaatttgggcagcgcgcgctgaaggagcagtctagcaaggccaagatcgtgaagaatcggtggggccgaaatgtggtgcagatttccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgggcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagtaaataatcacccgagagtgtacaaataaaagatcttgccacaaaatagttacaggaccaagcgagcgtgagagtccaaggcgtgcgcagtcgttgacgctctagaccgtgcaaaaggagagcctgtaagcgggcactcttccgtggtctggtggataaattcgcaagggtatcatggcggacgacccgggttcgaaccccggatcgggccgtccgccgtgatccatgcggttaccgcccgcgtgtcgaacccaggtgtgcgacgtcagacaacgggggagcgctcctttttggcttccttccaggcgcggcggctgctgcgctcttttttcggccggcggccgc

SEQ ID NO: 61 - DNA, 7290 bp, Synthetic construct
  source 1..7290: mol_type = other DNA; note = Rep Construct #3
caattgtcgcgaaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcgctagcctcgagcctgcaggcgacatgaagaatctgcttagggttaggcgttttgcgctgcttcgcgttgtacgggccagatatacgcgtatctgaggggactagggtgtgtttaggcgaaaagcggggcttcggttgtacgcggttaggagtcccctcaggatatagtagtttcgcttttgcatagggagggggattgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccttttatccaagctgtgcttgtgatactgtctgtcttcttgtacttgtttgcctacaaagagtacctcgcctgcctcgtgctggccttggccctgggctgggcgaacaagctctactatacgcggggtttccagtccatcggcttgtacagcgtcatcatccagaaggtgcgttgagagctgcgccggcgggaggggcctcggaggatggccatttttcagatggggaaactgagatctggagaggaagaggggtttaccaaggggcagttagggtggggggtgggagagggtgggaagcaggaagatcagggtgccgtgaatatgtgcttgcaccttacacttgaattatgtttatttccttataggtcatttgccaccatgttctggagttcttgtttgtatatatcgtgtttttgcttggatttggagttggtaattgatttataaataatagtaactcccacatttataaacactttatacagatgtcaaaatggcaaagtgcatggcggccttggcaacaattagaccagaaggaggcagcaggcttgtcatgtaggcaaatacacgcaagcttgtagagtaattaccaggcagagagctgaggagagcaccctgttatgttcccttagccttggcctcgcttatcgagaagtgtcccaaagacaacaaggactgcagctcctacggcagcttcagcgacgcagtgctggaactcttcaagctcaccattggcctgggtgacctggacatccagcagaactccaagtatcccattctctttctgttcctgctcatcacctttgtcatcctcacctttgttctcctcctcaacaagctcattgctcttatcggcgagactgtggagaacgtctccaaggagagcgaacgcatctggcgcctgcgtgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatctatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggattcaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccggaaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgagttctacgtcaaaaagggtggagccaagaaaagacccgcccccagtgacgcagatataagtgagcccaaacgggtgcgcgagtcagttgcgcagccatcgacgtcagacgcggaggcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaaggtaaacttgttaatcaataaaccgtttaattcgtttcagttgaacttttctctgctaactttctttctttctagtttggacttccctccaggtaaccggtcggggcaacatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggacttccctgcagtgtgggatttacgctgattcaggaatgggttgttccctgggatatggttctaacgcgggaggagcttgtaatcctgaggaagtgtatgcacgtgtgcctgtgttgtgccaacattgacatcatgacgagcatgatgatccacggttacgagtcctgggctctccactgtcattgttccagtcccggttccctgcagtgtatagccggcgggcaggttttggccagctggtttaggatggtggtggatggcgccatgtttaatcagaggtttatatggtatcgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagactaccgccatgttgtattcctgcaggacggagcggcggcggcagcagtttattcgcgcgctgctgcagcaccaccgccctatcctgatgcacgattatgactctacccccatgtaggttgtttgacccctcccccgtgccttccttgaccctggaaggtgccactcccactgtcctttcctaataaaatgaggaaattgcatcgcattgtctgagtaggtgtcattctattctggggggtggggtggggcaggacagcaagggggatccctgcaggcgcaggcggtcagcatcgccatggagaccaacacccttcccaccgccactcccccttcctctcagggtccctgtcccctccagtgaatcccagaaggactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctacctgaggagctgctgcgacagtccactacctttttcgaggtgactcccgttgtcccaaggcttcccagagcgaacctgtgcggctgcaggcaccggcgcgtcgagtttccggcgtccggaaggaccgagctcttctcgctgatccgtgttccgtttccgcccccaatctctcgttgccgacgcagggaaccggcggcaaccatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaggactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaaccctccggtgctaatcaagcatggcaaaggaggtaagcgcacagtgcggcggctgaatgaagatgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgggcgctgatggacaagtaccacgtggataacgatctaaaggcgaacttcaaactactgcctgaccaagtggaagctctggcagccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaatttgggcagcgcgcgctgaaggagcagtctagcaaggccaagatcgtgaagaatcggtggggccgaaatgtggtgcagatttccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgggcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagtaaataatcacccgagagtgtacaaataaaagatcttgccacaaaatagttacaggaccaagcgagcgtgagagtccaaggcgtgcgcagtcgttgacgctctagaccgtgcaaaaggagagcctgtaagcgggcactcttccgtggtctggtggataaattcgcaagggtatcatggcggacgacccgggttcgaaccccggatcgggccgtccgccgtgatccatgcggttaccgcccgcgtgtcgaacccaggtgtgcgacgtcagacaacgggggagcgctcctttttggcttccttccaggcgcggcggctgctgcgctcttttttcggccgcggccgc

SEQ ID NO: 62 - DNA, 6527 bp, Synthetic construct
  source 1..6527: mol_type = other DNA; note = Rep Construct #4
caattgagggtggaggtcctgtattagaggtcacgtgagtgttttgcgacattttgcgacaccatgtggtcacgctgggtatttaagcccgagtgagcacgcagggctctccattttgaagcgggaggtttgaacgcgcagccgccttttatccaagctgtgcttgtgatactgtctgtcttcttgtacttgtttgcctacaaagagtacctcgcctgcctcgtgctggccttggccctgggctgggcgaacaagctctactatacgcggggtttccagtccatcggcttgtacagcgtcatcatccagaaggtgcgttgagagctgcgccggcgggaggggcctcggaggatggccatttttcagatggggaaactgagatctggagaggaagaggggtttaccaaggggcagttagggtggggggtgggagagggtgggaagcaggaagatcagggtgccgtgaatatgtgcttgcaccttacacttgaattatgtttatttccttataggtcatttgccaccatgttctggagttcttgtttgtatatatcgtgtttttgcttggatttggagttggtaattgatttataaataatagtaactcccacatttataaacactttatacagatgtcaaaatggcaaagtgcatggcggccttggcaacaattagaccagaaggaggcagcaggcttgtcatgtaggcaaatacacgcaagcttgtagagtaattaccaggcagagagctgaggagagcaccctgttatgttcccttagccttggcctcgcttatcgagaagtgtcccaaagacaacaaggactgcagctcctacggcagcttcagcgacgcagtgctggaactcttcaagctcaccattggcctgggtgacctggacatccagcagaactccaagtatcccattctctttctgttcctgctcatcacctttgtcatcctcacctttgttctcctcctcaacaagctcattgctcttatcggcgagactgtggagaacgtctccaaggagagcgaacgcatctggcgcctgcgtgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatctatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggattcaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccggaaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgagttctacgtcaaaaagggtggagccaagaaaagacccgcccccagtgacgcagatataagtgagcccaaacgggtgcgcgagtcagttgcgcagccatcgacgtcagacgcggaggcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaaggtaaacttgttaatcaataaaccgtttaattcgtttcagttgaacttttctctgctaactttctttctttctagtttggacttccctccaggtaaccggtcggggcaacatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggacttccctgcagtgtgggatttacgctgattcaggaatgggttgttccctgggatatggttctaacgcgggaggagcttgtaatcctgaggaagtgtatgcacgtgtgcctgtgttgtgccaacattgacatcatgacgagcatgatgatccacggttacgagtcctgggctctccactgtcattgttccagtcccggttccctgcagtgtatagccggcgggcaggttttggccagctggtttaggatggtggtggatggcgccatgtttaatcagaggtttatatggtatcgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagactaccgccatgttgtattcctgcaggacggagcggcggcggcagcagtttattcgcgcgctgctgcagcaccaccgccctatcctgatgcacgattatgactctacccccatgtaggttgtttgacccctcccccgtgccttccttgaccctggaaggtgccactcccactgtcctttcctaataaaatgaggaaattgcatcgcattgtctgagtaggtgtcattctattctggggggtggggtggggcaggacagcaagggggatccctgcaggcgcaggcggtcagcatcgccatggagaccaacacccttcccaccgccactcccccttcctctcagggtccctgtcccctccagtgaatcccagaaggactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctacctgaggagctgctgcgacagtccactacctttttcgaggtgactcccgttgtcccaaggcttcccagagcgaacctgtgcggctgcaggcaccggcgcgtcgagtttccggcgtccggaaggaccgagctcttctcgctgatccgtgttccgtttccgcccccaatctctcgttgccgacgcagggaaccggcggcaaccatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaggactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaaccctccggtgctaatcaagcatggcaaaggaggtaagcgcacagtgcggcggctgaatgaagatgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgggcgctgatggacaagtaccacgtggataacgatctaaaggcgaacttcaaactactgcctgaccaagtggaagctctggcagccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaatttgggcagcgcgcgctgaaggagcagtctagcaaggccaagatcgtgaagaatcggtggggccgaaatgtggtgcagatttccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgggcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagtaaataatcacccgagagtgtacaaataaaagatcttgccacaaaatagttacaggaccaagcgagcgtgagagtccaaggcgtgcgcagtcgttgacgctctagaccgtgcaaaaggagagcctgtaagcgggcactcttccgtggtctggtggataaattcgcaagggtatcatggcggacgacccgggttcgaaccccggatcgggccgtccgccgtgatccatgcggttaccgcccgcgtgtcgaacccaggtgtgcgacgtcagacaacgggggagcgctcctttttggcttccttccaggcgcggcggctgctgcgctcttttttcggccggcggccgc

SEQ ID NO: 63 - DNA, 1812 bp, Synthetic construct
  source 1..1812: mol_type = other DNA; note = iRFP Construct #1
tcgcgaaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcgctagcctcgagcctgcaggcgacaattgcatgaagaatctgcttagggttaggcgttttgcgctgcttcgcgttgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattgcgcattgcagagataattgtatttaagtgcctagctcgatacaataaacgccatttgaccattcaccacattggtgtgcacctccaaggccacagatccgtttaaacaagcttggcaatccggtactgttggtaaagccaccatggcggatcctccccgaacaagatcaagacagttaagtgcgcgccgggcgtggacgtctgcaccgaggccgtgggggcggtggagaccatccacggacaattctcgctggcagtgcggggttgcggttcgggactccccggcaagagcggaaggatccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtcttcgactcggagacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctcgagctcgagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtctcgcccgtccatctggaattcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtgagggccc

SEQ ID NO: 64 - DNA, 6881 bp, Synthetic construct
  source 1..6881: mol_type = other DNA; note = iRFP Construct #2
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgaaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcgctagcctcgagcctgcaggcgacaattgcatgaagaatctgcttagggttaggcgttttgcgctgcttcgcgttgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattgcgcattgcagagataattgtatttaagtgcctagctcgatacaataaacgccatttgaccattcaccacattggtgtgcacctccaaggccacagatccgtttaaacaagcttggcaatccggtactgttggtaaagctgctgctgctgctgcttcgcggaggtgggtctgctgcccggagaggtggagggggtagagtgggggctcttaggtggacgctttagcaaggagggctcaggtctattcggctccgctcccccaggagcgcaggccctggagtgctacagctgcgtgcagagccaccatggcggatcctccccgaacaagatcaagacagttaagtgcgcgccgggcgtggacgtctgcaccgaggccgtgggggcggtggagaccagtgagtggggacccccgggccacgcacaccactgcaggctcctccggcccccatctgagaccacgcccacagctaatcccgcccttgtccctctccctagtccacggacaattctcgctggcagtgcggggttgcggttcgggactccccggcaagagtggcggaaggatccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtcttcgactcggagacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctcgagctcgagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtctcgcccgtccatctggaattcatgcgcaacataggcatgcacggcacgatgtcgatctcgattttgcgcggcgagcgactgtggggattgatcgtttgccatcaccgaacgccgtactacgtcgatctcgatggccgccaagcctgcaagagggtcgccgagaggctggccactcagatcggcgtgatggaagagtgagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 65 - DNA, 1668 bp, Synthetic construct
  source 1..1668: mol_type = other DNA; note = iRFP Construct #3
tcgcgaaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcgctagcctcgagcctgcaggcgacaattgcatgaagaatctgcttagggttaggcgttttgcgctgcttcgcgttgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattgcgcattgcagagataattgtatttaagtgcctagctcgatacaataaacgccatttgaccattcaccacattggtgtgcacctccaaggccacagatccgtttaaacaagcttggcaatccggtactgttggtaaagccaccatggcggaaggatccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtcttcgactcggagacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctcgagctcgagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtctcgcccgtccatctggaattcatgcgcaacataggcatgcacggcacgatgtcgatctcgattttgcgcggcgagcgactgtggggattgatcgtttgccatcaccgaacgccgtactacgtcgatctcgatggccgccaagcctgcaagagggtcgccgagaggctggccactcagatcggcgtgatggaagagtgagggccc

SEQ ID NO: 66 - DNA, 2072 bp, Synthetic construct
  source 1..2072: mol_type = other DNA; note = iRFP Construct #4
tcgcgaaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcgctagcctcgagcctgcaggcgacaattgcatgaagaatctgcttagggttaggcgttttgcgctgcttcgcgttgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattgcgcattgcagagataattgtatttaagtgcctagctcgatacaataaacgccatttgaccattcaccacattggtgtgcacctccaaggccacagatccgtttaaacaagcttggcaatccggtactgttggtaaatctactatacgcggggtttccagtccatcggcttgtacagcgtcatcatccagaaggtgcgttgagagctgcgccggcgggaggggcctcggaggatggccattttgtgaatatgtgcttgcaccttacacttgaattatgtttatttccttataggtcatttgccaccatgttctggagttcttgtttgtatatatcgtgtttttgcttggatttggagttggtaattgatttataaataatagtaactcccacatttataaacactttatagtaattaccaggcagagagctgaggagagcaccctgttatgttcccttagccttggcctcgcttatcgagaagtgtcccaaagacaacaaggactgcagctcctacggcagcttcagcgacgcagtgctggaactctgtggcggaaggatccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtcttcgactcggagacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctcgagctcgagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtctcgcccgtccatctggaattcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtgagggccc